(19) United States
(12) Patent Application Publication
(10) Pub. No.: US 2026/0167615 A1
(43) Pub. Date: Jun. 18, 2026

(54) COMPOUNDS AND COMPOSITIONS AS GPR52 MODULATORS

(71) Applicant: Neurocrine Biosciences, Inc., San Diego, CA (US)

(72) Inventors: Neil J. Ashweek, San Diego, CA (US); Manwika Charaschanya, San Diego, CA (US); Anushka Chathuranga Galasiti Kankanamalage, San Diego, CA (US); Disha M. Gandhi, San Diego, CA (US); Juan Pablo Cueva-Garcia, San Diego, CA (US); Collin Regan, San Diego, CA (US); Shawn Branum, San Diego, CA (US); Wei-Li Lee, San Diego, CA (US)

(21) Appl. No.: 19/124,526

(22) PCT Filed: Oct. 25, 2023
(86) PCT No.: PCT/US2023/035866

§ 371 Date: Apr. 25, 2025

Related U.S. Application Data
(60) Provisional application No. 63/419,384, filed on Oct. 26, 2022.

(51) Int. Cl. C07D 401/04; C07D 401/12; C07D 401/14

(52) U.S. Cl. CPC C07D 401/04; C07D 401/12; C07D 401/14

(57) ABSTRACT

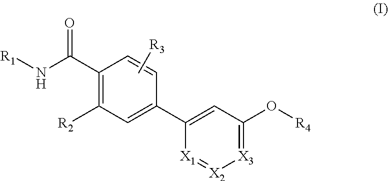

The present disclosure relates to compounds of Formula (I) capable of modulating the activity of GPR52. The present disclosure further provides a process for the preparation of compounds of Formula (1) and methods of using compounds of Formula (I) in the management of diseases or disorders associated with the activity of GPR52 including, but not limited to, the treatment of various neurological conditions.

### CROSS-REFERENCE TO RELATED APPLICATIONS

This application claims the benefit of priority to U.S. Provisional Patent Application No. 63/419,384 filed on Oct. 26, 2022. The full disclosure of this application is incorporated herein by reference in its entirety and for all purposes.

### BACKGROUND

### Technical Field

The present disclosure relates to compounds of Formula (I) capable of modulating the activity of GPR52. The present disclosure further provides a process for the preparation of compounds of Formula (I) and pharmaceutical preparations comprising such compounds. The present disclosure further provides methods of using compounds and compositions of Formula (I) in the management of diseases or disorders associated with the activity of GPR52 including, but not limited to, the treatment of various neurological conditions.

### Description of the Related Technology

GPR52 is an orphan GPCR that is highly conserved in vertebrates. The highest expression levels within the central nervous system (CNS) are found in the striatum. Lower, significant expression levels are found in other structures in the CNS, including in the cortex. Although GPR52 has been characterized, it remains an orphan receptor with no known endogenous ligand. Several surrogate ligands have been reported including GPR52's own extracellular loop 2 (ECL2).

GPR52 is often co-localized with dopamine receptors D1 and D2. GPR52 co-localizes almost exclusively with the D2 receptor in the human striatum, and with the D1 receptor in the cortex. The efficacy of existing antipsychotic drugs is mediated by D2 antagonist activity, but this activity comes with side effects such as motor symptoms and hyperprolactinemia. GPR52 modulators, by contrast, can function essentially as a D2 antagonist and therefore exhibit antipsychotic efficacy while avoiding D2 antagonist related side effects. As such, GPR52 modulators can improve the symptoms of various neurological conditions, diseases, and disorders. GPR52, therefore, represents an attractive target for the development of novel therapies for the treatment of various neurological and neuropsychiatric diseases and disorders. GPR52 agonists are particularly relevant to the treatment of schizophrenia, where they have the potential to improve cognition and negative symptoms indirectly, by potentiating D1 signaling, but alleviate positive symptoms, through inhibition of D2-mediated signaling in the striatum.

Despite the advances that have been made in this field, there is an unmet medical need for improved GPR52 agonists. The compounds, compositions, and methods related thereto, as evident by the following disclosure, fulfill these and other needs.

### SUMMARY

1. Some aspects provide a compound of Formula I

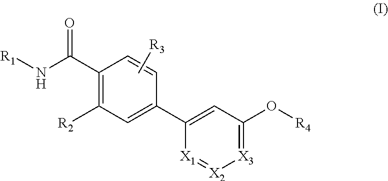

in which:
R₁ is selected from hydrogen and C₁₋₂alkyl;
R₂ is selected from C₁₋₂alkyl, halo, methyl-amino and halo-substituted-C₁₋₂alkyl;
R₃ is selected from hydrogen and halo;
R₄ is selected from:

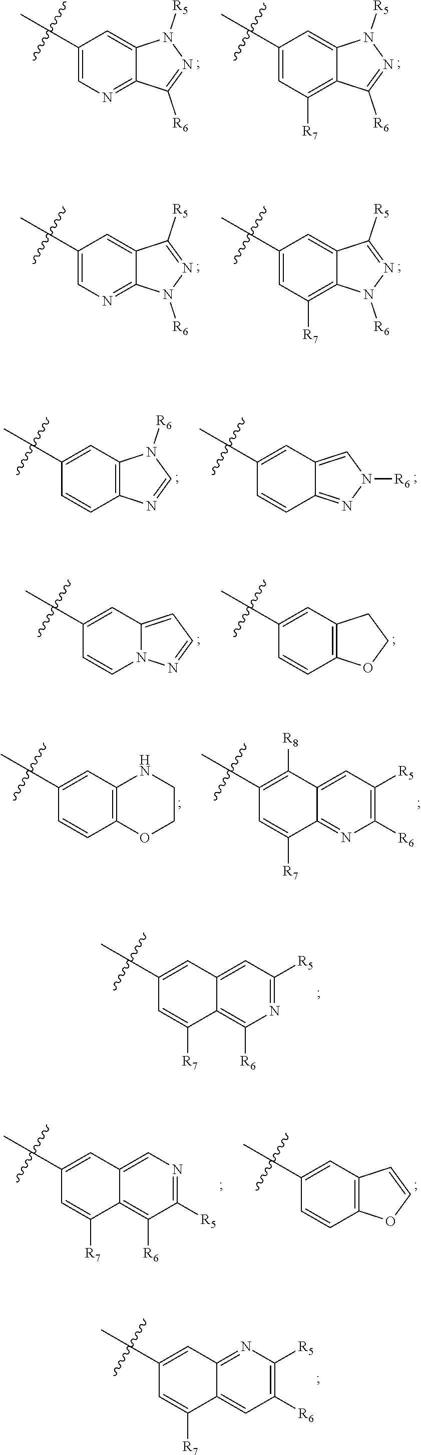

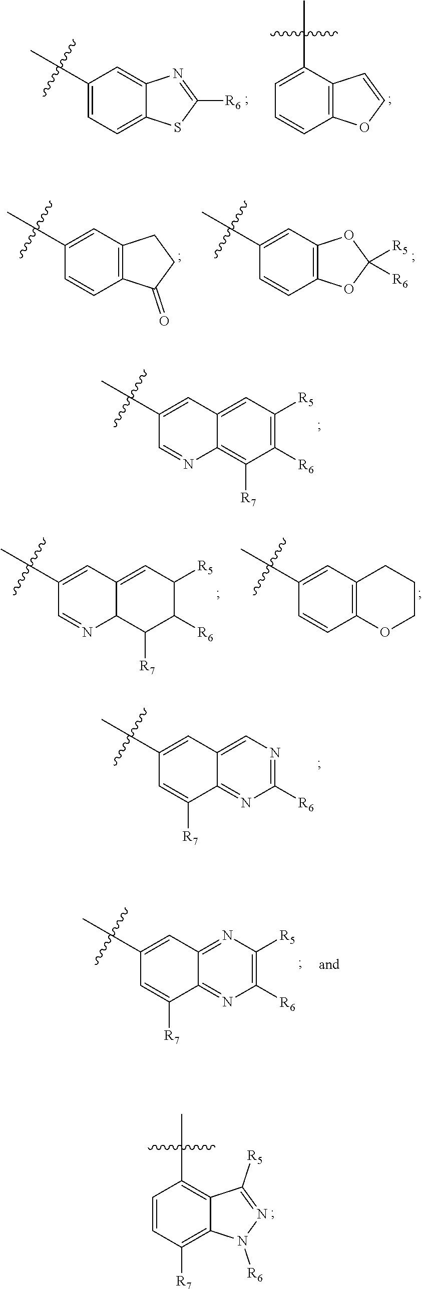

wherein:
R₅ when attached to a carbon atom is selected from hydrogen, C₁₋₂alkyl, halo and halo-substituted-C₁₋₂alkyl; and R₅ when attached to a nitrogen atom is selected from hydrogen, C₁₋₂alkyl, and halo-substituted-C₁₋₂alkyl;
R₆ when attached to a carbon atom is selected from hydrogen, amino, cyano, C₁₋₂alkyl, halo and halo-substituted-C₁₋₂alkyl; and R₆ when attached to a nitrogen atom is selected from hydrogen, C₁₋₂alkyl, and halo-substituted-C₁₋₂alkyl;
R₇ is selected from hydrogen, C₁₋₂alkyl, halo and halo-substituted-C₁₋₂alkyl;
R₈ is selected from hydrogen and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; or R₉ and the nitrogen of X₁ form a 5-member unsaturated ring containing up to two nitrogen atoms;
X₃ is selected from CR₉ₐ; wherein R₉ₐ is selected from hydrogen and methyl; and the pharmaceutically acceptable salts or hydrates thereof.

In some aspects, the compound of Formula (I), or a pharmaceutically acceptable salt thereof, is a compound described in the Examples, below, or a pharmaceutically acceptable salt thereof. In some aspects, the compound of Formula (I), or a pharmaceutically acceptable salt thereof, is a compound of Formula (Ia), Formula (Ib), Formula (Ic), Formula (Id), or Formula (Ie), or a pharmaceutically acceptable salt of any of the foregoing

Some aspects provide a pharmaceutical product selected from: a pharmaceutical composition, a formulation, a unit dosage form, and a kit; each comprising a compound of Formula (I), or a pharmaceutically acceptable salt thereof.

Some aspects provide a pharmaceutical composition comprising a compound of Formula (I), or a pharmaceutically acceptable salt thereof, and at least one pharmaceutically acceptable excipient.

Some aspects provide a method of modulating the activity of GPR52 comprising contacting the receptor with a compound of Formula (I), or a pharmaceutically acceptable salt thereof.

Some aspects provide a method of treating a disease or disorder associated with abnormal expression and/or activity of GPR52 in a patient, comprising administering to the patient a therapeutically effective amount of a compound of Formula (I), or a pharmaceutically acceptable salt thereof.

Some aspects provide a method of treating a neurological disorder, comprising administering to a subject in need thereof an effective amount of a compound of Formula (I), or a pharmaceutically acceptable salt thereof; wherein the neurological disorder is selected from the group consisting of: schizophrenia; cognitive impairment; a panic disorder; a phobic disorder; a drug-induced psychotic disorder; delusional psychosis; neuroleptic-induced dyskinesia; Parkinson's disease; drug-induced Parkinson's syndrome; extrapyramidal syndrome; Alzheimer's Disease; Lewy Body Dementia; bipolar disorder; attention-deficit/hyperactivity disorder (ADHD); Tourette's syndrome; an extrapyramidal or movement disorder; a motor disorder; a hyperkinetic movement disorder; a psychotic disorder; catatonia; a mood disorder; a depressive disorder; an anxiety disorder; obsessive-compulsive disorder (OCD); an autism spectrum disorder; a prolactin-related disorder (e.g., hyperprolactinemia); a neurocognitive disorder; a trauma- or stressor-related disorder (e.g., PTSD); a disruptive, impulse-control, or conduct disorder; a sleep-wake disorder; a substance-related disorder; an addictive disorder; a behavioral disorder; hypofrontality; an abnormality in the tuberoinfundibular, mesolimbic, mesocortical, or nigrostriatal pathway; decreased activity in the striatum; cortical dysfunction; neurocognitive dysfunction; cognitive deficits associated with schizophrenia; drug induced Parkinsonism (DIP); dyskinesias; dystonia; chorea; levodopa induced dyskinesia; cerebral palsy and progressive supranuclear palsy; and Huntington's disease, including chorea associated with Huntington's disease.

Some aspects provide a method of ameliorating one or more symptoms of a neurological disorder, comprising administering to a subject in need thereof an effective amount of a compound of Formula (I), or a pharmaceutically acceptable salt thereof; wherein the neurological disorder is selected from the group consisting of: schizophrenia; cognitive impairment; a panic disorder; a phobic disorder; a drug-induced psychotic disorder; delusional psychosis; neuroleptic-induced dyskinesia; Parkinson's disease; drug-induced Parkinson's syndrome; extrapyramidal syndrome; Alzheimer's Disease; Lewy Body Dementia; bipolar disorder; attention-deficit/hyperactivity disorder (ADHD); Tourette's syndrome; an extrapyramidal or movement disorder; a motor disorder; a hyperkinetic movement disorder; a psychotic disorder; catatonia; a mood disorder; a depressive disorder; an anxiety disorder; obsessive-compulsive disorder (OCD); an autism spectrum disorder; a prolactin-related disorder (e.g., hyperprolactinemia); a neurocognitive disorder; a trauma- or stressor-related disorder (e.g., PTSD); a disruptive, impulse-control, or conduct disorder; a sleep-wake disorder; a substance-related disorder; an addictive disorder; a behavioral disorder; hypofrontality; an abnormality in the tuberoinfundibular, mesolimbic, mesocortical, or nigrostriatal pathway; decreased activity in the striatum; cortical dysfunction; neurocognitive dysfunction; cognitive deficits associated with schizophrenia; drug induced Parkinsonism (DIP); dyskinesias; dystonia; chorea; levodopa induced dyskinesia; cerebral palsy and progressive supranuclear palsy; and Huntington's disease, including chorea associated with Huntington's disease.

Some aspects provide a method of manufacturing a medicament for ameliorating one or more symptoms of a neurological disorder, comprising administering to a subject in need thereof an effective amount of a compound of Formula (I), or a pharmaceutically acceptable salt thereof; wherein the neurological disorder is selected from the group consisting of: schizophrenia; cognitive impairment; a panic disorder; a phobic disorder; a drug-induced psychotic disorder; delusional psychosis; neuroleptic-induced dyskinesia; Parkinson's disease; drug-induced Parkinson's syndrome; extrapyramidal syndrome; Alzheimer's Disease; Lewy Body Dementia; bipolar disorder; attention-deficit/hyperactivity disorder (ADHD); Tourette's syndrome; an extrapyramidal or movement disorder; a motor disorder; a hyperkinetic movement disorder; a psychotic disorder; catatonia; a mood disorder; a depressive disorder; an anxiety disorder; obsessive-compulsive disorder (OCD); an autism spectrum disorder; a prolactin-related disorder (e.g., hyperprolactinemia); a neurocognitive disorder; a trauma- or stressor-related disorder (e.g., PTSD); a disruptive, impulse-control, or conduct disorder; a sleep-wake disorder; a substance-related disorder; an addictive disorder; a behavioral disorder; hypofrontality; an abnormality in the tuberoinfundibular, mesolimbic, mesocortical, or nigrostriatal pathway; decreased activity in the striatum; cortical dysfunction; neurocognitive dysfunction; cognitive deficits associated with schizophrenia; drug induced

Parkinsonism (DIP); dyskinesias; dystonia; chorea; levodopa induced dyskinesia; cerebral palsy and progressive supranuclear palsy; and Huntington's disease, including chorea associated with Huntington's disease.

### DETAILED DESCRIPTION

### Definitions

For clarity and consistency, the following definitions will be used throughout this patent document.

As used herein, “about” means ±20% of the stated value, and includes more specifically values of ±10%, ±5%, ±2% and ±1% of the stated value.

As used herein, “administering” refers to providing a compound described herein or other therapy to a subject in a form that can be introduced into that subject's body in a therapeutically useful form and therapeutically useful amount, including, but not limited to: oral dosage forms, such as, tablets, capsules, syrups, suspensions, and the like; injectable dosage forms, such as, IV, IM, IP, and the like; transdermal dosage forms, including creams, jellies, powders, and patches; buccal dosage forms; inhalation powders, sprays, suspensions, and the like; and rectal suppositories.

A health care practitioner can directly provide a compound described herein to a subject in the form of a sample or can indirectly provide a compound to a subject by providing an oral or written prescription for the compound. Also, for example, a subject can obtain a compound by themselves without the involvement of a health care practitioner. When the compound is administered to the subject, the body is transformed by the compound in some way. When a compound described herein is provided in combination with one or more other agents, “administration” is understood to include the compound and other agents are administered at the same time or at different times. When the agents of a combination are administered at the same time, they can be administered together in a single composition, or they can be administered separately. The preferred method of administration can vary depending on various factors, e.g., the components of the pharmaceutical formulation, the site of the disease, and the severity of the disease.

The term “ameliorating” in the context of treatment refers to, but is not limited to, bettering the symptoms of a disease, helping or improving the symptoms or making symptoms more tolerable or acceptable.

The term “composition” refers to a compound or crystalline form thereof, including but not limited to, salts, solvates, and hydrates of a compound described herein, in combination with at least one additional component, such as, a composition obtained/prepared during synthesis, preformulation, in-process testing (e.g., TLC, HPLC, NMR samples), and the like.

The term, “compound,” as used herein is meant to include all stereoisomers, geometric isomers, tautomers and isotopes of the structures depicted. The term is also meant to refer to compounds described herein, regardless of how they are prepared, e.g., synthetically, through biological process (e.g., metabolism or enzyme conversion), or a combination thereof. All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds can be in any solid-state form, such as a polymorph or solvate, so unless clearly indicated otherwise, reference in the specification to compounds and salts thereof should be understood as encompassing any solid-state form of the compound. In some aspects, the compounds described herein, or salts thereof, are substantially isolated. By “substantially isolated” is meant that the compound is at least partially or substantially separated from the environment in which it was formed or detected. Partial separation can include, e.g., a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salts thereof.

The term “hydrate” as used herein refers to a compound described herein or a salt thereof that further includes a stoichiometric or non-stoichiometric amount of water bound by non-covalent intermolecular forces.

The term “in need of treatment” and the term “in need thereof” when referring to treatment are used interchangeably to mean a judgment made by a caregiver (e.g. physician, nurse, nurse practitioner, etc. in the case of humans; veterinarian in the case of animals, including non-human mammals) that a subject or animal requires or will benefit from treatment. This judgment is made based on a variety of factors that are in the realm of a caregiver's expertise, but that includes the knowledge that the subject or animal is ill, or will become ill, as the result of a disease, condition or disorder that is treatable by the compound described herein. Accordingly, the compound described herein can be used in a protective or preventive manner; or compound described herein can be used to alleviate, inhibit, or ameliorate the disease, condition, or disorder.

The term “subject” refers to any animal, including mammals, such as, mice, rats, other rodents, rabbits, dogs, cats, swine, cattle, sheep, horses, primates, and humans. In the context of a clinical trial or screening or activity experiment the subject can be a healthy volunteer or healthy participant without an underlying GPR52 mediated disorder or condition or a volunteer or participant that has received a diagnosis for a disorder or condition in need of medical treatment as determined by a health care professional. In the context outside of a clinical trial a subject under the care of a health care professional who has received a diagnosis for a disorder or condition is typically described as a subject.

The term “pediatric subject” refers to a subject under the age of 21 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)) see e.g., Berhman et al., Textbook of Pediatrics, 15th Ed. Philadelphia: W.B. Saunders Company, 1996; Rudolph et al., Rudolph's Pediatrics, 21st Ed. New York: McGraw-Hill, 2002; and Avery et al., Pediatric Medicine, 2nd Ed. Baltimore: Williams & Wilkins; 1994.

The phrase “pharmaceutically acceptable” refers to compounds (and salts thereof), compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutical composition” refers to a specific composition comprising at least one active ingredient; including but not limited to, salts, solvates, and hydrates of compounds described herein, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The terms “prevent”, “preventing”, and “prevention” refer to the elimination or reduction of the occurrence or onset of one or more symptoms associated with a particular disorder. For example, the terms “prevent”, “preventing”, and “prevention” can refer to the administration of therapy on a prophylactic or preventative basis to a subject who may ultimately manifest at least one symptom of a disorder but who has not yet done so. Such subjects can be identified on the basis of risk factors that are known to correlate with the subsequent occurrence of the disease, such as the presence of a biomarker. Alternatively, prevention therapy can be administered as a prophylactic measure without prior identification of a risk factor. Delaying the onset of the at least one episode and/or symptom of a disorder can also be considered prevention or prophylaxis.

The term “solvate” as used herein refers to a solid-state form of a compound described herein, or a pharmaceutically acceptable salt thereof which includes a stoichiometric or non-stoichiometric amount of a solvent bound by non-covalent intermolecular forces. When the solvent is water, the solvate is a hydrate.

The terms “treat”, “treating”, and “treatment” refer to medical management of a disease, disorder, or condition of a subject (e.g., subject) (see, e.g., Stedman's Medical Dictionary). In general, an appropriate dose and treatment regimen provide the GPR52 agonist in an amount sufficient to provide therapeutic benefit. Therapeutic benefit for subjects to whom the GPR52 agonist compound(s) described herein are administered, includes, for example, an improved clinical outcome, wherein the object is to prevent or slow or retard (lessen) an undesired physiological change associated with the disease, or to prevent or slow or retard (lessen) the expansion or severity of such disease. The effectiveness of one or more GPR52 agonists may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival.

The term “therapeutically effective amount” refers to the amount of the compound described herein, or a pharmaceutically acceptable salt thereof, or an amount of a pharmaceutical composition comprising the compound described herein or a pharmaceutically acceptable salt thereof, that elicits the biological or medicinal response in a tissue, system, animal, or human that is being sought by a subject, researcher, veterinarian, medical doctor, or other clinician or caregiver, which can include one or more of the following:
(1) preventing the disorder, for example, preventing a disease, condition, or disorder in a subject who can be predisposed to the disease, condition, or disorder but does not yet experience or display the relevant pathology or symptomatology;
(2) inhibiting the disorder, for example, inhibiting a disease, condition, or disorder in a subject who is experiencing or displaying the relevant pathology or symptomatology (i.e., arresting further development of the pathology and/or symptomatology); and
(3) ameliorating the disorder, for example, ameliorating a disease, condition, or disorder in a subject who is experiencing or displaying the relevant pathology or symptomatology (i.e., reversing the pathology and/or symptomatology).

As used herein, the term “contacting” refers to the bringing together of indicated moieties in an in vitro system or an in vivo system. For example, “contacting” GPR52 with a compound provided herein includes the administration of a compound provided herein (or a pharmaceutically acceptable salt thereof) to a subject, such as a human, having a GPR52 protein, as well as, for example, introducing a compound provided herein into a sample containing a cellular or purified preparation containing the GPR52 protein.

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “n-membered” where n is an integer typically describes the number of ring-forming atoms in a moiety where the number of ring-forming atoms is n. For example, piperidinyl is an example of a 6-membered heterocyclyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of Formula (I), and pharmaceutically acceptable salts thereof, in which a variable appears more than once, each variable can be a different moiety independently selected from the group defining the variable. For example, where a structure is described having two R groups that are simultaneously present on the same compound, the two R groups can represent different moieties independently selected from the group defined for R.

Whenever a group is described as being “optionally substituted” that group can be unsubstituted or can be substituted with one or more of the indicated substituents. Likewise, when a group is described as being “unsubstituted or substituted” if substituted, the substituent(s) can be selected from one or more of the indicated substituents. It is to be understood that substitution at a given atom is limited by valency.

As used herein, “Cₐ-C_{b}” in which “a” and “b” are integers refer to the number of carbon atoms in an alkyl, alkenyl, or alkynyl group, or the number of carbon atoms in the ring of a cycloalkyl, cycloalkenyl, or aryl group. That is, these groups can contain from “a” to “b”, inclusive, carbon atoms. Thus, for example, a “C₁-C₄ alkyl” (or C₁₋₄alkyl) group refers to all alkyl groups having from 1 to 4 carbons, that is, CH₃-, CH₃CH₂—, CH₃CH₂CH₂, (CH₃)₂CH—, CH₃CH₂CH₂CH₂, CH₃CH₂CH(CH₃)- and (CH₃)₃C—. If no “a” and “b” are designated with regard to an alkyl, alkenyl, alkynyl, cycloalkyl, cycloalkenyl, or aryl group, the broadest range described in these definitions is to be assumed.

In addition to the foregoing, as used in the specification and appended claims, unless specified to the contrary, the following terms have the meaning indicated: The term “amino” refers to the group —NH₂.

The term “alkylamino” refers to a group of formula —NH(alkyl), where alkyl is as defined herein. Example alkylamino groups include methylamino, ethylamino, propylamino (e.g., n-propylamino and iso-propylamino), and the like.

The term “dialkylamino” refers to a group of formula —N(alkyl)₂, where alkyl is as defined herein. Example dialkylamino groups include dimethylamino, diethylamino, di-n-propylamino, di-iso-propylamino), and the like.

The term “alkenyl” refers to an alkyl group that contains in the straight or branched hydrocarbon chain one or more double bonds. Examples of alkenyl groups include allenyl, vinylmethyl, and ethenyl. In some aspects, an alkenyl group can be unsubstituted or substituted. In some aspects, the alkenyl group can have 2 to 6 carbon atoms. The alkenyl group of the compounds can be designated as “C₂-C₆ alkenyl” or similar designations.

The term “alkynyl” refers to an alkyl group that contains in the straight or branched hydrocarbon chain one or more triple bonds. Examples of alkynyls include ethynyl and propynyl. An alkynyl group can be unsubstituted or substituted. In some aspects, an alkynyl group can be unsubstituted or substituted. In some aspects, the alkynyl group can have 2 to 6 carbon atoms. The alkenyl group of the compounds can be designated as “C₂-C₆ alkynyl” or similar designations.

The term “aryl” refers to an aromatic ring system containing 6, 10 or 14 carbon atoms that can contain a single ring, two fused rings or three fused rings, such as phenyl, naphthalenyl and phenanthrenyl. In some aspects, the aryl group can have 6 or 10 carbon atoms (i.e., C₆ or C₁₀ aryl). When one or more substituents are present on the “aryl” ring, the substituent(s) can be bonded at any available ring carbon. In some aspects, an aryl group can be substituted or unsubstituted.

The term “alkyl” refers to a fully saturated straight or branched hydrocarbon radical. The alkyl group can have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms. In some aspects, the alkyl group can have 1 to 6 carbons (i.e., “C₁-C₆ alkyl”). Some aspects are 1 to 5 carbons (i.e., C₁-C₅ alkyl), some aspects are 1 to 4 carbons (i.e., C₁-C₄ alkyl), some aspects are 1 to 3 carbons (i.e., C₁-C₃ alkyl), and some aspects are 1 or 2 carbons. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Examples of an alkyl group include: methyl, ethyl, n-propyl, isopropyl, n-butyl, sec-butyl, isobutyl, tert-butyl, pentyl, isopentyl, tert-pentyl, neo-pentyl, 1-methylbutyl [i.e., —CH(CH₃) CH₂CH₂CH₃], 2-methylbutyl [i.e., —CH₂CH(CH₃) CH₂CH₃], n-hexyl and the like. When one or more substituents are present on the alkyl group, the substituent(s) can be bonded at any available carbon atom. In some aspects, an alkyl group can be substituted or unsubstituted. The term “haloalkyl” refers to an alkyl group, as defined herein, wherein one or more hydrogen atoms of the alkyl group have been replaced by a halogen atom (e.g., mono-haloalkyl, di-haloalkyl, and tri-haloalkyl). In some aspects, the haloalkyl group can have 1 to 6 carbons (i.e., “haloC₁—C₆ alkyl” or “halo-substituted-C₁₋₄alkyl”). The haloC₁—C₆ alkyl can be fully substituted in which case it can be represented by the formula CnL2n+1, wherein L is a halogen and “n” is 1, 2, 3, 4, 5, or 6. When more than one halogen is present then they can be the same or different and selected from: fluorine, chlorine, bromine, and iodine. In some aspects, haloalkyl contains 1 to 5 carbons (i.e., haloC₁—C₅ alkyl). In some aspects, haloalkyl contains 1 to 4 carbons (i.e., haloC₁—C₄ alkyl). In some aspects, haloalkyl contains 1 to 3 carbons (i.e., haloC₁—C₃ alkyl). In some aspects, haloalkyl contains 1 or 2 carbons. Examples of haloalkyl groups include fluoromethyl, difluoromethyl, trifluoromethyl, chlorodifluoromethyl, 1-fluoroethyl, 2,2,2-trifluoroethyl, pentafluoroethyl, 4,4,4-trifluorobutyl, and the like.

The term “carbonyl” refers to the group —C(═O)—.

The term “oxo” refers to the —O substituent.

The term “cycloalkyl” refers to a fully saturated all carbon mono- or multi-cyclic ring system. In some aspects, the cycloalkyl is a monocyclic ring containing 3 to 7 carbon atoms (i.e., “C₃-C₇ cycloalkyl”). Some aspects contain 3 to 6 carbons. Some aspects contain 3 to 5 carbons. Some aspects contain 5 to 7 carbons. Some aspects contain 3 to 4 carbons. Examples include cyclopropyl, cyclobutyl, cyclopentyl, and cyclohexyl. When one or more substituents are present on the alkyl group, the substituent(s) can be bonded at any available carbon atom. In some aspects, a cycloalkyl group can be substituted or unsubstituted.

The term “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (i.e., an aromatic system), otherwise the group would be “aryl,” as defined herein. When composed of two or more rings, the rings can be connected together in a fused, bridged, or spiro fashion. A cycloalkenyl can contain 3 to 12 atoms in the ring(s) or 3 to 8 atoms in the ring(s). In some aspects, a cycloalkenyl group can be unsubstituted or substituted. In some aspects, the cycloalkenyl group may have 4 to 8 carbon atoms (i.e., “C₄-C₈ cycloalkenyl”). An example is cyclohexenyl.

The term “heteroaryl” refers to a monocyclic or fused multicyclic aromatic ring system and having at least one heteroatom in the ring system, that is, an element other than carbon, including but not limited to, nitrogen, oxygen and sulfur. Some aspects are “5-6 membered heteroaryl” and refers to an aromatic ring containing 5 to 6 ring atoms in a single ring and having at least one heteroatom in the ring system. Examples of heteroaryl rings include, but are not limited to, pyridyl, pyrimidinyl, pyrazinyl, pyridazinyl, triazinyl, furyl, quinolyl, isoquinolyl, thienyl, imidazolyl, thiazolyl, indolyl, isoindolyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, dibenzo[b,d]furan, dibenzo[b,d]thiophene, phenanthridinyl, benzimidazolyl, pyrrolyl, quinolinyl, isoquinolinyl, benzisoxazolyl, imidazo[1,2-b]thiazolyl, and the like. A heteroaryl group can be substituted or unsubstituted. In some aspects, the heteroaryl group has 5 to 10 ring members or 5 to 7 ring members. The heteroaryl group can be designated as “5-7 membered heteroaryl,” “5-10 membered heteroaryl,” or similar designations. In some aspects, the heteroaryl can be a substituted or unsubstituted C₁-C₁₃ five-, six-, seven, eight-, nine-, ten-, up to 14-membered monocyclic, bicyclic, or tricyclic ring system including 1 to 5 heteroatoms selected from nitrogen, oxygen and sulfur. In some aspects, the heteroaryl can be a substituted or unsubstituted C₁-C₅ five- or six-membered monocyclic ring including 1 to 5 heteroatoms selected from nitrogen, oxygen and sulfur. In some aspects, the heteroaryl can be a substituted or unsubstituted C₅-C₉ eight-, nine- or ten-membered bicyclic ring system including 1 to 5 heteroatoms selected from nitrogen, oxygen and sulfur. In some aspects, the heteroaryl is a substituted or unsubstituted C₅-C₉ eight-, nine- or ten-membered heteroaryl. In some aspects, the C₅-C₉ eight-, nine- or ten-membered bicyclic heteroaryl is imidazo[2,1-b]thiazolyl, 1H-indolyl, isoindolyl, benzofuranyl, benzothienyl, benzimidazolyl, benzisoxazolyl, indazolyl, purinyl, quinolinyl, isoquinolinyl, quinoxalinyl, pyrido[3,4-b]pyrazinyl or pyrido[4,3-d]pyrimidinyl. In some aspects, the heteroaryl is a substituted or unsubstituted C₈-C_{13 13}- or 14-membered tricyclic ring system including 1 to 5 heteroatoms selected from nitrogen, oxygen and sulfur. In some aspects, the heteroaryl can be an azolyl such as imidazolyl, pyrazolyl, 1,2,3-triazolyl, 1,2,4-triazolyl, tetrazolyl, 1,2,4-thiadiazolyl, thiazolyl, isothiazolyl, oxazolyl, or isoxazolyl, each of which can be substituted or unsubstituted. In some aspects, the heteroaryl is a C₁-C₁₃5-membered heteroaryl. In some aspects, the C₁-C₄ 5-membered heteroaryl is furanyl, thienyl, 1,2,4-thiadiazolyl, 1,2,3-thiadiazolyl, isothiazolyl, thiazolyl, imidazolyl, pyrazolyl, isoxazolyl, oxazolyl, pyrrolyl, triazolyl, tetrazolyl. In some aspects, the heteroaryl is a C₃-C₅ 6-membered heteroaryl. In some aspects, the C₃-C₅ 6-membered heteroaryl is pyridinyl, pyrimidinyl, pyrazinyl, pyridazinyl, or triazinyl. In some aspects, “5-10 membered heteroaryl” refers to: furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, quinoxalinyl, triazinyl, benzofuranyl, 1H-indolyl, benzo[b]thiophenyl, and the like. In some aspects, “5-10 membered heteroaryl” refers to: pyrazinyl, pyridazinyl, pyridinyl, pyrimidinyl, 1H-indolyl, quinoxalinyl, thiadiazolyl, and the like. In some aspects, a heteroaryl group can be substituted or unsubstituted.

The position of the nitrogen in the pyridine ring, relative to the oxygen linker in the Formula I, changes the EC₅₀, for example

| | Act- |
|---|---|
| Compound Structure | ivity |
| 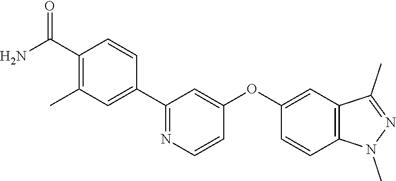 [SMILES: Cc1cc(-c2cc(Oc3ccc4c(c3)c(C)nn4C)ccn2)ccc1C(N)=O] | EC₅₀ is 89 nM |
| 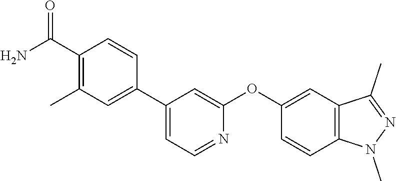 [SMILES: Cc1cc(-c2ccnc(Oc3ccc4c(c3)c(C)nn4C)c2)ccc1C(N)=O] | EC₅₀ is 2043 nM |

The term “heterocyclyl” refers to a three-, four-, five-, six-, seven-, eight-, nine-, ten-, up to 18-membered monocyclic, bicyclic, and tricyclic ring system wherein carbon atoms together with from 1 to 5 heteroatoms constitute said ring system and optionally containing one or more unsaturated bonds situated in such a way, however, that a fully delocalized pi-electron system (aromatic system) does not occur in the monocyclic ring or in at least one ring of the bicyclic or tricyclic ring system. The heteroatom(s) is an element other than carbon including, but not limited to, oxygen, sulfur, and nitrogen. In some aspects, the heterocyclyl can be a 3-7 membered saturated non-aromatic ring system containing 3 to 7 ring atoms, where at least one ring atom is a heteroatom. In some aspects, “3-6 membered heterocyclyl” refers to a saturated non-aromatic ring radical containing 3 to 6 ring atoms, where at least one ring atom is a heteroatom. In some aspects, “4-6 membered heterocyclyl” refers to a saturated non-aromatic ring radical containing 4 to 6 ring atoms, where at least one ring atom is a heteroatom. In some aspects, the one or two heteroatoms in the ring system are selected independently from: O (oxygen) and N(nitrogen). In some aspects, a heterocyclyl can include a carbonyl (C═O) group adjacent to a hetero atom, that can be substituted with an oxo on a carbon adjacent to a hetero atom, where the substituted ring system is a lactam, lactone, cyclic imide, cyclic thioimide or cyclic carbamate. Examples of unsubstituted or oxo substituted “heterocyclyl” groups include but are not limited to, aziridinyl, azetidinyl, tetrahydrofuranyl, 1,3-dioxinyl, 1,3-dioxanyl, 1,4-dioxanyl, 1,2-dioxolanyl, 1,3-dioxolanyl, 1,4-dioxolanyl, 1,3-oxathianyl, 1,4-oxathiinyl, 1,3-oxathiolanyl, 1,3-dithiolyl, 1,3-dithiolanyl, 1,4-oxathianyl, tetrahydro-1,4-thiazinyl, 2H-1,2-oxazinyl, maleimidyl, succinimidyl, dioxopiperazinyl, hydantoinyl, imidazolinyl, imidazolidinyl, isoxazolinyl, isoxazolidinyl, isoindolinyl, indolinyl, oxazolinyl, oxazolidinyl, oxazolidinonyl, thiazolinyl, thiazolidinyl, morpholinyl, oxiranyl, piperidinyl N-oxide, piperidinyl, piperazinyl, pyrrolidinyl, pyrrolidonyl, pyrrolidionyl, 4-piperidonyl, pyrazolinyl, pyrazolidinyl, 2-oxopyrrolidinyl, tetrahydropyranyl, 4H-pyranyl, tetrahydrothiopyranyl, 1,4-diazabicyclo[2.2.2]octane, 1,4-diazabicyclo[3.1.1]heptane, 2-azaspiro[3,3]heptane, 2,6-diazaspiro[3,3]heptane, 2-oxa-6-azaspiro[3,3]heptane, and benzo-fused analogs (e.g., their benzimidazolidinonyl, tetrahydroquinolinyl, and 3,4-methylenedioxyphenyl). The heterocyclyl group can be designated as “3-10 membered heterocyclyl” or similar designations. In some aspects, the heterocyclyl can be a C₂-C₁₂ three-, four-, five-, six-, seven-, eight-, nine-, ten-, up to 13-membered monocyclic, bicyclic, or tricyclic ring system including 1 to 5 heteroatoms selected from nitrogen, oxygen and sulfur. In some aspects, the heterocyclyl can be a substituted or unsubstituted C₂-C₆ three-, four-, five-, six-, or seven-membered monocyclic ring including 1 to 5 heteroatoms selected from nitrogen, oxygen and sulfur. In some aspects, the heterocyclyl can be a substituted or unsubstituted C₂-C₁₀ four-, five-, six-, seven-, eight-, nine-, ten- or eleven-membered bicyclic ring system including 1 to 5 heteroatoms selected from nitrogen, oxygen and sulfur. In some aspects, the heterocyclyl can be a substituted or unsubstituted C₇-C₁₂12- or 13-membered tricyclic ring system including 1 to 5 heteroatoms selected from nitrogen, oxygen and sulfur. In some aspects, the heteroatom(s) of six membered monocyclic heterocyclyls are selected from one up to three of O (oxygen), N(nitrogen) or S (sulfur), and the heteroatom(s) of five membered monocyclic heterocyclyls are selected from one or two heteroatoms selected from O (oxygen), N(nitrogen) or S (sulfur). In some aspects, the heterocyclyl can be aziridinyl, azetidinyl, tetrahydrofuranyl, 1,3-dioxinyl, 1,3-dioxanyl, 1,4-dioxanyl, 1,2-dioxolanyl, 1,3-dioxolanyl, 1,3-oxathianyl, 1,4-oxathianyl, 1,3-oxathiolanyl, 1,3-dithiolyl, 1,3-dithiolanyl, 1,4-oxathianyl, tetrahydro-1,4-thiazinyl, imidazolinyl, imidazolidinyl, isoxazolinyl, isoxazolidinyl, isoindolinyl, indolinyl, oxazolinyl, oxazolidinyl, thiazolinyl, thiazolidinyl, morpholinyl, oxiranyl, piperidinyl, piperazinyl, pyrrolidinyl, pyrazolinyl, pyrazolidinyl, tetrahydropyranyl, tetrahydrothiopyranyl, 1,4-diazabicyclo[2.2.2]octane, 1,4-diazabicyclo[3.1.1]heptane, 2-azaspiro[3,3]heptane, 2,6-diazaspiro[3,3]heptane, tetrahydroquinolinyl, 1,2,3,4-tetrahydroisoquinolinyl, 1,2,3,4-tetrahydro-2,6-naphthyridinyl, 1,2,3,4-tetrahydro-2,7-naphthyridinyl, 1,2,3,4-tetrahydro-1,7-naphthyridinyl, 1,2,3,4-tetrahydro-1,6-naphthyridinyl, 5,6,7,8-tetrahydropyrido[2,3-d]pyrimidinyl, 5,6,7,8-tetrahydropyrido[3,4-d]pyrimidinyl, [1,3]dioxolo[4,5-c]pyridinyl, [1,3]dioxolo[4,5-b]pyridinyl, [1,3]dioxolo[4,5-d]pyrimidinyl or 3,4-methylenedioxyphenyl. In some aspects, the unsubstituted or substituted heterocyclyl can be selected from aziridinyl, azetidinyl, piperidinyl, morpholinyl, oxetanyl, piperazinyl, pyrrolidinyl, thiomorpholinyl, 2-piperidone, 1,1-dioxidothiomorpholinyl, oxolanyl (tetrahydrofuranyl), and oxanyl (tetrahydropyranyl). When one or more substituents are present on the heterocyclyl group, the substituent(s) can be bonded at any available carbon atom and/or heteroatom. In some aspects, a heterocyclyl group can be substituted or unsubstituted.

The term “alkoxy” refers to the formula —OR wherein R is an alkyl defined herein. A non-limiting list of alkoxys are methoxy, ethoxy, n-propoxy, 1-methylethoxy (iso-propoxy), n-butoxy, iso-butoxy, sec-butoxy, and tert-butoxy. The alkoxy group of the compounds can be designated as “C₁-C₆ alkoxy” or similar designations. In some aspects, an alkoxy can be substituted or unsubstituted.

The term “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. In some aspects, the haloalkoxy group may have 1 to 6 carbon atoms.

The haloalkoxy group of the compounds can be designated as “haloC₁—C₆ alkoxy” or similar designations.

The term “cyano” refers to the group —CN.

The term “halogen” or “halo” refers to fluoro, chloro, bromo, or iodo group. In some aspects, halogen or halo is fluoro, chloro, or bromo. In some aspects, halogen or halo is fluoro or chloro. In some aspects, halogen or halo is fluoro.

A “C-amido” group refers to a “—C(═O)N(R^{A}R^{B})” group that is connected to the rest of the molecule via a carbon atom, and in which R^{A} and R^{B} can be independently hydrogen, C₁-C₆ alkyl, C₂-C₆ alkenyl, C₂-C₆ alkynyl, C₃-C₇ cycloalkyl, C₅-C₈ cycloalkenyl, C₆ or C₁₀ aryl, heteroaryl, or heterocyclyl.

An “N-amido” group refers to a “RC(═O)N(R^{A})—” group that is connected to the rest of the molecule via a nitrogen atom, and in which R and R^{A} can be independently hydrogen, C₁-C₆ alkyl, C₂-C₆ alkenyl, C₂-C₆ alkynyl, C₃-C₇ cycloalkyl, C₅-C₈ cycloalkenyl, C₆ or C₁₀ aryl, heteroaryl, or heterocyclyl.

The term “hydroxyalkyl” refers to an alkyl group in which one or more of the hydrogen atoms are replaced by a hydroxy group. In some aspects, the hydroxyalkyl group may have 1 to 6 carbon atoms (i.e., “hydroxyC₁—C₆ alkyl”). Exemplary hydroxyalkyl groups include, but are not limited to, 2-hydroxyethyl, 3-hydroxypropyl, 2-hydroxypropyl, and 2,2-dihydroxyethyl.

The term “hydroxy” refers to a —OH group.

The term “nitro” refers to a —NO₂ group.

As used herein, an “excipient” refers to a substance that is added to a composition to provide, without limitation, bulk, consistency, stability, binding ability, lubrication, disintegrating ability, etc., to the composition. A “diluent” is a type of excipient and refers to an ingredient in a pharmaceutical composition that lacks pharmacological activity but can be pharmaceutically necessary or desirable. For example, a diluent can be used to increase the bulk of a potent drug whose mass is too small for manufacture and/or administration. It may also be a liquid for the dissolution of a drug to be administered by injection, ingestion, or inhalation. A pharmaceutically acceptable excipient is a physiologically and pharmaceutically suitable non-toxic and inactive material or ingredient that does not interfere with the activity of the drug substance. Pharmaceutically acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21st Ed. Mack Pub. Co., Easton, PA (2005)). Preservatives, stabilizers, dyes, buffers, and the like can be provided in the pharmaceutical composition. In addition, antioxidants and suspending agents may also be used. For compositions formulated as liquid solutions, acceptable carriers and/or diluents include saline and sterile water, and may optionally include antioxidants, buffers, bacteriostats and other common additives. In some aspects, the diluents can be a buffered aqueous solution such as, without limitation, phosphate buffered saline. The compositions can also be formulated as capsules, granules, or tablets which contain, in addition to a compound as disclosed and described herein, diluents, dispersing and surface-active agents, binders, and lubricants. One skilled in this art may further formulate a compound as disclosed and described herein in an appropriate manner, and in accordance with accepted practices, such as those disclosed in Remington, supra.

As used herein, a “dose” or “dosage” refers to the measured quantity of drug substance to be taken at one time by a subject. In certain aspects, wherein the drug substance is not a free base or free acid, the quantity is the molar equivalent to the corresponding amount of free base or free acid.

As used herein, a “pharmaceutically acceptable salt” refers to salts of a compound having an acidic or basic moiety which are not biologically or otherwise undesirable for use in a pharmaceutical. In many cases, the compounds disclosed herein are capable of forming acid and/or base salts by virtue of the presence of an acidic or basic moiety (e.g. amino and/or carboxyl groups or groups similar thereto). Pharmaceutically acceptable acid addition salts can be formed by combining a compound having a basic moiety with inorganic acids and organic acids. Inorganic acids which can be used to prepare salts include, for example, hydrochloric acid, hydrobromic acid, sulfuric acid, nitric acid, phosphoric acid, and the like. Organic acids which can be used to prepare salts include, for example, methanesulfonic acid, ethanesulfonic acid, p-toluenesulfonic acid, and the like. Pharmaceutically acceptable base addition salts can be formed by combining a compound having an acidic moiety with inorganic and organic bases. Inorganic bases which can be used to prepare salts include, for example, sodium, potassium, lithium, ammonium, calcium, magnesium, iron, zinc, manganese, aluminum hydroxides, carbonates, bicarbonates, phosphates, and the like. In some aspects, the inorganic base salt is ammonium, potassium, sodium, calcium, and magnesium hydroxides, carbonates, bicarbonates, or phosphates. Organic bases from which can be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Generally, such salts can be prepared by reacting the free acid or base forms of these compounds with at least a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN). Lists of suitable salts are found in WO 87/05297; Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977); each of which is incorporated herein by reference in its entirety. A reference for the preparation and selection of pharmaceutical salts of the present disclosure is P. H. Stahl & C. G. Wermuth, Handbook of Pharmaceutical Salts, Verlag Helvetica Chimica Acta, Zurich, 2002 which is incorporated herein by reference in its entirety.

The compounds described herein can be asymmetric (e.g., having one or more stereocenters). All stereoisomers, such as enantiomers and diastereomers, are intended unless otherwise indicated. It is understood that, in any compound described herein having one or more chiral centers, if an absolute stereochemistry is not expressly indicated, then each center may independently be the (R)-configuration, or the(S)-configuration, or a mixture thereof. Thus, the compounds provided herein can be enantiomerically pure, enantiomerically enriched, a racemic mixture, diastereomerically pure, diastereomerically enriched, or a stereoisomeric mixture. Preparation of enantiomerically pure or enantiomerically enriched forms can be accomplished by resolution of racemic mixtures or by using enantiomerically pure or enriched starting materials or by stereoselective or stereospecific synthesis. Stereochemical definitions are available in E. L. Eliel, S. H. Wilen & L. N. Mander, Stereochemistry of Organic Compounds, John Wiley & Sons, Inc., New York, NY, 1994 which is incorporated herein by reference in its entirety. In some aspects, where the compound described herein is chiral or otherwise includes one or more stereocenters, the compound can be prepared with an enantiomeric excess or diastereomeric excess of greater than about 75%, greater than about 80%, greater than about 85%, greater than about 90%, greater than about 95%, or greater than about 99%.

Resolution of racemic mixtures of compounds can be carried out by any of numerous methods known in the art. An example method includes fractional recrystallizaion using a chiral resolving organic acid with a racemic compound containing a basic group. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other chiral resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Similarly, fractional recrystallization using a chiral resolving base can be utilized with a racemic compound containing a basic group.

Resolution of racemic mixtures can also be carried out by elution on a column packed with an optically active resolving agent (e.g., dinitrobenzoylphenylglycine). A suitable elution solvent composition can be determined by one skilled in the art.

In some aspects, a compound described herein can be prepared having at least about 5%, at least about 10%, at least about 20%, at least about 30%, at least about 40%, at least about 50%, at least about 60%, at least about 70%, at least about 80%, at least about 90%, at least about 95%, at least about 99%, or at least about 99.9% enantiomeric excess, or an enantiomeric excess within a range defined by any of the preceding numbers.

In addition, it is understood that, when a compound described herein contain one or more double bond(s) (e.g., C═C, C═N, and the like) or other centers of geometric asymmetry, and unless specified otherwise, it is understood that the compound includes both E and Z geometric isomers (e.g., cis or trans). Cis and trans geometric isomers of the compounds described herein can be isolated as a mixture of isomers or as separated isomeric form.

The compounds described herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H—, 2H—and 4H-1,2,4-triazole, 1H- and 2H-isoindole, and 1H- and 2H-pyrazole. Tautomeric forms can be in equilibrium or sterically locked into one form by appropriate substitution.

The compounds described herein and their pharmaceutically acceptable salts can be found together with other substances such as water and solvents, for example, in the form of hydrates or solvates. When in the solid-state, the compounds described herein and salts thereof may occur in various forms and may, e.g., take the form of solvates, including hydrates. The compounds can be in any solid-state form, such as a crystalline form, amorphous form, solvated form, etc, and unless clearly indicated otherwise, reference in the specification to compounds and salts thereof should be understood as reading on any solid-state form of the compound.

The compounds described herein can be used in a neutral form, such as, a free acid or free base form. Alternatively, the compounds can be used in the form of pharmaceutically acceptable salts, such as pharmaceutically acceptable addition salts of acids or bases.

In some aspects, the compounds described herein, or salts thereof, are substantially isolated. The phrase “substantially isolated” refers to the compound that is at least partially or substantially separated from the environment in which it was formed or detected. Partial separation can include, for example, a composition enriched in the compound described herein. Substantial separation can include compositions containing at least about 50%, at least about 60%, at least about 65%, at least about 70%, at least about 75%, at least about 80%, at least about 85%, at least about 90%, at least about 95%, at least about 97%, or at least about 99% by weight of the compound described herein, or salt thereof.

The compounds disclosed and described herein allow atoms at each position of the compound independently to have: 1) an isotopic distribution for a chemical element in proportional amounts to those usually found in nature or 2) an isotopic distribution in proportional amounts different to those usually found in nature unless the context clearly dictates otherwise. A particular chemical element has an atomic number defined by the number of protons within the atom's nucleus. Each atomic number identifies a specific element, but not the isotope; an atom of a given element may have a wide range in its number of neutrons. The number of both protons and neutrons in the nucleus is the atom's mass number, and each isotope of a given element has a different mass number. A compound wherein one or more atoms have an isotopic distribution for a chemical element in proportional amounts different to those usually found in nature is commonly referred to as being an isotopically-labeled compound. Each chemical element as represented in a compound structure may include any isotopic distribution of said element. For example, in a compound structure a hydrogen atom can be explicitly disclosed or understood to be present in the compound. At any position of the compound that a hydrogen atom can be present, the hydrogen atom can be an isotopic distribution of hydrogen, including but not limited to protium (1H) and deuterium (²H) in proportional amounts to those usually found in nature and in proportional amounts different to those usually found in nature. Thus, reference herein to a compound encompasses all potential isotopic distributions for each atom unless the context clearly dictates otherwise. Examples of isotopes include isotopes of hydrogen, carbon, nitrogen, oxygen, phosphorous, sulfur, fluorine, chlorine, bromine, and iodine. As one of skill in the art would appreciate, any of the compounds as disclosed and described herein may include radioactive isotopes. Accordingly, also contemplated is use of compounds as disclosed and described herein, wherein one or more atoms have an isotopic distribution different to those usually found in nature, such as having ²H or ³H in greater proportion, or ¹¹C, ¹³C, or ¹⁴C in greater proportion than found in nature. By way of general example, and without limitation, isotopes of hydrogen include protium (¹H), deuterium (²H), and tritium (³H). Isotopes of carbon include carbon-11 (¹¹C), carbon-12 (¹²C), carbon-13 (¹³C), and carbon-14 (¹⁴C). Isotopes of nitrogen include nitrogen-13 (¹³N), nitrogen-14 (¹⁴N) and nitrogen-15 (¹⁵N). Isotopes of oxygen include oxygen-14 (¹⁴O), oxygen-15 (¹⁵O), oxygen-16 (¹⁶O)), oxygen-17 (¹⁷O), and oxygen-18 (¹⁸O). Isotope of fluorine include fluorine-17 (¹⁷F), fluorine-18 (¹⁸F) and fluorine-19 (¹⁹F). Isotopes of phosphorous include phosphorus-31 (³¹P), phosphorus-32 (³²P), phosphorus-33 (³³P), phosphorus-34 (³⁴P), phosphorus-35 (³⁵P) and phosphorus-36 (³⁶P). Isotopes of sulfur include sulfur-32 (³²S), sulfur-33 (³³S), sulfur-34 (³⁴S), sulfur-35 (³⁵S), sulfur-36 (³⁶S) and sulfur-38 (³⁸S). Isotopes of chlorine include chlorine-35 (³⁵Cl), chlorine-36 (³⁶Cl) and chlorine-37 (³⁷Cl). Isotopes of bromine include bromine-75 (¹⁵Br), bromine-76 (¹⁶Br), bromine-77 (⁷⁷Br), bromine-79 (¹⁹Br), bromine-81 (⁸¹Br) and bromine-82 (⁸²Br). Isotopes of iodine include iodine-123 (¹²³I), iodine-124 (¹²⁴I)), iodine-125 (¹²⁵I), iodine-131 (¹³¹I) and iodine-135 (¹³⁵I). In some aspects, atoms at every position of the compound have an isotopic distribution for each chemical element in proportional amounts to those usually found in nature. In some aspects, an atom in one position of the compound has an isotopic distribution for a chemical element in proportional amounts different to those usually found in nature (remainder atoms having an isotopic distribution for a chemical element in proportional amounts to those usually found in nature). In some aspects, atoms in at least two positions of the compound independently have an isotopic distribution for a chemical element in proportional amounts different to those usually found in nature (remainder atoms having an isotopic distribution for a chemical element in proportional amounts to those usually found in nature). In some aspects, atoms in at least three positions of the compound independently have an isotopic distribution for a chemical element in proportional amounts different to those usually found in nature (remainder atoms having an isotopic distribution for a chemical element in proportional amounts to those usually found in nature). In some aspects, atoms in at least four positions of the compound independently have an isotopic distribution for a chemical element in proportional amounts different to those usually found in nature (remainder atoms having an isotopic distribution for a chemical element in proportional amounts to those usually found in nature). In some aspects, atoms in at least five positions of the compound independently have an isotopic distribution for a chemical element in proportional amounts different to those usually found in nature (remainder atoms having an isotopic distribution for a chemical element in proportional amounts to those usually found in nature). In some aspects, atoms in at least six positions of the compound independently have an isotopic distribution for a chemical element in proportional amounts different to those usually found in nature (remainder atoms having an isotopic distribution for a chemical element in proportional amounts to those usually found in nature).

Certain compounds, for example those having incorporated radioactive isotopes such as ³H and ¹⁴C, are also useful in drug or substrate tissue distribution assays. Tritium (³H) and carbon-14 (¹⁴C) isotopes are particularly preferred for their ease of preparation and detectability. Compounds with isotopes such as deuterium (²H) in proportional amounts greater than usually found in nature may afford certain therapeutic advantages resulting from greater metabolic stability, such as, for example, increased in vivo half-life or reduced dosage requirements. Isotopically-labeled compounds can generally be prepared by performing procedures routinely practiced in the chemical art. Methods are readily available to measure such isotope perturbations or enrichments, such as, mass spectrometry, and for isotopes that are radio-isotopes additional methods are available, such as, radio-detectors used in connection with HPLC or GC.

As used herein, “isotopic variant” means a compound that contains an unnatural proportion of an isotope at one or more of the atoms that constitute such a compound. In certain aspects, an “isotopic variant” of a compound contains unnatural proportions of one or more isotopes, including, but not limited to, protium (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O)), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷C₁), bromine-79 (¹⁹Br), bromine-81 (⁸¹Br), iodine-123 (¹²³I), iodine-125 (¹²⁵I), iodine-127 (¹²⁷I), iodine-129 (¹²⁹I)), and iodine-131 (¹³¹I)). In certain aspects, an “isotopic variant” of a compound is in a stable form, that is, non-radioactive. In certain aspects, an “isotopic variant” of a compound contains unnatural proportions of one or more isotopes, including, but not limited to, hydrogen (1H), deuterium (²H), carbon-12 (¹²C), carbon-13 (¹³C), nitrogen-14 (¹⁴N), nitrogen-15 (¹⁵N), oxygen-16 (¹⁶O)), oxygen-17 (¹⁷O), and oxygen-18 (¹⁸O). In certain aspects, an “isotopic variant” of a compound is in an unstable form, that is, radioactive. In certain aspects, an “isotopic variant” of a compound described herein contains unnatural proportions of one or more isotopes, including, but not limited to, tritium (3H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can include 2H as the major isotopic form, as example, or any carbon include be ¹³C as the major isotopic form, as example, or any nitrogen can include ¹⁵N as the major isotopic form, as example, and any oxygen can include ¹⁸O as the major isotopic form, as example. In certain aspects, an “isotopic variant” of a compound contains an unnatural proportion of deuterium (²H).

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain aspects, at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position.

Synthetic methods for incorporating radio-isotopes into organic compounds are applicable to compound described herein and are well known in the art. These synthetic methods, for example, incorporating activity levels of tritium into target molecules, are as follows:
A. Catalytic Reduction with Tritium Gas: This procedure normally yields high specific activity products and requires halogenated or unsaturated precursors.
B. Reduction with Sodium Borohydride [3H]: This procedure is rather inexpensive and requires precursors containing reducible functional groups such as aldehydes, ketones, lactones, esters and the like.
C. Reduction with Lithium Aluminum Hydride [3H]: This procedure offers products at almost theoretical specific activities. It also requires precursors containing reducible functional groups such as aldehydes, ketones, lactones, esters and the like.
D. Tritium Gas Exposure Labeling: This procedure involves exposing precursors containing exchangeable protons to tritium gas in the presence of a suitable catalyst.
E. N-Methylation using Methyl Iodide [3H]: This procedure is usually employed to prepare O-methyl or N-methyl (3H) products by treating appropriate precursors with high specific activity methyl iodide (3H). This method in general allows for higher specific activity, such as for example, about 70-90 Ci/mmol.

Synthetic methods for incorporating activity levels of ¹²⁵I into target molecules include:
A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A representative procedure was reported by Zhu, G-D. and co-workers in J. Org. Chem., 2002, 67, 943-948.
B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two-step process. The first step is the conversion of the aryl or heteroaryl bromide to the corresponding tri-alkyltin intermediate using for example, a Pd catalyzed reaction [i.e. Pd(Ph3P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm., 2001, 44, S280-S282.

A radiolabeled form of a compound described herein can be used in a screening assay to identify/evaluate compounds. In general terms, a newly synthesized or identified compound (i.e., test compound) can be evaluated for its ability to reduce binding of a radiolabeled form of a compound disclosed herein to GPR52. The ability of a test compound to compete with a radiolabeled form of a compound described herein for the binding to GPR52 correlates to its binding affinity.

### Aspects of the Disclosure

The present disclosure relates to compounds capable of modulating the activity of GPR52. In one aspect of the disclosure, with respect to compounds of formula (I), are compounds of Formula (Ia)

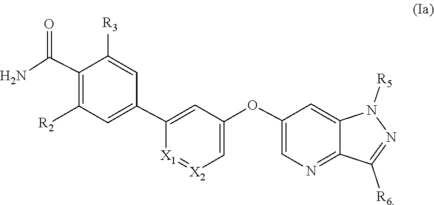

In a further aspect are compounds of Formula (Ia) wherein R₂ is selected from methyl, ethyl, methyl-amino, chloro and trifluoro-methyl.

In a further aspect are compounds of Formula (Ia) wherein R₃ is selected from hydrogen and halo.

In a further aspect are compounds of Formula (Ia) wherein R₅ is selected from hydrogen, methyl and ethyl.

In a further aspect are compounds of Formula (Ia) wherein R₆ is selected from hydrogen, methyl, fluoro, amino, cyano, and trifluoromethyl.

In a further aspect are compounds of Formula (Ia) wherein X₁ is selected from N and CH.

In a further aspect are compounds of Formula (Ia) wherein X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect is a compound, or a pharmaceutically acceptable salt form, of Formula (Ia), in which:
R₂ is selected from C₁₋₂alkyl, halo, methyl-amino and halo-substituted-C₁₋₂alkyl;
R₃ is selected from hydrogen and halo;
R₅ is selected from hydrogen and C₁₋₂alkyl;
R₆ is selected from hydrogen, amino, cyano, C₁₋₂alkyl, halo and halo-substituted-C₁₋₂alkyl;
X₁ is selected from N and CH; and
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect are compounds of Formula (Ia) wherein:
R₂ is selected from methyl, ethyl, methyl-amino, chloro and trifluoro-methyl;
R₃ is selected from hydrogen and halo;
R₅ is selected from hydrogen, methyl and ethyl;
R₆ is selected from hydrogen, methyl, fluoro, amino, cyano, and trifluoromethyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect are compounds of Formula (Ia) wherein: R₂ is selected from methyl, ethyl and trifluoromethyl;
R₃ is selected from hydrogen and fluoro;
X₁ is selected from N and CH; and
X₂ is CR₉; wherein R₉ is selected from hydrogen and amino; and the pharmaceutically acceptable salts thereof.

In a further aspect are compounds of Formula (Ia), or a pharmaceutically acceptable salt thereof, selected from

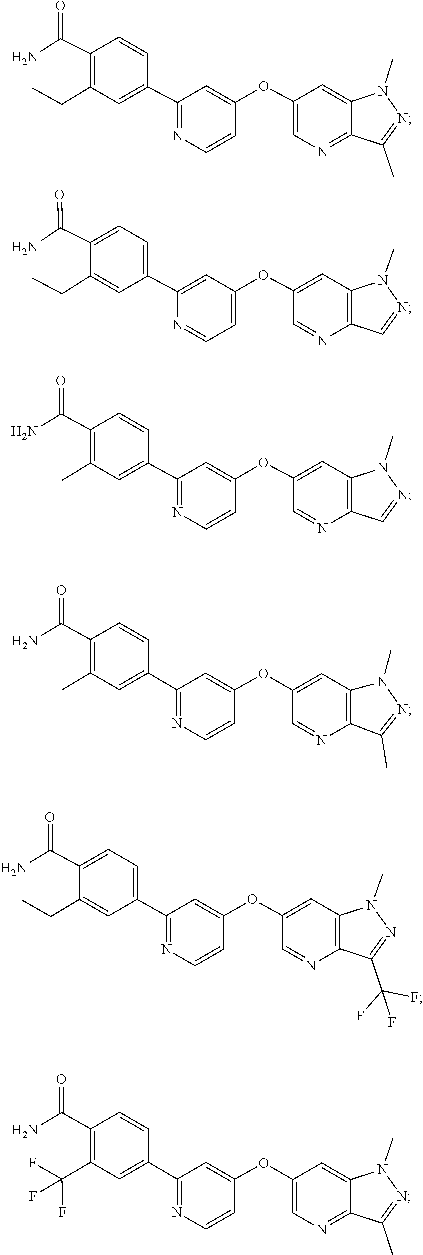

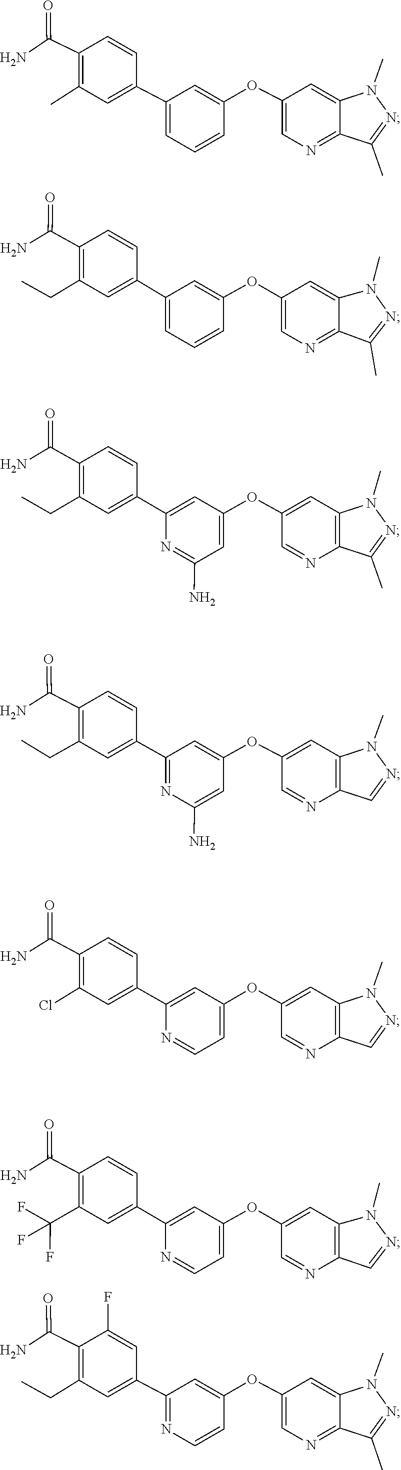

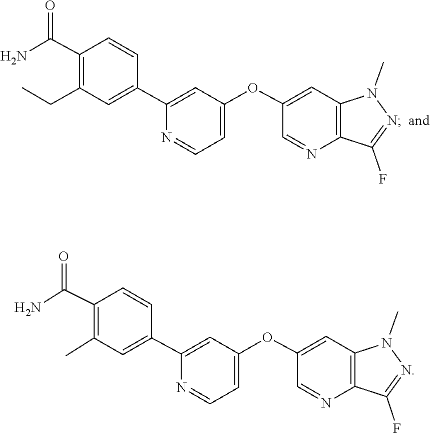

In another aspect of the disclosure, with respect to compounds of formula (I), are compounds of Formula (Ib)

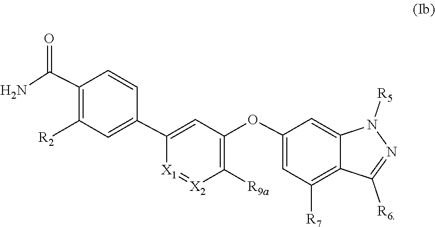

In a further aspect of the disclosure are compounds of Formula Ib in which R₂ is selected from C₁₋₂alkyl, halo, methyl-amino and halo-substituted-C₁₋₂alkyl.

In a further aspect of the disclosure are compounds of Formula Ib in which R₅ is selected from hydrogen, C₁₋₂alkyl and halo-substituted-C₁₋₂alkyl.

In a further aspect of the disclosure are compounds of Formula Ib in which R₆ is selected from hydrogen, amino, cyano, halo, C₁-2alkyl and halo-substituted-C₁₋₂alkyl.

In a further aspect of the disclosure are compounds of Formula Ib in which R₇ is selected from hydrogen, C₁₋₂alkyl and halo.

In a further aspect of the disclosure are compounds of Formula Ib in which R₉ₐ is selected from hydrogen and methyl.

In a further aspect of the disclosure are compounds of Formula Ib in which X₁ is selected from N and CH.

In a further aspect of the disclosure are compounds of Formula Ib in which X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula Ib in which: R₂ is selected from C₁₋₂alkyl, halo, methyl-amino and halo-substituted-C₁₋₂alkyl;
R₅ is selected from hydrogen, C₁₋₂alkyl and halo-substituted-C₁₋₂alkyl;
R₆ is selected from hydrogen, amino, cyano, halo, C₁-2alkyl and halo-substituted-C₁₋₂alkyl;
R₇ is selected from hydrogen, C₁₋₂alkyl and halo;
R₉ₐ is selected from hydrogen and methyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Ib) in which:
R₂ is selected from methyl, ethyl, methyl-amino, chloro and trifluoro-methyl;
R₅ is selected from hydrogen, methyl, ethyl and trifluoromethyl;
R₆ is selected from hydrogen, methyl, amino, cyano, fluoro and trifluoromethyl;
R₇ is selected from hydrogen, methyl and fluoro;
R₉ₐ is selected from hydrogen and methyl;
X₁ is selected from N and CH;
X₂ is selected from N and CR₈; wherein R₅ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Ib) wherein: X₂ is CR₈; wherein R₈ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Ib), or a pharmaceutically acceptable salt thereof, selected from

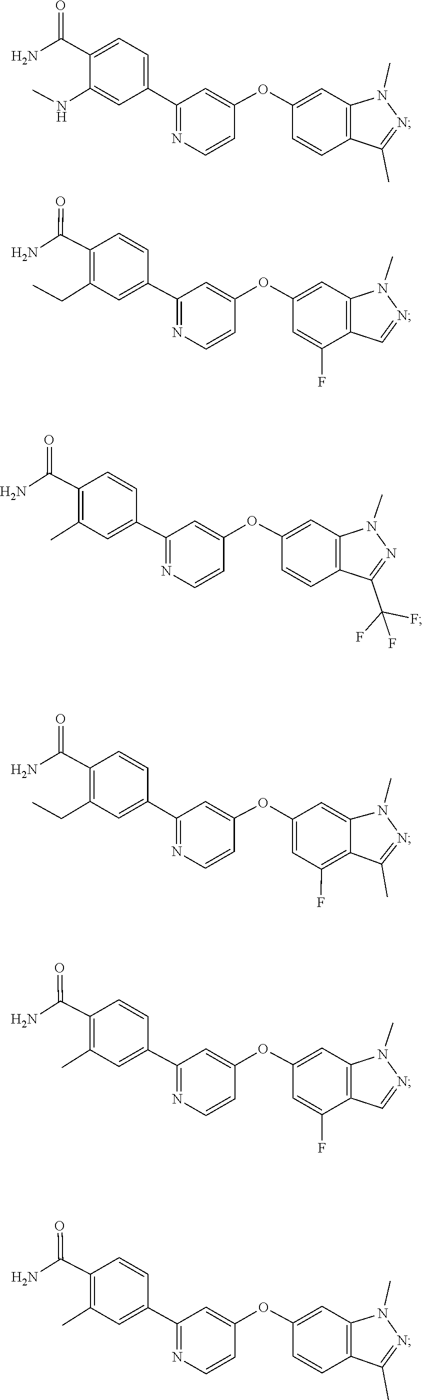

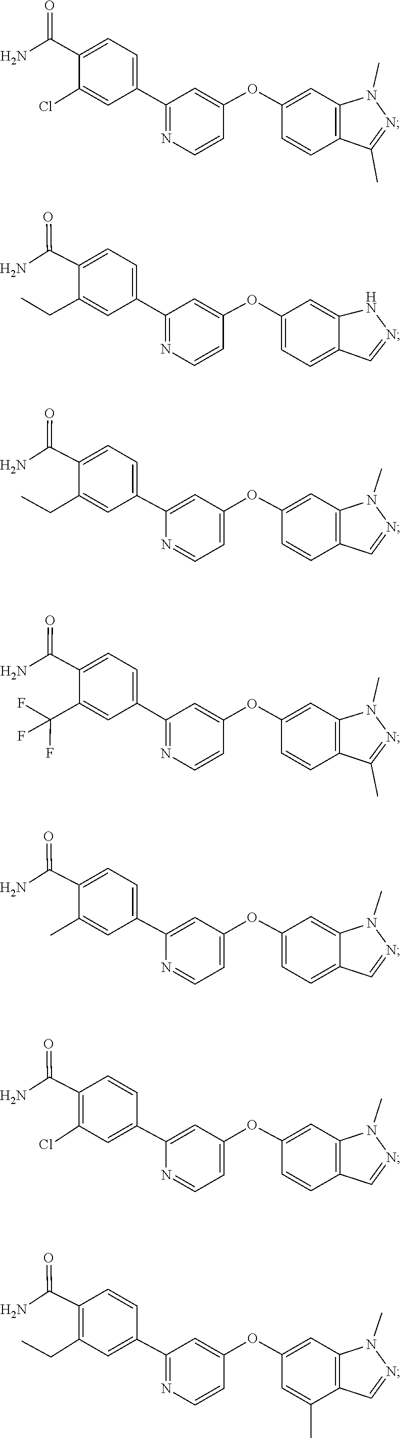

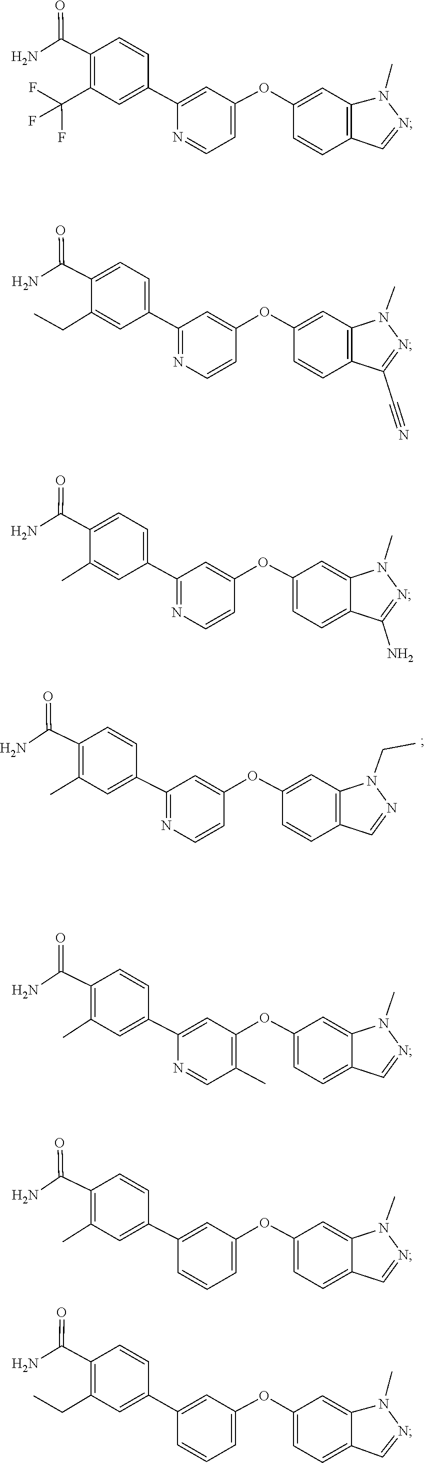

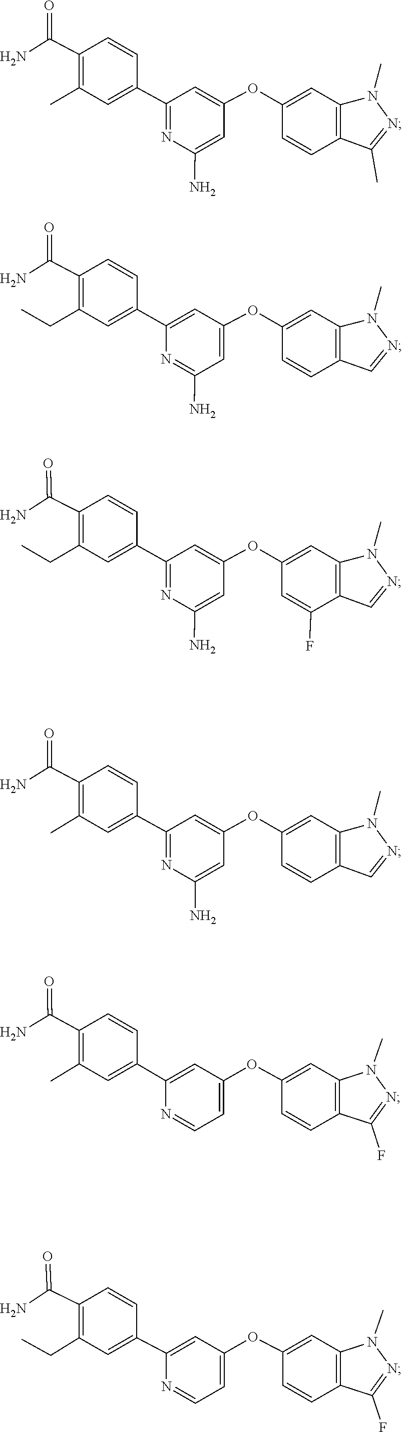

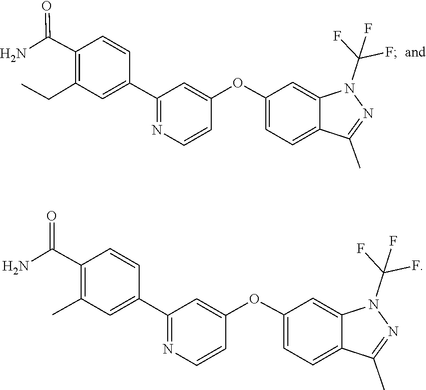

In a further aspect of the disclosure are compounds, or the pharmaceutically acceptable salts thereof, of Formula (Ic)

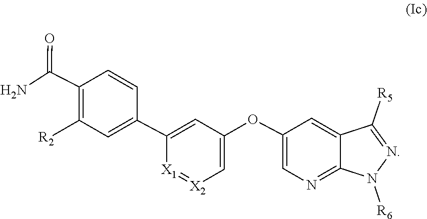

In a further aspect of the disclosure are compounds of Formula (Ic) in which R₂ is selected from C₁₋₂alkyl, halo, methyl-amino and halo-substituted-C₁₋₂alkyl. In a further aspect of the disclosure are compounds of Formula (Ic) in which R₅ is selected from hydrogen, C₁₋₂alkyl and halo-substituted-C₁₋₂alkyl.

In a further aspect of the disclosure are compounds of Formula (Ic) in which R₆ is selected from hydrogen and C₁₋₂alkyl.

In a further aspect of the disclosure are compounds of Formula (Ic) in which X₁ is selected from N and CH.

In a further aspect of the disclosure are compounds of Formula (Ic) in which X₂ is selected from N and CR₉; wherein R₉ is selected from hydrogen, amino, amino-methyl, methyl-amino-methyl, azetidin-2-yl and azetidin-3-yl.

In a further aspect of the disclosure are compounds of Formula (Ic) in which:
R₂ is selected from C₁₋₂alkyl, halo, methyl-amino and halo-substituted-C₁₋₂alkyl;
R₅ is selected from hydrogen, C₁₋₂alkyl and halo-substituted-C₁₋₂alkyl;
R₆ is selected from hydrogen and C₁₋₂alkyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Ic) in wherein:
R₂ is selected from methyl, ethyl, methyl-amino, chloro and trifluoro-methyl;
R₅ is selected from hydrogen, fluoro, methyl, ethyl and trifluoromethyl;
R₆ is selected from hydrogen, methyl, amino, cyano, and trifluoromethyl;
X₁ is selected from N and CH;
X₂ is selected from N and CR₈; wherein R₅ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Ic) wherein:
R₂ is selected from methyl and ethyl;
X₁ is N;
X₂ is CR₉; wherein R₉ is selected from hydrogen and amino; and the pharmaceutically acceptable salts thereof.

In a further aspect of the disclosure are compounds of Formula (Ic), or a pharmaceutically acceptable salt thereof, selected from

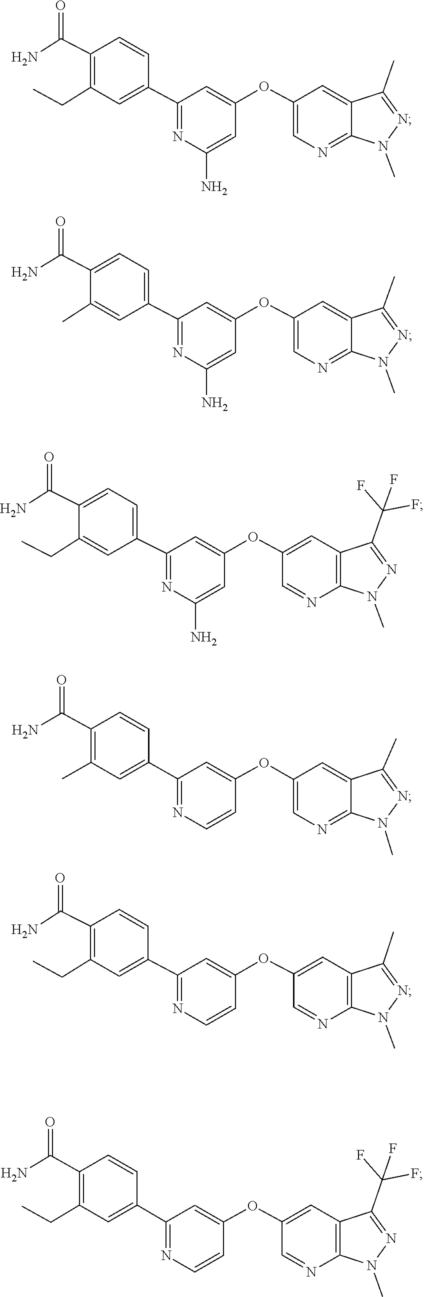 [SMILES: CCc1cc(-c2cc(Oc3cnc4c(c3)c(C)nn4C)cc(N)n2)ccc1C(N)=O ; Cc1cc(-c2cc(Oc3cnc4c(c3)c(C)nn4C)cc(N)n2)ccc1C(N)=O ; CCc1cc(-c2cc(Oc3cnc4c(c3)c(C(F)(F)F)nn4C)cc(N)n2)ccc1C(N)=O ; Cc1cc(-c2cc(Oc3cnc4c(c3)c(C)nn4C)ccn2)ccc1C(N)=O ; CCc1cc(-c2cc(Oc3cnc4c(c3)c(C)nn4C)ccn2)ccc1C(N)=O ; CCc1cc(-c2cc(Oc3cnc4c(c3)c(C(F)(F)F)nn4C)ccn2)ccc1C(N)=O]

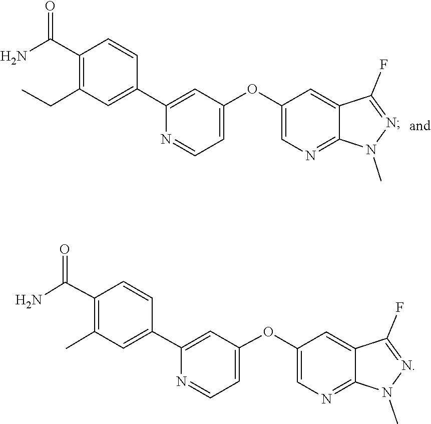 [SMILES: CCc1cc(-c2cc(Oc3cnc4c(c3)c(F)nn4C)ccn2)ccc1C(N)=O ; Cc1cc(-c2cc(Oc3cnc4c(c3)c(F)nn4C)ccn2)ccc1C(N)=O]

In another aspect of the disclosure are compounds, or a pharmaceutically acceptable salt thereof, of Formula (Id):

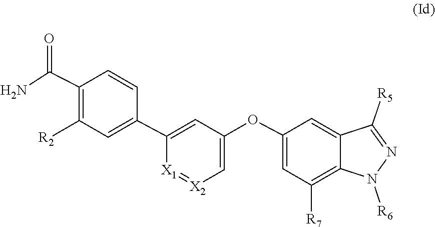

In a further aspect of the disclosure are compounds of Formula (Id) in which R₂ is selected from C₁₋₂alkyl, halo, methyl-amino and halo-substituted-C₁₋₂alkyl. In a further aspect of the disclosure are compounds of Formula (Id) in which R₅ is selected from hydrogen, C₁₋₂alkyl, halo and halo-substituted-C₁₋₂alkyl.

In a further aspect of the disclosure are compounds of Formula (Id) in which R₆ is selected from hydrogen and C₁₋₂alkyl.

In a further aspect of the disclosure are compounds of Formula (Id) in which R₇ is selected from hydrogen, C₁₋₂alkyl and halo.

In a further aspect of the disclosure are compounds of Formula (Id) in which X₁ is selected from N and CH.

In a further aspect of the disclosure are compounds of Formula (Id) in which X₂ is selected from N and CR₉; wherein R₉ is selected from hydrogen, amino, amino-methyl, methyl-amino-methyl, azetidin-2-yl and azetidin-3-yl.

In a further aspect of the disclosure are compounds of Formula (Id) in which:
R₂ is selected from C₁₋₂alkyl, halo, methyl-amino and halo-substituted-C₁₋₂alkyl;
R₅ is selected from hydrogen, C₁₋₂alkyl, halo and halo-substituted-C₁₋₂alkyl;
R₆ is selected from hydrogen and C₁₋₂alkyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl;
and the pharmaceutically acceptable salts thereof.

In a further aspect of the disclosure are compounds of Formula (Id) in which:
R₂ is selected from methyl, ethyl, methyl-amino, chloro and trifluoro-methyl;
R₅ is selected from hydrogen, fluoro, methyl and ethyl;
R₆ is selected from hydrogen, methyl, amino, cyano, and trifluoromethyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

In a further aspect of the disclosure are compounds of Formula (Id) in which R₂ is selected from methyl, ethyl and chloro; X₁ is N; X₂ is CH; and the pharmaceutically acceptable salts thereof.

In a further aspect of the disclosure are compounds of Formula (Id), or a pharmaceutically acceptable salt thereof, selected from

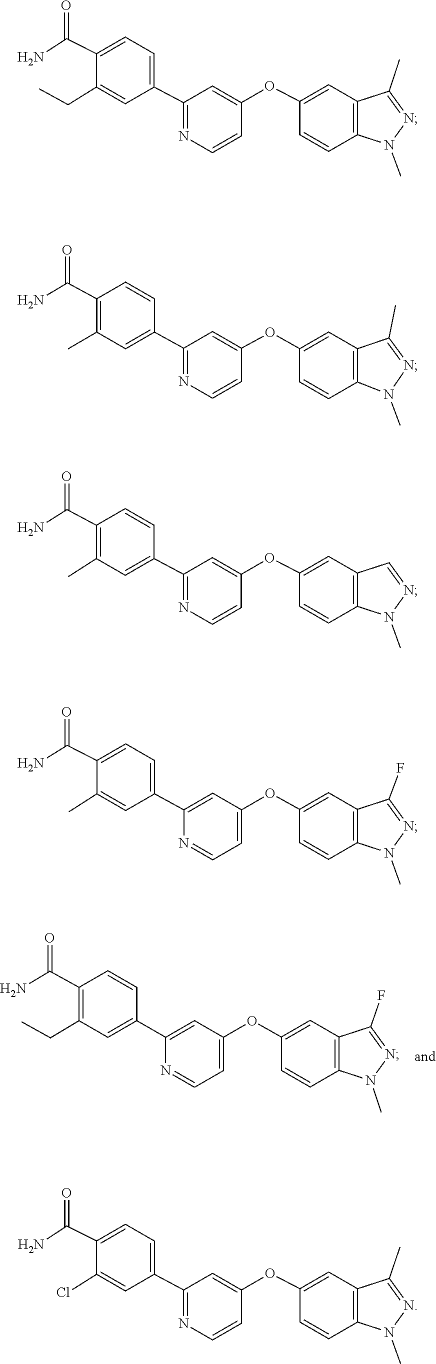

In another aspect of the disclosure are compounds, or pharmaceutically acceptable salts thereof, of Formula (Ie):

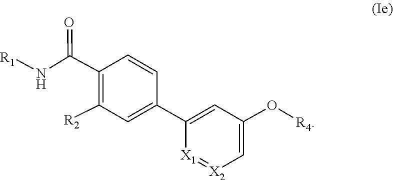

In a further aspect of the disclosure are compounds of Formula (Ie) in which R₁ is selected from hydrogen and C₁₋₂alkyl.

In a further aspect of the disclosure are compounds of Formula (Ie) in which R₂ is selected from C₁₋₂alkyl, halo, methyl-amino and halo-substituted-C₁₋₂alkyl.

In a further aspect of the disclosure are compounds of Formula (Ie) in which R₄ is selected from

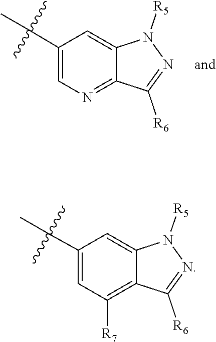

In a further aspect of the disclosure are compounds of Formula (Ie) in which R₅ is selected from hydrogen and C₁₋₂alkyl.

In a further aspect of the disclosure are compounds of Formula (Ie) in which R₆ is selected from hydrogen, amino, cyano, C₁₋₂alkyl and halo-substituted-C₁₋₂alkyl.

In a further aspect of the disclosure are compounds of Formula (Ie) in which R₇ is selected from hydrogen, C₁₋₂alkyl and halo.

In a further aspect of the disclosure are compounds of Formula (Ie) in which X₁ is selected from N and CH.

In a further aspect of the disclosure are compounds of Formula (Ie) in which X₂ is selected from N and CR₉; wherein R₉ is selected from hydrogen, amino, amino-methyl, methyl-amino-methyl, azetidin-2-yl and azetidin-3-yl; or R₉ and the nitrogen of X₁ form a 5-member unsaturated ring containing up to two nitrogen atoms.

In a further aspect of the disclosure are compounds of Formula (Ie) in which:
R₁ is selected from hydrogen and C₁₋₂alkyl;
R₂ is selected from C₁₋₂alkyl, halo, methyl-amino and halo-substituted-C₁₋₂alkyl;
R₄ is selected from

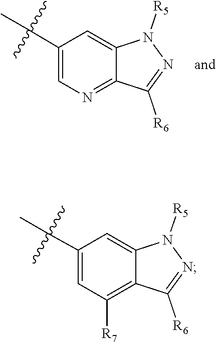

R₅ is selected from hydrogen and C₁₋₂alkyl;
R₆ is selected from hydrogen, amino, cyano, C₁₋₂alkyl and halo-substituted-C₁₋₂alkyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; or R₉ and the nitrogen of X₁ form a 5-member unsaturated ring containing up to two nitrogen atoms; and the pharmaceutically acceptable salts thereof.

In a further aspect of the disclosure are compounds of Formula (Ie) wherein:
R₁ is selected from hydrogen, methyl and ethyl;
R₂ is selected from methyl and ethyl;
R₄ is selected from

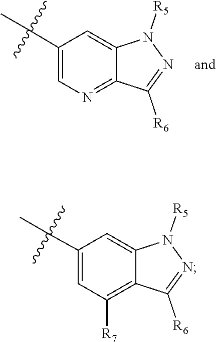

R₅ is methyl;
R₆ when attached to a carbon atom is selected from hydrogen, methyl and trifluoromethyl;
R₇ is hydrogen;
X₁ is selected from N and CH;
X₂ is CR₉; wherein R₉ is hydrogen; and the pharmaceutically acceptable salts thereof.

In a further aspect of the disclosure are compounds of Formula (Ie), or a pharmaceutically acceptable salt thereof, selected from

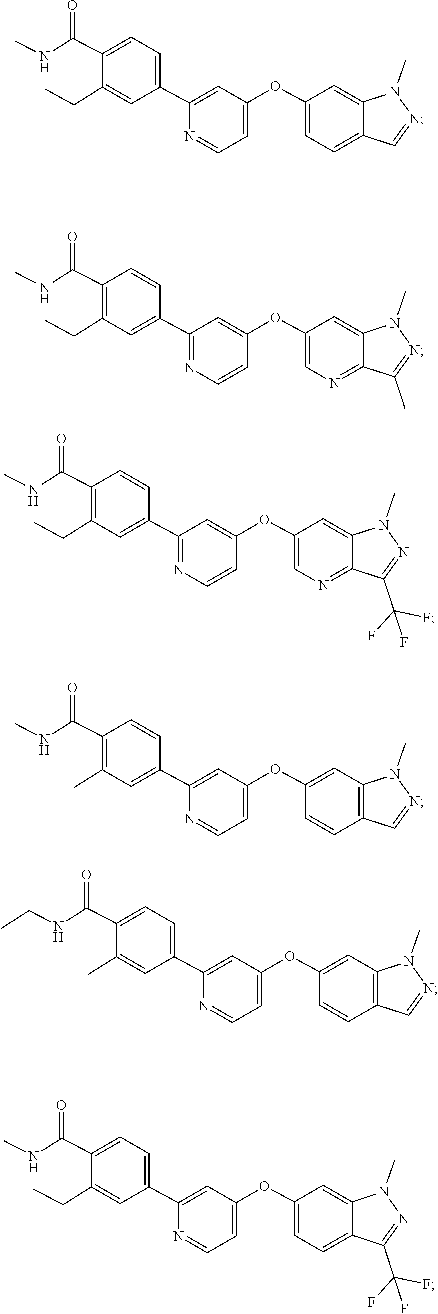 [SMILES: CCc1cc(-c2cc(Oc3ccc4cnn(C)c4c3)ccn2)ccc1C(=O)NC ; CCc1cc(-c2cc(Oc3cnc4c(C)nn(C)c4c3)ccn2)ccc1C(=O)NC ; CCc1cc(-c2cc(Oc3cnc4c(C(F)(F)F)nn(C)c4c3)ccn2)ccc1C(=O)NC ; CNC(=O)c1ccc(-c2cc(Oc3ccc4cnn(C)c4c3)ccn2)cc1C ; CCNC(=O)c1ccc(-c2cc(Oc3ccc4cnn(C)c4c3)ccn2)cc1C ; CCc1cc(-c2cc(Oc3ccc4c(C(F)(F)F)nn(C)c4c3)ccn2)ccc1C(=O)NC]

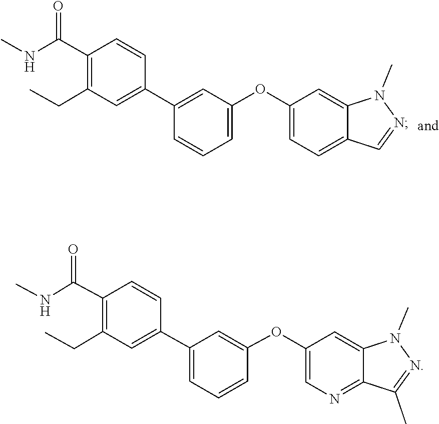 [SMILES: CCc1cc(-c2cccc(Oc3ccc4cnn(C)c4c3)c2)ccc1C(=O)NC ; CCc1cc(-c2cccc(Oc3cnc4c(C)nn(C)c4c3)c2)ccc1C(=O)NC]

In another aspect of the disclosure are compounds, or a pharmaceutically acceptable salt thereof, of Formula If:

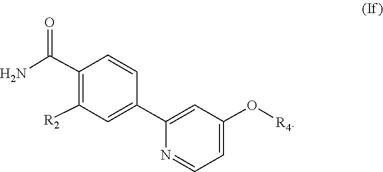

In a further aspect of the disclosure are compounds of Formula (If) in which R₂ is selected from C₁₋₂alkyl, halo, methyl-amino and halo-substituted-C₁₋₂alkyl.

In a further aspect of the disclosure are compounds of Formula (If) in which R₄ is selected from

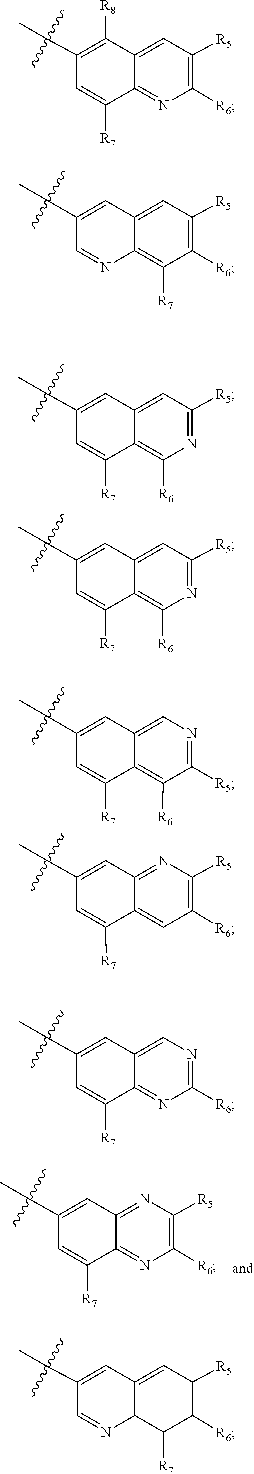

In a further aspect of the disclosure are compounds of Formula (If) in which R₅ is selected from hydrogen, C₁₋₂alkyl, halo and halo-substituted-C₁₋₂alkyl.

In a further aspect of the disclosure are compounds of Formula (If) in which R₆ is selected from hydrogen, amino, cyano, C₁₋₂alkyl, halo and halo-substituted-C₁₋₂alkyl.

In a further aspect of the disclosure are compounds of Formula (If) in which R₇ is selected from hydrogen, C₁₋₂alkyl, halo and halo-substituted-C₁₋₂alkyl.

In a further aspect of the disclosure are compounds of Formula (If) in which R₈ is selected from hydrogen and halo.

In a further aspect of the disclosure are compounds of Formula (If) in which:
R₂ is selected from C₁₋₂alkyl, halo, methyl-amino and halo-substituted-C₁₋₂alkyl;
R₄ is selected from

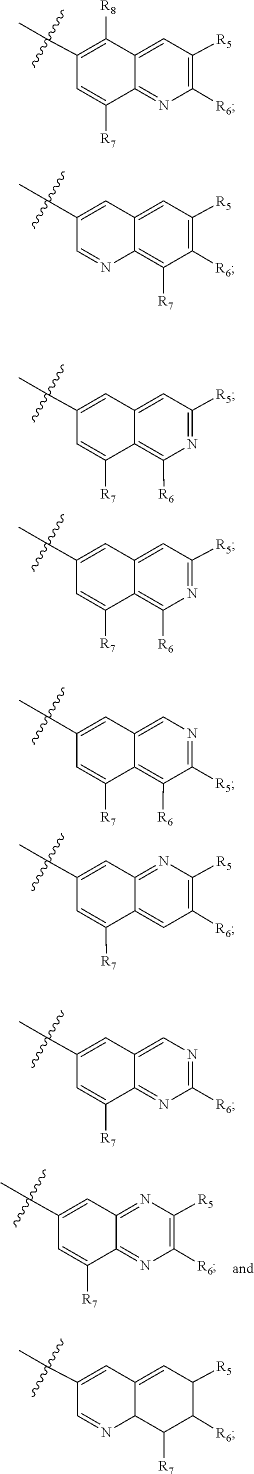

R₅ is selected from hydrogen, C₁₋₂alkyl, halo and halo-substituted-C₁₋₂alkyl;
R₆ is selected from hydrogen, amino, cyano, C₁₋₂alkyl, halo and halo-substituted-C₁₋₂alkyl;
R₇ is selected from hydrogen, C₁₋₂alkyl, halo and halo-substituted-C₁₋₂alkyl;
R₈ is selected from hydrogen and halo;
and the pharmaceutically acceptable salts thereof.

In a further aspect of the disclosure are compounds of Formula (If) in which R₂ is selected from methyl, ethyl, chloro, fluoro and trifluoromethyl; R₄ is selected from:

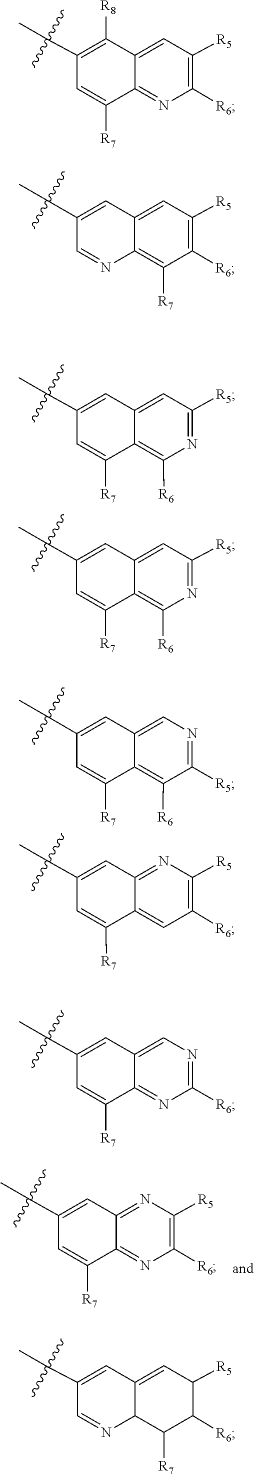

R₅ when attached to a carbon atom is selected from hydrogen, fluoro, chloro, and methyl;
R₆ is selected from hydrogen and fluoro;
R₇ is selected from hydrogen, fluoro, chloro and trifluoromethyl;
R₈ is selected from hydrogen and fluoro;
and the pharmaceutically acceptable salts thereof.

In a further aspect of the disclosure are compounds of Formula (If), or a pharmaceutically acceptable salt thereof, selected from

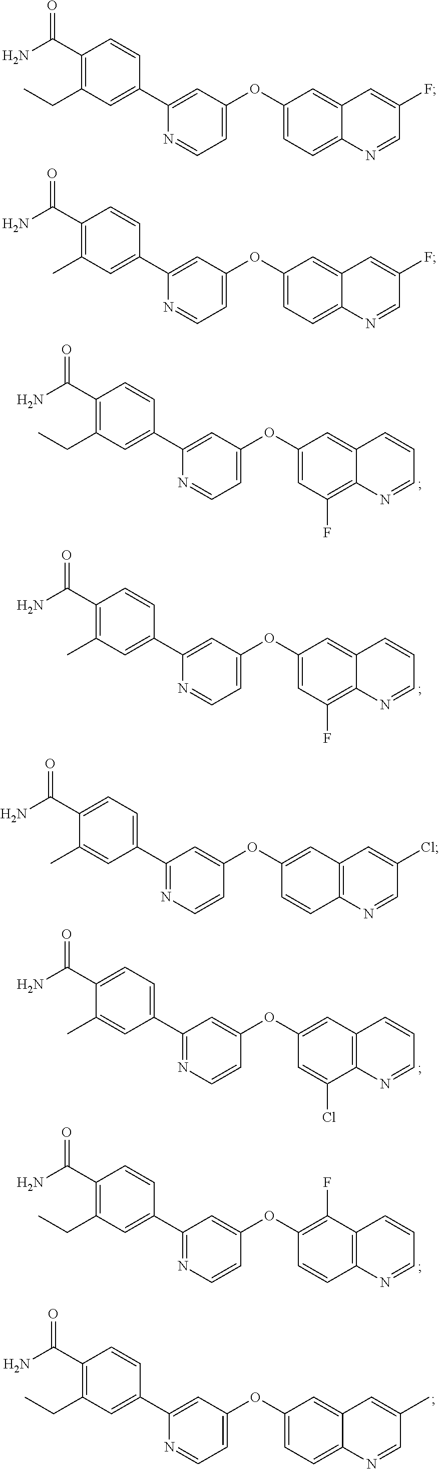

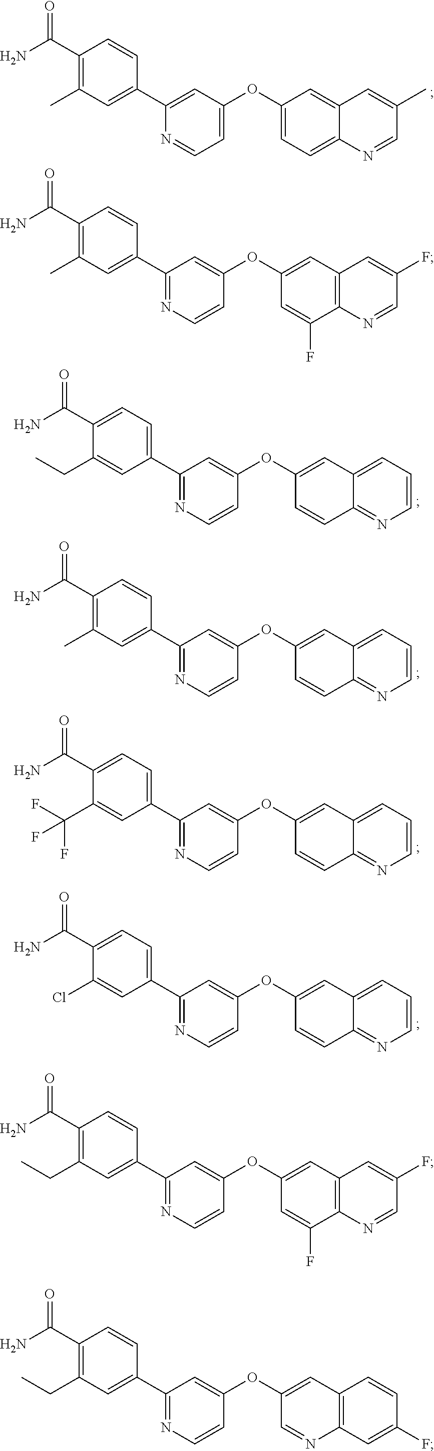

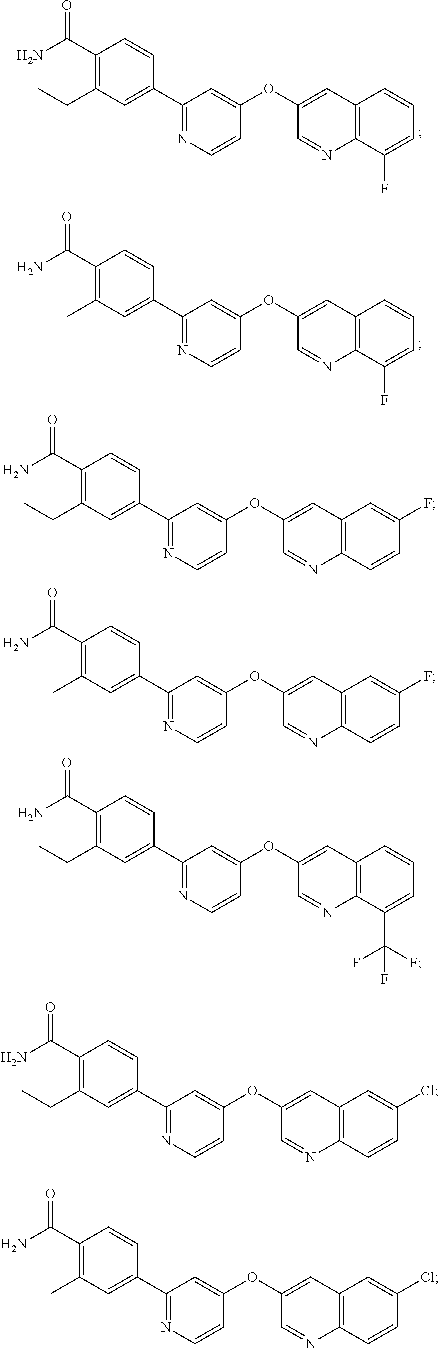

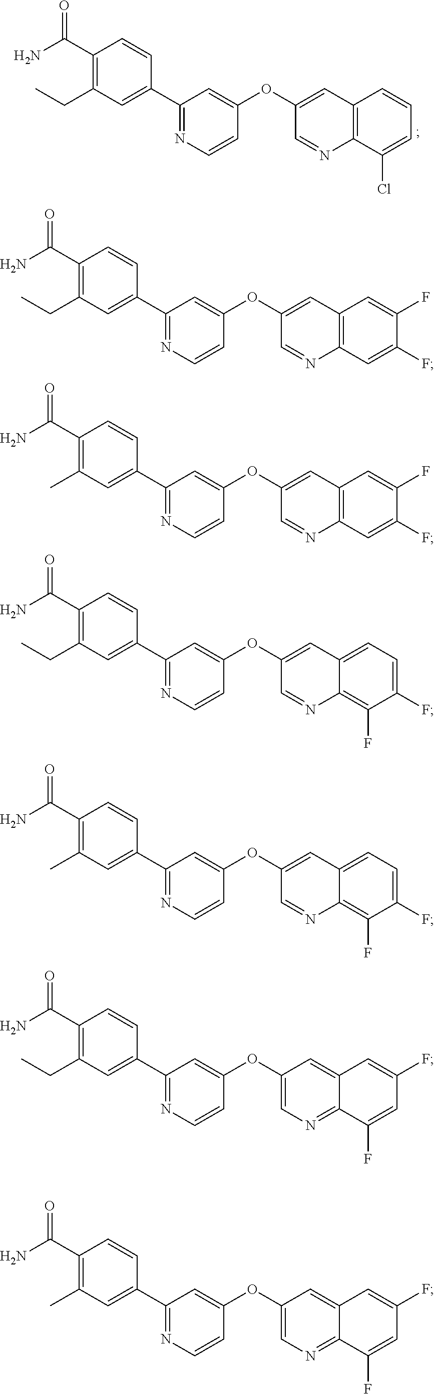

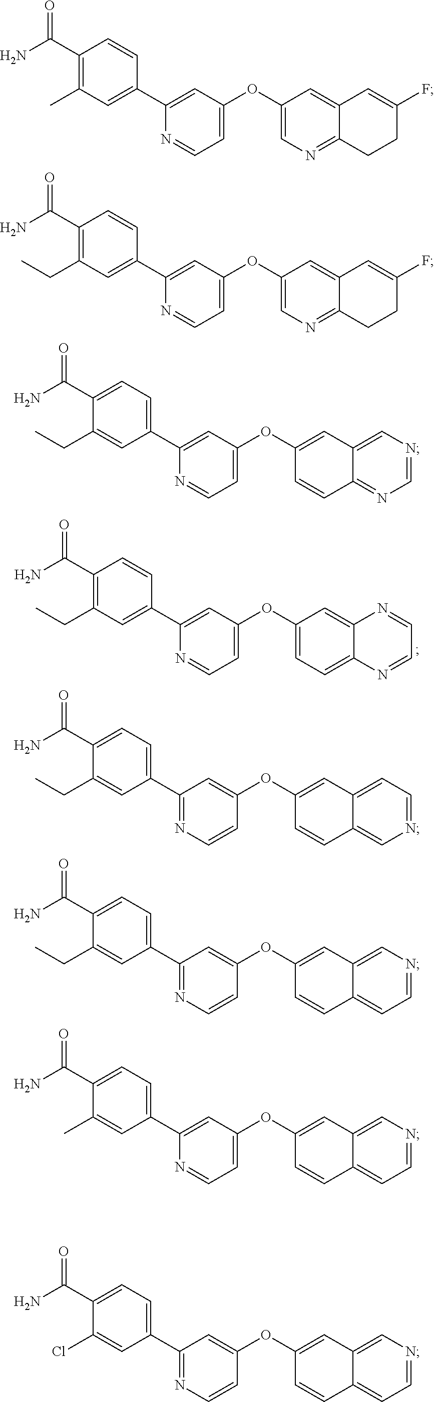

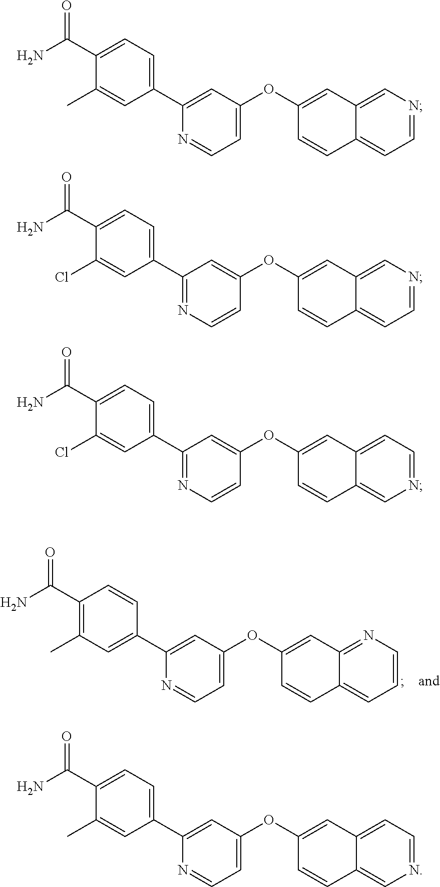 [SMILES: Cc1cc(-c2cc(Oc3ccc4ccncc4c3)ccn2)ccc1C(N)=O ; NC(=O)c1ccc(-c2cc(Oc3ccc4ccncc4c3)ccn2)cc1Cl ; NC(=O)c1ccc(-c2cc(Oc3ccc4cnccc4c3)ccn2)cc1Cl ; Cc1cc(-c2cc(Oc3ccc4cccnc4c3)ccn2)ccc1C(N)=O ; Cc1cc(-c2cc(Oc3ccc4cnccc4c3)ccn2)ccc1C(N)=O]

In a further aspect of the disclosure are compounds, or a pharmaceutically acceptable salt thereof, selected from:

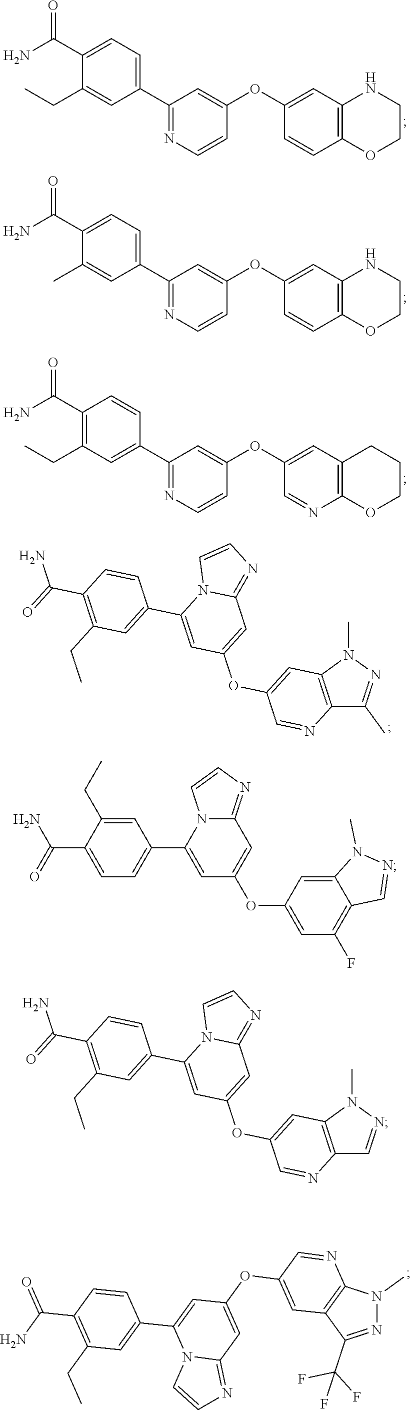

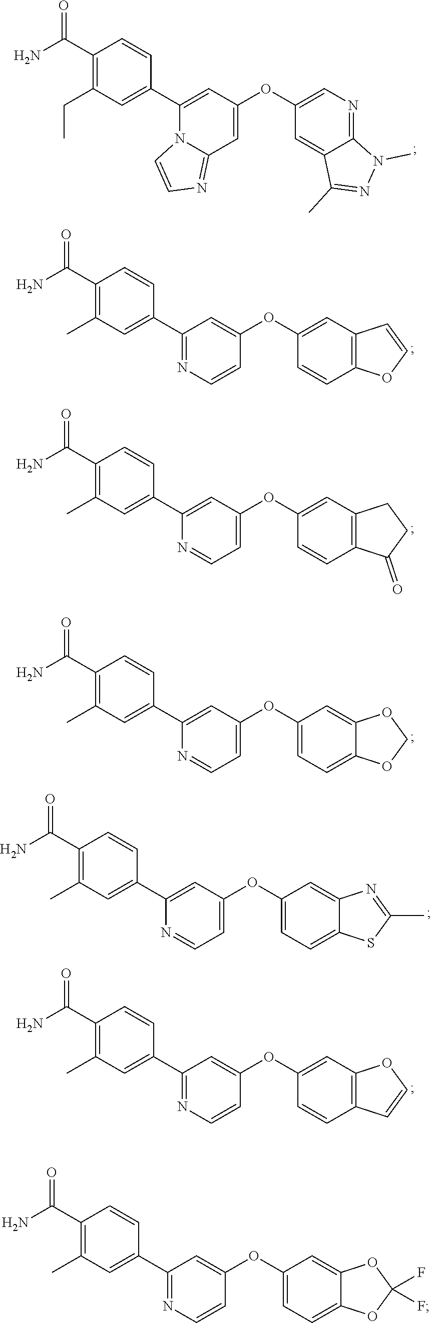

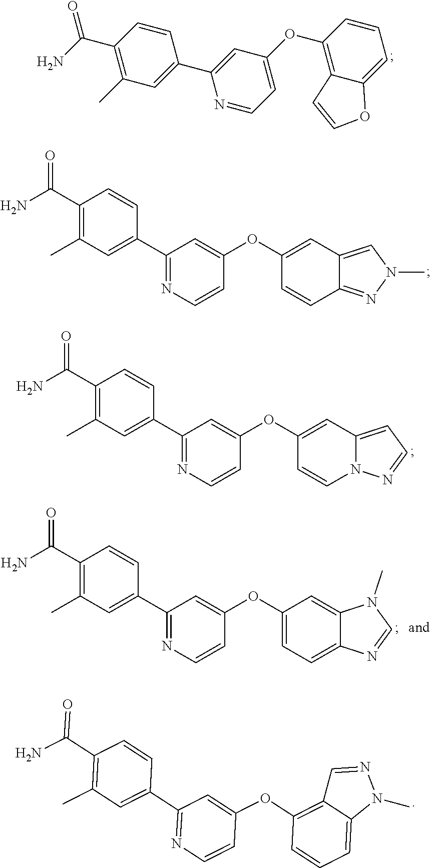 [SMILES: Cc1cc(-c2cc(Oc3cccc4occc34)ccn2)ccc1C(N)=O ; Cc1cc(-c2cc(Oc3ccc4nn(C)cc4c3)ccn2)ccc1C(N)=O ; Cc1cc(-c2cc(Oc3ccn4nccc4c3)ccn2)ccc1C(N)=O ; Cc1cc(-c2cc(Oc3ccc4ncn(C)c4c3)ccn2)ccc1C(N)=O ; Cc1cc(-c2cc(Oc3cccc4c3cnn4C)ccn2)ccc1C(N)=O]

### Pharmaceutical Compositions, Formulation, and Dosage Forms

The present disclosure further provides for pharmaceutical products such as pharmaceutical compositions, formulations, unit dosage forms, and kits; each comprising a compound of Formula (I), or a pharmaceutically acceptable salt thereof.

The present disclosure also provides for pharmaceutical compositions comprising any of the compounds described herein (e.g., a compound of Formula (I), including specific compounds described herein) or pharmaceutically acceptable salts thereof, and an excipient such as a pharmaceutically acceptable excipient. A pharmaceutically acceptable excipient is a physiologically and pharmaceutically suitable non-toxic and inactive material or ingredient that does not interfere with the activity of the drug substance; an excipient also can be called a carrier. The formulation methods and excipients described herein are exemplary and are in no way limiting. Pharmaceutically acceptable excipients are well known in the pharmaceutical art and described, for example, in Rowe et al., Handbook of Pharmaceutical Excipients: A Comprehensive Guide to Uses, Properties, and Safety, 5th Ed., 2006, and in Remington: The Science and Practice of Pharmacy (Gennaro, 21st Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like can be provided in the pharmaceutical composition. In addition, antioxidants and suspending agents may also be used.

In another aspect of the disclosure, with respect to compounds of Formula I, is a pharmaceutical composition comprising a compound of Formula I, or a pharmaceutically acceptable salt thereof, and one or more excipients.

For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a GPR52 agonist, diluents, dispersing and surface-active agents, binders, and lubricants. One skilled in this art may further formulate the GPR52 agonist in an appropriate manner, and in accordance with accepted practices, such as those disclosed in Remington, supra.

Methods of administration include systemic administration of a GPR52 agonist described herein, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds described herein (or pharmaceutically acceptable salts thereof) can be prepared in aqueous injection solutions which may contain, in addition to the GPR52 agonist, buffers, antioxidants, bacteriostats, and other additives commonly employed in such solutions.

Pharmaceutical preparations for oral administration can be obtained by any suitable method, typically by uniformly mixing the compound(s) with liquids or finely divided solid carriers, or both, in the required proportions and then, if necessary, processing the mixture, after adding suitable auxiliaries, if desired, forming the resulting mixture into a desired shape to obtain tablets or dragee cores.

Conventional excipients, such as binding agents, fillers, adjuvant, carrier, acceptable wetting agents, tableting lubricants and disintegrants can be used in tablets and capsules for oral administration. Liquid preparations for oral administration can be in the form of solutions, emulsions, aqueous or oily suspensions and syrups.

Alternatively, the oral preparations can be in the form of dry powder that can be reconstituted with water or another suitable liquid vehicle before use. Additional additives such as suspending or emulsifying agents, non-aqueous vehicles (including edible oils), preservatives and flavorings and colorants can be added to the liquid preparations. Parenteral dosage forms can be prepared by dissolving the compound described herein in a suitable liquid vehicle and filter sterilizing the solution before lyophilization, or simply filling and sealing an appropriate vial or ampule.

Some aspects provide methods for preparing a pharmaceutical composition comprising the step of admixing a compound of Formula (I), or a pharmaceutically acceptable salt thereof, and a pharmaceutically acceptable carrier.

In making pharmaceutical compositions comprising a compound of Formula (I), or pharmaceutically acceptable salts thereof, the drug substance is typically mixed (i.e., admixed) with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier, or medium for the drug substance. Thus, the compositions can be in the form of tablets, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

For preparing solid form pharmaceutical compositions such as powders, tablets, capsules, cachets, suppositories and dispersible granules an excipient can be one or more substances which may also act as diluents, flavoring agents, solubilizers, lubricants, suspending agents, binders, preservatives, tablet disintegrating agents, or an encapsulating material. Also included are solid form preparations which are intended to be converted, shortly before use, to liquid form preparations for oral administration. Such liquid forms include solutions, suspensions and emulsions. These preparations may contain, in addition to the drug substance, colorants, flavors, stabilizers, buffers, artificial and natural sweeteners, dispersants, thickeners, solubilizing agents and the like.

For preparing suppositories, a low melting wax, such as an admixture of fatty acid glycerides or cocoa butter, is first melted and the drug substance is dispersed homogeneously therein, as by stirring. The molten homogenous mixture is then poured into convenient sized molds, allowed to cool and thereby to solidify.

Formulations suitable for vaginal administration can be presented as pessaries, tampons, creams, gels, pastes, foams or sprays containing in addition to the drug substance such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The pharmaceutical compositions may take such forms as suspensions, solutions, or emulsions in oily or aqueous vehicles and may contain formulatory agents such as suspending, stabilizing and/or dispersing agents. Alternatively, the pharmaceutical compositions can be in powder form, obtained by aseptic isolation of sterile solid or by lyophilization from solution, for constitution with a suitable vehicle, e.g., sterile, pyrogen-free water, before use.

The pharmaceutical compositions can be formulated as an aqueous solution, an aqua-alcoholic solution, a solid suspension, an emulsion, a liposomal suspension, or a freeze-dried powder for reconstitution. Such pharmaceutical compositions can be administered directly or as an admixture for further dilution/reconstitution. Route of administration includes intravenous bolus, intravenous infusion, irrigation, and instillation. Suitable solvents include water, alcohols, PEG, propylene glycol, and lipids; pH adjustments using an acid, e.g., HCl or citric acid, can be used to increase solubility and resulting compositions subjected to suitable sterilization procedures know in the art, such as, aseptic filtration. In some aspects, the pH of the aqueous solution is about 2.0 to about 4.0. In some aspects, the pH of the aqueous solution is about 2.5 to about 3.5.

Aqueous formulations suitable for oral use can be prepared by dissolving or suspending the drug substance in water and adding suitable colorants, flavors, stabilizing and thickening agents, as desired.

Aqueous suspensions suitable for oral use can be made by dispersing the finely divided drug substance in water with viscous material, such as natural or synthetic gums, resins, methylcellulose, sodium carboxymethylcellulose, or other well-known suspending agents.

For topical administration to the epidermis the compounds described herein, or pharmaceutically acceptable salts thereof can be formulated as gels, ointments, creams or lotions, or as a transdermal patch. Also, formulations suitable for topical administration in the mouth include lozenges comprising drug substance in a flavored base, usually sucrose and acacia or tragacanth; pastilles comprising the drug substance in an inert base such as gelatin and glycerin or sucrose and acacia; and mouthwashes comprising the drug substance in a suitable liquid carrier. Ointments and creams may, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. In some aspects, topical formulations can contain one or more conventional carriers. In some aspects, ointments can contain water and one or more hydrophobic carriers selected from, for example, liquid paraffin, polyoxyethylene alkyl ether, propylene glycol, white vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like.

Solutions or suspensions can be applied directly to the nasal cavity by conventional means, for example with a dropper, pipette or spray. The formulations can be provided in single or multi-dose form. In the latter case of a dropper or pipette, this can be achieved by the subject administering an appropriate, predetermined volume of the solution or suspension. In the case of a spray, this can be achieved for example by means of a metering atomizing spray pump.

Administration to the respiratory tract may also be achieved by means of an aerosol formulation provided in a pressurized pack with a suitable propellant. If the compounds described herein, or pharmaceutically acceptable salts thereof or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds described herein (or pharmaceutically acceptable salts thereof), as an aerosol can be prepared by processes well known to the person skilled in the art. For their preparation, for example, solutions or dispersions of the compounds described herein (or pharmaceutically acceptable salts thereof), in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug can be controlled by provision of a metered valve.

Alternatively, the pharmaceutical composition can be provided in the form of a dry powder, for example, a powder mix of the compound in a suitable, powder base such as lactose, starch, starch derivatives such as hydroxypropylmethyl cellulose and polyvinylpyrrolidone (PVP). Conveniently the powder carrier will form a gel in the nasal cavity. The powder composition can be presented in unit dose form for example in capsules or cartridges of, e.g., gelatin, or blister packs from which the powder can be administered by means of an inhaler.

The compounds of Formula (I), or pharmaceutically acceptable salts thereof may also be administered via a rapid dissolving or a slow release composition, wherein the composition includes a biodegradable rapid dissolving or slow release carrier (such as a polymer carrier and the like). Rapid dissolving or slow release carriers are well known in the art and are used to form complexes that capture therein compounds of

Formula (I), or pharmaceutically acceptable salts thereof and either rapidly or slowly degrade/dissolve in a suitable environment (e.g., aqueous, acidic, basic, etc.).

The pharmaceutical preparations are preferably in unit dosage forms. In such form, the preparation is subdivided into unit doses containing appropriate quantities of the drug substance. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form. In some aspects, the pharmaceutical preparation is a tablet or capsule for oral administration. In some aspects, the pharmaceutical preparation is a liquid formulated for intravenous administration.

The compositions can be formulated in a unit dosage form, each dosage containing the drug substance or equivalent mass of the drug substance. The term “unit dosage forms” refers to physically discrete units of a formulation suitable as unitary dosages for human subjects and other mammals, each unit containing a predetermined quantity of drug substance calculated to produce the desired therapeutic effect, in association with a suitable excipient, as described herein.

The compositions described herein can be formulated to provide immediate and/or timed release (also called extended release, sustained release, controlled release, or slow release) of the drug substance after administration to a subject by employing procedures known in the art. For example, the tablets including compounds of Formula (I), or pharmaceutically acceptable salts thereof, can be coated or otherwise compounded to provide a dosage form affording the advantage of prolonged action. For example, the tablet can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including several polymeric acids and mixtures of polymeric acids with such materials as shellac, cetyl alcohol, and cellulose acetate.

The liquid forms including the drug substance can be incorporated for administration orally or by injection include aqueous solutions, suitably flavored syrups, aqueous or oil suspensions, and flavored emulsions with edible oils such as cottonseed oil, sesame oil, coconut oil, or peanut oil, and similar excipients.

The pharmaceutical compositions described herein can be sterilized by conventional sterilization techniques, or can be sterile filtered. Aqueous solutions can be packaged for use as is, or lyophilized, the lyophilized preparation being combined with a sterile aqueous carrier prior to administration. The pH of the compound preparations is typically between 3 and 11, more preferably from 5 to 9 and most preferably from 7 to 8. It will be understood that use of certain of the foregoing excipients may result in the formation of pharmaceutically acceptable salts.

Compositions for inhalation or insufflation include solutions and suspensions in pharmaceutically acceptable, aqueous or organic solvents, or mixtures thereof, and powders. The liquid or solid compositions may contain suitable excipients as described herein. In some aspects, the compositions are administered by the oral or nasal respiratory route for local or systemic effect. Compositions can be nebulized by use of inert gases. Nebulized solutions can be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner. The compositions may, if desired, be presented in a pack or dispenser device which may contain one or more-unit dosage forms containing the drug substance. The pack may for example comprise metal or plastic foil, such as a blister pack. The pack or dispenser device can be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

For preparing solid compositions such as tablets, the drug substance can be mixed with an excipient to form a solid preformulation composition containing a homogeneous mixture of components. When referring to these preformulation compositions as homogeneous, the drug substance is typically dispersed evenly throughout the composition so that the composition can be readily subdivided into equally effective unit dosage forms such as tablets and capsules.

Kits with unit doses of one or more of the compounds described herein, usually in oral or injectable doses, are provided. Such kits may include a container containing the unit dose, an informational package insert describing the use and attendant benefits of the drugs in treating pathological condition of interest, and optionally an appliance or device for delivery of the composition.

The compounds described herein, or a pharmaceutically acceptable salt thereof, can be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The amount of compound or composition administered to a subject will also vary depending upon what is being administered, the purpose of the administration, such as prophylaxis or therapy, the state of the subject, the manner of administration, and the like. In therapeutic applications, compositions can be administered to a subject already suffering from a disease in an amount sufficient to cure or at least partially arrest the symptomology and/or pathology of the disease and its complications.

Therapeutically effective doses will depend on the disease condition being treated as well as by the judgment of the attending clinician depending upon factors such as the severity of the disease, the age, weight and general condition of the subject, and the like.

The desired dose may conveniently be presented in a single dose or presented as divided doses administered at appropriate intervals, for example, as two, three, four, or more sub-doses per day. The sub-dose itself can be further divided, e.g., into a number of discrete loosely spaced administrations. The daily dose can be divided, especially when relatively large amounts are administered as deemed appropriate, into several, for example two, three, or four-part administrations. If appropriate, depending on individual behavior, it can be necessary to deviate upward or downward from the daily dose indicated.

It will be apparent to those skilled in the art that the dosage forms described herein may comprise a compound described herein or pharmaceutically acceptable salt thereof.

Some aspects provide use of a least one compound as disclosed and described herein, or a pharmaceutically acceptable salt thereof, or a pharmaceutical composition as disclosed and described herein, in the manufacture of a medicament for treating a neurological disorder, wherein the neurological disorder is selected from the group consisting of schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome,

Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder (OCD), an autism spectrum disorder, a prolactin-related disorder (e.g., hyperprolactinemia), a neurocognitive disorder, a trauma- or stressor-related disorder (e.g., PTSD); a disruptive, impulse-control, or conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia; Parkinson's Disease, drug induced Parkinsonism, dyskinesias, dystonia, chorea, levodopa induced dyskinesia, cerebral palsy and progressive supranuclear palsy, and Huntington's disease, including chorea associated with Huntington's disease.

Some aspects provide use of a least one compound as disclosed and described herein, or a pharmaceutically acceptable salt thereof, or a pharmaceutical composition as disclosed and described herein, in the manufacture of a medicament for ameliorating one or more symptoms of a neurological disorder, wherein the neurological disorder is selected from the group consisting of schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder (OCD), an autism spectrum disorder, a prolactin-related disorder (e.g., hyperprolactinemia), a neurocognitive disorder, a trauma- or stressor-related disorder (e.g., PTSD); a disruptive, impulse-control, or conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia; Parkinson's Disease, drug induced Parkinsonism, dyskinesias, dystonia, chorea, levodopa induced dyskinesia, cerebral palsy and progressive supranuclear palsy, and Huntington's disease, including chorea associated with Huntington's disease.

Some aspects provide use of a least one compound as disclosed and described herein, or a pharmaceutically acceptable salt thereof, or a pharmaceutical composition as disclosed and described herein, in the manufacture of a medicament for treating a neurological disorder, wherein the neurological disorder is schizophrenia or cognitive impairment associate with schizophrenia (CIAS).

Some aspects provide use of a least one compound as disclosed and described herein, or a pharmaceutically acceptable salt thereof, or a pharmaceutical composition as disclosed and described herein, as standalone or an add-on therapy to standard of care with antipsychotics for the treatment of cognitive impairment associated with Schizophrenia (CIAS).

Some aspects provide use of a least one compound as disclosed and described herein, or a pharmaceutically acceptable salt thereof, or a pharmaceutical composition as disclosed and described herein, as standalone or an add-on therapy to standard of care with antipsychotics for the treatment of negative symptoms of schizophrenia, disorders of impulsivity or compulsivity, non-motor symptoms of Parkinson's Disease, autism spectrum disorder, other CNS disorders with associated cognitive dysfunction (such as Huntington's Disease, Multiple Sclerosis, etc.)

Some aspects provide use of a least one compound as disclosed and described herein, or a pharmaceutically acceptable salt thereof, or a pharmaceutical composition as disclosed and described herein, as standalone or an add-on therapy to standard of care for the treatment psychosis (positive symptoms).

### Pharmacology and Utility

G-protein coupled receptors (GPCRs) possess seven conserved membrane-spanning domains connecting at least eight cytoplasmic loops. The transmembrane regions are designated as TM1, TM2, TM3, TM4, TM5, TM6, and TM7. Most GPCRs contain potential phosphorylation sites within the third cytoplasmic loop and/or the carboxy terminus. GPCRs are critical components of many cell-signaling pathways. GPCRs are coupled to various enzymes, ion channels, and transporters. Different G-protein subunits may stimulate effectors to modulate various downstream functions in a cell.

Ligand binding causes a conformational change in a GPCR, allowing the GPCR to function as a guanine nucleotide exchange factor (GEF). The GPCR can then activate an associated G protein by exchanging the GDP bound to the G protein for GTP. This GTP, together with the a subunit of the G protein, then dissociate from the β and γ subunits to further modulate intracellular signaling pathways.

GPR52 is a GPCR that is highly conserved in vertebrates with over 90% of amino acid sequence identity. The highest expression levels within the central nervous system (CNS) are found in the striatum. Lower, significant expression levels are found in other structures in the CNS, including in the cortex. GPR52 tissue distribution has no significant differences between human, rat and mouse suggesting common functions for GPR52 that are independent of species.

In rat brain, GPR52 is expressed in neurons in a variety of regions, including the medial prefrontal cortex, basolateral amygdaloid, and habenular nuclei, which are responsible for manifestations of psychiatric diseases. Further, GPR52 knockout and transgenic mice exhibited psychosis-related and antipsychotic-like behaviors, respectively (Hidetoshi Komatsu, et al., February 2014, Volume 9, Issue 2, PLOS ONE, e90134).

While GPR52 has been characterized, it remains an orphan receptor, that is, it has no known endogenous ligand. Several surrogate ligands have been reported including GPR52's own extracellular loop 2 (ECL2) (Pingyuan Wang, et al., J. Med. Chem., 2020, 63, 13951-72). GPR52 is often co-localized with dopamine receptors (D1 and D2). (See PLOS One, Vol. 9, No. 2, e90134). GPR52 co-localizes almost exclusively with the D2 receptor in the human striatum, and with the D1 receptor in the cortex. The efficacy of existing antipsychotic drugs is mediated by D2 antagonist activity, but this activity comes with side effects such as motor symptoms and hyperprolactinemia. Antipsychotic drugs are also associated with significant side effect profiles, including weight gain, metabolic syndrome, diabetes, hyperlipidemia, hyperglycemia, insulin resistance, extrapyramidal symptoms, and tardive dyskinesia. GPR52 modulators, by contrast, can function essentially as a D2 antagonist and therefore exhibit antipsychotic efficacy while avoiding D2 antagonist related side effects. As such, GPR52 modulators can improve the symptoms of various neurological conditions, diseases, and disorders and represent a target for treating various neurological diseases including, but not limited to, psychotic disorders, detachment, anxiety, anxiety/tension associated with psychoneurosis, acute mania, agitation, mania in bipolar disorder, dysthymia, dyspepsia, and drug associated addictions, such as cocaine, amphetamine or the like.

GPR52 is co-localized with the D1 receptor in the medial prefrontal cortex, but co-localized with the D2 receptor in the basal ganglia, suggesting that GPR52 may be involved in dopaminergic transmission at D1 receptor-expressing neurons in cortex and D2 receptor-expressing neurons in striatum (Hidetoshi Komatsu, et al., February 2014, Volume 9, Issue 2, PLOS ONE, e90134).

Hypofrontality, the decreased blood flow in the prefrontal cortex, is symptomatic of several neurological conditions, including the cognitive and negative symptoms associated with schizophrenia, attention deficit/hyperactivity disorder (ADHD), bipolar disorder, major depressive disorder, and hypofrontality associated with substance abuse. Increasing function in the prefrontal cortex with a GPR52 modulator would, therefore, be useful for the treatment of symptoms associated with hypofrontality.

In one aspect of the disclosure is a method of treating a hypofrontality related disease or disorder comprising administering to a patient in need thereof an effective amount of a compound of Formula I, or a pharmaceutically acceptable salt thereof.

In a further aspect of the disclosure, the hypofrontality related disease or disorder is selected from the cognitive and negative symptoms associated with schizophrenia, attention deficit/hyperactivity disorder (ADHD), bipolar disorder, major depressive disorder, and hypofrontality associated with substance abuse.

In a further aspect, the negative symptoms associated with Schizophrenia, which interrupt a person's typical emotions, behaviors, and abilities are selected from reduction in speaking, odd emotional responses to situations, a lack of emotion or expressions, loss of interest or excitement for life, social isolation, trouble experiencing pleasure, difficulty beginning or following through with plans, and difficulty completing normal everyday activities.

Further, in relation to GPR52 agonists functionally resembling D1 agonists, GPR52 agonists have the potential to be useful for the treatment of disorders treatable by D1 agonists, including but not limited to drug related addictions (e.g., cocaine addiction), hypertension, restless leg syndrome, Parkinson's disease, and depression. Furthermore, based on its expression pattern and functional coupling, GPR52 agonists are useful for the treatment of the cognitive deficits associated with schizophrenia, schizoaffective, schizophreniform and schizotypal disorders, treatment resistant schizophrenia, attenuated psychosis syndrome and autism-spectrum disorder, bipolar disease, Alzheimer's disease, Parkinson's disease, Frontotemporal dementia (Pick's disease), Lewy-body dementia, Vascular dementia, post-stroke dementia, and Creutzfeldt-Jakob disease.

The striatum is involved in the control of movement, including, but not limited to, hyperkinetic movement disorders characterized by excessive abnormal involuntary movements (known as hyperkinesias). Examples of hyperkinetic movement disorders include tremors, dystonia, chorea, ballism, athetosis, tics/Tourette's syndrome, Huntington's disease, myoclonus and startle syndromes, stereotypies, and akathisia. Hyperkinesias are associated with the dysfunction of inhibitory, D2-expressing neurons of this pathway. This dysfunction leads to the inability to inhibit movement, resulting in tics, chorea, vocalizations, tremors, and other hyperkinetic symptoms. For example, early hyperkinetic motor symptoms in Huntington's disease are the result of selective damage to the indirect, D2-containing pathway. Further, D2 receptor binding in the striatum is associated with the severity of Tourette syndrome symptoms. The modulation of GPR52 activity can activate the indirect striatal pathway, leading to more inhibitory control over movement and the resolution of hyperkinetic symptoms.

In one aspect of the disclosure is a method of treating a hyperkinetic movement disorder comprising administering to a patient in need thereof an effective amount of a compound of Formula I, or a pharmaceutically acceptable salt thereof.

In a further aspect of the disclosure, the hyperkinetic movement disorder is selected from tremors, dystonia, chorea, ballism, athetosis, tics/Tourette's syndrome, Huntington's disease, myoclonus and startle syndromes, stereotypies, and akathisia.

Huntington's disease is mainly caused by cytotoxicity of the mutant HTT protein with an expanded polyglutamine repeat tract. Lowering the soluble mutant HTT may reduce its downstream toxicity and provide a potential treatment for Huntington's Disease. Knocking out GPR52 significantly reduces mutant HTT levels in the striatum and rescues Huntington's disease associated behavioral phenotypes in a knock-in Huntington's disease mouse model. Further, a GPR52 antagonist reduces mutant HTT levels and rescues Huntington's disease associated phenotypes in cellular and mouse models (Haikun Song, et al., June 2018, Brain, Vol. 141, Issue 6, P. 1782-98).

In one aspect of the disclosure is a method of treating a Huntington's disease comprising administering to a patient in need thereof an effective amount of a compound of Formula I, or a pharmaceutically acceptable salt thereof.

Schizophrenia is a complex neuropsychiatric disorder that affects around 0.3% of the population. It is a severe, chronic, and disabling mental disorder. The core clinical features of schizophrenia include positive, negative and cognitive symptoms. Cognitive impairments associated with schizophrenia (CIAS) are highly detrimental to functional capacity, and the severity of CIAS is the most accurate predictor of patient outcomes. Antipsychotic drugs can reduce the severity of positive symptoms via dopamine D2 receptor antagonism but do not demonstrate significant efficacy for negative and cognitive symptoms. A selective GPR52 agonist showed therapeutic properties for the treatment of positive and cognitive symptoms of schizophrenia (Keiji Nishiyama, et al., J. Pharm. Exp. Ther., November 2017, 363 (2)₂₅₃-64).

The major clinical unmet need in schizophrenia is the treatment of negative and cognitive symptoms, as the currently approved antipsychotics offer little improvement. Notably, a cognitive deficit in patients with schizophrenia is recognized as a core part of the disorder and is believed to have a significant bearing on the patients' recovery and re-integration into society.

In one aspect of the disclosure is a method of treating Schizophrenia comprising administering to a patient in need thereof an effective amount of a compound of Formula I, or a pharmaceutically acceptable salt thereof.

In another aspect of the disclosure is a method of treating CIAS comprising administering to a patient in need thereof an effective amount of a compound of Formula I, or a pharmaceutically acceptable salt thereof.

The psychotic symptoms of schizophrenia result from overactive presynaptic dopamine activity in the striatum. The clinical efficacy of existing antipsychotic drugs for treating psychotic symptoms is dependent on blockade of the D2 receptor. All known antipsychotic drugs with efficacy for the treatment of psychosis are either antagonists or partial agonists at the dopamine D2 receptor. While these antipsychotic drugs can treat the positive (or psychotic) symptoms of schizophrenia, they do not treat other aspects of schizophrenia, such as the negative symptoms or cognitive impairment. Based on the co-expression of the GPR52 and the dopamine D2 receptor, GPR52 agonists should treat the psychotic symptoms associated with schizophrenia. Additionally, since the mechanism of action of GPR52 agonists is unique to known D2 receptor associated antipsychotic drugs, it would be anticipated that GPR52 agonists augment the anti-psychotic efficacy of known neuroleptics. This should result not only in improved anti-psychotic efficacy but could be used to lower the dose of anti-psychotic drugs, thereby lowering their associated side effects. Increased serum prolactin levels is one of the prominent side effect profiles of known D2 receptor antagonist anti-psychotics, whereas GPR52 agonists have been demonstrated to lower serum prolactin levels, therefore, co-application of GPR52 agonists with D2 receptor antagonist anti-psychotics may normalize serum prolactin levels, thereby lowering the side effects associated with the D2 receptor antagonist anti-psychotics. In addition, GPR52 agonists should treat the psychotic symptoms associated with various psychiatric indications, including schizoaffective disorder, schizotypal disorder, schizophreniform disorder, treatment resistant schizophrenia, drug-induced psychotic disorder, bipolar disorder, autism-spectrum disorder, and attenuated psychosis syndrome.

In one aspect of the disclosure is a method of treating the psychiatric indications comprising administering to a patient in need thereof an effective amount of a compound of Formula I, or a pharmaceutically acceptable salt thereof.

In a further aspect, the psychiatric indications are selected from schizoaffective disorder, schizotypal disorder, schizophreniform disorder, treatment resistant schizophrenia, drug-induced psychotic disorder, bipolar disorder, autism-spectrum disorder, and attenuated psychosis syndrome.

In one aspect of the disclosure is a method of treating psychotic and neuropsychiatric symptoms associated with various neurodegenerative indications comprising administering to a patient in need thereof an effective amount of a compound of Formula I, or a pharmaceutically acceptable salt thereof.

In a further aspect, the psychotic and neuropsychiatric symptoms associated with various neurodegenerative indications are selected from Parkinson's disease, Alzheimer's disease, Frontotemporal dementia, Vascular cognitive impairment and

Dementia with Lewy Bodies.

The present disclosure further provides for methods of treating a neurological disease in a subject in need thereof, comprising administering to the subject a therapeutically effective amount of a compound as disclosed and described herein, or a pharmaceutically acceptable salt thereof (e.g. a compound of Formulae (I), or a pharmaceutically acceptable salt thereof), or a pharmaceutical composition comprising a compound as disclosed and described herein, or a pharmaceutically acceptable salt thereof (e.g. a compound of Formulae (I), or a pharmaceutically acceptable salt thereof), and a pharmaceutically acceptable excipient. The present disclosure also provides use of a compound as disclosed and described herein, or a pharmaceutically acceptable salt thereof (e.g. a compound of Formulae (I), or a pharmaceutically acceptable salt thereof) for treating a neurological disease in a subject in need thereof. The present disclosure also provides the manufacture of a medicament as disclosed and described herein, or a pharmaceutically acceptable salt thereof (e.g. a compound of Formulae (I), or a pharmaceutically acceptable salt thereof) for treating a neurological disease in a subject in need thereof.

In some aspects, the subject has been previously diagnosed with a neurological disorder. In some aspects, the subject is currently suffering from a neurological disorder. In some aspects, the subject is suspected of having a neurological disorder. In some aspects, the subject has been previously treated with one or more therapeutic agents approved for the treatment of a neurological disorder.

In some aspects, the neurological disorder is selected from schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder (OCD), an autism spectrum disorder, a prolactin-related disorder (e.g., hyperprolactinemia), a neurocognitive disorder, a trauma- or stressor-related disorder (e.g., PTSD); a disruptive, impulse-control, or conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia; Parkinson's Disease, drug induced Parkinsonism, dyskinesias, dystonia, chorea, levodopa induced dyskinesia, cerebral palsy, progressive supranuclear palsy, Huntington's disease, and chorea associated with Huntington's disease.

In some aspects, the neurological disorder is selected from schizophrenia, cognitive impairment, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, attention-deficit/hyperactivity disorder (ADHD), Tourette's syndrome, catatonia, a mood disorder, obsessive-compulsive disorder (OCD), hyperprolactinemia, PTSD, hypofrontality, Parkinson's Disease, drug induced Parkinsonism, dyskinesias, dystonia, chorea, levodopa induced dyskinesia, cerebral palsy, progressive supranuclear palsy, Huntington's disease, and chorea associated with Huntington's disease.

In some aspects, the neurological disorder is the neurological disorder is selected from schizophrenia. In some aspects, the neurological disorder is cognitive impairment. In some aspects, the neurological disorder is a panic disorder. In some aspects, the neurological disorder is a phobic disorder. In some aspects, the neurological disorder is drug-induced psychotic disorder. In some aspects, the neurological disorder is delusional psychosis. In some aspects, the neurological disorder is neuroleptic-induced dyskinesia. In some aspects, the neurological disorder is Parkinson's disease. In some aspects, the neurological disorder is drug-induced Parkinson's syndrome. In some aspects, the neurological disorder is extrapyramidal syndrome. In some aspects, the neurological disorder is Alzheimer's Disease. In some aspects, the neurological disorder is Lewy Body Dementia. In some aspects, the neurological disorder is bipolar disorder. In some aspects, the neurological disorder is attention-deficit/hyperactivity disorder (ADHD). In some aspects, the neurological disorder is Tourette's syndrome. In some aspects, the neurological disorder is an extrapyramidal or movement disorder. In some aspects, the neurological disorder is a motor disorder. In some aspects, the neurological disorder is a hyperkinetic movement disorder. In some aspects, the neurological disorder is a psychotic disorder. In some aspects, the neurological disorder is catatonia. In some aspects, the neurological disorder is a mood disorder. In some aspects, the neurological disorder is a depressive disorder. In some aspects, the neurological disorder is an anxiety disorder. In some aspects, the neurological disorder is obsessive-compulsive disorder (OCD). In some aspects, the neurological disorder is an autism spectrum disorder. In some aspects, the neurological disorder is a prolactin-related disorder. In some aspects, the neurological disorder is hyperprolactinemia). In some aspects, the neurological disorder is a neurocognitive disorder. In some aspects, the neurological disorder is a trauma- or stressor-related disorder. In some aspects, the neurological disorder is PTSD. In some aspects, the neurological disorder is impulse-control. In some aspects, the neurological disorder is or conduct disorder. In some aspects, the neurological disorder is a sleep-wake disorder. In some aspects, the neurological disorder is a substance-related disorder. In some aspects, the neurological disorder is an addictive disorder. In some aspects, the neurological disorder is a behavioral disorder. In some aspects, the neurological disorder is hypofrontality. In some aspects, the neurological disorder comprises an abnormality in the tuberoinfundibular pathway. In some aspects, the neurological disorder comprises an abnormality in the mesolimbic pathway. In some aspects, the neurological disorder comprises decreased activity in the striatum. In some aspects, the neurological disorder is cortical dysfunction. In some aspects, the neurological disorder is neurocognitive dysfunction and the cognitive deficits associated with schizophrenia or Parkinson's Disease. In some aspects, the neurological disorder is drug induced Parkinsonism. In some aspects, the neurological disorder is dyskinesias. In some aspects, the neurological disorder is dystonia. In some aspects, the neurological disorder is chorea. In some aspects, the neurological disorder is levodopa induced dyskinesia. In some aspects, the neurological disorder is cerebral palsy. In some aspects, the neurological disorder is progressive supranuclear palsy. In some aspects, the neurological disorder is Huntington's disease. In some aspects, the neurological disorder is and chorea associated with Huntington's disease.

In some aspects, the panic disorder comprises panic attacks. In some aspects, the phobic disorder is related to a situation (e.g., social phobia). In some aspects, the phobic disorder is related to an object (e.g., arachnophobia). In some aspects, the extrapyramidal syndrome comprises continuous spasms or muscle contractions, motor restlessness, muscle rigidity, slowed muscle response, tremors, or irregular, jerky movements. In some aspects, the extrapyramidal or movement disorder is tardive dyskinesia, an acute dystonic reaction, akathisia, or pseudo-Parkinsonism. In some aspects, the motor disorder is developmental coordination disorder, stereotypic movement disorder, or Tourette syndrome. In some aspects, the hyperkinetic movement disorder comprises athetosis, ballism, chorea, dystonia, myoclonus, restless leg syndrome, stereopathy, tics, or tremors. In some aspects, the psychotic disorder is schizophrenia, schizophreniform disorder, delusional disorder, or chronic hallucinatory psychosis. In some aspects, the mood disorder is major depression or bipolar depression. In some aspects, the depressive disorder is major depression, atypical depression, melancholic depression, catatonic major depression, post-partum depression, seasonal affective disorder, or double depression. In some aspects, the anxiety disorder is generalized anxiety disorder, post-traumatic stress disorder, obsessive compulsive disorder, a phobic disorder, or a panic disorder. In some aspects, the autism spectrum disorder is autism or Asperger syndrome. In some aspects, the neurocognitive disorder is major neurocognitive disorder or mild neurocognitive disorder. In some aspects, the disruptive, impulse-control, or conduct disorder is attention deficit disorder, attention deficit hyperactivity disorder, oppositional defiant disorder, sexual compulsion, internet addiction, pyromania, intermittent explosive disorder, compulsive shopping, or kleptomania. In some aspects, the sleep-wake disorder is insomnia, narcolepsy, or night terrors. In some aspects, the substance-related disorder is alcoholism, opioid addiction, prescription drug addiction, and/or illegal drug addiction. In some aspects, the addictive disorder comprises substance addition (e.g., alcoholism) or experiential additional (e.g., gambling addiction). In some aspects, the behavioral disorder is attention deficit disorder, attention deficit hyperactivity disorder, or oppositional defiant disorder.

It is understood in the art that some of the syndromes and symptoms described herein may have overlapping symptoms, and/or some of the particular disorders described herein may fall under multiple categories of disorders described herein. For example, tardive dyskinesia can be categorized at least as an extrapyramidal or movement disorder, a hyperkinetic movement disorder, a motor disorder, or an extrapyramidal syndrome.

Some aspects provide a method for modulating GPR52 in a cell comprising contacting the cell with a compound of Formula (I) or a pharmaceutically acceptable salt thereof. Without being bound by any theory, the compound and the receptor can be in contact for a time sufficient and under appropriate conditions to permit interaction between the cell and the compound.

In some aspects, the contacting is in vitro. In some aspects, the contacting is in vivo. In some aspects, the contacting is in vivo, wherein the method comprises administering a therapeutically effective amount of a compound of Formula (I), or a pharmaceutically acceptable salt thereof, to a subject having a cell having GPR52 activity.

In certain aspects, the cell is in a subject who is in need of treatment with a compound disclosed herein. In certain aspects, the cell is from a subject who is in need of treatment with a compound disclosed herein. In some aspects, the subject has a neurological disease, condition, or disorder. In some aspects, the subject is at risk for developing a neurological disease, condition, or disorder. In some aspects, the subject has been previously diagnosed with a neurological disease, condition, or disorder. In some aspects, the subject is currently being treated for a neurological disease, condition, or disorder. In some aspects, the subject is suffering from a neurological disease, condition, or disorder. In some aspects, the subject is suspected of having a neurological disease, condition, or disorder. In some aspects, the neurological disease, condition, or disorder is Alzheimer's Disease, Lewy Body Dementia, bipolar disorder, attention-deficit/hyperactivity disorder (ADHD), Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder (OCD), an autism spectrum disorder, a prolactin-related disorder (e.g., hyperprolactinemia), a neurocognitive disorder, a trauma- or stressor-related disorder (e.g., PTSD); a disruptive, impulse-control, or conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia, Parkinson's Disease, drug induced Parkinsonism, dyskinesias, dystonia, chorea, levodopa induced dyskinesia, cerebral palsy and progressive supranuclear palsy, and Huntington's disease, particularly chorea associated with Huntington's disease.

The cardiac potassium channel hERG (human ether-a-go-go-related gene) is responsible for a rapid delayed rectifier current (/Kr) in human ventricles. Inhibition of IKr is the most common cause of cardiac action potential prolongation by non-cardiac drugs (Brown, A. M., and Rampe, D., (2000), “Drug-induced long QT syndrome: is HERG the root of all evil?”, Pharmaceutical News, 7, 15-20; Weirich, J., and Antoni, H., (1998), “Rate-dependence of antiarrhythmic and proarrhythmic properties of class I and class III antiarrhythmic drugs”, Basic Res. Cardiol., 93 Suppl 1, 125-132; Yap, Y. G., and Camm, A. J. (1999), “Arrhythmogenic mechanisms of non-sedating antihistamines”, Clin Exp. Allergy, 29 Suppl 3, 174-181). Increased action potential duration causes prolongation of the QT interval and is associated with torsade de pointes (Brown, A. M., and Rampe, D., (2000), “Drug-induced long QT syndrome: is HERG the root of all evil?”, Pharmaceutical News, 7, 15-20). Compounds of Formula I were assessed for their in vitro effects on the hERG channel current (a surrogate for IKr, the rapidly activating delayed rectifier cardiac potassium current (Redfern, W. S., et al., “Relationships between preclinical cardiac electrophysiology, clinical QT interval prolongation and torsade de pointes for a broad range of drugs: evidence for a provisional safety margin in drug development”, Cardiovascular Research, Volume 58, Issue 1, April 2003, Pages 32-45). See “activity of compounds of Formula I against hERG” example, below.

Animal models can be used to model the cognitive disturbances in Schizophrenia. Administration of the glutamate/NMDA antagonist phencyclidine (PCP) provides a model of schizophrenia that can induce both negative symptoms as well as the positive symptoms associated with amphetamine psychosis (Jentsch and Roth, “The neuropsychopharmacology of phencyclidine: from NMDA receptor hypofunction to the dopamine hypothesis of schizophrenia”, Neuropsychopharmacology, March 1999, 20 (3), 201-225). This approach has pathological validity in that there is evidence of abnormalities of glutamatergic systems in the brain in schizophrenia; such changes include deficits in cortico-striatal innervation that can contribute to, if not underlie, cognitive dysfunction in the disease (Aparicio-Legarza, et al., “Deficits of [³H]D-aspartate binding to glutamate uptake sites in striatal and accumbens tissue in patients with schizophrenia”, Neuroscience Letters, 22 Aug. 1997, pages 13-16). In addition, some PCP-induced behaviors are reversed by certain atypical, but not typical antipsychotics (Geyer, M. A., et al., Startle response models of sensorimotor gating and habituation deficits in schizophrenia”, Brain Research Bulletin, Vol. 25, Issue 3, September 1990, 485-498). This suggests a potential correlation with effects on negative and cognitive symptoms that respond less effectively to the typical antipsychotics.

Certain pre-clinical tests allow the observation of relatively subtle cognitive deficits in the rat that resemble cognitive symptoms in subjects with a range of CNS disorders. These cognitive impairments include visual memory deficits which can be measured by recognition tasks such as the Novel Object Recognition (NOR) paradigm. A recognition memory task allows the comparison between presented stimuli and previously stored information. The NOR test in rats, which was based on the differential exploration of familiar and novel objects, was described by Ennaceur & Delacour (“A new one-trial test for neurobiological studies of memory in rats: I. Behavioral data”, Behavioral Brain Research, 31 (1), 47-59, 1988). The NOR test is a non-rewarded, ethologically relevant paradigm based on the spontaneous exploratory behavior of rats that measures episodic memory. Each session consists of two trials.

In the first trial, the rats are exposed to two identical objects in an open field. During the second trial, rats are exposed to two dissimilar objects, one familiar object from the first trial and one new object. Object recognition in rats can be measured as the difference in time spent exploring the familiar and the novel object. Rats have been shown to spend more time exploring the novel object. It was found that rats are able to discriminate between the familiar and the novel object when the inter-trial interval is between 3 minutes and 1-3 hours, but not when it is greater than 24 hours, although this effect may be sex dependent in rats (Sutcliffe et al, “Influence of gender on working and spatial memory in the novel object recognition task in the rat”, Behavioral Brain Research, 2007 Feb. 12; 177 (1): 117-25). The duration of each trial is also important, as a preference for the novel object only lasts during the first 3 minutes, after which the preference diminishes as both objects become familiar and are explored equally.

The effects of PCP. The sub-chronic (sc) treatment with PCP produces neuropathological changes of relevance to schizophrenia. This regimen produces a selective deficit in reversal learning in an operant reversal learning test and in novel object recognition. (scPCP)-induced deficits are robust and long-lasting in female rats and this dosing regimen also produces a reduction in social behavior in female hooded-Lister rats. A PCP-induced object recognition deficit is accompanied by a lack of dopamine release in the prefrontal cortex and hippocampus and this effect can be attenuated by dopamine D1 receptor activation. (Abdul-Monim, et al., “Sub-chronic psychotomimetic phencyclidine induces deficits in reversal learning and alterations in parvalbumin-immunoreactive expression in the rat”, Psychopharmacology, 2007 (March), 21 (2): 198-205; and Snigdha, et al., “PCP-Induced Disruption in Cognitive Performance is Gender-Specific and Associated with A Reduction in Brain-Derived Neurotrophic Factor (BDNF) in Specific Regions of the Female rat Brain”, J. Mol. Neurosci., 2011, 43:337-345; Abdul-Monim, et al., “The effect of atypical and classical antipsychotics on sub-chronic PCP-induced cognitive deficits in a reversal-learning paradigm”, Behavioral Brain Research, 169 (2006), 263-273; Abdul-Monim, et al., “Sub-chronic psychotomimetic phencyclidine induces deficits in reversal learning and alterations in parvalbumin-immunoreactive expression in the rat”, Psychopharmacology, 2007 (March), 21 (2): 198-205; McLean, et al., “D1-like receptor activation improves PCP-induced cognitive deficits in animal models: Implications for mechanisms of improved cognitive function in schizophrenia”, Vol.19, Issue 6, June 2009, Pages 440-450; and Idris, et al., “Sertindole improves sub-chronic PCP-induced reversal learning and episodic memory deficits in rodents: involvement of 5-HT6 and 5-HT2A receptor mechanisms”, Psychopharmacology, 208 (23), 2010; Grayson, et al., “Atypical antipsychotics attenuate a sub-chronic PCP-induced cognitive deficit in the novel object recognition task in the rat”, Behavioral Brain Research, Vol.184, Issue 1, 2007; Snigdha, et al., “Improvement of phencyclidine-induced social behavior deficits in rats: Involvement of 5-HT1A receptors”, Behavioral Brain Research, Vol. 191, Issue 1,2008, P26-31).

Behavioral tests and methods (NOR and social interaction paradigms) are provided in the examples, below.

### Pharmaceutical Combinations

It is further appreciated that certain features of the present disclosure, which are, for clarity, described in the context of separate aspects, can also be provided in combination in a single aspect. Conversely, various features of the present disclosure which are, for brevity, described in the context of a single aspect, can also be provided separately or in any suitable sub combination.

### EXAMPLES

Detailed compound synthesis methods are described in the Examples provided herein. A person having ordinary skill in the chemical art would be able to make a compound of Formula (I) and the formulae related thereto, including specific compounds described herein, by these methods or similar methods or other methods practiced by a person skilled in the art. In general, starting components are commercially available chemicals and can be obtained from commercial sources or can be made according to organic synthesis techniques known to those skilled in this art, starting from commercially available chemicals and/or from compounds described in the chemical literature. The compounds described herein are named according to MarvinSketch 18.24.0 or ChemDraw Professional 20.1.1.125 or later versions. In certain instances, when common names are used it is understood that these common names would be recognized by those skilled in the art.

“Commercially available chemicals” can be obtained from standard commercial sources including Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hanover, Germany), Spectrum Quality Product, Inc. (New Brunswick, NJ), TCI America (Portland OR), Trans World Chemicals, Inc. (Rockville MD), and Wako Chemicals USA, Inc. (Richmond VA).

Methods known to one of ordinary skill in the art can be identified through various reference books and databases. Suitable reference books and treatise that detail the synthesis of reactants useful in the preparation of compounds of the present disclosure, or provide references to articles that describe the preparation, include for example, Synthetic Organic Chemistry, John Wiley & Sons, Inc., New York; S. R. Sandler et al., Organic Functional Group Preparations, 2nd Ed., Academic Press, New York, 1983; H. O. House, Modern Synthetic Reactions, 2nd Ed., W. A. Benjamin, Inc. Menlo Park, Calif. 1972; T. L. Gilchrist, Heterocyclic Chemistry, 2nd Ed., John Wiley & Sons, New York, 1992; J. March, Advanced Organic Chemistry: Reactions, Mechanisms and Structure, 4th Ed., Wiley Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. Organic Synthesis: Concepts, Methods, Starting Materials, Second, Revised and Enlarged Edition (1994) John Wiley & Sons ISBN: 3 527-29074-5; Hoffman, R. V. Organic Chemistry, An Intermediate Text (1996) Oxford University Press, ISBN 0-19-509618-5; Larock, R. C. Comprehensive Organic Transformations: A Guide to Functional Group Preparations, 2nd Edition (1999) Wiley-VCH, ISBN: 0-471-19031-4; March, J. Advanced Organic Chemistry: Reactions, Mechanisms, and Structure, 4th Edition (1992) John Wiley & Sons, ISBN: 0-471-60180-2; Otera, J. (editor) Modern Carbonyl Chemistry, (2000) Wiley-VCH, ISBN: 3-527-29871-1; Patai, S., Patai's 1992 Guide to the Chemistry of Functional Groups, (1992) Interscience ISBN: 0-471-93022-9; Quin, L. D. et al. A Guide to Organophosphorus Chemistry, (2000) Wiley-Interscience, ISBN: 0-471-31824-8; Solomons, T. W. G. Organic Chemistry, 7th Edition (2000) John Wiley & Sons, ISBN: 0-471-19095-0; Stowell, J. C., Intermediate Organic Chemistry, 2nd Edition (1993) Wiley-Interscience, ISBN: 0-471-57456-2; Industrial Organic Chemicals: Starting Materials and Intermediates: An Ullmann's Encyclopedia, (1999) John Wiley & Sons, ISBN: 3-527-29645-X, in 8 volumes; Organic Reactions, (1942-2019) John Wiley & Sons, in over 95 volumes; and Chemistry of Functional Groups, John Wiley & Sons, in hardcover volumes (86) and electronic volumes (26).

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., can be contacted for more details). Chemicals that are known but not commercially available in catalogs can be prepared by custom chemical synthesis houses according to known methods, where many of the standard chemical supply houses (e.g., those listed above) provide custom synthesis services.

The term “reducing agent” refers to a compound that contributes a hydride to an electrophilic position of a reactant compound such as an unsaturated carbon (e.g. carbon of a carbonyl moiety) such as converting a ketone containing reactant compound to an alcohol product compound or converting an ester containing reactant compound to an alcohol product compound. The reducing agent can be a hydride reducing agent. Example hydride reducing agents include, but are not limited to, diborane, borane (e.g. borane tetrahydrofuran complex), 9-borabicyclo[3.3.1]nonane, lithium aluminum hydride, diisobutylaluminum hydride, lithium diisobutyl-tert-butoxyaluminum hydride, lithium tri-tert-butoxyaluminum hydride, lithium tris [(3-ethyl-3-pentyl)oxy]aluminohydride, sodium bis(2-methoxyethoxy)aluminum dihydride, sodium aluminum hydride, calcium borohydride, lithium borohydride, magnesium borohydride, potassium borohydride, tetrabutylammonium borohydride, tetraethylammonium borohydride, tetramethylammonium borohydride, bis(triphenylphosphine) copper (I) borohydride, lithium 9-borabicyclo[3.3.1]nonane hydride, sodium triacetoxyborohydride, potassium tri-sec-butylborohydride, sodium tri-sec-butylborohydride, potassium trisiamylborohydride, lithium triethylborohydride, potassium triethylborohydride, sodium triethylborohydride, potassium triphenylborohydride, lithium dimethylaminoborohydride, lithium pyrrolidinoborohydride, sodium cyanoborohydride, sodium trimethoxyborohydride, sodium borohydride, and the like.

The term “halogenating agent” refers to a compound that contributes a halogen atom to a reactant compound such as converting an alcohol reactant compound to an alkyl halide product compound. Examples of halogenating agents include, but not limited to, thionyl chloride, oxalyl chloride, phosphorus oxychloride, phosphorus pentachloride, phosphorus trichloride, methanesulfonyl chloride and Nal, p-toluenesulfonyl chloride and Nal, phosphorus tribromide, triphenylphosphine dibromide, phosphorus pentabromide or thionyl bromide, and the like.

The term “amide coupling agent” refers to a compound that facilitates formation of an amide bond where carboxylic acid activation is required to promote coupling with an amine. Examples of amide coupling agents include, but not limited to, thionyl chloride, oxalyl chloride, phosphorus oxychloride, Vilsmeier reagent, propylphosphonic anhydride, ethylmethylphosphinic anhydride (EMPA), Ac₂O, pivaloyl chloride, ethyl chloroformate (ECF), isobutyl chloroformate (IBCF), 2-ethoxy-1-ethoxycarbonyl-1,2-dihydroquinoline (EEDQ), methanesulfonyl chloride (MsCl), p-toluenesulfonyl chloride (TsCl), pentafluorophenyl trifluoroacetate, cyanuric chloride, 2-chloro-4,6-dimethoxy-1,3,5-triazine (CDMT), 4-(4,6-dimethoxy-1,3,5-triazin-2-yl)-4-methyl morpholinium chloride (DMTMM), 1-tert-butyl-3-ethylcarbodiimide, 1,1′-carbonyldiimidazole (CDI), N,N′-dicyclohexylcarbodiimide (DCC), N,N′-diisopropylcarbodiimide (DIC), N-(3-dimethylaminopropyl)-N′-ethylcarbodiimide (EDC), 1,3-di-p-tolylcarbodiimide, benzotriazole-1-yl-oxy-tris-(dimethylamino)-phosphonium hexafluorophosphate (BOP), benzotriazole-1-yl-oxy-tris-pyrrolidino-phosphonium hexafluorophosphate (PyBOP), 6-chloro-benzotriazole-1-yloxy-tris-pyrrolidinophosphonium hexafluorophosphate (PyClock), (7-azabenzotriazol-1-yloxy)trispyrrolidinophosphonium hexafluorophosphate (PyAOP), 1-cyano-2-ethoxy-2-oxoethylideneaminooxy-tris-pyrrolidino-phosphonium hexafluorophosphate (PyOxim), 1-[(1-(cyano-2-ethoxy-2-oxoethylideneaminooxy)dimethylaminomorpholino)]uronium hexafluorophosphate (COMU), 3-(diethoxy-phosphoryloxy)-1,2,3-benzo[d]triazin-4 (3H)-one (DEPBT), O-[(ethoxycarbonyl) cyanomethylenamino]-N,N,N′,N′-tetramethyluronium tetrafluoroborate (TOTU), O-(2-Oxo-1 (²H)pyridyl)-N,N,N′,N′-tetramethyluronium tetrafluoroborate (TPTU), 2-(1H-benzotriazole-1-yl)-1,1,3,3-tetramethylaminium tetrafluoroborate (TBTU), N,N,N′,N′-tetramethyl-O—(N-succinimidyl) uronium hexafluorophosphate (HSTU), 2-(1H-benzotriazole-1-yl)-1,1,3,3-tetramethyluronium hexafluorophosphate (HBTU), 2-(6-Chloro-1H-benzotriazole-1-yl)-1,1,3,3-tetramethylaminium hexafluorophosphate (HCTU), and 1-[bis(dimethylamino)methylene]-1H-1,2,3-triazolo[4,5-b]pyridinium 3-oxide hexafluorophosphate (HATU).

The term “base” refers to a compound that is an electron pair donor in an acid-base reaction.

The base can be an inorganic base or an organic base.

The term “organic base” refers to a base including at least one C—H bond (e.g. an amine base). In some aspects, the amine base can be a primary, secondary, or tertiary amine. Examples of an amine base include, but are not limited to, methylamine, dimethylamine, diethylamine, diphenylamine, trimethylamine, triethylamine, N,N-diisopropylethylamine, diisopropylamine, piperidine, 2,2,6,6-tetramethylpiperidine, pyridine, 2,6-lutidine, 4-methylmorpholine, 4-ethylmorpholine, 1,5-diazabicyclo[4.3.0]non-5-ene, 1,8-diazabicyclo[5.4.0]undec-7-ene, 1,8-diazabicyclo[5.4.0]undec-7-ene, 1,4-diazabicyclo[2.2.2]octane, 1,8-bis(dimethylamino) naphthalene, 4-(dimethylamino)pyridine, and the like. In some aspects, the amine base can include one alkali metal or alkaline earth metal. Examples of an amine base including one alkali metal include, but are not limited to, sodium bis(trimethylsilyl)amide, lithium bis(trimethylsilyl)amide, potassium bis(trimethylsilyl)amide, lithium dicyclohexylamide, lithium dimethylamide, lithium diethylamide, lithium diisopropylamide, lithium 2,2,6,6-tetramethylpiperidide, and the like. In some aspects, the organic base can be a metal alkoxide base. Examples of a metal alkoxide base include, but are not limited to, barium tert-butoxide, lithium tert-amoxide, lithium tert-butoxide, lithium ethoxide, lithium isopropoxide, lithium methoxide, magnesium di-tert-butoxide, magnesium ethoxide, magnesium methoxide, potassium tert-butoxide, potassium ethoxide, potassium methoxide, potassium tert-pentoxide, sodium tert-butoxide, sodium ethoxide, sodium methoxide, sodium tert-pentoxide, and the like. In some aspects, the organic base can be an organometal base (e.g. organolithium base or organomagnesium base). Examples of an organolithium base include, but are not limited to, n-butyllithium, sec-butyllithium, tert-butyllithium, ethyllithium, hexyllithium, isobutyllithium, isopropyllithium, methyllithium, hexyllithium, phenyllithium, and the like. Examples of an organomagnesium base include, but are not limited to, methylmagnesium bromide, methylmagnesium chloride, methylmagnesium iodide, ethylmagnesium bromide, ethylmagnesium chloride, isopropylmagnesium bromide, isopropylmagnesium chloride, n-propylmagnesium chloride, propylmagnesium chloride, isobutylmagnesium bromide, isobutylmagnesium chloride, butylmagnesium chloride, sec-butylmagnesium chloride, tert-butylmagnesium chloride, cyclopentylmagnesium bromide, cyclopentylmagnesium chloride, 2-pentylmagnesium bromide, 3-pentylmagnesium bromide, isopentylmagnesium bromide, pentylmagnesium bromide, phenylmagnesium bromide, phenylmagnesium chloride, cyclohexylmagnesium chloride, pentadecylmagnesium bromide, octadecylmagnesium chloride, and the like.

The term “inorganic base” refers to a base that does not include at least one C—H bond and includes at least one alkali metal or alkaline earth metal. Examples of an inorganic base include, but are not limited to, sodium hydride, potassium hydride, lithium hydride, calcium hydride, barium carbonate, calcium carbonate, cesium carbonate, lithium carbonate, magnesium carbonate, potassium carbonate, sodium carbonate, cesium hydrogen carbonate, potassium hydrogen carbonate, sodium hydrogen carbonate, barium hydroxide, calcium hydroxide, cesium hydroxide, lithium hydroxide, magnesium hydroxide, potassium hydroxide, sodium hydroxide, and the like.

The term “acid” refers to a compound that is an electron pair acceptor in an acid-base reaction.

The acid can be an inorganic acid or organic acid.

The term “inorganic acid” refers to an acid that does not include a carbon bond. Inorganic acids can be a strong acid or a weak acid. Examples of inorganic acids include, but are not limited to, sulfamic acid, hydrochloric acid, hydriodic acid, hydrobromic acid, perchloric acid, sulfuric acid, nitric acid, boric acid, fluorophosphoric acid, phosphoric acid, and the like.

The term “organic acid” refers to an acid including at least one C—H bond, C—F bond, or C—C bond. Examples of organic acid include but not limited to acetic acid, benzenesulfonic acid, benzoic acid, camphorsulfonic acid, citric acid, difluoroacetic acid, ethanesulfonic acid, formic acid, fumaric acid, gallic acid, glycolic acid, lactic acid, maleic acid, malonic acid, methanesulfonic acid, nitrilotriacetic acid, oxalic acid, phthalic acid, propionic acid, salicylic acid, succinic acid, 5-sulfosalicylic acid, L-(+)-tartaric acid, p-toluenesulfonic acid, trifluoroacetic acid, trifluoromethanesulfonic acid, and the like.

### General Reactions Schemes

The present disclosure also includes processes for the preparation of compounds of Formula (I). In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula (I) can be prepared by proceeding as in the following Reaction scheme 1:

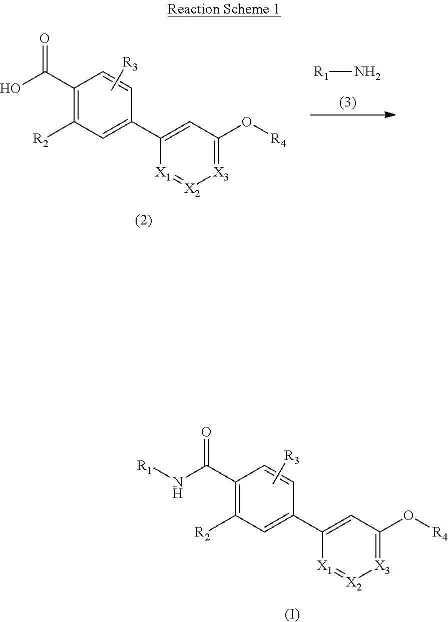

in which R₁, R₂, R₃, R₄, X₁, X₂ and X₃ are as defined in the Summary of the Disclosure, above. A compound of formula I can be synthesized by combining a compound of formula (2) and a compound of formula (3) in the presence of a suitable solvent (such as DCM, DCE, NMP, DMF, EtOAc, Toluene, Dioxane, Ethanol, water, and the like), optionally a suitable base (such as DIEA, TEA, and the like), and a suitable coupling agent (such as EDC/HOBt, HATU, HBTU, HCTU, and the like). The reaction proceeds at a temperature from about 0° C. to about 80° C. and can take up to about 24 hours to complete. See specific examples, below.

Compounds of Formula (I) can be prepared by proceeding as in the following Reaction scheme 2

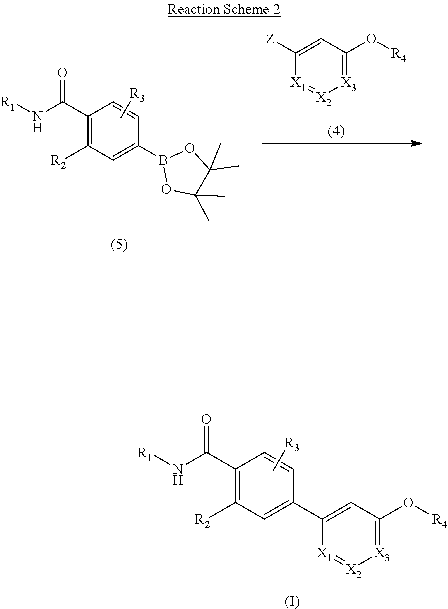

in which R₁, R₂, R₃, R₄, X₁, X₂ and X₃ are as defined in the Summary of the Disclosure, above, and Z is a suitable leaving group such as halogen (e.g., chloro), and the like. A compound of formula I can be synthesized by combining a compound of formula (4) and a compound of formula (5) in the presence of a suitable solvent (such as Toluene, Dioxane, Ethanol, DMF, EtOAc, and the like), a suitable base such as (Sodium Carbonate, Sodium hydroxide, Potassium carbonate, Sodium t-Butoxide, Potassium t-Butoxide, and the like), and a suitable coupling agent (such as tetrakis-triphenylphosphine Palladium (0) [CAS: 14221 Jan. 3], X-Phos-Pd-G2 [CAS: 1310584-14-5], X-Phos-Pd-G3 [CAS: 1445085-55-1], X-Phos Pd-G4 [CAS: 1599466-81-5]and the like). The reaction proceeds at a temperature from about 50° C. to about 120° C. and can take up to about 24 hours to complete. See specific examples, below.

Compounds of Formula (I) can be prepared by proceeding as in the following Reaction scheme 3

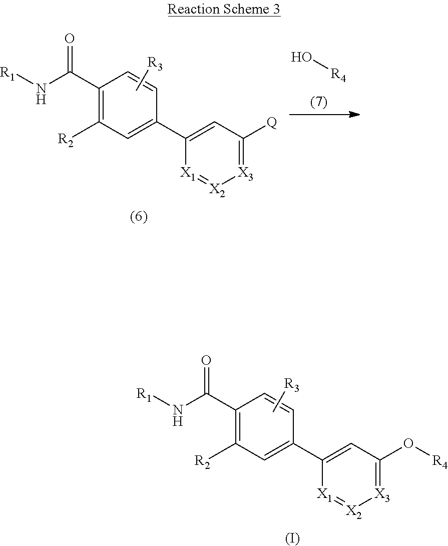

in which R₁, R₂, R₃, R₄, X₁, X₂ and X₃ are as defined in the Summary of the Disclosure, above, and Q is chloro, fluoro or bromo. A compound of formula I can be synthesized by combining a compound of formula (6) and a compound of formula (7) in the presence of a suitable solvent (such as DMF, NMP, THF, dioxane, DMA, EtOH, MeOH, IPA, BuOH and the like), and a suitable base (such as potassium carbonate, sodium carbonate, cesium carbonate, NaH, and the like). The reaction proceeds at a temperature from about 20° C. to about 100° C. and can take up to about 24 hours to complete. See specific examples, below.

### Additional Processes for Making Compounds of the Disclosure

A compound of the disclosure can be prepared as a pharmaceutically acceptable acid addition salt by reacting the free base form of the compound with a pharmaceutically acceptable inorganic or organic acid. Alternatively, a pharmaceutically acceptable base addition salt of a compound of the disclosure can be prepared by reacting the free acid form of the compound with a pharmaceutically acceptable inorganic or organic base.

Compounds of the formula I can also be modified by appending appropriate functionalities to enhance selective biological properties. Modifications of this kind are known in the art and include those that increase penetration into a given biological system (e.g. blood, lymphatic system, central nervous system, testis), increase bioavailability, increase solubility to allow parenteral administration (e.g. injection, infusion), alter metabolism and/or alter the rate of secretion. Examples of this type of modifications include but are not limited to esterification, e.g., with polyethylene glycols, derivatization with pivaloyloxy or fatty acid substituents, conversion to carbamates, hydroxylation of aromatic rings and heteroatom substitution in aromatic rings.

Wherever compounds of the formula I, and/or N-oxides, tautomers and/or (preferably pharmaceutically acceptable) salts thereof are mentioned, this comprises such modified formulae, while preferably the molecules of the formula I, their N-oxides, their tautomers and/or their salts are meant.

Alternatively, the salt forms of the compounds of the disclosure can be prepared using salts of the starting materials or intermediates. In view of the close relationship between the novel compounds of the formula I in free form and those in the form of their salts, including those salts that can be used as intermediates, for example in the purification or identification of the novel compounds, any reference to the compounds or a compound of the formula I hereinbefore and hereinafter is to be understood as referring to the compound in free form and/or also to one or more salts thereof, as appropriate and expedient, as well as to one or more solvates, e.g. hydrates.

Salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, malonic acid, adipic acid, pimelic acid, suberic acid, azelaic acid, malic acid, tartaric acid, citric acid, amino acids, such as glutamic acid or aspartic acid, maleic acid, hydroxymaleic acid, methyl maleic acid, cyclohexane carboxylic acid, adamantane carboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 4-toluenesulfonic acid, 2-naphthalenesulfonic acid, 1,5-naphthalene-disulfonic acid, 2- or 3-methylbenzenesulfonic acid, methyl sulfuric acid, ethyl sulfuric acid, dodecyl sulfuric acid, N-cyclohexyl sulfamic acid, N-methyl- or N-ethyl-sulfamic acid, or other organic protonic acids, such as ascorbic acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

The free acid or free base forms of the compounds of the disclosure can be prepared from the corresponding base addition salt or acid addition salt from, respectively. For example, a compound of the disclosure in an acid addition salt form can be converted to the corresponding free base by treating with a suitable base (e.g., ammonium hydroxide solution, sodium hydroxide, and the like). A compound of the disclosure in a base addition salt form can be converted to the corresponding free acid by treating with a suitable acid (e.g., hydrochloric acid, etc.).

Compounds of the disclosure in unoxidized form can be prepared from oxides of compounds of the disclosure by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the disclosure can be prepared by methods known to those of ordinary skill in the art (e.g., for further details see Saulnier et al., (1994), Bioorganic and Medicinal Chemistry Letters, Vol. 4, p. 1985).

For example, appropriate prodrugs can be prepared by reacting a non-derivatized compound of the disclosure with a suitable carbamylating agent (e.g., 1,1-acyloxyalkylcarbanochloridate, para-nitrophenyl carbonate, or the like).

Protected derivatives of the compounds of the disclosure can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999.

Compounds of the present disclosure can be conveniently prepared, or formed during the process of the disclosure, as solvates (e.g., hydrates). Hydrates of compounds of the present disclosure can be conveniently prepared by recrystallization from an aqueous/organic solvent mixture, using organic solvents such as dioxin, tetrahydrofuran or methanol.

Compounds of the disclosure can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the disclosure, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen,

“Enantiomers, Racemates and Resolutions”, John Wiley and Sons, Inc., 1981.

In summary, the compounds of Formula I can be made by a process, which involves:
(a) that of reaction schemes I, II or III; and
(b) optionally converting a compound of the disclosure into a pharmaceutically acceptable salt;
(c) optionally converting a salt form of a compound of the disclosure to a non-salt form;
(d) optionally converting an unoxidized form of a compound of the disclosure into a pharmaceutically acceptable N-oxide;
(e) optionally converting an N-oxide form of a compound of the disclosure to its unoxidized form;
(f) optionally resolving an individual isomer of a compound of the disclosure from a mixture of isomers;
(g) optionally converting a non-derivatized compound of the disclosure into a pharmaceutically acceptable prodrug derivative; and
(h) optionally converting a prodrug derivative of a compound of the disclosure to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present disclosure, and that other well-known methods can similarly be used.

The following examples are included to demonstrate aspects of the disclosure. However, those of skill in the art should, considering the present disclosure, appreciate that many changes can be made in the specific aspects which are disclosed and still obtain a like or similar result without departing from the spirit and scope of the disclosure.

The specification includes numerous abbreviations, whose definitions are listed in the following Table

| Abbreviation | Definition |
|---|---|
| ACN or CH₃CN | Acetonitrile |
| BOC | tert-Butyloxycarbonyl |
| CDI | 1,1′-Carbonyldiimidazole |
| EtOAc | Ethyl acetate |
| DBU | 1,8-Diazabicyclo[5.4.0]undec-7-ene |
| DCC | Dicyclohexylcarbodiimide |
| DCE | Dichloroethane |
| DCM | Dichloromethane or methylene chloride |
| de | Diastereomeric excess |
| DIPEA | N,N-Diisopropylethylamine |
| DMSO | Dimethylsulfoxide |
| DMSO-d₆ | Dimethylsulfoxide-d₆ |
| ee | Enantiomeric excess |
| FC | Flash chromatography |
| HPLC | High-performance liquid chromatography |
| KHMDS | Potassium bis(trimethylsilyl)amide |
| LCMS | Liquid chromatography-mass spectrometry |
| min. | Minute(s) |
| NH₄Cl | Ammonium chloride |
| Pd(PPh₃)₄ | Palladium-tetrakis(triphenylphosphine) |
| TEA | Triethylamine |
| TFA | Trifluoroacetic acid |
| THF | Tetrahydrofuran |

Analytical HPLC analyses were performed on an LC-MS system with a UV Detector (Dionex™ UVD 170u UV/VIS Detector), Corona array detector (Thermo™ Veo™ RS), and mass spectrometer (Dionex MSQ Plus™). Reverse-phase preparative HPLC purifications were performed on an LCMS system C18 Kinetix 5u 100 A 150×21.2 mm column by Phenomenex using ACN/water gradient containing 0.05% TFA. All final compounds were analyzed by analytical HPLC and peaks were monitored at 210, 254 and 280 nM for purity. 1H was recorded in an appropriate NMR solvent, such as, DMSO-d₆, on a Bruker 400 MHz spectrometer equipped with a Broad Band NMR probe. The 1H chemical signals are given in parts per million (ppm) with the residual solvent signal used as reference. The chemical shifts are expressed in ppm (δ) and coupling constants (J) are reported in hertz (Hz). Reactions were performed under an atmosphere of dry nitrogen unless otherwise stated.

Additionally, the following LCMS methods were employed:

LCMS method 1A:
Platform: Agilent 1260 UPLC with a Thermo MSQ mass detector and Agilent DAD (220 and 254 nm);
HPLC column: Waters XBridge BEH C18, 2.5 μM, 50×3.0 mm XP;
HPLC Gradient: 1.5 mL/min, 10% acetonitrile (with 0.025% TFA) in water (with 0.025% TFA) for 6 seconds, then increase to 90% acetonitrile over 1.5 minutes. Increase to 99% acetonitrile over 6 seconds, then hold at 99% acetonitrile for 12 seconds. Return to 10% acetonitrile over 6 seconds and hold at 10% for 30 seconds.

### LCMS Method 1B:

Platform: Agilent 1260 UPLC with an Thermo MSQ mass detector and Agilent DAD (220 and 254 nm);
HPLC column: Waters XBridge BEH C18, 2.5 μM, 50×3.0 mm XP;
HPLC Gradient: 1.5 mL/min, 10% acetonitrile (with 0.025% TFA) in water (with 0.025% TFA) for 6 seconds, then increase to 90% acetonitrile over 6.5 minutes. Increase to 99% acetonitrile over 6 seconds, then hold at 99% acetonitrile for 12 seconds. Return to 10% acetonitrile over 6 seconds and hold at 10% for 30 seconds.

### LCMS Method 2A:

Platform: Thermo Vanquish UHPLC with Thermo ISQEC mass detector, Thermo DAD (212, 220, 254 and 270 nm) and Thermo Charged Aerosol Detector;
HPLC column: Waters ACQUITY UPLC BEH C18, 1.7 μM, 50×2.1 mm;
HPLC Gradient: 1.1 mL/min, 10% acetonitrile (with 0.025% TFA) in water (with 0.025% TFA) for 6 seconds, then increase to 90% acetonitrile over 1.35 minutes. Increase to 99% acetonitrile over 6 seconds, then hold at 99% acetonitrile for 9 seconds. Return to 10% acetonitrile over 6 seconds and hold at 10% for 12 seconds.

### LCMS Method 2B:

Platform: Thermo Vanquish UHPLC with Thermo ISQEC mass detector, Thermo DAD (212, 220, 254 and 270 nm) and Thermo Charged Aerosol Detector;
HPLC column: Waters ACQUITY UPLC BEH C18, 1.7 UM, 50×2.1 mm;
HPLC Gradient: 1.0 mL/min, 5% acetonitrile (with 0.025% TFA) in water (with 0.025% TFA) for 6 seconds, then increase to 90% acetonitrile over 6.35 minutes. Increase to 99% acetonitrile over 6 seconds, then hold at 99% acetonitrile for 9 seconds. Return to 5% acetonitrile over 6 seconds and hold at 5% for 12 seconds.

### LCMS Method 3A:

Platform: Thermo Vanquish UHPLC with Thermo ISQEC mass detector, Thermo DAD (212, 220, 254 and 270 nm) and Thermo Charged Aerosol Detector;
HPLC column: Waters ACQUITY UPLC BEH C18, 1.7 UM, 50×2.1 mm;
HPLC Gradient: 1.1 mL/min, 2% acetonitrile (with 0.025% TFA) in water (with 0.025% TFA) for 42 seconds, then increase to 90% acetonitrile over 2.8 minutes. Increase to 99% acetonitrile over 6 seconds. Return to 2% acetonitrile over 6 seconds and hold at 2% for 9 seconds.

The Examples illustrate, without limitation, the synthesis of compounds of Formula (I)

### Intermediate Example 1

### 1,3-dimethyl-1H-pyrazolo[4,3-b]pyridin-6-ol

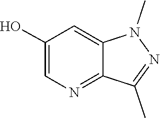

### Synthesis of 1,3-dimethyl-1H-pyrazolo[4.3-b]pyridin-6-ol

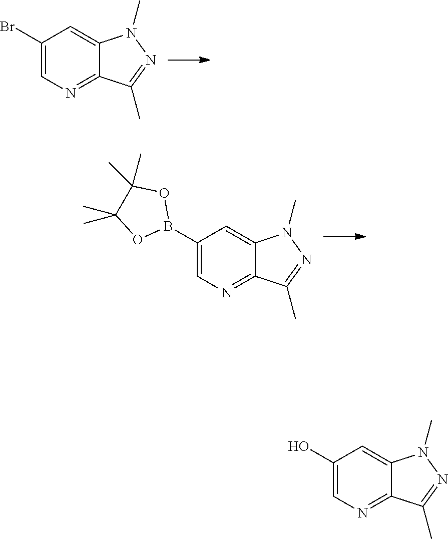

Step A: 6-Bromo-1,3-dimethyl-1H-pyrazolo[4,3-b]pyridine (10.0 g, 44.23 mmol), 4,4,5,5-tetramethyl-2-(tetramethyl-1,3,2-dioxaborolan-2-yl)-1,3,2-dioxaborolane (16.85 g, 66.35 mmol), Pd(dppf)Cl₂*CH₂Cl₂ (3.61 g, 4.42 mmol), potassium acetate (13.02 g, 132.7 mmol) were suspended in degassed dioxane. The mixture was heated under argon at 100° C. for 2 h. After cooling to room temperature, the mixture was diluted with ethyl acetate/hexane, filtered off through a pad of silica and concentrated in vacuo. The residue was used in the next step without purification.

Step B: 1,3-Dimethyl-6-(4,4,5,5-tetramethyl-1,3,2-dioxaborolan-2-yl)-1H-pyrazolo[4,3-b]pyridine (10.78 g, 39.46 mmol) was dissolved in THF/H₂O (3/1) and cooled with ice, then NaBO_{3·}4H₂O (17.0 g, 110.49 mmol) was added and left to stirred overnight. After solution Na₂S₂O₃ was added and organic layer had been separated. Water extracted with EtOAc, combined organic layers washed with brine, dried under Na₂SO₄ and evaporated. The residue was purified on FC (ISCO® Interchim; 220 g SiO₂, Acetonitrile/Methanol with Methanol from 0-95%, flow rate=80 mL/min, Rt=20-55 min.) to give 1,3-dimethyl-1H-pyrazolo[4,3-b]pyridin-6-ol (3.0 g, 18.38 mmol, 46.6% yield). LC/MS [M+H]164.0.

The following intermediate examples of table 1 were made according to the procedure in Intermediate Example 1 using the appropriate starting materials

TABLE 1

| | |
|---|---|
| 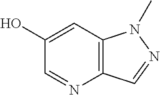 | 1-methyl-1H-pyrazolo [4,3-b]pyridin-6-ol |
| 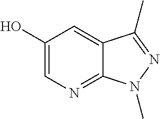 | 1,3-dimethyl-1H-pyrazolo [3,4-b]pyridin-5-ol |
| 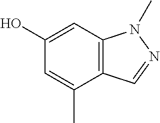 | 1,4-dimethyl-1H-indazol-6-ol |
| 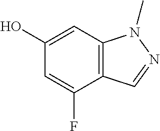 | 4-fluoro-1-methyl-1H-indazol-6-ol |
| 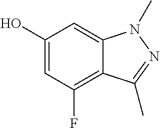 | 4-fluoro-1,3-dimethyl-1H- indazol-6-ol |
| 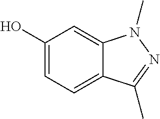 | 1,3-dimethyl-1H-indazol-6-ol |
| 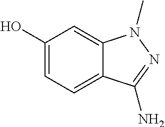 | 3-amino-1-methyl-1H-indazol-6-ol |
| 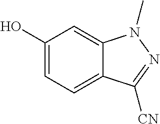 | 6-hydroxy-1-methyl-1H-indazole- 3-carbonitrile |
| 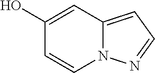 | 1,3-dimethyl-1H-indazol-5-ol |
| 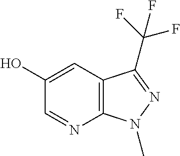 | 1-methyl-3-(trifluoromethyl)-1H- pyrazolo[3,4-b] pyridin-5-ol |
| 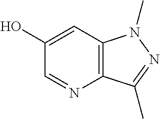 | 1,3-dimethyl-1H-pyrazolo [4,3-b]pyridin-6-ol |

### Intermediate Example 2

### 1-methyl-3-(trifluoromethyl)-1H-pyrazolo[3,4-b]pyridin-5-ol

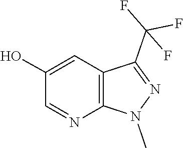

### Synthesis of 1-methyl-3-(trifluoromethyl)-1H-pyrazolo[3,4-b]pyridin-5-ol

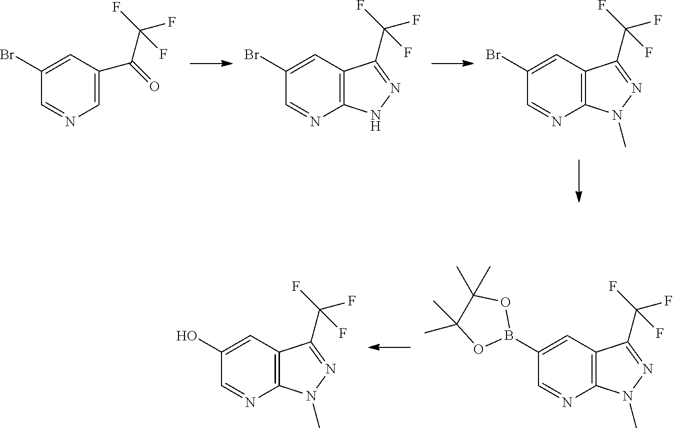 [SMILES: O=C(c1cncc(Br)c1)C(F)(F)F ; FC(F)(F)c1n[nH]c2ncc(Br)cc12 ; Cn1nc(C(F)(F)F)c2cc(Br)cnc21 ; Cn1nc(C(F)(F)F)c2cc(O)cnc21 ; Cn1nc(C(F)(F)F)c2cc(B3OC(C)(C)C(C)(C)O3)cnc21]

Step A: Hydrazine hydrate (18.77 g, 375.01 mmol) was added to 1-(5-bromo-2-fluoropyridin-3-yl)-2,2,2-trifluoroethan-1-one (5.1 g, 18.75 mmol) in ethanol (50 mL), and the mixture was heated to reflux overnight. The cooled reaction mixture was evaporated to afford a solid. Water (100 ml) was added, and the mixture was filtered to yield 5-bromo-3-(trifluoromethyl)-1H-pyrazolo[3,4-b]pyridine (4.4 g, 80.0% purity, 13.23 mmol, 70.6% yield) as a solid.

Step B: A solution of 5-bromo-3-(trifluoromethyl)-1H-pyrazolo[3,4-b]pyridine (2.4 g, 9.02 mmol) in DMF (30 ml) was cooled to 0° C. and sodium hydride (281.51 mg, 11.73 mmol) was added. The reaction mixture was stirred for 30 min at 0° C. and iodomethane (2.69 g, 18.95 mmol, 1.18 ml, 2.1 equiv.) was added. The reaction mixture was stirred for 10 min at 0° C. and at room temperature for 16 h. The reaction mixture was quenched with ice/water and diluted with ethyl acetate (50 ml). The organic layer was separated. The aqueous layer was again extracted with ethyl acetate (2×50 ml). The ethyl acetate layer was washed with brine, dried over Na₂SO₄, filtered, and concentrated under reduced pressure. The crude material was purified via silica gel chromatography to afford 5-bromo-1-methyl-3-(trifluoromethyl)-1H-pyrazolo[3,4-b]pyridine (1.5 g, 5.36 mmol, 59.4% yield).

Step C: 5-Bromo-1-methyl-3-(trifluoromethyl)-1H-pyrazolo[3,4-b]pyridine (2.0 g, 7.14 mmol), 4,4,5,5-tetramethyl-2-(tetramethyl-1,3,2-dioxaborolan-2-yl)-1,3,2-dioxaborolane (2.72 g, 10.71 mmol), potassium acetate (2.1 g, 21.42 mmol), Pd(dppf)Cl₂*CH₂Cl₂ (583.19 mg, 714.13 μmol) were suspended in dry dioxane. The mixture was degassed and heated under argon at 100° C. 1 h. After cooling to room temperature, the mixture was filtered through silica gel, and concentrated in vacuo to afford the 1-methyl-5-(4,4,5,5-tetramethyl-1,3,2-dioxaborolan-2-yl)-3-(trifluoromethyl)-1H-pyrazolo[3,4-b]pyridine (4.45 g, 35.0% purity, 4.76 mmol, 66.7% yield) used in next step without purification.

Step D: 1-Methyl-5-(4,4,5,5-tetramethyl-1,3,2-dioxaborolan-2-yl)-3-(trifluoromethyl)-1H-pyrazolo[3,4-b]pyridine (4.45 g, 13.6 mmol) was dissolved in THF/H₂O (150/50 ml), NaBO_{3·}4H₂O (2.05 g, 13.33 mmol) was added portions at 15-20° C., and the resulting mixture was stirred at room temperature for 16 hours. After Na₂S₂O₃ aqueous solution was added and mixture extracted with ethyl acetate (150 ml×3). The extracts were combined, washed with a saturated solution NaCl, dried over anhydrous sodium sulfate, and then the solvent was removed under reduced pressure crude product purified by column chromatography to give 1-methyl-3-(trifluoromethyl)-1H-pyrazolo[3,4-b]pyridin-5-ol (530.0 mg, 2.44 mmol, 51.3% yield). ¹H NMR (400 MHz, DMSO-d, 27° C.): δ=10.25 (br s, 1H), 8.38 (d, J=2.1 Hz, 1H), 7.40-7.45 (m, 1H), 4.12 ppm (s, 3H).

### Intermediate Example 3

### 1-methyl-3-(trifluoromethyl)-1H-pyrazolo[4,3-b]pyridin-6-ol

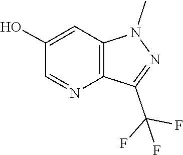

### Synthesis of 1-methyl-3-(trifluoromethyl)-1H-pyrazolo[4,3-b]pyridin-6-ol

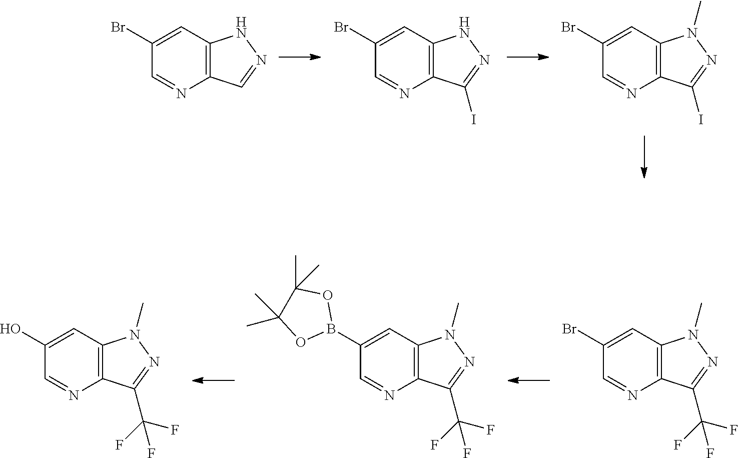 [SMILES: Brc1cnc2cn[nH]c2c1 ; Brc1cnc2c(I)n[nH]c2c1 ; Cn1nc(I)c2ncc(Br)cc21 ; Cn1nc(C(F)(F)F)c2ncc(O)cc21 ; Cn1nc(C(F)(F)F)c2ncc(B3OC(C)(C)C(C)(C)O3)cc21 ; Cn1nc(C(F)(F)F)c2ncc(Br)cc21]

Step A: A solution of 6-bromo-1H-pyrazolo[4,3-b]pyridine (8.0 g, 40.4 mmol), iodine (20.1 g, 79.18 mmol) and sodium hydroxide (6.06 g, 151.5 mmol) in DMF (80 mL) was stirred for 12 hours at room temperature. The reaction was quenched by dilution with saturated solution of sodium bisulfite (350 mL) and a precipitate was formed. The precipitate was filtered off under vacuum and washed with water (3×100 mL). The solid was left to dry at 30° C. in a vacuum oven overnight obtaining as an orange solid (8.12 g).

Step B: 6-Bromo-3-iodo-1H-pyrazolo[4,3-b]pyridine (8.1 g, 25.01 mmol) and iodomethane (7.1 g, 50.01 mmol, 3.11 ml, 2.0 eq.) were suspended in dry DMF (100 mL), then cesium carbonate (24.44 g, 75.02 mmol) was added at 20° C. The reaction mixture was stirred at r.t overnight. The resulting solution was concentrated under reduced pressure. The residue was taken up in 500 mL of water, solid was filtered off, washed 3 times with water, dried on air at 50° C. The product 6-bromo-3-iodo-1-methyl-1H-pyrazolo[4,3-b]pyridine (3.5 g, 10.36 mmol, 41.4% yield) was obtained as a brown solid after FC purification. LC/MS [M+H]337.8.

Step C: 6-Bromo-3-iodo-1-methyl-1H-pyrazolo[4,3-b]pyridine (9.1 g, 26.93 mmol), methyl 2,2-difluoro-2-(fluorosulfonyl)acetate (25.87 g, 134.64 mmol) and copper (I) iodide (25.64 g, 134.64 mmol) were combined in dimethylformamide (100 mL). The reaction mixture was stirred at 80° C. for 12 hours. Then the mixture was concentrated in vacuo, and the residue was purified by FC (Companion combiflash; 80 g SiO₂, CHCl₃/MeCN with MeCN from 0-95%, flow rate=60 mL/min, Rf=3-4 CV) to give 6-bromo-1-methyl-3-(trifluoromethyl)-1H-pyrazolo[4,3-b]pyridine (3.2 g, 95.0% purity, 10.86 mmol, 40.3% yield). LC/MS [M+H]280.0.

Step D: To a solution of 6-bromo-1-methyl-3-(trifluoromethyl)-1H-pyrazolo[4,3-b]pyridine (2.5 g, 8.93 mmol) and 4,4,5,5-tetramethyl-2-(4,4,5,5-tetramethyl-1,3,2-dioxaborolan-2-yl)-1,3,2-dioxaborolane (3.4 g, 13.39 mmol) in 1,4-dioxane (100 mL) was added potassium acetate (1.75 g, 17.85 mmol). The resulting mixture was degassed and charged with nitrogen for three times. Then [1,1′-bis(diphenylphosphino) ferrocene]dichloropalladium (II) dichloromethane adduct (728.98 mg, 892.67 μmol) was added and the reaction mixture was stirred at 110° C. under nitrogen atmosphere overnight, then cooled to rt, and concentrated in vacuo. The residue was diluted with EtOAc (500 mL), filtered through celite pad and washed with brine (3×300 mL). The separated organic phase was dried over anhydrous Na₂SO₄, and concentrated in vacuo. The 1-methyl-6-(4,4,5,5-tetramethyl-1,3,2-dioxaborolan-2-yl)-3-(trifluoromethyl)-1H-pyrazolo[4,3-b]pyridine (5.7 g, 35.0% purity, 6.1 mmol, 68.3% yield) was used in the next step without further purification. LC/MS [M+H]246.0.

Step E: To a solution of the crude 1-methyl-6-(4,4,5,5-tetramethyl-1,3,2-dioxaborolan-2-yl)-3-(trifluoromethyl)-1H-pyrazolo[4,3-b]pyridine (5.7 g, 17.42 mmol) in THF-H₂O (50+25 mL) was added NaBO_{3·}4H₂O (2.63 g, 17.07 mmol). The mixture was stirred at room temperature overnight. THF was removed under reduced pressure and the residue was stirred with saturated NH₄Cl (25 mL) and methylene chloride (150 mL). The organic layers were separated, dried over Na₂SO₄ and concentrated. The residue was purified by column chromatography (Interchim; 40 g SiO₂, CHCl₃/MeCN with MeCN from 0-95%, flow rate=40 mL/min, Rf=4-6 CV.) to give 1-methyl-3-(trifluoromethyl)-1H-pyrazolo[4,3-b]pyridin-6-ol (710.0 mg, 95.0% purity, 3.11 mmol, 50.9% yield). LC/MS [M+H]218.0.

The following Intermediate Examples of table 2 were made according to the procedure in Intermediate Example 3 using the appropriate starting materials

TABLE 2

| | |
|---|---|
| 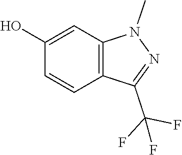 | 1-methyl-3-(trifluoromethyl)- 1H-indazol-6-ol |

### Intermediate Example 4

### tert-butyl (6-chloro-4-fluoropyridin-2-yl)carbamate

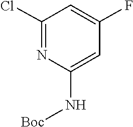 [SMILES: CC(C)(C)OC(=O)Nc1cc(F)cc(Cl)n1]

### Synthesis of tert-butyl (6-chloro-4-fluoropyridin-2-yl)carbamate

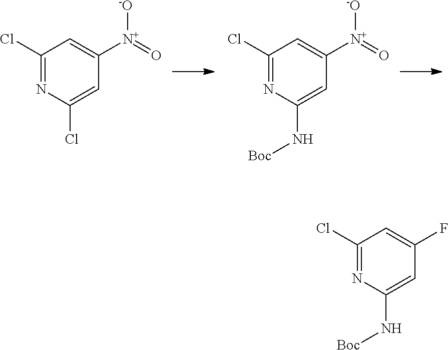

Step A: A suspension of 2,6-dichloro-4-nitropyridine (15.0 g, 77.73 mmol), tert-butyl carbamate (5.46 g, 46.63 mmol), tris ((1E,4E)-1,5-diphenylpenta-1,4-dien-3-one) dipalladium (3.56 g, 3.89 mmol), XantPhos (4.5 g, 7.77 mmol) and cesium carbonate (37.99 g, 116.59 mmol) in a degassed of dioxane was stirred under argon at 80° C. overnight. After cooling to r.t., the mixture was diluted with EtOAc and water. The organic layer was separated, washed with water and brine, dried over sodium sulfate, filtered, and concentrated in vacuo. The residue was purified by FC to afford tert-butyl N-(6-chloro-4-nitropyridin-2-yl)carbamate (10.0 g, 36.54 mmol, 47% yield).

Step B: To a cooled to 10° C. stirring solution of tert-butyl N-(6-chloro-4-nitropyridin-2-yl)carbamate (10.0 g, 36.54 mmol) in anhydrous THF (200 mL), tetrabutyl ammonium fluoride (76.73 ml 1M solution, 76.73 mmol, 2.1 equiv.) was added dropwise. The reaction mixture was stirred for 16 h at r.t. and then concentrated under reduced pressure. The residue was diluted with EtOAc and water, organic layer was separated, washed with water and brine, dried over sodium sulfate, filtered, and concentrated in vacuo. The residue was purified by FC to afford tert-butyl N-(6-chloro-4-fluoropyridin-2-yl)carbamate (4.7 g, 19.05 mmol, 52.1% yield). LC/MS [M+H]191.2.

### Intermediate Example 5

### di-tert-butyl (4-chloro-6-fluoropyridin-2-yl)iminodicarbonate

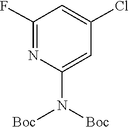 [SMILES: CC(C)(C)OC(=O)N(C(=O)OC(C)(C)C)c1cc(Cl)cc(F)n1]

### Synthesis of di-tert-butyl (4-chloro-6-fluoropyridin-2-yl)iminodicarbonate

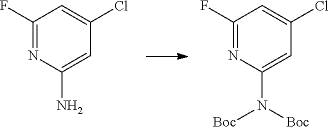 [SMILES: Nc1cc(Cl)cc(F)n1 ; CC(C)(C)OC(=O)N(C(=O)OC(C)(C)C)c1cc(Cl)cc(F)n1]

1M solution lithium (1+)bis(trimethylsilyl) azanide (6.28 g, 37.5 mmol, 37.5 mL, 1.1 equiv.) in THF was added to a solution 4-chloro-6-fluoropyridin-2-amine (5.0 g, 34.1 mmol) in THF at −78° C., and the mixture was stirred for 1 h. Then a solution of di-tert-butyl dicarbonate (17.1 g, 78.4 mmol) in THF was added dropwise at −78° C. The reaction mass was stirred for 1 h at −78° C. and then overnight at r.t. After that NH₄Cl was added, and the mixture was extracted with EtOAc. The organic layer was dried end evaporated. The crude product was purified by column chromatography to give tert-butyl N-[(tert-butoxy)carbonyl]-N-(4-chloro-6-fluoropyridin-2-yl)carbamate (7.71 g, 22.2 mmol, 65% yield).

### Intermediate Example 6

### 6-((2-chloropyridin-4-yl)oxy)-1,3-dimethyl-1H-pyrazolo[4,3-b]pyridine

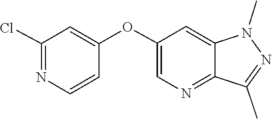 [SMILES: Cc1nn(C)c2cc(Oc3ccnc(Cl)c3)cnc12]

### Synthesis 6-((2-chloropyridin-4-yl)oxy)-1,3-dimethyl-1H-pyrazolo[4,3-b]pyridine

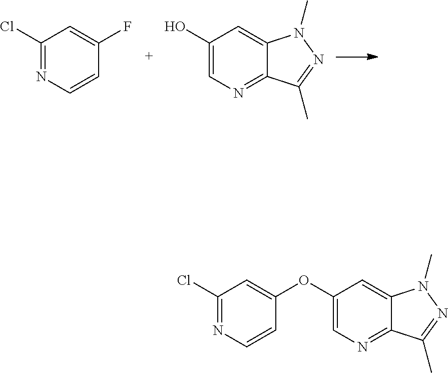 [SMILES: Fc1ccnc(Cl)c1 ; Cc1nn(C)c2cc(O)cnc12 ; Cc1nn(C)c2cc(Oc3ccnc(Cl)c3)cnc12]

1,3-dimethyl-1H-pyrazolo[4,3-b]pyridin-6-ol (10g) and 2-chloro-4-fluoropyridine (8.06g) were dissolved in 50 mL DMF then added powdered K₂CO₃ (6.47g). The mixture was heated to 75° C. overnight with stirring. The reaction was cooled and then to the reaction mixture was added ˜70 mL of water slowly while stirring. The mixture was then left to sit for 2 hours. The solids were collected by filtration and dried on filter by suction. Recovered 14.25 g of the product as a brown solid which was used without further purification. ¹H NMR (400 MHz, DMSO-d, 27° C.): δ=8.40 (d, J=2.2 Hz, 1H), 8.33 (d, J=5.7 Hz, 1H), 8.08 (d, J=2.3 Hz, 1H), 7.18 (d, J=2.2 Hz, 1H), 7.08 (dd, J=5.7, 2.2 Hz, 1H), 3.97 (s, 3H), 2.53 ppm (s, 3H). LCMS Method 1A: r.t. =1.75 min, m/z (M+H+)=274.93 observed mass, exact mass 274.06.

The following Intermediate Examples of table 3 were made according to the procedure in Intermediate Example 6 using the appropriate starting materials:

TABLE 3

| | |
|---|---|
| 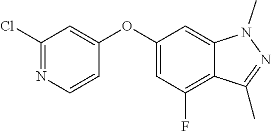 [SMILES: Cc1nn(C)c2cc(Oc3ccnc(Cl)c3)cc(F)c12] | 6-((2-chloropyridin-4- yl)oxy)-4-fluoro-1,3- dimethyl-1H-indazole LCMS Method 2A: r.t. = 1.32 min, m/z (M + H+) = 292.10 observed mass, exact mass 291.06 |
| 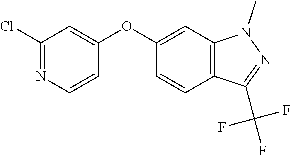 [SMILES: Cn1nc(C(F)(F)F)c2ccc(Oc3ccnc(Cl)c3)cc21] | 6-((2-chloropyridin-4- yl)oxy)-1-methyl-3- (trifluoromethyl)-1H- indazole LCMS Method 1A: r.t. = 2.23 min, m/z (M + H+) = 327.95 observed mass, exact mass 327.04 |
| 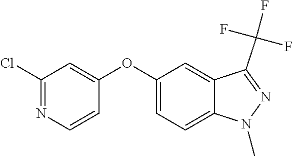 [SMILES: Cn1nc(C(F)(F)F)c2cc(Oc3ccnc(Cl)c3)ccc21] | 5-((2-chloropyridin-4- yl)oxy)-1-methyl-3- (trifluoromethyl)-1H- indazole LCMS Method 1A: r.t. = 2.19 min, m/z (M + H+) = 327.95 observed mass, exact mass 327.04 |
| 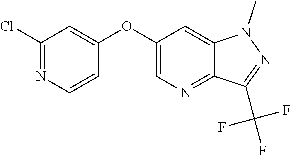 [SMILES: Cn1nc(C(F)(F)F)c2ncc(Oc3ccnc(Cl)c3)cc21] | 6-((2-chloropyridin-4- yl)oxy)-1-methyl-3- (trifluoromethyl)-1H- pyrazolo[4,3-b] pyridine LCMS Method 1A: r.t. = 2.18 min, m/z (M+) = 327.95 observed mass, exact mass 328.03 |
| 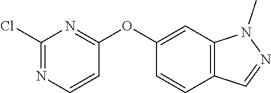 [SMILES: Cn1ncc2ccc(Oc3ccnc(Cl)n3)cc21] | 6-((2-chloropyrimidin- 4-yl)oxy)-1- methyl-1H-indazole (starting with 4-bromo-2- chloropyrimidine) |
| 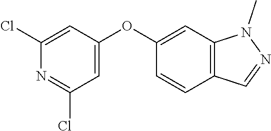 [SMILES: Cn1ncc2ccc(Oc3cc(Cl)nc(Cl)c3)cc21] | 6-((2,6-dichloropyridin- 4-yl)oxy)-1- methyl-1H-indazole (starting with 2,4,6- trichloropyridine) 1H NMR (400 MHz, DMSO-d, 27° C.): δ = 8.12 (s, 1H), 7.89 (d, J = 8.7 Hz, 1H), 7.83 |
| | (d, J = 2.1 Hz, 1H), |
| | 7.62 (s, 1H), 7.40 (d, |
| | J = 2.1 Hz, 1H), 7.01 |
| | (d, J = 8.8 Hz, 1H), |
| | 4.02 ppm (s, 3H) |
| | LCMS Method 2A: |
| | r.t. = 1.24 min, m/z |
| | (M + H+) =294.07 |
| | observed mass, exact |
| | mass 293.01 |

### Intermediate Example 7

### 6-((2-chloropyridin-4-yl)oxy)-1-methyl-1H-indazole

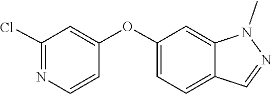 [SMILES: Cn1ncc2ccc(Oc3ccnc(Cl)c3)cc21]

### Synthesis of 6-((2-chloropyridin-4-yl)oxy)-1-methyl-1H-indazole

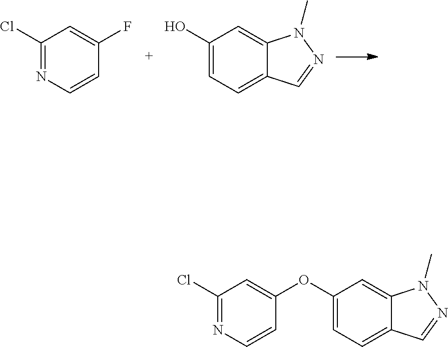 [SMILES: Fc1ccnc(Cl)c1 ; Cn1ncc2ccc(O)cc21 ; Cn1ncc2ccc(Oc3ccnc(Cl)c3)cc21]

1-methyl-1H-indazol-6-ol (0.444 g, 3.00 mmoles, 1.2 equiv) was stirred with 60% w/v NaH (1.2 eq; alternatively K₂CO₃ can be used-1-1.5 eq) in dry DMF (2.5 ml, 1 M) for 30 min at 0° C., followed by addition of 2-chloro-4-fluoropyridine (0.330 g, 2.50 mmoles, 1.0 equiv, 1 mL in DMF for transfer). The resultant mixture was warmed to room temperature over 30 minutes and stirred at 80° C. overight (15-18 h). Reaction was cooled to room temperature. The reaction was quenched with H₂O (2 mL) and extracted with EtOAc (4×). The organic layer was dried over sodium sulfate, filtered, and concentrated. Purification was carried out via automated Combiflash column chromatography on 12 g silica gel column with gradient elution of 0-60% EtOAc/hexanes. The title compound was obtained as a white solid (562.3 mg, 2.16 mmol, 87% yield).

The following Intermediate Examples of table 4 were made according to the procedure in Intermediate Example 3 using the appropriate starting materials

TABLE 4

| 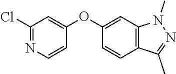 [SMILES: Cc1nn(C)c2cc(Oc3ccnc(Cl)c3)ccc12] | 6-((2-chloropyridin-4-yl) oxy)-1,3-dimethyl-1H- indazole 1H NMR (400 MHz, DMSO-d, 27° C.): δ = 8.29 |
|---|---|
| | (d, J = 5.7 Hz, 1H), 7.81 (d, |
| | J = 8.6 Hz, 1H), 7.50 (d, J = |
| | 1.5 Hz, 1H), 7.02 (d, J = 2.1 |
| | Hz, 1H), 6.98 (dd, J = 5.7, |
| | 2.2 Hz, 1H), 6.93 (dd, J = |
| | 8.6, 1.9 Hz, 1H), |
| | 3.93 (s, 3H), 2.49-2.53 |
| | ppm (s, overlapped with |
| | DMSO solvent peak, 3H) |
| | LCMS Method 2A: r.t. = |
| | 1.23 min, m/z |
| | (M + H+) = 274.10 |
| | observed mass, exact mass |
| | 273.07 |
| 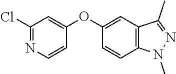 [SMILES: Cc1nn(C)c2ccc(Oc3ccnc(Cl)c3)cc12] | 5-((2-chloropyridin-4-yl) oxy)-1,3-dimethyl-1H- indazole 1H NMR (400 MHz, DMSO-d, 27° C.): δ = 8.27 |
| | (d, J = 5.6 Hz, 1H), 7.69 |
| | (d, J = 8.9 Hz, 1H), 7.61 |
| | (d, J = 2.2 Hz, 1H), 7.25 |
| | (dd, J = 9.0, 2.3 Hz, 1H), |
| | 6.91-6.96 (m, 2H), 4.00 |
| | (s, 3H), 2.46 ppm (s, 3H) |
| | LCMS Method 2A: r.t. = 1.22 |
| | min, m/z (M + H+) = 274.10 |
| | observed mass, exact mass |
| | 273.07 |
| 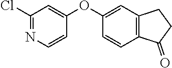 [SMILES: O=C1CCc2cc(Oc3ccnc(Cl)c3)ccc21] | 5-((2-chloropyridin-4-yl)oxy)- 2,3-dihydro-1H-inden-1-one LCMS Method 2A: r.t. = 1.24 min, m/z (M + H+) = 260.15 observed mass, exact mass 259.04 |
| 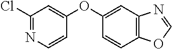 [SMILES: Clc1cc(Oc2ccc3ocnc3c2)ccn1] | 5-((2-chloropyridin-4- yl)oxy)benzo[d]oxazole 1H NMR (400 MHz, DMSO-d, 27° C.): δ = 8.86 (s, 1H), 8.28-8.31 |
| | (m, 1H), 7.91 (d, J = 8.8 Hz, 1H), |
| | 7.76 (d, J = 2.3 Hz, 1H), 7.34 (dd, |
| | J = 8.7, 2.4 Hz, 1H), 7.02 (d, J = |
| | 2.2 Hz, 1H), 6.95-6.98 ppm (m, |
| | 1H) |
| | LCMS Method 2A: r.t. = 1.07 |
| | min, m/z (M + H+) = 247.04 |
| | observed mass, exact mass |
| | 246.02 |
| 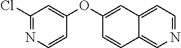 [SMILES: Clc1cc(Oc2ccc3cnccc3c2)ccn1] | 6-((2-chloropyridin-4- yl)oxy)isoquinoline 1H NMR (400 MHz, DMSO-d, 27° C.): δ = 9.36 (s, 1H), 8.53 (dd, |
| | J = 5.5, 1.7 Hz, 1H), 8.37 (dd, J = |
| | 5.6, 2.0 Hz, 1H), 8.29 (br d, J = |
| | 8.7 Hz, 1H), 7.84 (br d, J = 5.4 |
| | Hz, 1H), 7.78 (br s, 1H), 7.57 (br |
| | d, J = 8.8 Hz, 1H), 7.23 (s, 1H), |
| | 7.09-7.14 ppm (m, 1H) |
| | LCMS Method 2A: r.t. = 0.734 |
| | min, m/z (M + H+) = 257.07 |
| | observed mass, exact mass |
| | 256.04 |
| 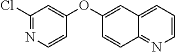 [SMILES: Clc1cc(Oc2ccc3ncccc3c2)ccn1] | 6-((2-chloropyridin-4- yl)oxy)quinoline 1H NMR (400 MHz, DMSO-d, 27° C.): δ = 8.93 (br s, 1H), 8.39 |
| | (br d, J = 8.1 Hz, 1H), 8.34 (br d, |
| | J = 5.1 Hz, 1H), 8.15 (br d, J = 8.8 |
| | Hz, 1H), 7.84 (br s, 1H), 7.66 (br |
| | d, J = 7.7 Hz, 1H), 7.56-7.62 (m, |
| | 1H), 7.17 (br s, 1H), 7.02-7.12 |
| | ppm (m, 1H) |
| | LCMS Method 2A: r.t. = 0.843 |
| | min, m/z (M + H+) = 257.07 |
| | observed mass, exact mass |
| | 256.04 |
| 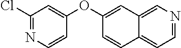 [SMILES: Clc1cc(Oc2ccc3ccncc3c2)ccn1] | 7-((2-chloropyridin-4- yl)oxy)isoquinoline 1H NMR (400 MHz, DMSO-d, 27° C.): δ = 9.33 (br s, 1H), 8.50- |
| | 8.59 (m, 1H), 8.30-8.40 (m, 1H), |
| | 8.14 (br d, J = 8.7 Hz, 1H), 7.98 |
| | (br s, 1H), 7.90-7.97 (M, 1H), |
| | 7.64-7.74 (m, 1H), 7.18 (br s, |
| | 1H), 7.03-7.13 ppm (m, 1H) |
| | LCMS Method 2A: r.t. = 0.732 |
| | min, m/z (M + H+) = 257.07 |
| | observed mass, exact mass |
| | 256.04 |
| 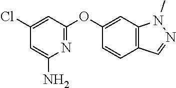 [SMILES: Cn1ncc2ccc(Oc3cc(Cl)cc(N)n3)cc21] | 4-chloro-6-((1-methyl-1H- indazol-6-yl)oxy)pyridin-2-amine (starting with 4,6- dichloropyridin-2-amine) |
| 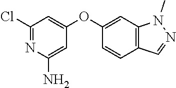 [SMILES: Cn1ncc2ccc(Oc3cc(N)nc(Cl)c3)cc21] | 6-chloro-4-((1-methyl-1H- indazol-6-yl)oxy)pyridin-2-amine (starting with 4,6- dichloropyridin-2-amine) 1H NMR (400 MHz, DMSO-d, 27° C.): δ = 8.08 (s, 1H), 7.84 (d, |
| | J = 8.6 Hz, 1H), 7.52 (s, 1H), 6.93 |
| | (dd, J = 8.7, 2.0 Hz, 1H), 6.37 (s, |
| | 2H), 6.22 (d, J = 1.7 Hz, 1H), |
| | 5.74 (d, J = 1.8 Hz, 1H), 4.02 |
| | ppm (s, 3H) |
| | LCMS Method 2A: r.t. = 0.993 |
| | min, m/z (M + H+) = 275.10 |
| | observed mass, exact mass |
| | 274.07 |
| 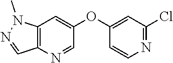 [SMILES: Cn1ncc2ncc(Oc3ccnc(Cl)c3)cc21] | 6-((2-chloropyridin-4-yl)oxy)-1- methyl-1H-pyrazolo[4,3- b]pyridine 1H NMR (400 MHz, DMSO-d, |
| | 27° C.): δ = 8.44-8.50 (m, 1H), |
| | 8.29-8.39 (m, 2H), 8.15-8.20 (m, |
| | 1H), 7.19-7.22 (m, 1H), 7.05-7.15 |
| | (m, 1H), 4.05-4.08 ppm (m, 3H) |
| 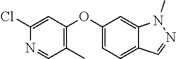 [SMILES: Cc1cnc(Cl)cc1Oc1ccc2cnn(C)c2c1] | 6-((2-chloro-5-methylpyridin-4- yl)oxy)-1-methyl-1H-indazole 1H NMR (400 MHz, DMSO-d, 27° C.): δ = 8.28 (s, 1H), 8.11 (s, |
| | 1H), 7.87 (d, J = 8.7 Hz, 1H), |
| | 7.52-7.57 (m, 1H), 6.98 (dd, J = |
| | 8.6, 2.0 Hz, 1H), 6.58 (s, 1H), |
| | 4.02 (s, 3H), 2.30 ppm (s, 3H) |
| | LCMS Method 2A: r.t. = 1.11 |
| | min, m/z (M + H+) = 274.08 |
| | observed mass, exact mass |
| | 273.07 |

### Intermediate Example 8

### tert-butyl (6-chloro-4-((1,3-dimethyl-1H-pyrazolo[3,4-b]pyridin-5-yl)oxy)pyridin-2-yl)carbamate

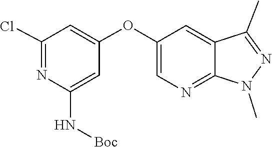 [SMILES: Cc1nn(C)c2ncc(Oc3cc(Cl)nc(NC(=O)OC(C)(C)C)c3)cc12]

### Synthesis of tert-butyl (6-chloro-4-((1,3-dimethyl-1H-pyrazolo[3,4-b]pyridin-5-yl)oxy)pyridin-2-yl)carbamate

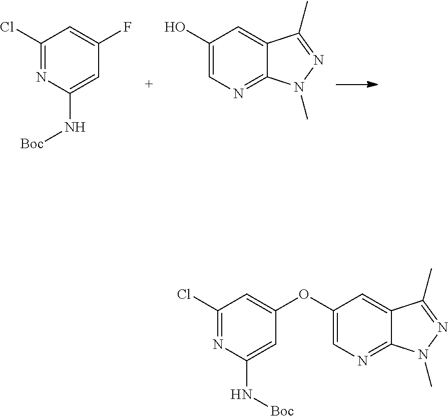 [SMILES: CC(C)(C)OC(=O)Nc1cc(F)cc(Cl)n1 ; Cc1nn(C)c2ncc(O)cc12 ; Cc1nn(C)c2ncc(Oc3cc(Cl)nc(NC(=O)OC(C)(C)C)c3)cc12]

1,3-dimethyl-1H-pyrazolo[3,4-b]pyridin-5-ol (0.3 g) was stirred with 60% w/v NaH (1.2 eq) in dry DMF (2.5 ml) for 30 min at 0° C., followed by addition of tert-butyl (6-chloro-4-fluoropyridin-2-yl)carbamate (0.454g). The resultant mixture was warmed to room temperature over 30 minutes and stirred at 90° C. for 4 h. Reaction was cooled to room temperature. The reaction was quenched with H₂O (2 mL) and extracted with EtOAc (4×). The organic layer was dried over magnesium sulfate, filtered, and concentrated. Purification was carried out via automated Combiflash column chromatography on silica gel column with gradient elution of EtOAc/hexanes. The title compound was obtained as a white foam. LCMS Method 2A: r.t. =1.43 min, m/z (M+H+)=390.16 observed mass, exact mass 389.12.

The following Intermediate Examples of table 5 were made according to the procedure in Intermediate Example 8 using the appropriate starting materials

TABLE 5

| | |
|---|---|
| 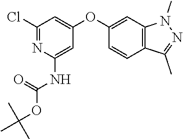 [SMILES: Cc1nn(C)c2cc(Oc3cc(Cl)nc(NC(=O)OC(C)(C)C)c3)ccc12] | tert-butyl (6-chloro-4-((1,3- dimethyl-1H-indazol-6- yl)oxy)pyridin-2-yl)carbamate LCMS Method 2A: r.t. = 1.53 min, m/z (M + H+) = 389.12 observed mass, exact mass 388.13 |
| 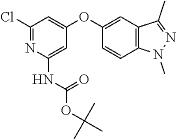 [SMILES: Cc1nn(C)c2ccc(Oc3cc(Cl)nc(NC(=O)OC(C)(C)C)c3)cc12] | tert-butyl (6-chloro-4-((1,3- dimethyl-1H-indazol-5- yl)oxy)pyridin-2-yl)carbamate LCMS Method 2A: r.t. = 1.51 min, m/z (M + H+) = 389.13 observed mass, exact mass 388.13 |
| 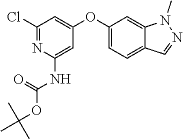 [SMILES: Cn1ncc2ccc(Oc3cc(Cl)nc(NC(=O)OC(C)(C)C)c3)cc21] | tert-butyl (6-chloro-4-((1-methyl- 1H-indazol-6-yl)oxy)pyridin-2- yl)carbamate 1H NMR (400 MHz, DMSO-d, 27° C.): δ = 10.15 (s, 1H), 8.11 (s, 1H), 7.87 (d, J = 8.7 Hz, 1H), 7.58 (s, 1H), 7.32 (d, J = 1.8 Hz, 1H), 6.99 (dd, J = 8.7, 2.0 Hz, 1H), 6.71 (d, J = 1.8 Hz, 1H), 4.03 (s, 3H), |
| | 1.37 ppm (s, 9H) |
| | LCMS Method 1A: r.t. = 2.26 min, |
| | m/z (M + H+) = 375.10 observed |
| | mass, exact mass 374.11 |
| 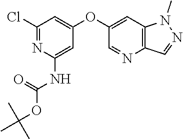 [SMILES: Cn1ncc2ncc(Oc3cc(Cl)nc(NC(=O)OC(C)(C)C)c3)cc21] | tert-butyl (6-chloro-4-((1-methyl- 1H-pyrazolo[4,3-b]pyridin-6- yl)oxy)pyridin-2-yl)carbamate LCMS Method 2A: r.t. = 1.27 min, m/z (M + H+) = 376.16 observed mass, exact mass 375.11 |
| 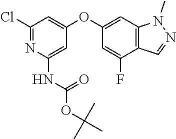 | tert-butyl (6-chloro-4-((4-fluoro-1- methyl-1H-indazol-6- yl)oxy)pyridin-2-yl)carbamate LCMS Method 2A: r.t. = 1.10 min, m/z (M + H+) = 384.18 observed mass, exact mass 383.14 |
| 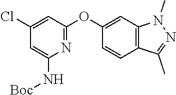 [SMILES: Cc1nn(C)c2cc(Oc3cc(Cl)cc(NC(=O)OC(C)(C)C)n3)ccc12] | tert-butyl (4-chloro-6-((1,3- dimethyl-1H-indazol-6- yl)oxy)pyridin-2-yl)carbamate (starting with tert-butyl (tert- butoxycarbonyl)(4-chloro-6- fluoropyridin-2-yl)carbamate) |
| | LCMS Method 2A: r.t. = 1.62 min, |
| | m/z (M + H+) = 389.14 observed |
| | mass, exact mass 388.13 |
| 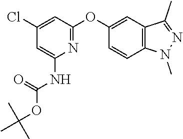 [SMILES: Cc1nn(C)c2ccc(Oc3cc(Cl)cc(NC(=O)OC(C)(C)C)n3)cc12] | tert-butyl (4-chloro-6-((1,3- dimethyl-1H-indazol-5- yl)oxy)pyridin-2-yl)carbamate (starting with tert-butyl (tert- butoxycarbonyl)(4-chloro-6- fluoropyridin-2-yl)carbamate) LCMS Method 2A: r.t. = 1.60 min, m/z (M + H+) = 389.14 observed mass, exact mass 388.13 |
| 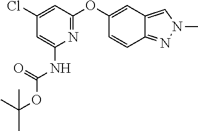 [SMILES: Cn1cc2cc(Oc3cc(Cl)cc(NC(=O)OC(C)(C)C)n3)ccc2n1] | tert-butyl (4-chloro-6-((2-methyl- 2H-indazol-5-yl)oxy)pyridin-2- yl)carbamate |
| 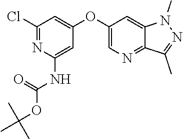 [SMILES: Cc1nn(C)c2cc(Oc3cc(Cl)nc(NC(=O)OC(C)(C)C)c3)cnc12] | tert-butyl (6-chloro-4-((1,3- dimethyl-1H-pyrazolo[4,3- b]pyridin-6-yl)oxy)pyridin-2- yl)carbamate 1H NMR (400 MHz, DMSO-d , 27° C.): δ = 10.21 (s, 1H), 8.39 (d, J = 2.3 Hz, 1H), 8.06 (d, J = 2.2 Hz, 1H), 7.33 (d, J = 2.0 Hz, 1H), 6.80 (d, J = 1.8 Hz, 1H), 3.97 (s, 3H), |
| | 2.52-2.54 (overlapped with DMSO |
| | solvent peak, 3H), 1.37 ppm (s, |
| | 9H) |

### Intermediate Example 9

### 6-(3-bromophenoxy)-1,3-dimethyl-1H-pyrazolo[4,3-b]pyridine

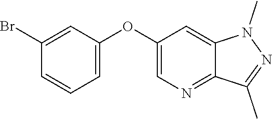 [SMILES: Cc1nn(C)c2cc(Oc3cccc(Br)c3)cnc12]

### Synthesis of 6-(3-bromophenoxy)-1,3-dimethyl-1H-pyrazolo[4,3-b]pyridine

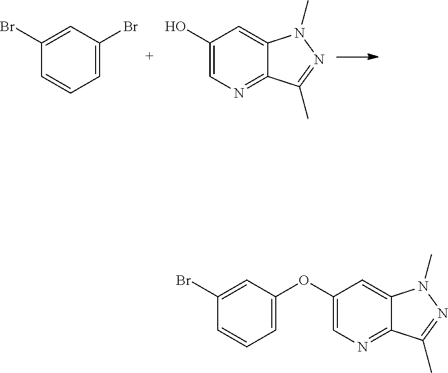 [SMILES: Brc1cccc(Br)c1 ; Cc1nn(C)c2cc(O)cnc12 ; Cc1nn(C)c2cc(Oc3cccc(Br)c3)cnc12]

In a glass vial with stirrer bar 1,3-dibromobenzene (1 mmol) in NMP (10 ml) was treated with 1,3-dimethyl-1H-pyrazolo[4,3-b]pyridin-6-ol (0.95 mmol) followed by Cu₂₀ (15 mol %) and Cs₂CO₃ (1 mmol). The reaction mixture in a sealed vial was heated at 210° C. for 5 min then to 195° C. for another 30 min. After completion of reaction, the mixture was cooled to room temperature and filtered through a celite plug and was washed with EtOAc. The organic layer was diluted with another 100 mL portion of EtOAc and was extracted with 200 ml of water twice. The Organic layers were combined and washed with brine. The crude material was purified by silica chromatography with EtOAC/hexane gradient up to 60% EtOAc to give 6-(3-bromophenoxy)-1,3-dimethyl-1H-pyrazolo[4,3-b]pyridine as a colorless semi solid. LCMS Method 1A: r.t. =2.00 min, m/z (M+H+, Br isotope effect)=319.90 observed mass, exact mass 317.02.

The following Intermediate Examples of table 6 were made according to the procedure in Intermediate Example 9 using the appropriate starting materials:

TABLE 6

| | |
|---|---|
| 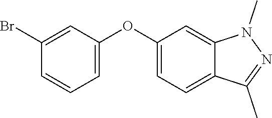 [SMILES: Cc1nn(C)c2cc(Oc3cccc(Br)c3)ccc12] | 6-(3-bromophenoxy)-1,3- dimethyl-1H-indazole LCMS Method 1A: r.t. = 2.30 min, m/z (M + H+, Br isotop effect) = 318.90 observed mass, exact mass 316.02 |
| 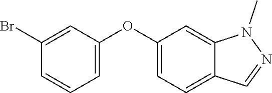 [SMILES: Cn1ncc2ccc(Oc3cccc(Br)c3)cc21] | 6-(3-bromophenoxy)-1- methyl-1H-indazole LCMS Method 1A: r.t. = 2.21 min, m/z (M + H+, Br isotope effect) = 302.95 observed |
| | mass, exact mass 302.00 |

### Intermediate Example 10

### 2-ethyl-4-(4-fluoropyridin-2-yl)benzamide

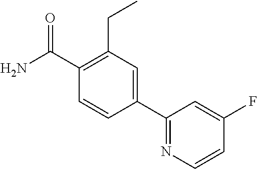 [SMILES: CCc1cc(-c2cc(F)ccn2)ccc1C(N)=O]

### Synthesis of 2-ethyl-4-(4-fluoropyridin-2-yl)benzamide

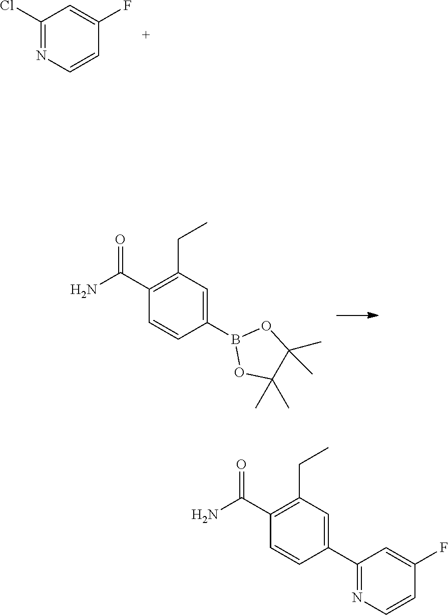 [SMILES: Fc1ccnc(Cl)c1 ; CCc1cc(B2OC(C)(C)C(C)(C)O2)ccc1C(N)=O ; CCc1cc(-c2cc(F)ccn2)ccc1C(N)=O]

2-chloro-4-fluoropyridine (0.48 g, 1 eq), 2-ethyl-4-(tetramethyl-1,3,2-dioxaborolan-2-yl)benzamide (1 eq), and tetrakis(triphenylphosphine) palladium (0) (0.05 eq) were added to a round bottom flask followed by addition of 1,4-dioxane (10 mL) and Na₂CO₃ (2N, 1 eq). Reaction mixture was degassed with bubbling N₂ for 15 min prior to heating to 90° C. and was stirred overnight. The mixture was then cooled to room temperature and the solvent was removed via reduced pressure. The crude reaction mixture was re-dissolved in DCM/MeOH (4:1)₅₀₀ mL and was washed with water (2×150 mL). The organic layer was dried over Na₂SO₄, filtered and 50 g of silica was added to crude followed by concentration under reduced pressure. The silica-loaded crude was purified by silica gel column chromatography using DCM/MeOH 1% to 10% gradient over 30 min to elute the product which was obtained as a solid after drying. LCMS Method 2A: r.t. =0.802 min, m/z (M+H+)=245.15 observed mass, exact mass 244.10.

The following Intermediate Examples of table 7 were made according to the procedure in Intermediate Example 10 using the appropriate starting materials

TABLE 7

| | |
|---|---|
| 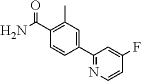 [SMILES: Cc1cc(-c2cc(F)ccn2)ccc1C(N)=O] | 4-(4-fluoropyridin-2-yl)-2-methylbenzamide 1H NMR (400 MHz, DMSO-d, 27° C.): δ = 8.71 (dd, J = 9.1, 5.6 Hz, 1H), 8.02 (s, 1H), 7.93-8.00 (m, 2H), 7.79 (m, 1H), 7.49 (d, J = 8.1 Hz, 1H), 7.42 (br s, 1H), 7.32 (ddd, J = 8.5, 5.8, 2.3 Hz, 1H), 2.44-2.48 ppm (s, 3H). |
| | LCMS Method 1A: r.t. = 1.42 min, m/z |
| | (M + H+) = 230.99 observed mass, exact |
| | mass 230.09 |
| 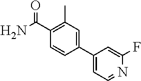 [SMILES: Cc1cc(-c2ccnc(F)c2)ccc1C(N)=O] | 4-(2-fluoropyridin-4-yl)-2-methylbenzamide (Starting with 4-bromo-2-fluoropyridine) 1H NMR (400 MHz, DMSO-d, 27° C.): δ = 8.32 (d, J = 5.3 Hz, 1H), 7.81 (M, 1H), 7.76 (s, 1H), 7.69-7.75 (m, 1H), 7.56 (s, 1H), 7.49-7.52 (m, 1H), 7.44-7.47 (m, 1H), 2.46 |
| | ppm (s, 3H). |
| | LCMS Method 1A: r.t. = 1.49 min, m/z |
| | (M + H+) = 231.01 observed mass, exact |
| | mass 230.09 |
| 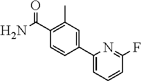 [SMILES: Cc1cc(-c2cccc(F)n2)ccc1C(N)=O] | 4-(6-fluoropyridin-2-yl)-2-methylbenzamide (Starting with 2-chloro-6-fluoropyridine) LCMS Method 1A: r.t. = 1.49 min, m/z (M + H+) = 231.00 observed mass, exact mass 230.09 |
| 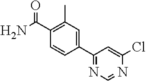 [SMILES: Cc1cc(-c2cc(Cl)ncn2)ccc1C(N)=O] | 4-(6-chloropyrimidin-4-yl)-2- methylbenzamide (Starting with 4,6-dichloropyrimidine) LCMS Method 2A: r.t. = 0.984 min, m/z (M + H+) = 248.07 observed mass, exact mass 247.05 |
| 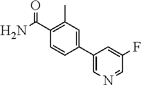 [SMILES: Cc1cc(-c2cncc(F)c2)ccc1C(N)=O] | 4-(5-fluoropyridin-3-yl)-2-methylbenzamide (Starting with 3-bromo-5-fluoropyridine) 1H NMR (400 MHz, DMSO-d, 27° C.): δ = 8.83 (s, 1H), 8.60 (s, 1H), 8.10 (br d, J = 10.4 Hz, 1H), 7.78 (br s, 1H), 7.69 (s, 1H), 7.65 (br d, J = 7.9 Hz, 1H), 7.49 (d, J = 7.9 |
| | Hz, 1H), 7.42 (br s, 1H), 2.46 ppm (s, 3H) |
| | LCMS Method 1A: r.t. = 1.31 min, m/z |
| | (M + H+) = 231.03 observed mass, exact |
| | mass 230.08 |
| 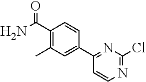 [SMILES: Cc1cc(-c2ccnc(Cl)n2)ccc1C(N)=O] | 4-(2-chloropyrimidin-4-yl)-2- methylbenzamide (starting with 4-bromo-2-chloropyrimidine) |
| 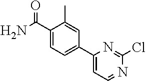 [SMILES: Cc1cc(-c2ccnc(Cl)n2)ccc1C(N)=O] | 4-(6-chloropyrazin-2-yl)-2- methylbenzamide (Starting with 2,6-dichloropyrazine) LCMS Method 2A: r.t. = 0.867 min, m/z (M + H+) = 248.05 observed mass, exact mass 247.05 |
| 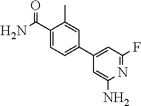 [SMILES: Cc1cc(-c2cc(N)nc(F)c2)ccc1C(N)=O] | 4-(2-amino-6-fluoropyridin-4-yl)-2- methylbenzamide Starting with tert-butyl (tert- butoxycarbonyl)(4-chloro-6-fluoropyridin-2- yl)carbamate followed by Boc-deprotection (4N HCl in dioxane) 1H NMR (400 MHz, DMSO-d, 27° C.): δ = |
| | 7.77 (br s, 1H), 7.43-7.55 (m, 3H), 7.41 (br |
| | s, 1H), 6.59 (s, 1H), 6.43 (br d, J = 8.2Hz, |
| | 3H), 2.43 ppm (s, 3H) |
| | LCMS Method 2A: r.t. = 0.764 min, m/z |
| | (M + H+) = 246.13 observed mass, exact |
| | mass 245.10 |

### Intermediate Example 11

### 3′-((1,3-dimethyl-1H-pyrazolo[4,3-b]pyridin-6-yl)oxy)-3-ethyl-[1,1′-biphenyl]-4-carboxylic acid

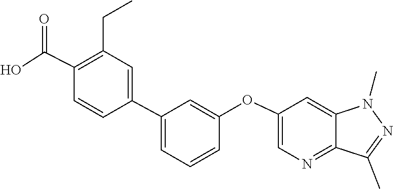 [SMILES: CCc1cc(-c2cccc(Oc3cnc4c(C)nn(C)c4c3)c2)ccc1C(=O)O]

### Synthesis of 3′-((1,3-dimethyl-1H-pyrazolo[4,3-b]pyridin-6-yl)oxy)-3-ethyl-[1,1′-biphenyl]-4-carboxylic acid

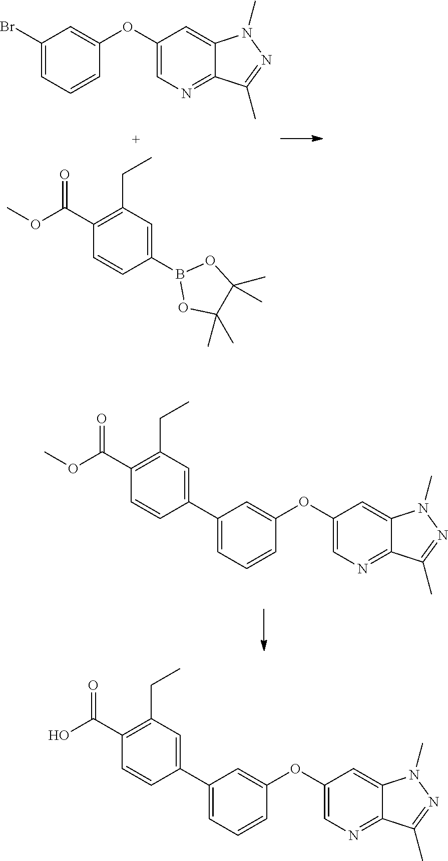

Step 1: (Suzuki coupling) In a vial 6-(3-bromophenoxy)-1,3-dimethyl-1H-pyrazolo[4,3-b]pyridine (1 mmol) was treated with methyl 2-ethyl-4-(4,4,5,5-tetramethyl-1,3,2-dioxaborolan-2-yl)benzoate (1 mmol) in 1,4-dioxane (1 mL), Pd(PPh3)₄ (5 mol %) and a 2 M aqueous solution of K₂CO₃ (0.25 mL). The resulting mixture was heated to 95° C., and cooled to rt. The crude was filtered through celite plug and was purified by silica gel column chromatography with EtOAC/hexane gradient up to 100% EtOAc to give methyl 3′-((1,3-dimethyl-1H-pyrazolo[4,3-b]pyridin-6-yl)oxy)-3-ethyl-[1,1′-biphenyl]-4-carboxylate as colorless thick oily product.

Step2: (saponification) Methyl 3′-((1,3-dimethyl-1H-pyrazolo[4,3-b]pyridin-6-yl)oxy)-3-ethyl-[1,1′-biphenyl]-4-carboxylate (1 mmol) was dissolved in THF: Methanol (3:1) (2 mL) and was treated with a 2M aqueous solution of LiOH (0.5 mL). The reaction mixture was stirred overnight at rt. The mixture was treated with a aqueous solution of 2N HCl to adjust pH ˜1. The acidified mixture was diluted with EtOAC (10 mL) and was extracted with water (10 mL) twice. The Organic layers were combined, washed with brine, dried with anhydrous Na₂CO₃, and concentrated in-vacuo to give 3′-((1,3-dimethyl-1H-pyrazolo[4,3-b]pyridin-6-yl)oxy)-3-ethyl-[1,1′-biphenyl]-4-carboxylic acid as a white solid.

The following examples of table 8 were made according to the procedure in Example 9 using the appropriate starting materials

TABLE 8

| | |
|---|---|
| 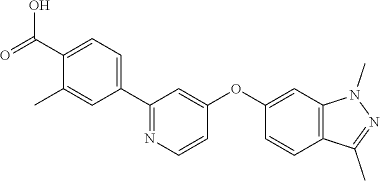 [SMILES: Cc1cc(-c2cc(Oc3ccc4c(C)nn(C)c4c3)ccn2)ccc1C(=O)O] | 4-(4-((1,3-dimethyl-1H-indazol-6- yl)oxy)pyridin-2-yl)-2- methylbenzoic acid LCMS Method 1A: r.t. = 1.57 min, m/z (M + H+) = 374.18 observed mass, exact mass 373.14 |
| 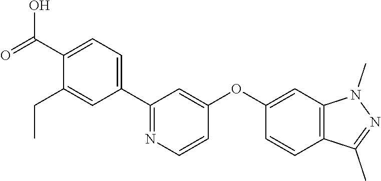 [SMILES: CCc1cc(-c2cc(Oc3ccc4c(C)nn(C)c4c3)ccn2)ccc1C(=O)O] | 4-(4-((1,3-dimethyl-1H-indazol-6- yl)oxy)pyridin-2-yl)-2-ethylbenzoic acid LCMS Method 1A: r.t. = 1.76 min, m/z (M + H+) = 388.21 observed mass, exact mass 387.16 |
| 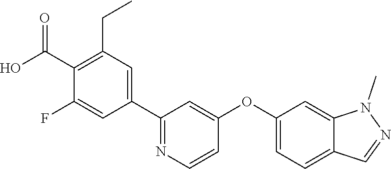 [SMILES: CCc1cc(-c2cc(Oc3ccc4cnn(C)c4c3)ccn2)cc(F)c1C(=O)O] | 2-ethyl-6-fluoro-4-(4-((1-methyl- 1H-indazol-6-yl)oxy)pyridin-2- yl)benzoic acid LCMS Method 1A: r.t. = 1.63 min, m/z (M + H+) = 392.22 observed mass, exact mass 391.13 |
| 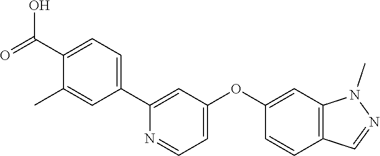 [SMILES: Cc1cc(-c2cc(Oc3ccc4cnn(C)c4c3)ccn2)ccc1C(=O)O] | 2-methyl-4-(4-((1-methyl-1H- indazol-6-yl)oxy)pyridin-2- yl)benzoic acid LCMS Method 2A: r.t. = 0.890 min, m/z (M + H+) = 360.14 observed mass, exact mass 359.13 |

### Intermediate Example 12

### 3-((2-chloropyridin-4-yl)oxy)-6,6-difluoro-5,6,7,8-tetrahydroquinoline

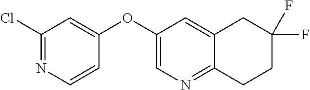 [SMILES: FC1(F)CCc2ncc(Oc3ccnc(Cl)c3)cc2C1]

### Synthesis of 3-((2-chloropyridin-4-yl)oxy)-6,6-difluoro-5,6,7,8-tetrahydroquinoline

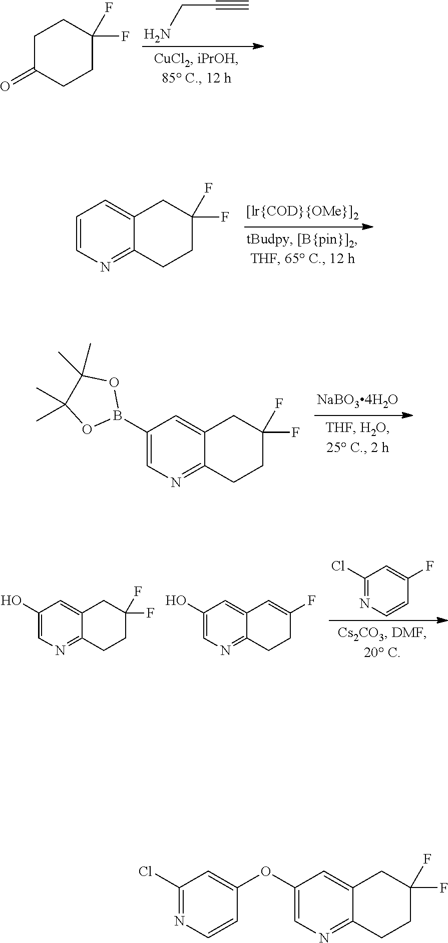

To a mixture of prop-2-yn-1-amine (4.11 g, 74.56 mmol, 4.78 mL) in isopropanol (i-PrOH, 40 mL) was added copper chloride (CuCl₂, 501.22 mg, 3.73 mmol) and a solution of 4,4-difluorocyclohexan-1-one (5 g, 37.28 mmol) in isopropanol (i-PrOH, 20 mL) at 85° C. The mixture was stirred at 85° C. for 12 h. The mixture was filtered, and the filtrate was concentrated under reduced pressure to give a residue, which was purified by column chromatography (SiO₂, petroleum ether/ethyl acetate=1/10) to give 6,6-difluoro-5,6,7,8-tetrahydroquinoline (2.5 g, yield 39.64%) as red oil. ¹H NMR: 400 MHz, CDCl₃ δ=2.34 (tt, J=13.55, 6.96 Hz, 2H), 3.19 (t, J=7.00 Hz, 2H), 3.28 (t, J=14.45 Hz, 2H), 7.12 (dd, J=7.69, 4.82 Hz, 1H), 7.40 (d, J=7.63 Hz, 1H), 8.45 (d, J=4.50 Hz, 1H).

To a mixture of 6,6-difluoro-5,6,7,8-tetrahydroquinoline (2.5 g, 14.78 mmol) in tetrahydrofuran (THF, 25 mL) was added 4,4,5,5-tetramethyl-2-(4,4,5,5-tetramethyl-1,3,2-dioxaborolan-2-yl)-1,3,2-dioxaborolane (4.13 g, 16.26 mmol), [Ir(COD)(OMe)]₂ (293.87 mg, 443.34 μmol) and 4-tert-butyl-2-(4-tert-butyl-2-pyridyl)pyridine (237.98 mg, 886.68 μmol). The mixture was stirred at 70° C. for 12 h under nitrogen atmosphere. The reaction was quenched with water (200 mL), and the aqueous layer was extracted with ethyl acetate (2×10⁰ mL). The organic layer was dried with anhydrous sodium sulfate, filtered and the filtrate was concentrated under reduced pressure to give a residue, which was triturated with methyl tert-butyl ether (10 mL) for 1 h. After filtered, the filter cake was dried to give 6,6-difluoro-3-(4,4,5,5-tetramethyl-1,3,2-dioxaborolan-2-yl)-5,6,7,8-tetrahydroquinoline (2 g, yield 45.86%) as a black solid. ¹H NMR: 400 MHz, MeOD, δ=1.34 (s, 12H), 2.37 (td, J=13.35, 6.69 Hz, 2H), 3.15 (t, J=7.00 Hz, 2H), 3.33-3.39 (m, 2H), 7.91 (s, 1H), 8.58 (s, 1H).

To a mixture of 6,6-difluoro-3-(4,4,5,5-tetramethyl-1,3,2-dioxaborolan-2-yl)-5,6,7,8-tetrahydroquinoline (2 g, 6.78 mmol) in tetrahydrofuran (20 mL) and water (5 mL) was added NaBO_{3·}4H₂O (3.13 g, 20.33 mmol). The mixture was stirred at 20° C. for 2 h. The reaction mixture was diluted with water (100 mL) and acidified by 1 M hydrochloric acid to PH=7. The aqueous layer was extracted with ethyl acetate (2×100 mL), and the organic layer was dried with anhydrous sodium sulfate, filtered and the filtrate was concentrated under reduced pressure to give a residue. The residue was triturated with methyl tert-butyl ether (5 mL) for 1 h, filtered and the filter cake was dried to give 6,6-difluoro-5,6,7,8-tetrahydroquinolin-3-ol (921 mg, yield 73.03%, purity 99.5%) as a white solid. The reaction mixture was not acidified to give 6-fluoro-7,8-dihydroquinolin-3-ol (189 mg, yield 17.4%, purity 93%) as a yellow solid.

For 6,6-difluoro-5,6,7,8-tetrahydroquinolin-3-ol, ¹H NMR: 400 MHz, DMSO-d₆, δ=2.28 (tt, J=14.01, 7.00 Hz, 2H), 2.90 (t, J=7.00 Hz, 2H), 3.27 (br t, J=15.01 Hz, 2H), 6.91 (d, J=2.63 Hz, 1H), 7.96 (d, J=2.75 Hz, 1H), 9.75 (s, 1H). LCMS (ESI+): m/z 186.1 (M+H)⁺, Rt: 1.305 min. LC/MS (The gradient was 0% B in 0.40 min and 0-60% B at 0.4-3.0 min,60-100% B at 3.0-4.0 min, and then 100-0% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Kinetex C18 2.1*50 mm, 5 μm. Detection methods were diode array (DAD), and evaporative light scattering detection (ELSD). MS mode was positive electrospray ionization. MS range was 100-1000.

For 6-fluoro-7,8-dihydroquinolin-3-ol, ¹H NMR: 400 MHz, DMSO-d₆, δ=2.65 (td, J=8.54, 3.44 Hz, 2H), 2.97 (td, J=8.63, 2.50 Hz, 2H), 6.14 (d, J=13.01 Hz, 1H), 6.84 (d, J=2.63 Hz, 1H), 7.76 (d, J=2.63 Hz, 1H), 9.62 (s, 1H). LCMS (ESI+): m/z 166.2 (M+H)⁺, Rt: 1.328 min. LC/MS (The gradient was 0% B in 0.40 min and 0-60% B at 0.4-3.0 min, 60-100% B at 3.0-4.0 min, and then 100-0% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Kinetex C18 2.1*50 mm, 5 μm. Detection methods were diode array (DAD), and evaporative light scattering detection (ELSD). MS mode was positive electrospray ionization. MS range was 100-1000.

To a mixture of 6,6-difluoro-5,6,7,8-tetrahydroquinolin-3-ol (400 mg, 2.16 mmol) in dimethylformamide (4 mL) was added 2-chloro-4-fluoro-pyridine (568.28 mg, 4.32 mmol) and cesium carbonate (Cs₂CO₃, 914.98 mg, 2.81 mmol). The mixture was stirred at 20° C. for 4 h. The reaction was quenched with water (80 mL), the aqueous layer was extracted with ethyl acetate (2×30 mL), the organic layer was dried with anhydrous sodium sulfate, and filtered. The filtrate was concentrated under reduced pressure to give a residue, which was purified by prep-TLC (petroleum ether/ethyl acetate=1/1) to give 3-((2-chloropyridin-4-yl)oxy)-6,6-difluoro-5,6,7,8-tetrahydroquinoline (303 mg, yield 47.13%, purity 99.7%) as a white solid. ¹H NMR: 400 MHz, MeOD, δ=2.39 (tt, J=13.57, 6.94 Hz, 2H), 3.17 (t, J=7.00 Hz, 2H), 3.38 (t, J=14.45 Hz, 2H), 6.97 (dd, J=5.75, 2.25 Hz, 1H), 7.04 (d, J=2.25 Hz, 1H), 7.50 (d, J=2.38 Hz, 1H), 8.26 (d, J=5.88 Hz, 1H), 8.29 (d, J=2.63 Hz, 1H). LCMS (ESI+): m/z 297.1 (M+H)⁺, Rt: 1.865 mn. LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization. MS range was 100-1000.

### Intermediate Example 13

### 3-((2-chloropyridin-4-yl)oxy)-6,8-difluoroquinoline

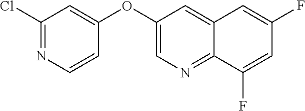 [SMILES: Fc1cc(F)c2ncc(Oc3ccnc(Cl)c3)cc2c1]

### Synthesis of 3-((2-chloropyridin-4-yl)oxy)-6,8-difluoroquinoline

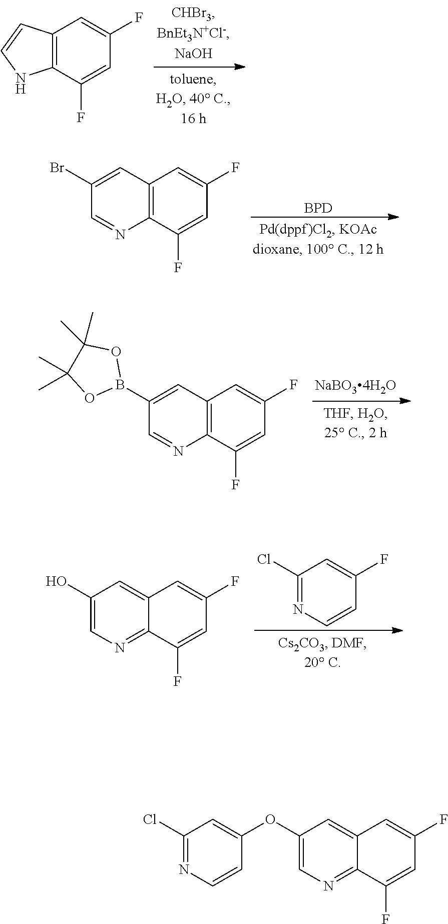

To a mixture of 5,7-difluoro-1H-indole (2.2 g, 14.37 mmol) and benzyl (triethyl) ammonium chloride (163.62 mg, 718.35 μmol) in toluene (2 mL) was added bromoform (3.63 g, 14.37 mmol) and the temperature was warmed to 40° C. A solution of sodium hydroxide (4.31 g, 107.75 mmol) in water (12 mL) was added dropwise over 0.25 h, causing a dark color to form. The reaction was stirred as a biphasic mixture at 40° C. for 16 h. One additional vial was set up as described above and both mixtures were combined for work up. The reaction was quenched with water (300 mL), the aqueous phase was extracted with ethyl acetate (3× 100 mL), the organic layer was dried with anhydrous sodium sulfate, filtered and the filtrate was concentrated under reduced pressure to give a residue. The residue was purified by column chromatography (SiO₂, petroleum ether/ethyl acetate=5/1) to give 3-bromo-6,8-difluoroquinoline (900 mg, yield 12.83%) as a white solid. ¹H NMR: 400 MHz, CDCl₃, δ=7.20-7.24 (m, 1H), 7.27-7.30 (m, 1H), 8.32 (s, 1H), 8.92 (d, J=1.88 Hz, 1H).

To a mixture of 3-bromo-6,8-difluoroquinoline (900 mg, 3.69 mmol) in 1,4-dioxane (10 mL) was added 4,4,5,5-tetramethyl-2-(4,4,5,5-tetramethyl-1,3,2-dioxaborolan-2-yl)-1,3,2-dioxaborolane (BPD, 1.40 g, 5.53 mmol), potassium acetate (KOAc, 1.09 g, 11.06 mmol) and Pd(dppf)Cl₂ (301.18 mg, 368.80 μmol). The mixture was stirred at 100° C. for 12 h under a nitrogen atmosphere. The reaction was quenched with water (100 mL), the aqueous layer was extracted with ethyl acetate (2×30 mL), the organic layer was dried with anhydrous sodium sulfate, filtered and the filtrate was concentrated under reduced pressure to give 6,8-difluoro-3-(4,4,5,5-tetramethyl-1,3,2-dioxaborolan-2-yl)quinoline (2 g, crude) as black oil. The crude product was used for the next step directly without purification. LC/MS description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 mL/min; Column: Halo C18, 3.0*30 mm, 5 μm; Wavelength: UV 220 nm&254 nm Column temperature: 40° C.; MS ionization: ESI.

To a mixture of 6,8-difluoro-3-(4,4,5,5-tetramethyl-1,3,2-dioxaborolan-2-yl)quinoline (2.150 g, 7.39 mmol) in tetrahydrofuran (16 mL) and water (4 mL) was added NaBO_{3·}4H₂O (4.55 g, 29.54 mmol). The mixture was stirred at 25° C. for 2 h. The reaction was quenched with water (100 mL), the aqueous layer was extracted with ethyl acetate (2× 30 mL), the organic phase was dried over anhydrous sodium sulfate, filtered and the filtrate was concentrated under reduced pressure to give a residue. The residue was purified by column chromatography (SiO₂, petroleum ether/ethyl acetate=1/1) to give 6,8-difluoroquinolin-3-ol (700 mg, yield 52.06%) as a white solid. ¹H NMR: 400 MHz, DMSO-d₆, δ=7.36-7.45 (m, 1H), 7.46-7.52 (m, 1H), 7.55 (dd, J=2.25, 1.63 Hz, 1H), 8.57 (d, J=2.63 Hz, 1H), 10.75 (br s, 1H). LCMS (ESI+): m/z 182.1 (M+H)⁺, Rt: 2.554 min. LC/MS (The gradient was 0% B in 0.40 min and 0-30% B at 0.4-3.0 min, 30-100% B at 3.0-4.0 min, and then 100-0% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Luna C18 50*2.0 mm column (5 μm particles). Detection methods were diode array (DAD) and evaporative light scattering (ELSD) detection. MS mode was positive electrospray ionization. MS range was 100-1000.

To a mixture of 6,8-difluoroquinolin-3-ol (200 mg, 1.10 mmol) in dimethylformamide (2 mL) was added 2-chloro-4-fluoro-pyridine (290.46 mg, 2.21 mmol) and cesium carbonate (467.67 mg, 1.44 mmol). The mixture was stirred at 110° C. for 2 h. The reaction was quenched with water (30 mL), the aqueous layer was extracted with ethyl acetate (2× 10 mL), the organic layer was dried with anhydrous sodium sulfate, filtered and the filtrate was concentrated under reduced pressure to give a residue, which was purified by prep-TLC (petroleum ether/ethyl acetate=2/1) to give 3-((2-chloropyridin-4-yl)oxy)-6,8-difluoroquinoline (243 mg, yield 74.75%) as a white solid. ¹H NMR: 400 MHz, DMSO-d₆, δ=7.14-7.26 (m, 1H), 7.35 (d, J=1.38 Hz, 1H), 7.67 (br d, J=9.13 Hz, 1H), 7.70-7.81 (m, 1H), 8.29 (br s, 1H), 8.39 (d, J=5.63 Hz, 1H), 8.91 (d, J=1.88 Hz, 1H). LCMS (ESI+): m/z 293.1 (M+H)⁺, Rt: 2.096 min. LC/MS (The gradient was 5% B in 0.40 min and 5-95% B at 0.40-3.00 min, hold on 95% B for 1.00 min, and then 95-5% B in 0.01 min, the flow rate was 1.0 mL/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods are diode array (DAD) and evaporative light scattering (ELSD) detection as well as positive electrospray ionization. MS range was 100-1000.

### Intermediate Example 14

### 3-methyl-1-(trifluoromethyl)-1H-indazol-5-ol

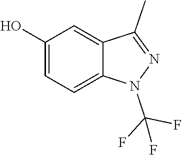

### Synthesis of 3-methyl-1-(trifluoromethyl)-1H-indazol-5-ol

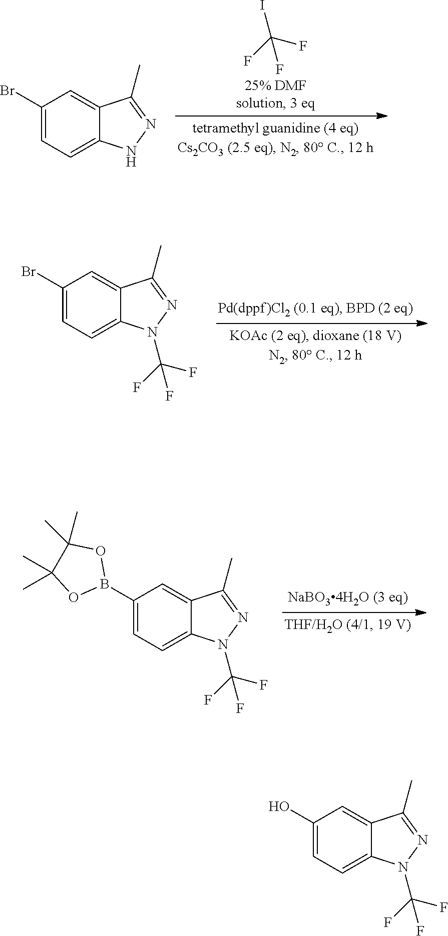 [SMILES: Cc1n[nH]c2ccc(Br)cc12 ; FC(F)(F)I ; Cc1nn(C(F)(F)F)c2ccc(Br)cc12 ; Cc1nn(C(F)(F)F)c2ccc(B3OC(C)(C)C(C)(C)O3)cc12 ; Cc1nn(C(F)(F)F)c2ccc(O)cc12]

A solution of 5-bromo-3-methyl-1H-indazole (50 mg, 236.90 μmol), 1,1,3,3-tetramethylguanidine (109.14 mg, 947.61 μmol, 119.15 μL) and Cs₂CO₃ (192.97 mg, 592.25 μmol) in trifluoro (iodo) methane (556.94 mg, 710.70 μmol) (25% of dimethyl formamide solution) was stirred at 80° C. under nitrogen for 12 h. Twenty-nine additional vials were set up as described above. Reaction mixtures were combined and purified. The reaction was quenched by addition of water (30 mL), and then extracted by ethyl acetate (3× 8 mL). The combined organic phase was dried with anhydrous sodium sulfate, filtered, and concentrated in vacuum. The crude product was purified by silica gel column chromatography (petroleum ether/ethyl acetate=I/O to 6/1) to afford 5-bromo-3-methyl-1-(trifluoromethyl)-1H-indazole (270 mg, yield 12.57%) as a yellow solid. ¹H NMR: 400 MHz, CD3OD, δ=1.79 (s, 3H), 7.91 (dd, J=8.25, 1.63 Hz, 1H), 7.98 (s, 1H), 8.14 (d, J=8.38 Hz, 1H)

5-bromo-3-methyl-1-(trifluoromethyl)-1H-indazole (140 mg, 501.69 μmol), BPD (254.80 mg, 1.00 mmol), potassium acetate (98.47 mg, 1.00 mmol) and Pd(dppf)Cl₂ (36.71 mg, 50.17 μmol) in dioxane (1.4 mL) was de-gassed and then heated to 80° C. for 12 h under nitrogen. Two additional vials were set up as described above (to give a total of 270 mg of 5-bromo-3-methyl-1-(trifluoromethyl)-1H-indazole). The reaction mixture was concentrated to give the crude product (151.92 mg) which was used in the next step without further purification. Crude product (163 mg) was purified by silica gel column chromatography (petroleum ether/ethyl acetate=I/O to 0/1) to afford 3-methyl-5-(4,4,5,5-tetramethyl-1,3,2-dioxaborolan-2-yl)-1-(trifluoromethyl)-1H-indazole (71 mg, yield 41.31%) as a yellow solid. 1H NMR: 400 MHz, CDCl₃, δ=1.38 (s, 12H), 1.79 (s, 3H), 8.04 (s, 1H), 8.08 (d, J=7.75 Hz, 1H), 8.14-8.21 (m, 1H). LCMS (ESI+): m/z 327.3 (M+H)⁺, Rt: 2.693 min, m/z 245.1 (M−83+H)⁺, Rt: 1.666 min. LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization.MS range was 1001000.

NaBO_{3·}4H₂O (181.87 mg, 1.18 mmol, 3 eq) was added to a stirred mixture of 3-methyl-5-(4,4,5,5-tetramethyl-1,3,2-dioxaborolan-2-yl)-1-(trifluoromethyl)-1H-indazole (128.55 mg, 394.18 μmol, 1 eq) in tetrahydrofuran (2 mL), and water (0.4 mL). The mixture was stirred at 20° C. for 2 h. The reaction was combined to give a total of 151.92 mg of crude 3-methyl-5-(4,4,5,5-tetramethyl-1,3,2-dioxaborolan-2-yl)-1-(trifluoromethyl)-1H-indazole). Water (2 mL) was added, followed by extraction with ethyl acetate (3×5 mL). The combined organic phase was dried with anhydrous Na₂SO₄, filtered, and concentrated in vacuum. The crude product was purified by silica gel column chromatography (petroleum ether/ethyl acetate=I/O to 0/1) to afford 3-methyl-1-(trifluoromethyl)-1H-indazol-5-ol (70 mg, yield 60.61%) as a white solid. ¹H NMR: 400 MHz, CD3OD, δ=1.71 (s, 3H), 6.95-7.14 (m, 2H), 8.00 (d, J=8.26 Hz, 1H). LCMS (ESI+): m/z 217.2 (M+H)⁺, Rt: 1.705 min. LC/MS (the gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization. MS range was 100-1000.

### Intermediate Example 15

### 3-methyl-1-(trifluoromethyl)-1H-indazol-6-ol

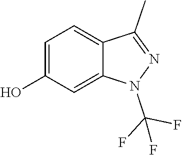

### Synthesis of 3-methyl-1-(trifluoromethyl)-1H-indazol-6-ol

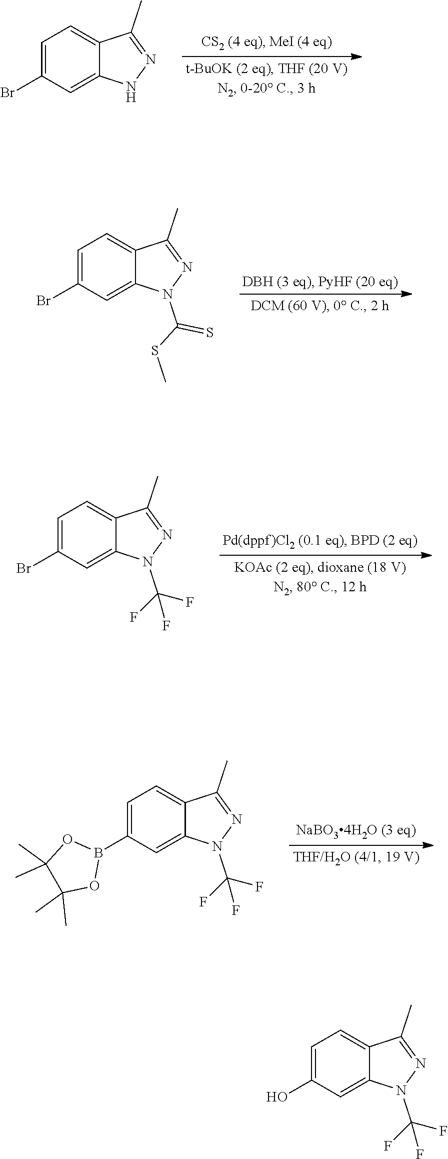 [SMILES: Cc1n[nH]c2cc(Br)ccc12 ; CSC(=S)n1nc(C)c2ccc(Br)cc21 ; Cc1nn(C(F)(F)F)c2cc(Br)ccc12 ; Cc1nn(C(F)(F)F)c2cc(B3OC(C)(C)C(C)(C)O3)ccc12 ; Cc1nn(C(F)(F)F)c2cc(O)ccc12]

A mixture of 6-bromo-3-methyl-1H-indazole (3.1 g, 14.69 mmol) and potassium tert-butoxide (3.30 g, 29.38 mmol) in tetrahydrofuran (124 mL) was stirred at 20° C. for 30 minutes under nitrogen. Methanedithione (4.47 g, 58.75 mmol) was added at 20° C. and the reaction was stirred at 20° C. for 2 h. Iodomethane (8.34 g, 58.75 mmol) was added at 20° C., and the mixture was stirred at 20° C. for 1 h. An additional vial was set up as described above (total of 4.3 g of 6-bromo-3-methyl-1H-indazole). The reaction was quenched by addition of water (400 mL) and extracted with ethyl acetate (3×50 mL). The combined organic phase was concentrated, filtrated, and the filter cake was dried to give methyl 6-bromo-3-methyl-1H-indazole-1-carbodithioate (3.9 g, yield 70.07%) as a yellow solid. ¹H NMR: 400 MHz, DMSO-d₆, δ=2.57 (br s, 3H), 2.63 (br s, 3H), 7.69 (br d, J=7.63 Hz, 1H), 7.90 (br d, J=8.00 Hz, 1H), 9.26 (br s, 1H). LCMS (ESI+): m/z 301.0&303.0 (M+H)⁺, Rt: 0.697 min. Description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 ml/min; Column: Halo C18, 3.0*30 mm, 5 μm; Wavelength: UV 220 nm&254 nm. Column temperature: 40° C.; MS ionization: ESI.

To a solution of 1,3-dibromo-5,5-dimethyl-imidazolidine-2,4-dione (1.14 g, 3.98 mmol) in dichloromethane (16 mL) was added pyridine-HF complex (2.63 g, 26.56 mmol, 2.39 mL,) at 0° C. The mixture was stirred at 0° C. for 10 min. A solution of methyl 6-bromo-3-methyl-1H-indazole-1-carbodithioate (400 mg, 1.33 mmol) in dichloromethane (8 mL) was added to the above solution dropwise at 0° C. The reaction was stirred at 0° C. for 2 h. Eight additional vials were set up as described above (total of 3.4 g of methyl 6-bromo-3-methyl-1H-indazole-1-carbodithioate). The mixture was combined and concentrated. The residue was purified by prep-TLC (petroleum ether/ethyl acetate=8/1) to give 6-bromo-3-methyl-1-(trifluoromethyl)-1H-indazole (500 mg, yield 15.22%) as a white solid. ¹H NMR: 400 MHz, CDCl₃, δ=2.59 (s, 3H), 7.46 (dd, J=8.51, 1.50 Hz, 1H), 7.55-7.59 (m, 1H), 7.83 (s, 1H). LCMS (ESI+): m/z 279.1&281.1 (M+H)⁺, Rt: 0.608 min. Description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 ml/min; Column: Halo C18, 3.0*30 mm, 5 μm; Wavelength: UV 220 nm&254 nm; Column temperature: 40° C.; MS ionization: ESI.

A mixture of 6-bromo-3-methyl-1-(trifluoromethyl)-1H-indazole (420 mg, 1.51 mmol), BPD (764.39 mg, 3.01 mmol), potassium acetate (295.42 mg, 3.01 mmol) and Pd(dppf)Cl₂ (110.13 mg, 150.51 μmol) in dioxane (4.2 mL) was de-gassed and then heated to 80° C. for 12 h under N₂. Another additional vial was set up as described above (total of 500 mg of 6-bromo-3-methyl-1-(trifluoromethyl)-1H-indazole). The reaction mixture was concentrated. 338 mg of the crude product was used into the next step without further purification. 245 mg of the crude product was purified by prep-TLC (petroleum ether/ethyl acetate=10/1) to give 3-methyl-6-(4,4,5,5-tetramethyl-1,3,2-dioxaborolan-2-yl)-1-(trifluoromethyl)-1H-indazole (201 mg, yield 79.53%) as a white solid. ¹H NMR: 400 MHz, CDCl₃, δ=1.39 (s, 12H), 2.62 (s, 3H), 7.67-7.72 (m, 1H), 7.73-7.78 (m, 1H), 8.10 (s, 1H). LCMS (ESI+): m/z 327.2 (M+H)⁺, Rt: 2.850 min, m/z 245.2 (M−83+H)⁺, Rt: 1.736 min. LCMS: (the gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization.MS range was 100-1000.

NaBO_{3·}4H₂O (346.76 mg, 2.25 mmol) was added into a stirred mixture of 3-methyl-6-(4,4,5,5-tetramethyl-1,3,2-dioxaborolan-2-yl)-1-(trifluoromethyl)-1H-indazole (245 mg, 751.25 μmol) in tetrahydrofuran (5 mL) and water (1 mL). The mixture was stirred at 20° C. for 2 h. The reaction was filtered and concentrated in a vacuum. The crude product was combined (total of 338.49 mg of 3-methyl-6-(4,4,5,5-tetramethyl-1,3,2-dioxaborolan-2-yl)-1-(trifluoromethyl)-1H-indazole) and purified by prep-TLC (petroleum ether/ethyl acetate=4/1) to afford 3-methyl-1-(trifluoromethyl)-1H-indazol-6-ol (177 mg, yield 75.82%) as a white solid. ¹H NMR: 400 MHz, DMSO-d₆, δ=2.47 (s, 3H), 6.89 (dd, J=8.69, 1.94 Hz, 1H), 6.94 (d, J=1.38 Hz, 1H), 7.68 (d, J=8.63 Hz, 1H), 10.30 (s, 1H). LCMS (ESI+): m/z 217.2 (M+H)⁺, Rt: 1.824 min. LCMS: (the gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.037% trifluoroacetic acid in water, mobile phase B was 0.018% trifluoroacetic acid in acetonitrile. The column used for chromatography was a Kinetex C18 50*2.1 mm column (5 μm particles). Detection methods were diode array (DAD) and evaporative light scattering (ELSD) detection as well as positive electrospray ionization.MS range was 100-1000.

### Intermediate Example 16

### 3-methyl-1-(trifluoromethyl)-1H-pyrazolo[4,3-b]pyridin-6-ol

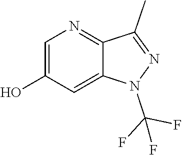

### Synthesis of 3-methyl-1-(trifluoromethyl)-1H-pyrazolo[4,3-b]pyridin-6-ol

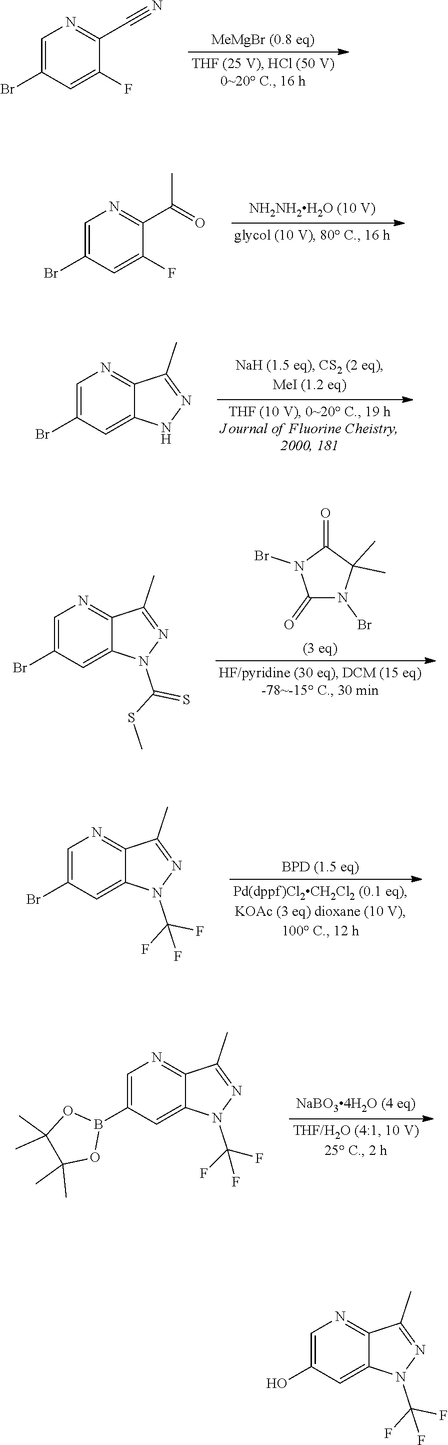

To a solution of 5-bromo-3-fluoropicolinonitrile (10 g, 49.75 mmol) in tetrahydrofuran (250 mL) was added methyl magnesium chloride (13.27 mL, 3 M, 39.80 mmol) dropwise over 15 min at 0° C. under nitrogen atmosphere. The mixture was stirred at 0° C. for 15 min. The reaction mixture was added into a stirring solution of HCl (500 mL, 3 M, 1.5 mol) at 0° C. The mixture was stirred at 20° C. for 15 h. The solution was then reverse quenched by addition to a saturated aqueous sodium carbonate solution (500 mL) at 0° C. The aqueous layer was extracted with ethyl acetate (2×500 mL). The organic layers were combined, dried over anhydrous sodium sulfate and concentrated to give crude product, which was purified by column chromatography (SiO₂, petroleum ether/ethyl acetate=6/1) to give 1-(5-bromo-3-fluoropyridin-2-yl)ethan-1-one (6 g, yield 55.31%, purity 99%) as a yellow solid. 1H NMR: 400 MHz, MeOD, δ=2.64 (d, J=0.88 Hz, 3H), 8.06 (dd, J=10.26, 1.75 Hz, 1H), 8.62 (s, 1H).

To a solution of 1-(5-bromo-3-fluoropyridin-2-yl)ethan-1-one (4 g, 18.35 mmol) in ethylene glycol (40 mL) was added hydrazine hydrate (NH₂NH₂: H₂O, 40.00 mL, 808.12 mmol, 98% purity) at 20° C. The reaction mixture was stirred at 100° C. for 12 h. The reaction was quenched with saturated ammonium chloride aqueous solution (300 mL), the aqueous layer was extracted with ethyl acetate (2× 150 mL), the organic layer was dried with anhydrous sodium sulfate, filtered and the filtrate was concentrated under reduced pressure to give a residue, which was purified by column chromatography (SiO₂, petroleum ether/ethyl acetate=3/1) to give 6-bromo-3-methyl-1H-pyrazolo[4,3-b]pyridine (2 g, yield 50.38%, purity 98%) as a yellow solid. ¹H NMR: 400 MHz, MeOD, δ=2.59 (s, 3H), 8.16 (d, J=1.75 Hz, 1H), 8.51 (d, J=1.88 Hz, 1H).

To a solution of 6-bromo-3-methyl-1H-pyrazolo[4,3-b]pyridine (2.8 g, 13.20 mmol) in tetrahydrofuran (30 mL) was added sodium hydride (792.20 mg, 19.81 mmol, 60% purity) at 0° C. The mixture was stirred for 1 h at 20° C. Methanedithione (2.01 g, 26.41 mmol) was added dropwise at 0° C. and the resulting mixture was stirred at 20° C. for 16 h. The mixture was cooled to 0° C. again and iodomethane (2.25 g, 15.85 mmol) was added to the above solution dropwise. The resulting mixture was stirred at 20° C. for 2 h. The reaction was quenched with saturated ammonium chloride aqueous solution (150 mL), the aqueous layer was extracted with ethyl acetate (3× 50 mL), the organic layer was dried with anhydrous sodium sulfate, filtered and the filtrate was concentrated under reduced pressure to give a residue, which was triturated with ethyl acetate (10 mL) and acetonitrile (2 mL) for 1 h. After filtering, the filter cake was dried to give methyl 6-bromo-3-methyl-1H-pyrazolo[4,3-b]pyridine-1-carbodithioate (2 g, yield 48.11%, purity 96%) as a yellow solid. 1H NMR: 400 MHz, DMSO-d₆, δ=2.60 (s, 3H), 2.66 (s, 3H), 8.87 (d, J=2.00 Hz, 1H), 9.44 (d, J=2.00 Hz, 1H).

To a solution of 1,3-dibromo-5,5-dimethyl-imidazolidine-2,4-dione (5.45 g, 19.06 mmol) in dichloromethane (80 mL) was added pyridine hydrofluoride (17.17 mL, 190.59 mmol) at −78° C. The mixture was stirred for 10 min at −78° C. A solution of methyl 6-bromo-3-methyl-1H-pyrazolo[4,3-b]pyridine-1-carbodithioate (2 g, 6.35 mmol) in dichloromethane (40 mL) was added to the above solution dropwise at −78° C. After the addition, the resulting mixture was stirred at −15° C. for 20 min. The reaction was quenched with saturated sodium bicarbonate aqueous solution (200 mL), the aqueous layer was extracted with dichloromethane (3×50 mL). The organic layer was dried with anhydrous sodium sulfate, filtered and the filtrate was concentrated under reduced pressure to give a residue, which was purified by column chromatography (SiO₂, petroleum ether/ethyl acetate=6/1) to give 6-bromo-3-methyl-1-(trifluoromethyl)-1H-pyrazolo[4,3-b]pyridine (600 mg, yield 30.35%, purity 90%) as a white solid. ¹H NMR: 400 MHz, MeOD, δ=2.63 (s, 3H), 8.40 (s, 1H), 8.78 (d, J=1.75 Hz, 1H).

To a mixture of 6-bromo-3-methyl-1-(trifluoromethyl)-1H-pyrazolo[4,3-b]pyridine (600 mg, 1.93 mmol) in 1,4-dioxane (6 mL) was added 4,4,5,5-tetramethyl-2-(4,4,5,5-tetramethyl-1,3,2-dioxaborolan-2-yl)-1,3,2-dioxaborolane (BPD 734.49 mg, 2.89 mmol), potassium acetate (KOAc 567.72 mg, 5.78 mmol) and Pd(dppf)Cl₂: CH₂Cl₂ (157.47 mg, 192.83 μmol). The mixture was stirred at 100° C. for 12 h under nitrogen atmosphere. The reaction was quenched with water (50 mL), the aqueous layer was extracted with ethyl acetate (2× 20 mL), the organic layer was dried with anhydrous sodium sulfate, filtered and the filtrate was concentrated under reduced pressure to give 3-methyl-6-(4,4,5,5-tetramethyl-1,3,2-dioxaborolan-2-yl)-1-(trifluoromethyl)-1H-pyrazolo[4,3-b]pyridine (1.1 g, crude) as black oil, which was used for next step directly without purification. LC/MS: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 mL/min; Column: Halo C18, 3.0*30 mm, 5 μm; Wavelength: UV 220 nm&254 nm Column temperature: 40° C.; MS ionization: ESI.

To a mixture of 3-methyl-6-(4,4,5,5-tetramethyl-1,3,2-dioxaborolan-2-yl)-1-(trifluoromethyl)-1H-pyrazolo[4,3-b]pyridine (1.1 g, 1.68 mmol, purity 50%) in tetrahydrofuran (8.8 mL) and water (2.2 mL) was added NaBO_{3·}4H₂O (1.03 g, 6.73 mmol). The mixture was stirred at 25° C. for 2 h. The reaction was quenched with water (50 mL), the aqueous layer was extracted with ethyl acetate (2× 20 mL), the organic layer was dried with anhydrous sodium sulfate, filtered and the filtrate was concentrated under reduced pressure to give a residue, which was purified by column chromatography (SiO₂, petroleum ether/ethyl acetate=1/1) to give 3-methyl-1-(trifluoromethyl)-1H-pyrazolo[4,3-b]pyridin-6-ol (215 mg, yield 58.71%, purity 99.7%) as a white solid. ¹H NMR: 400 MHz, MeOD, δ=2.56 (s, 3H), 7.27-7.36 (m, 1H), 8.28 (d, J=2.25 Hz, 1H). LCMS (ESI+): m/z 218.1 (M+H)⁺, Rt: 2.086 min. LC/MS (The gradient was 0% B in 0.40 min and 0-60% B at 0.4-3.0 min, 60-100%

B at 3.0-4.0 min, and then 100-0% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Kinetex C18 2.1*50 mm, 5 μm. Detection methods were diode array (DAD), and evaporative light scattering detection (ELSD). MS mode was positive electrospray ionization. MS range was 100-1000.

### Intermediate Example 17

### 3-fluoro-1-methyl-1H-indazol-6-ol

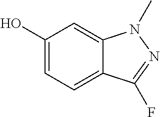

### Synthesis of 3-fluoro-1-methyl-1H-indazol-6-ol

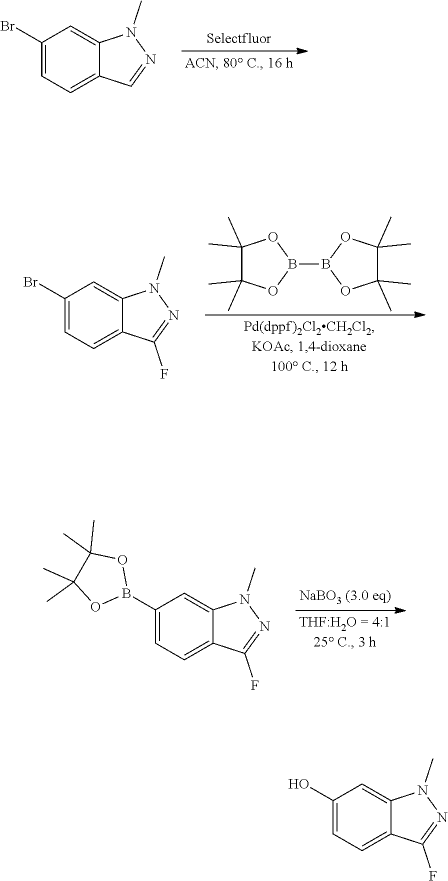

To a solution of 6-bromo-1-methyl-1H-indazole (7.3 g, 34.59 mmol, 1 eq) in ACN(110 mL) was added Selectfluor (15.93 g, 44.96 mmol, 1.3 eq). The mixture was stirred at 90° C. for 16 h. The mixture was filtered, and the filtrate was concentrated under reduced pressure to give a residue. The reaction mixture was purified by prep HPLC (HCl) to give 6-bromo-3-fluoro-1-methyl-1H-indazole (1.9 g, 22.78% yield, 95% purity) as a yellow solid. LCMS (ESI+): m/z 227.97, (M+H)⁺, Rt: 0.499 min. Description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 ml/min; Column: Halo C18, 3.0*30 mm, 5 μm; Wavelength: UV 220 nm&254 nm Column temperature: 40° C.; MS ionization: ESI. ¹H NMR: 400 MHz, MeOD, δ=3.88-3.90 (m, 3H), 7.27 (dd, J=8.69, 1.44 Hz, 1H), 7.54 (d, J=8.76 Hz, 1H), 7.75 (t, J=1.38 Hz, 1H).

To a solution of 6-bromo-3-fluoro-1-methyl-1H-indazole (1.9 g, 7.88 mmol, 1 eq) in 1, 4-dioxane (20 mL) was added 4,4,5,5-tetramethyl-2-(4,4,5,5-tetramethyl-1,3,2-dioxaborolan-2-yl)-1,3,2-dioxaborolane (3.00 g, 11.82 mmol, 1.5 eq), potassium acetate (2.32 g, 23.64 mmol, 3 eq) and cyclopentyl (diphenyl) Pd(dppf)₂Cl₂. CH₂Cl₂ (643.54 mg, 788.04 μmol, 0.1 eq) under N₂. The mixture was stirred at 100° C. for 12 h. The reaction mixture was quenched by water (2 mL) at 20° C., and then extracted with ethyl acetate (3×1 mL). The combined organic layers were filtered and concentrated under reduced pressure. The residue was purified by prep-TLC (petroleum ether/ethyl acetate=2/1) to give the 3-fluoro-1-methyl-6-(4,4,5,5-tetramethyl-1,3,2-dioxaborolan-2-yl)-1H-indazole (1.4 g, 45.04% yield, 70% purity) was obtained as a yellow solid. LCMS (ESI+): m/z 276.14, (M+H)⁺, Rt: 0.596 min. Description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 ml/min; Column: Halo C18, 3.0*30 mm, 5 μm; Wavelength: UV 220 nm&254 nm Column temperature: 40° C.; MS ionization: ESI. ¹H NMR: 400 MHz, MeOD, δ=1.39 (s, 12H), 3.96 (d, J=0.63 Hz, 3H), 7.55 (d, J=8.25 Hz, 1H), 7.65 (d, J=8.13 Hz, 1H), 7.82 (s, 1H).

To a solution of 3-fluoro-1-methyl-6-(4,4,5,5-tetramethyl-1,3,2-dioxaborolan-2-yl)-1H-indazole (1.7 g, 4.31 mmol, 1 eq) in THF (17 mL) and H₂O (4.3 mL) was added NaBO_{3·}4H₂O (2.65 g, 17.24 mmol, 3.32 mL, 4 eq). The mixture was stirred at 25° C. for 16 h. The reaction was quenched with 1 M HCl (500 mL), the aqueous layer was extracted with ethyl acetate (3× 200 mL), the organic layer was dried with

Na₂SO₄, filtered and the filtrate was concentrated under reduced pressure to give a residue. The reaction mixture was purified by prep HPLC (NH₄HCO₃) to give 3-fluoro-1-methyl-1H-indazol-6-ol (0.5049 g, yield 70.51%) as a white solid. LCMS (ESI+): m/z 166.05, (M+H)⁺, Rt: 1.449 min. Description: Mobile Phase: 0.04% TFA in water (solvent A) and 0.02% TFA in acetonitrile (solvent B), using the elution gradient 5%-95% (solvent B) over 3.0 minutes and holding at 95% for 1.0 minutes at a flow rate of 1.0 ml/min; ¹H NMR: ET68711-172-P1A1, 400 MHz, MeOD, δ=3.76 (d, J=0.88 Hz, 3H), 6.67 (t, J=2.00 Hz, 1H), 6.71 (dd, J=8.76, 1.88 Hz, 1H), 7.42 (d, J=8.76 Hz, 1H).

### Intermediate Example 18

### 3-fluoro-1-methyl-1H-indazol-5-ol

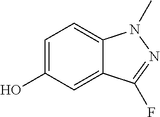

### Synthesis of 3-fluoro-1-methyl-1H-indazol-5-ol

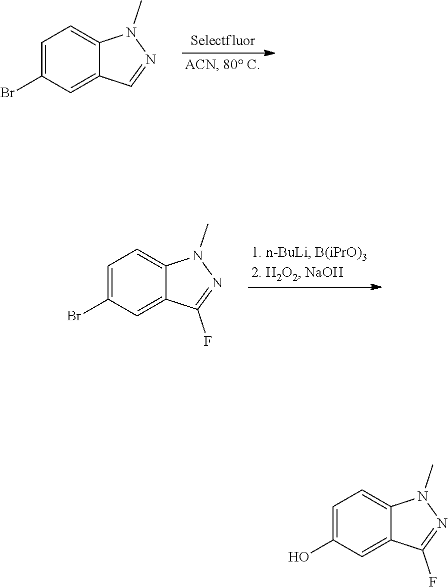

To a solution of 5-bromo-1-methyl-1H-indazole (5 g, 23.69 mmol, 1 eq) in ACN(50 mL) was added Selectfluor (16.78 g, 47.38 mmol, 2 eq) and HOAc (0.1 mL). The mixture was stirred at 80° C. for 14 h. Water (100 mL) was added to the reaction. The mixture was extracted with ethyl acetate (3×50 mL). The combined organic layers were washed with brine and dried over Na₂SO₄. The organic layer was concentrated under a high vacuum. The residue was purified by column chromatography (SiO₂, Petroleum ether/Ethyl acetate=I/O to 3/1) to give 5-bromo-3-fluoro-1-methyl-1H-indazole (2.5 g, 5.46 mmol, 23.04% yield, 50% purity) as yellow oil. LCMS (ESI+): m/z 229.0/231.0 [M+H]⁺Rt: 0.530 min.

To a solution of 5-bromo-3-fluoro-1-methyl-1H-indazole (1 g, 4.37 mmol, 1 eq) and triisopropyl borate (821.09 mg, 4.37 mmol, 1.00 mL, 1 eq) in THF (10 mL) was added n-BuLi (2.5 M, 4.37 mL, 2.5 eq) at −78° C. under nitrogen. The mixture was stirred at −78° C. for 30 min. Then the mixture was stirred at 25° C. for 2 h. The reaction was cooled to 0° C. and was added H₂O₂ (1.83 g, 16.15 mmol, 1.55 mL, 30% purity, 3.7 eq) and NaOH (2 M, 2.18 mL, 1 eq). The reaction was stirred at 0° C. for 12 min. The mixture was stirred at 25° C. for 12 h. The pH of mixture was adjusted to around 7 with the HCl (1 M). Then the reaction was quenched with saturated Na₂S₂O₃ aqueous solution (30 mL), the aqueous layer was extracted with ethyl acetate (2× 40 mL), the organic layer was dried with Na₂SO₄, filtered, and the filtrate was concentrated under reduced pressure to give a residue. The residue was purified by prep-HPLC (HCl condition) to give 3-fluoro-1-methyl-1H-indazol-5-ol (216 mg, 1.27 mmol, 11.62% yield, 97.6% purity) as a yellow solid. LCMS (ESI+): m/z 167.2 [M+H]⁺Rt: 1.351 min. ¹H NMR: 400 MHz, CDCl₃, δ=3.89 (d, J=1.13 Hz, 3H), 4.69-4.82 (m, 1H), 6.98 (d, J=2.25 Hz, 1H), 7.05 (dd, J=9.07, 2.31 Hz, 1H), 7.20 (dd, J=9.01, 2.13 Hz, 1H).

### Intermediate Example 19

### 3-fluoro-1-methyl-1H-pyrazolo[4,3-b]pyridin-6-ol

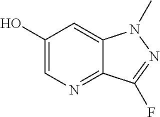

### Synthesis of 3-fluoro-1-methyl-1H-pyrazolo[4,3-b]pyridin-6-ol

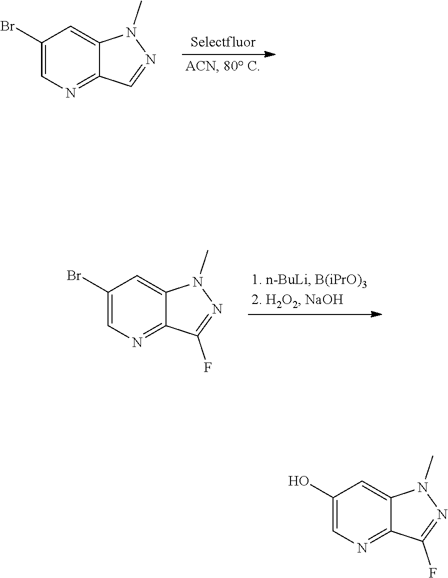

3-Fluoro-1-methyl-1H-pyrazolo[4,3-b]pyridin-6-ol was prepared following a procedure similar to that of intermediate example 18, using appropriate staring materials. LCMS (ESI+): m/z 168.2 [M+H]⁺Rt: 1.074 min. ¹H NMR: 400 MHz, CD₃OD, δ=3.91 (s, 3H), 7.47-7.48 (m, 1H), 8.30 (d, J=2.40 Hz, 1H).

### Intermediate Example 20

### 3-fluoro-1-methyl-1H-pyrazolo[3,4-b]pyridin-5-ol

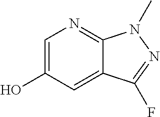

### Synthesis of 3-fluoro-1-methyl-1H-pyrazolo[3,4-b]pyridin-5-ol

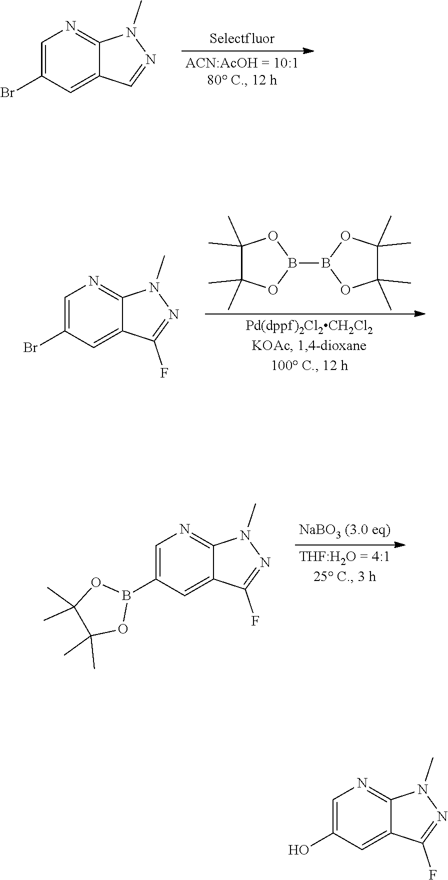

To a solution of 5-bromo-1-methyl-1H-pyrazolo[3,4-b]pyridine (4.3 g, 20.23 mmol) in acetonitrile (50 mL) and acetic acid (5 mL) was added Selectfluor (3.04 g, 60.84 mmol). The mixture was stirred at 80° C. for 12 h. The reaction mixture was filtered and concentrated under reduced pressure to give a residue. The residue was purified by column chromatography (SiO₂, Petroleum ether/Ethyl acetate=100/0 to 90/10) to afford 5-bromo-3-fluoro-1-methyl-1H-pyrazolo[3,4-b]pyridine (2.4 g, yield 51.45%) as a white solid. ¹H NMR: 400 MHz, CDCl₃, δ=3.94 (d, J=1.00 Hz, 3H), 8.05-8.09 (m, 1H), 8.50 (d, J=2.13 Hz, 1H).

To a solution of 5-bromo-3-fluoro-1-methyl-1H-pyrazolo[3,4-b]pyridine (100 mg, 434.71 μmol,) in 1,4-dioxane (1 mL) was added potassium acetate (127.99 mg, 1.30 mmol,) and Pd(dppf)₂Cl₂. CH₂Cl₂ (35.50 mg, 43.47 μmol,) and 4,4,5,5-tetramethyl-2-(4,4,5,5-tetramethyl-1,3,2-dioxaborolan-2-yl)-1,3,2-dioxaborolane (165.58 mg, 652.07 μmol) under nitrogen. The mixture was stirred at 100° C. for 12 h. The reaction mixture was quenched by addition sodium hydroxide (3×2 mL) at 0° C., then diluted with water (3×2 mL) and extracted with dichloromethane (3×2 mL). The combined organic layers were washed with sodium bicarbonate (3×2 mL), dried over with sodium sulfate, filtered and concentrated under reduced pressure to give a residue. The residue was purified by prep-TLC (SiO₂, Petroleum ether/Ethyl acetate =4:1) to afford 3-fluoro-1-methyl-5-(4,4,5,5-tetramethyl-1,3,2-dioxaborolan-2-yl)-1H-pyrazolo[3,4-b]pyridine (54 mg, yield 44.83%) as a white solid. ¹H NMR: 400 MHz, CDCl₃, δ=1.30 (s, 12H), 3.96 (d, J=0.63 Hz, 3H), 8.40 (s, 1H), 8.81 (d, J=1.50 Hz, 1H). LCMS (ESI+): m/z 278.1 (M+H)⁺, Rt: 0.565 min. (The column used for chromatography was a HALO AQ-C18 2.1*30 mm, (2.7 μm particles). Detection methods were diode array (DAD). MS mode was positive electrospray ionization. MS range was 100-1000. Mobile phase A was 0.037% TFA in water, and mobile phase B was 0.018% TFA in HPLC grade acetonitrile. The gradient was 5-95% B in 2.20 min. 5% B in 0.01 min, 5-95% B (0.01-1.00 min), 95-100% B (1.00-1.80 min), 5% B in 1.81 min, with a hold at 5% B for 0.40 min. The flow rate was 1.0 mL/min. To a solution of 3-fluoro-1-methyl-5-(4,4,5,5-tetramethyl-1,3,2-dioxaborolan-2-yl)-1H-pyrazolo[3,4-b]pyridine (1.0 g, 3.61 mmol, 1 eq) in tetrahydrofuran (10 mL) and water (2.5 mL) was added NaBO_{3·}4H₂O (2.22 g, 14.44 mmol, 2.78 mL, 4 eq). The mixture was stirred at 25° C. for 4 h. The reaction mixture was concentrated under reduced pressure and residue was diluted with water (2×30 mL) and extracted with ethyl acetate (2×30 mL). The combined organic layers were washed with brine (30 mL), dried over with sodium sulfate, filtered, and concentrated under reduced pressure to give a residue. The residue was purified by column chromatography (SiO₂, Petroleum ether/Ethyl acetate=100/0 to 85/15) to afford 3-fluoro-1-methyl-1H-pyrazolo[3,4-b]pyridin-5-ol (1.6 g, yield 28.52%) as a white solid. ¹H NMR: 400 MHz, DMSO-d₆, δ=3.90 (s, 3H), 7.39 (d, J=1.88 Hz, 1H), 8.30 (d, J=2.50 Hz, 1H), 9.97 (s, 1H). LCMS (ESI+): m/z 399.1 (M+H)⁺, Rt: 0.543 min. Description: Mobile Phase: 0.04% TFA in water (solvent A) and 0.02% TFA in acetonitrile (solvent B), using the elution gradient 10%-100% (solvent B) over 0.5 minutes and holding at 100% for 0.4 minutes at a flow rate of 2.0 ml/min; Column: Halo C18, 3.0*30 mm, 5 μm; Wavelength: UV 220 nm&254 nm Column temperature: 40° C.; MS ionization: ESI.

### Intermediate Example 21

### 2-ethyl-4-(4,4,5,5-tetramethyl-1,3,2-dioxaborolan-2-yl)benzamide

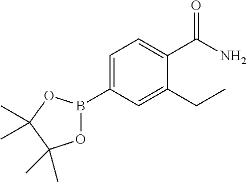 [SMILES: CCc1cc(B2OC(C)(C)C(C)(C)O2)ccc1C(N)=O]

### Synthesis of 2-ethyl-4-(4,4,5,5-tetramethyl-1,3,2-dioxaborolan-2-yl)benzamide

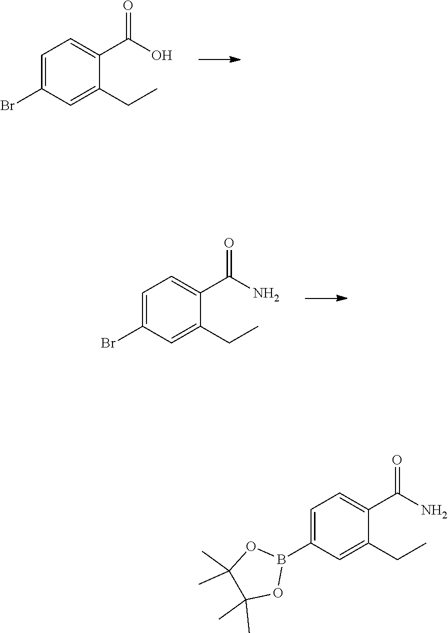 [SMILES: CCc1cc(Br)ccc1C(=O)O ; CCc1cc(Br)ccc1C(N)=O ; CCc1cc(B2OC(C)(C)C(C)(C)O2)ccc1C(N)=O]

4-Bromo-2-ethyl benzoic acid (0.750g) was dissolved in SOCl₂ (2 mL) and heated to 45° C. overnight. Toluene (5 mL) was added and the solvents were dried down. Added 5 mL of DCM (5 mL) was added and the reaction vial was cooled to 0° C. in an ice bath. NH₄OH was slowly added while stirring until no more precipitate formed. The solids were filtered, the mother liquor separated, and the aqueous layer was washed with DCM. The organic layer was dried, filtered, and concentrated under vacuum. Combined solids gave 4-bromo-2-ethyl benzamide of a white powder (750 mg; 100% yield).

4-Bromo-2-ethyl benzamide (0.6g), 4,4,5,5-tetramethyl-2-(4,4,5,5-tetramethyl-1,3,2-dioxaborolan-2-yl)-1,3,2-dioxaborolane (0.73g), Pd(dppf)Cl₂. CH₂Cl₂ (0.04 eq) and KOAc (3 eq) were dissolved in 10 mL of N₂-flushed dioxane. The reaction was heated to 95° C. overnight. The reaction was diluted with EtOAc and the solids were filtered. The product was concentrated under high vacuum and purified via column chromatography (0-60% Hexanes/EtOAc) to obtain 2-ethyl-4-(4,4,5,5-tetramethyl-1,3,2-dioxaborolan-2-yl)benzamide as a white powder (500 mg; 69% yield).

### Example 1

### 4-(4-((1,3-dimethyl-1H-pyrazolo[4,3-b]pyridin-6-yl)oxy)pyridin-2-yl)-2-ethylbenzamide

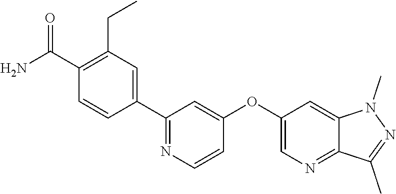 [SMILES: CCc1cc(-c2cc(Oc3cnc4c(C)nn(C)c4c3)ccn2)ccc1C(N)=O]

### Synthesis of 4-(4-((1,3-dimethyl-1H-pyrazolo[4,3-b]pyridin-6-yl)oxy)pyridin-2-yl)-2-ethylbenzamide

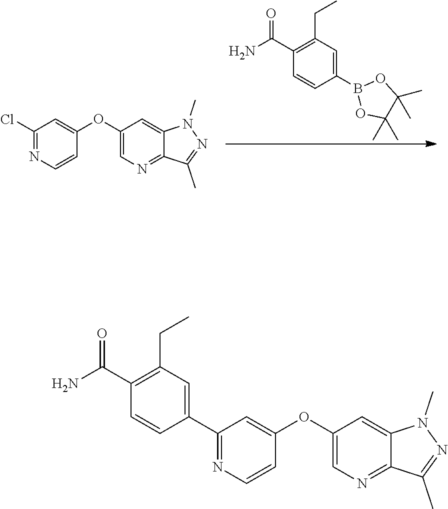 [SMILES: Cc1nn(C)c2cc(Oc3ccnc(Cl)c3)cnc12 ; CCc1cc(B2OC(C)(C)C(C)(C)O2)ccc1C(N)=O ; CCc1cc(-c2cc(Oc3cnc4c(C)nn(C)c4c3)ccn2)ccc1C(N)=O]

In a 1 L flask the 6-((2-chloropyridin-4-yl)oxy)-1,3-dimethyl-1H-pyrazolo[4,3-b]pyridine (24.1g), 2-ethyl-4-(4,4,5,5-tetramethyl-1,3,2-dioxaborolan-2-yl)benzamide (24.2g) and tetrakis(triphenylphosphine) palladium (0) (4 mol %, 4.06g) were combined together followed by addition of 44 mL of 2 M Na₂CO₃, and then 110 mL of dioxane. The reaction was degassed with bubbling N₂ and then heated to 80° C. for 20 h. The reaction was then cooled to ˜60° C., then filtered, washing the aqueous suspension with hot dioxane. Then ˜ 120 g of silica gel was added to the crude mixture and the material was rotovapped to dryness and loaded onto 2×330g ISCO silica gel cartridges. Material was purified by column chromatography eluting with hexanes/acetone gradient. The material was re-dissolved in MeOH and solid loaded again for a second column with MeOH/DCM to remove additional impurities. After drying down the pure fractions the solid material was then recrystallized from MeOH to give 15.503 g of an off-white powder. To remove palladium residues the material was re-dissolved in 150 mL of EtOAc, 2 mL of diethylenetriamine were added and the solution was washed with 350 mL of water. This was repeated three times. The organic phase was washed with water and then brine, dried over MgSO₄, and dried down. To reduce Pd levels further the remaining solids were dissolved in 80 mL of warm DMF and diluted to 2 L with EtOAc. Into this was added 20 mL of diethylenetriamine and the solution was then washed with 120 mL of water. This wash with diethylenetriamine was repeated two additional times and then the organic phase was finally washed once with 120 mL of water, and 120 mL of brine, dried over MgSO₄, filtered, and evaporated to approximately ˜50 mL of a suspension. To this mixture was added 50 mL of diethyl ether. The solids were collected by filtration, washing with more ether. The solids were dried on the filter by suction and then under vacuum at 65° C. overnight. This resulted in 14.96 g of a white solid. Pd level was measured by ICP-MS at 3 ppm. 1H NMR (400 MHz, DMSO-d, 27° C.): δ=8.59 (d, J=5.6 Hz, 1H), 8.42 (d, J=2.3 Hz, 1H), 8.04 (d, J=2.3 Hz, 1H), 7.99 (d, J=1.6 Hz, 1H), 7.89 (dd, J=8.0, 1.8 Hz, 1H), 7.78 (s, 1H), 7.71 (d, J=2.3 Hz, 1H), 7.41 (d, J=7.9 Hz, 2H), 6.96 (dd, J=5.6, 2.3 Hz, 1H), 3.97 (s, 3H), 2.82 (q, J=7.5 Hz, 2H), 2.52-2.55 (m, 3H), 1.07-1.23 (t, J=7.5 Hz, 3H). LCMS Method 3A: r.t. =1.7 min, m/z (M+H+)=388.1 observed mass, exact mass 387.17.

Alternatively, in a 1-L flask the 6-((2-chloropyridin-4-yl)oxy)-1,3-dimethyl-1H-pyrazolo[4,3-b]pyridine (48 g), 2-ethyl-4-(4,4,5,5-tetramethyl-1,3,2-dioxaborolan-2-yl)benzamide (48 g) and tetrakis(triphenylphosphine) palladium (0) (1.5 mol %, 3.0 g) were combined together in 1-butanol (480 mL) followed by addition of sodium tert-butoxide (18.5 g). The reaction was degassed with bubbling N₂ then heated at 95° C. for 20 h. The reaction was cooled to 50° C. and quenched with 0.5M HCl (480 mL), stirring for 30 minutes. The layers were separated, and the aqueous layer was extracted with 0.5M HCl (480 mL and 240 mL). The combined aqueous was washed with methyl tert-butyl ether (240 mL). The aqueous phase was placed into a 2-L flask and the pH was adjusted to 11-13 using 10N NaOH (˜70 mL). The slurry was stirred for 2 h. The solids were collected by filtration, washed with H₂O (240 mL) and hexanes (240 mL). The solids were dried under vacuum at 55° C. overnight resulting in 59.5 g (88% isolated yield) as an off-white solid. Pd level was measured by ICP-MS at 20 ppm. NMR conforms and LCMS method: r.t=6.7 min, m/z (M+H+)=388.1 observed mass, exact mass 387.17.

### Example 2

### 2-ethyl-4-{4-[(1-methyl-1H-indazol-6-yl)oxy]pyridin-2-yl}benzamide

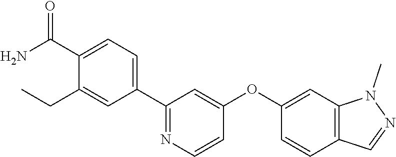 [SMILES: CCc1cc(-c2cc(Oc3ccc4cnn(C)c4c3)ccn2)ccc1C(N)=O]

### Synthesis of 2-ethyl-4-(4-((1-methyl-1H-indazol-6-yl)oxy)pyridin-2-yl)benzamide

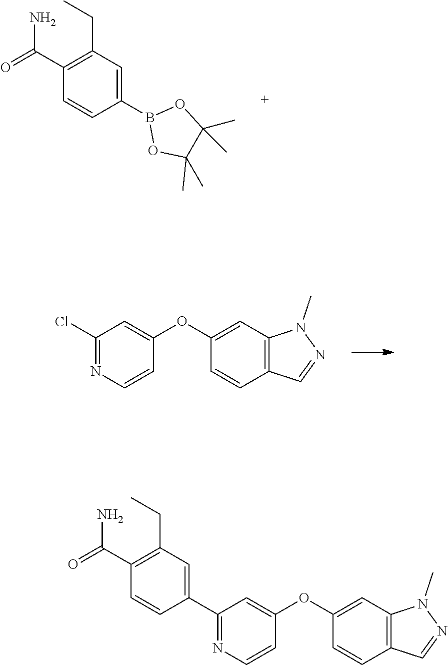 [SMILES: CCc1cc(B2OC(C)(C)C(C)(C)O2)ccc1C(N)=O ; Cn1ncc2ccc(Oc3ccnc(Cl)c3)cc21 ; CCc1cc(-c2cc(Oc3ccc4cnn(C)c4c3)ccn2)ccc1C(N)=O]

6-((2-chloropyridin-4-yl)oxy)-1-methyl-1H-indazole (20g) was added to dioxane (230 mL) and N₂ was bubbled through suspension for 30 min. 2-ethyl-4-(4,4,5,5-tetramethyl-1,3,2-dioxaborolan-2-yl)benzamide (22.25g) then tetrakis (triphenylphosphine) palladium (0) (0.04 eq) were added and the reaction was heated for 8 hours at 85° C. in a R^{B} flask equipped with a condenser under N₂. Once complete, the reaction was filtered through a pad of celite while still hot. Once cooled, 30 mL of brine was added, and the mixture was shaken, and the aqueous fraction discarded. The organic layer was concentrated and then purified by chromatography on silica gel (2×220 g silica gel ISCO columns) eluting with Acetone/Hexanes. The pure fractions were concentrated under reduced pressure to approximately 75 mL. After resting the mixture overnight, the solid precipitate was collected by filtration. This solid was re-dissolved in 50 mL of warm DMF and diluted to 2 L with EtOAc. 20 mL of liquid tris-amine was added followed by 500 mL of water. The mixture was shaken vigorously, and the aqueous layer discarded. This was repeated three times followed by washing with brine (300 mL). This solution was concentrated under vacuum to leave approximately 75 mL and a suspension was formed. After storing overnight at r.t. the precipitate was collected by filtration to yield 20.53 g of a white solid. ¹H NMR (400 MHz, DMSO-d, 27° C.): δ=8.56 (d, J=5.6 Hz, 1H), 8.11 (d, J=0.7 Hz, 1H), 7.98 (d, J=1.7 Hz, 1H), 7.83-7.91 (m, 2H), 7.79 (br s, 1H), 7.64 (d, J=2.3 Hz, 1H), 7.55-7.60 (m, 1H), 7.36-7.46 (m, 2H), 7.01 (dd, J=8.6, 2.0 Hz, 1H), 6.87 (dd, J=5.6, 2.3 Hz, 1H), 4.03 (s, 3H), 2.82 (q, J=7.5 Hz, 2H), 1.20 (t, J=7.6 Hz, 3H). LCMS Method 2A: r.t. =1.83 min, m/z (M+H+)=373.20 observed mass, exact mass 372.16.

### Example 3

4-(4-((1,3-dimethyl-1H-pyrazolo[4,3-b]pyridin-6-yl)oxy)pyridin-2-yl)-2-ethylbenzamide

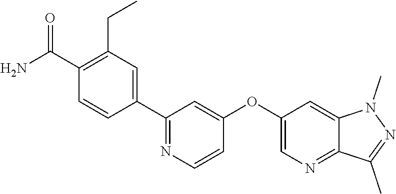 [SMILES: CCc1cc(-c2cc(Oc3cnc4c(C)nn(C)c4c3)ccn2)ccc1C(N)=O]

Synthesis of 4-(4-((1,3-dimethyl-1H-pyrazolo[4,3-b]pyridin-6-yl)oxy)pyridin-2-yl)-2-ethylbenzamide

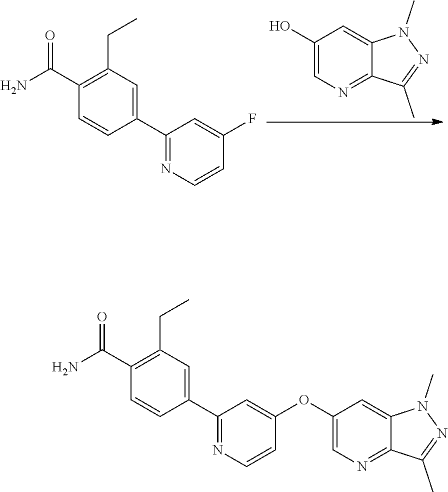 [SMILES: CCc1cc(-c2cc(F)ccn2)ccc1C(N)=O ; Cc1nn(C)c2cc(O)cnc12 ; CCc1cc(-c2cc(Oc3cnc4c(C)nn(C)c4c3)ccn2)ccc1C(N)=O]

In a 20 mL microwave vial, NaH (0.416 g, 10.4 mmol, 1.6 eq, 60% dispersion in mineral oil) was added to a solution of 1,3-dimethyl-1H-pyrazolo[4,3-b]pyridin-6-ol (1.58 g, 9.77 mmol, 1.4 eq) in anhydrous DMF (10 mL) at rt. The mixture was stirred for 20 min. To the reaction mixture was added 2-ethyl-4-(4-fluoropyridin-2-yl)benzamide (1.50 g, 6.51 mmol, 1 eq) and the reaction mixture was heated to 90° C. in a heating block. After 5 hours, the brown solution was cooled to rt then added dropwise to water (˜5 mL) with stirring for 15 min. The clumped solid was filtered off and triturated with Et20 (20 mL) and filtered. The solid was triturated with dichloromethane (˜10 mL) and sonicated for ˜3 min, and filtered. The filtrate was filtered through a larger 0.45 μm HPLC filter, conc. in vacuo to ˜6 mL and purified via flash column chromatography (ISCO 40g, 0-100% ethyl acetate/hexanes) and the product was ˜90-95% pure. The solid was dissolved in ˜2 mL of DMSO and purified via reverse phase column chromatography (ISCO 30 g, 10-60% acetonitrile/water) to afford 4-(4-((1,3-dimethyl-1H-pyrazolo[4,3-b]pyridin-6-yl)oxy)pyridin-2-yl)-2-ethylbenzamide as a white solid.

### Example 4

### 4-(4-((1,3-dimethyl-1H-indazol-6-yl)oxy)pyridin-2-yl)-2-methylbenzamide

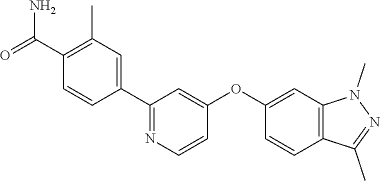 [SMILES: Cc1cc(-c2cc(Oc3ccc4c(C)nn(C)c4c3)ccn2)ccc1C(N)=O]

### Synthesis of 4-(4-((1,3-dimethyl-1H-indazol-6-yl)oxy)pyridin-2-yl)-2-methylbenzamide

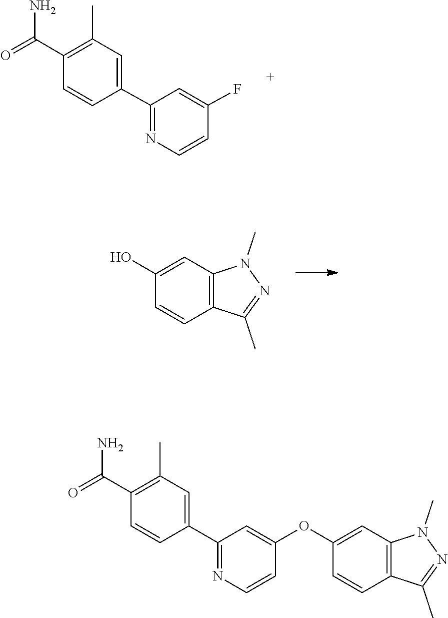 [SMILES: Cc1cc(-c2cc(F)ccn2)ccc1C(N)=O ; Cc1nn(C)c2cc(O)ccc12 ; Cc1cc(-c2cc(Oc3ccc4c(C)nn(C)c4c3)ccn2)ccc1C(N)=O]

In a 20 mL microwave vial, NaH (0.416 g, 10.4 mmol, 1.6 eq, 60% dispersion in mineral oil) was added to a solution of 1,3-dimethyl-1H-indazol-6-ol (1.48 g, 9.11 mmol, 1.4 eq) in anhydrous DMF (10 mL) at rt. The mixture was stirred for 20 min. 4-(4-fluoropyridin-2-yl)-2-methylbenzamide (1.50 g, 6.51 mmol, 1 eq) was added and the reaction mixture was heated to 90° C. in a heating block. After 6 hours, the brown solution was cooled to rt then added dropwise to water (˜5 mL) and stirred for 15 min. The clumped solid was filtered off and triturated with Et2O (20 mL) and filtered. The solid was triturated with dichloromethane (˜10 mL) and sonicated for ˜3 min, and filtered. The filtrate was filtered through a larger 0.45 μm HPLC filter, conc. in vacuo to ˜6 mL and purified via flash column chromatography (ISCO 40g, 0-100% ethyl acetate/hexanes) and the product was ˜90-95% pure. So the solid was dissolved in ˜2 mL of DMSO and purified via reverse phase column chromatography (ISCO 30 g, 10-60% acetonitrile/water) to afford 4-(4-((1,3-dimethyl-1H-indazol-6-yl)oxy)pyridin-2-yl)-2-methylbenzamide as a white solid. ¹H NMR (400 MHz, DMSO-d, 27° C.): δ=8.55 (d, J=5.6 Hz, 1H), 7.94 (s, 1H), 7.86 (dd, J=8.0, 1.4 Hz, 1H), 7.81 (d, J=8.7 Hz, 1H), 7.76 (br s, 1H), 7.61 (d, J=2.3 Hz, 1H), 7.48 (d, J=1.8 Hz, 1H), 7.45 (d, J=7.9 Hz, 1H), 7.40 (br s, 1H), 6.96 (dd, J=8.6, 2.0 Hz, 1H), 6.86 (dd, J=5.6, 2.3 Hz, 1H), 3.93 (s, 3H), 2.48-2.50 (overlapped with solvent DMSO peak, 3H), 2.45 (br s, 3H). LCMS Method 1A: r.t. =1.38 min, m/z (M+H+)=373.20 observed mass, exact mass 372.16.

The following examples of table 9 were made according to the procedure in Examples, above, using the appropriate starting materials

TABLE 9

| | |
|---|---|
| 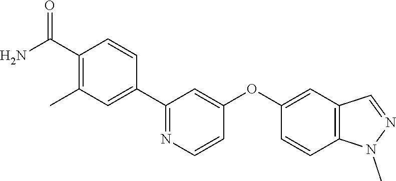 [SMILES: Cc1cc(-c2cc(Oc3ccc4c(cnn4C)c3)ccn2)ccc1C(N)=O] | 2-methyl-4-(4-((1-methyl-1H- indazol-5-yl)oxy)pyridin-2- yl)benzamide LCMS Method 3A: r.t. = 0.98 min, m/z (M + H+) = 359.2 observed mass, exact mass 358.14. |
| Example 5 | |
| 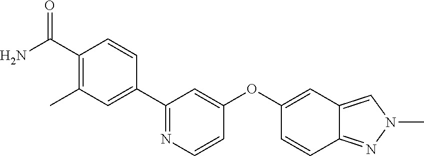 [SMILES: Cc1cc(-c2cc(Oc3ccc4nn(C)cc4c3)ccn2)ccc1C(N)=O] | 2-methyl-4-(4-((2-methyl-2H- indazol-5-yl)oxy)pyridin-2- yl)benzamide LCMS Method 3A: r.t. = 0.86 min, m/z (M + H+) = 359.2 observed mass, exact mass 358.14. |
| Example 6 | |
| 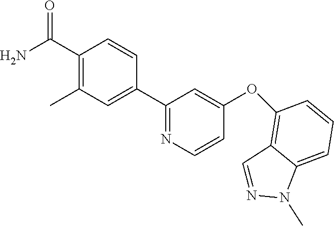 [SMILES: Cc1cc(-c2cc(Oc3cccc4c3cnn4C)ccn2)ccc1C(N)=O] | 2-methyl-4-(4-((1-methyl-1H- indazol-4-yl)oxy)pyridin-2- yl)benzamide LCMS Method 3A: r.t. = 1.06 min, m/z (M + H+) = 359.2 observed mass, exact mass 358.14. |
| Example 7 | |
| 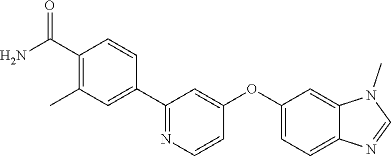 [SMILES: Cc1cc(-c2cc(Oc3ccc4ncn(C)c4c3)ccn2)ccc1C(N)=O] | 2-methyl-4-(4-((1-methyl-1H- benzo[d]imidazol-6- yl)oxy)pyridin-2-yl)benzamide LCMS Method 3A: r.t. = 0.37 min, m/z (M + H+) = 359.2 observed mass, exact mass 358.14. |
| Example 8 | |
| 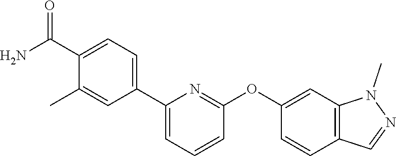 [SMILES: Cc1cc(-c2cccc(Oc3ccc4cnn(C)c4c3)n2)ccc1C(N)=O] | 2-methyl-4-(6-((1-methyl-1H- indazol-6-yl)oxy)pyridin-2- yl)benzamide LCMS Method 3A: r.t. = 1.6 min, m/z (M + H+) = 359.2 observed mass, exact mass 358.14. |
| Example 9 | |
| 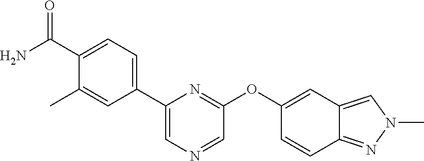 [SMILES: Cc1cc(-c2cncc(Oc3ccc4nn(C)cc4c3)n2)ccc1C(N)=O] | 2-methyl-4-(6-((2-methyl-2H- indazol-5-yl)oxy)pyrazin-2- yl)benzamide LCMS Method 3A: r.t. = 1.15 min, m/z (M + H+) = 360.1 observed mass, exact mass 359.14. |
| Example 10 | |
| 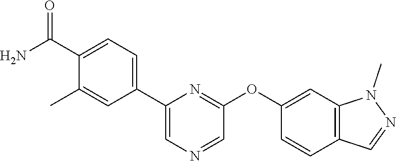 [SMILES: Cc1cc(-c2cncc(Oc3ccc4cnn(C)c4c3)n2)ccc1C(N)=O] | 2-methyl-4-(6-((1-methyl-1H- indazol-6-yl)oxy)pyrazin-2- yl)benzamide LCMS Method 3A: r.t. = 1.28 min, m/z (M + H+) = 360.1 observed mass, exact mass 359.14. |
| Example 11 | |

### Example 12

### 2-methyl-4-(4-((1-methyl-1H-pyrazolo[4,3-b]pyridin-6-yl)oxy)pyridin-2-yl)benzamide

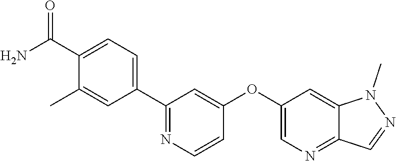 [SMILES: Cc1cc(-c2cc(Oc3cnc4cnn(C)c4c3)ccn2)ccc1C(N)=O]

### Synthesis of 2-methyl-4-(4-((1-methyl-1H-pyrazolo[4,3-b]pyridin-6-yl)oxy)pyridin-2-yl)benzamide

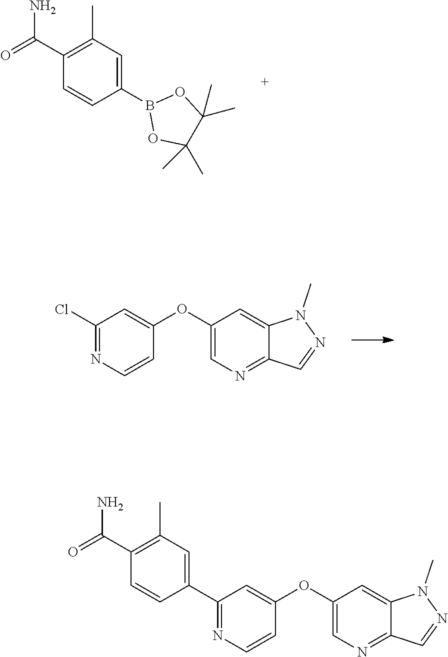 [SMILES: Cc1cc(B2OC(C)(C)C(C)(C)O2)ccc1C(N)=O ; Cn1ncc2ncc(Oc3ccnc(Cl)c3)cc21 ; Cc1cc(-c2cc(Oc3cnc4cnn(C)c4c3)ccn2)ccc1C(N)=O]

2-chloro-4-({1-methyl-1H-pyrazolo[4,3-b]pyridin-6-yl}oxy)pyridine (8 mg) and 2-methyl-4-(tetramethyl-1,3,2-dioxaborolan-2-yl)benzamide (10 mg) were dissolved in 350 μL of dioxane, and then 150 μL of 2 M aqueous Na₂CO₃ solution was added followed by X-Phos Pd-G3 catalyst (1.5 mg). The reaction was sealed in a small glass vial and heated to 95° C. overnight. After cooling 500 μL of DMF was added, the reaction was filtered, and the mixture was purified by HPLC to give 2-methyl-4-(4-((1-methyl-1H-pyrazolo[4,3-b]pyridin-6-yl)oxy)pyridin-2-yl)benzamide. An alternate to the catalyst, X-Phos Pd-G3 catalyst, was tetrakis(triphenyl-phosphine) palladium (0) (0.02-0.1 eqs) in dioxane with aqueous sodium carbonate. An alternate to dioxane with aqueous sodium carbonate was EtOH/Toluene solvent with aqueous sodium carbonate. LCMS Method 3A: r.t. =0.73 min, m/z (M+H+)=360.2 observed mass, exact mass 359.14.

The following examples of table 10 were made according to the procedure in Example 3 using the appropriate starting materials:

TABLE 10

| | |
|---|---|
| 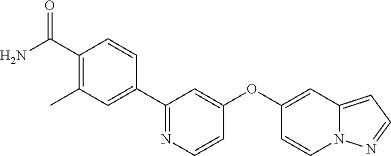 [SMILES: Cc1cc(-c2cc(Oc3ccn4nccc4c3)ccn2)ccc1C(N)=O] | 2-methyl-4-(4-(pyrazolo[1,5- a]pyridin-5-yloxy)pyridin-2- yl)benzamide LCMS Method 3A: r.t. = 0.86 min, m/z (M + H+) = 345.2 observed mass, exact mass 344.13. |
| Example 13 | |
| 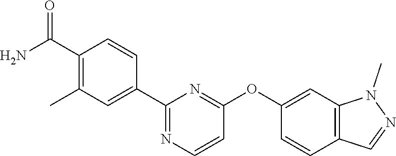 [SMILES: Cc1cc(-c2nccc(Oc3ccc4cnn(C)c4c3)n2)ccc1C(N)=O] | 2-methyl-4-(4-((1-methyl-1H- indazol-6-yl)oxy)pyrimidin-2- yl)benzamide LCMS Method 3A: r.t. = 1.3 min, m/z (M + H+) = 360.2 observed mass, exact mass 359.14. |
| Example 14 | |

### Example 15

### 4-(2-amino-6-((2-methyl-2H-indazol-5-yl)oxy)pyridin-4-yl)-2-methylbenzamide

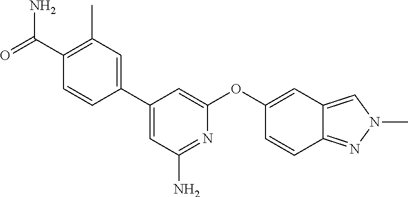 [SMILES: Cc1cc(-c2cc(N)nc(Oc3ccc4nn(C)cc4c3)c2)ccc1C(N)=O]

### Synthesis of 4-(2-amino-6-((2-methyl-2H-indazol-5-yl)oxy)pyridin-4-yl)-2-methylbenzamide

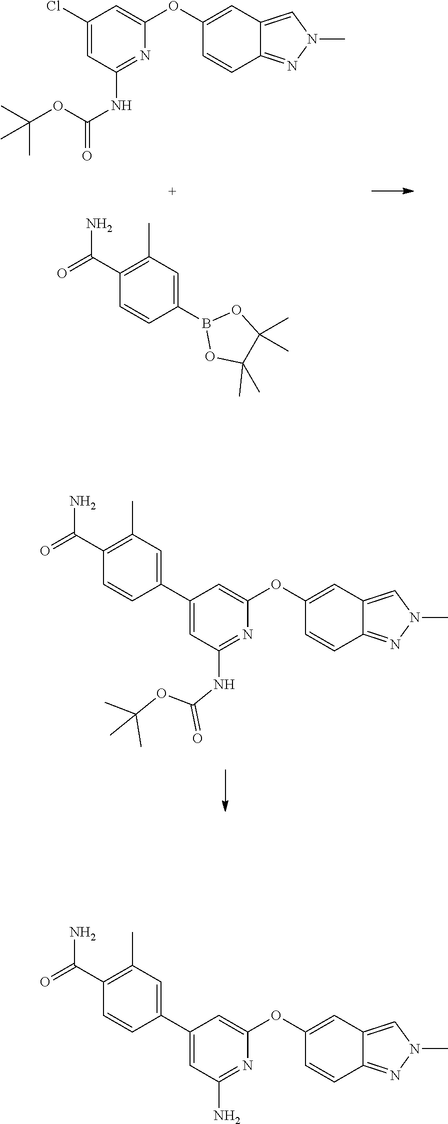 [SMILES: Cn1cc2cc(Oc3cc(Cl)cc(NC(=O)OC(C)(C)C)n3)ccc2n1 ; Cc1cc(B2OC(C)(C)C(C)(C)O2)ccc1C(N)=O ; Cc1cc(-c2cc(NC(=O)OC(C)(C)C)nc(Oc3ccc4nn(C)cc4c3)c2)ccc1C(N)=O ; Cc1cc(-c2cc(N)nc(Oc3ccc4nn(C)cc4c3)c2)ccc1C(N)=O]

tert-butyl (4-chloro-6-((2-methyl-2H-indazol-5-yl)oxy)pyridin-2-yl)carbamate (27 mg, 1 eq), 2-methyl-4-(tetramethyl-1,3,2-dioxaborolan-2-yl)benzamide (1 eq) and

Pd(PPh3)₄ (5 mol %) were added to 10% w/v Na₂CO₃ (2 eq) and dioxane (1 ml). Reaction was purged with N₂ and heated at 90° C. overnight. After cooling, the reaction was treated with EtOAc, water was added and the organic layer separated. After drying under vacuum the crude product was re-suspended in MeCN: TFA (1 ml, 1:1) and stirred at 45° C. for 4 h. Reaction was neutralized with 10% Na₂CO₃ solution, extracted with EtOAc, and crude was purified by chromatography on silica gel (0-15% MeOH-DCM). LCMS Method 3A: r.t. =0.85 min, m/z (M+H+)=374.2 observed mass, exact mass 373.15.

The following examples of table 11 were made according to the procedure in Example 4 using the appropriate starting materials:

TABLE 11

| 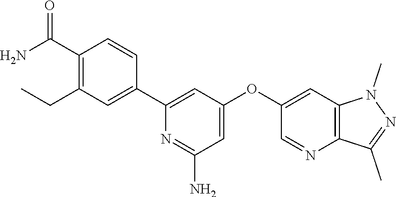 [SMILES: CCc1cc(-c2cc(Oc3cnc4c(C)nn(C)c4c3)cc(N)n2)ccc1C(N)=O] | 4-(6-amino-4-((1,3-dimethyl- 1H-pyrazolo[4,3-b]pyridin-6- yl)oxy)pyridin-2-yl)-2- ethylbenzamide 1H NMR (400 MHz, DMSO-d, 27° C.): δ = 8.39 (d, J = 2.1 Hz, 1H), 8.02 (s, 1H), 7.90 (s, 1H), 7.75-7.82 (m, 2H), 7.38 (br d, J = 8.2 Hz, 2H), 6.91 (br s, 1H), 6.12 (br s, 2H), 5.82 (s, 1H), 3.98 (s, 3H), 2.76-2.84 (m, 2H), 2.54 (s, 3H), 1.15-1.24 (overlapped with solvent ethyl |
|---|---|
| Example 16 | acetate peak, m, 3H) |
| | LCMS Method 1A: r.t. = 1.33 |
| | min, m/z (M + H+) = 403.1 |
| | observed mass, exact mass |
| | 402.18. |
| 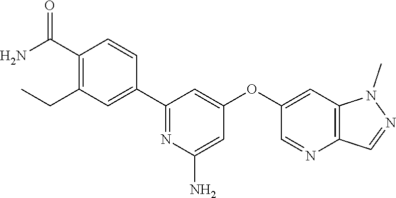 [SMILES: CCc1cc(-c2cc(Oc3cnc4cnn(C)c4c3)cc(N)n2)ccc1C(N)=O] | 4-(6-amino-4-((1-methyl-1H- pyrazolo[4,3-b]pyridin-6- yl)oxy)pyridin-2-yl)-2- ethylbenzamide LCMS Method 2A: r.t. = 0.72 min, m/z (M + H+) = 389.2 observed mass, exact mass 388.16. |
| Example 17 | |
| 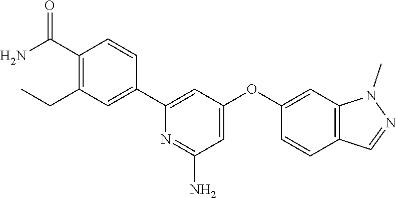 [SMILES: CCc1cc(-c2cc(Oc3ccc4cnn(C)c4c3)cc(N)n2)ccc1C(N)=O] | 4-(6-amino-4-((1-methyl-1H- indazol-6-yl)oxy)pyridin-2-yl)- 2-ethylbenzamide LCMS Method 3A: r.t. = 1.44 min, m/z (M + H+) = 387.9 observed mass, exact mass 387.17. |
| Example 18 | |
| 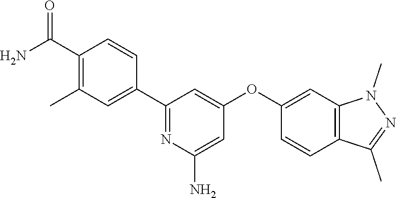 [SMILES: Cc1cc(-c2cc(Oc3ccc4c(C)nn(C)c4c3)cc(N)n2)ccc1C(N)=O] | 4-(6-amino-4-((1,3-dimethyl- 1H-indazol-6-yl)oxy)pyridin-2- yl)-2-methylbenzamide LCMS Method 3A: r.t. = 1.01 min, m/z (M + H+) = 388.2 observed mass, exact mass 387.17. |
| Example 19 | |
| 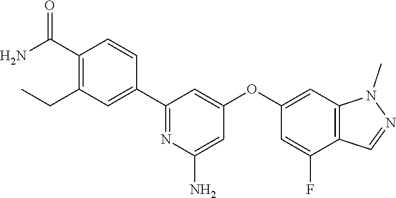 [SMILES: CCc1cc(-c2cc(Oc3cc(F)c4cnn(C)c4c3)cc(N)n2)ccc1C(N)=O] | 4-(6-amino-4-((4-fluoro-1- methyl-1H-indazol-6- yl)oxy)pyridin-2-yl)-2- ethylbenzamide LCMS Method 1A: r.t. = 1.52 min, m/z (M + H+) = 406.1 observed mass, exact mass 405.16. |
| Example 20 | |
| 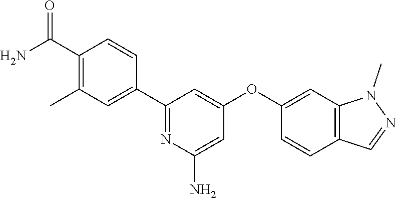 [SMILES: Cc1cc(-c2cc(Oc3ccc4cnn(C)c4c3)cc(N)n2)ccc1C(N)=O] | 4-(6-amino-4-((1-methyl-1H- indazol-6-yl)oxy)pyridin-2-yl)- 2-methylbenzamide LCMS Method 3A: r.t. = 0.91 min, m/z (M + H+) = 374.2 observed mass, exact mass 373.15. |
| Example 21 | |
| 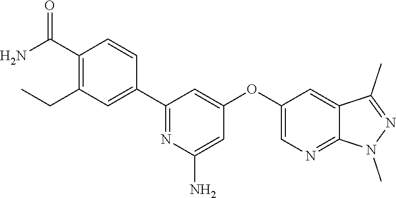 [SMILES: CCc1cc(-c2cc(Oc3cnc4c(c3)c(C)nn4C)cc(N)n2)ccc1C(N)=O] | 4-(6-amino-4-((1,3-dimethyl- 1H-pyrazolo[3,4-b]pyridin-5- yl)oxy)pyridin-2-yl)-2- ethylbenzamide LCMS Method 1A: r.t. = 1.39 min, m/z (M + H+) = 402.2 observed mass, exact mass 402.18. |
| Example 22 | |
| 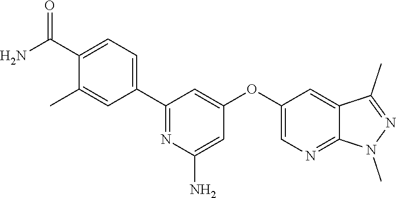 [SMILES: Cc1cc(-c2cc(Oc3cnc4c(c3)c(C)nn4C)cc(N)n2)ccc1C(N)=O] | 4-(6-amino-4-((1,3-dimethyl- 1H-pyrazolo[3,4-b]pyridin-5- yl)oxy)pyridin-2-yl)-2- methylbenzamide LCMS Method 3A: r.t. = 0.87 min, m/z (M + H+) = 389.2 observed mass, exact mass 388.16. |
| Example 23 | |
| 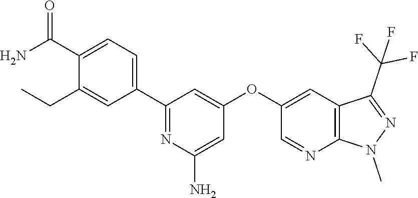 [SMILES: CCc1cc(-c2cc(Oc3cnc4c(c3)c(C(F)(F)F)nn4C)cc(N)n2)ccc1C(N)=O] | 4-(6-amino-4-((1-methyl-3- (trifluoromethyl)-1H- pyrazolo[3,4-b]pyridin-5- yl)oxy)pyridin-2-yl)-2- ethylbenzamide 1H NMR (400 MHz, DMSO-d, 27° C.): δ = 8.74 (d, J = 2.4 Hz, 1H), 8.23 (d, J = 1.8 Hz, 1H), 7.89 (s, 1H), 7.75-7.81 (m, 2H), 7.37 (d, J = 7.8 Hz, 2H), 6.90 (d, J = 2.0 Hz, 1H), 6.11 (s, 2H), 5.79 (d, J = 1.8 Hz, 1H), 4.23 (s, 3H), 2.75-2.84 (m, 2H), 1.19 (t, |
| Example 24 | J = 7.5 Hz, 3H) |
| | LCMS Method 1A: r.t. = 1.62 |
| | min, m/z (M + H+) = 457.2 |
| | observed mass, exact mass |
| | 456.15. |

### Example 25

### 4-(4-((1,3-dimethyl-1H-indazol-6-yl)oxy)pyridin-2-yl)-2-methylbenzamide

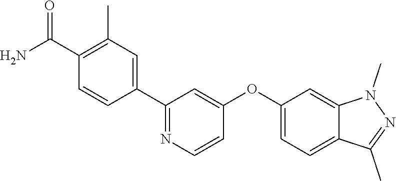 [SMILES: Cc1cc(-c2cc(Oc3ccc4c(C)nn(C)c4c3)ccn2)ccc1C(N)=O]

### Synthesis of 4-(4-((1,3-dimethyl-1H-indazol-6-yl)oxy)pyridin-2-yl)-2-methylbenzamide

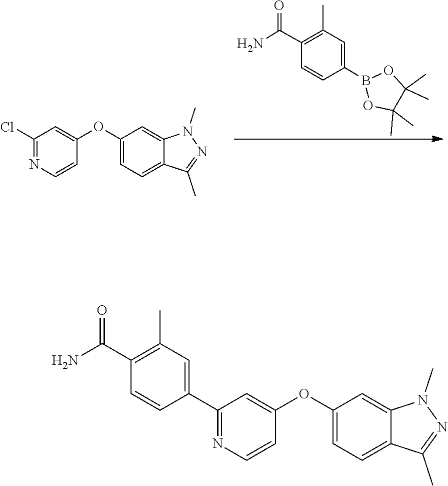 [SMILES: Cc1nn(C)c2cc(Oc3ccnc(Cl)c3)ccc12 ; Cc1cc(B2OC(C)(C)C(C)(C)O2)ccc1C(N)=O ; Cc1cc(-c2cc(Oc3ccc4c(C)nn(C)c4c3)ccn2)ccc1C(N)=O]

6-[(2-chloropyridin-4-yl)oxy]-1,3-dimethyl-1H-indazole (26.8g), 2-methyl-4-(tetramethyl-1,3,2-dioxaborolan-2-yl)benzamide (26.9g), and tetrakis (triphenylphosphine) palladium (0) (0.05 eq) was transferred to a round bottom flask followed by addition of 1,4-dioxane and Na₂CO₃ (2N, 1 eq). Reaction mixture was degassed for 15 min prior to heat to 90° C. Reaction mixture was stirred overnight at 90° C.

After completion of the reaction, mixture was cooled to room temperature. Residual solvent was removed via reduced pressure. The crude was redissolved in

DCM/MeOH (4:1)₅₀₀ mL and washed with water (150 mL×2). Organic solvent was dried with anhydrous Na₂SO₄, filtered, and concentrated via reduced pressure. Silica (50g) was added to crude and silica loaded crude was purified by silica column chromatography using DCM/MeOH 1% to 10% gradient over 30 min.

After purification, purified product was collected, combined, and concentrated to obtain a pale yellowish solid. The yellowish solid was thoroughly washed with EtOAc until all the color impurities were removed to yield 4-{4-[(1,3-dimethyl-1H-indazol-6-yl)oxy]pyridin-2-yl}-2-methylbenzamide (26.7 g, 73%) as a white solid.

White solid was redissolved in DCM/MeOH (4:1)₁ L and Trisamine (50 mL) and water 500 mL were added. The mixture was washed, and the organic layer was isolated. The washing procedure was repeated three times to remove all trace Pd contamination. After completion of washing with trisamine, organic layer was dried with anhydrous Na₂SO₄ and concentrated to get the final product. The white product was dried under high vacuum overnight at 40° C. ¹H NMR (400 MHz, DMSO-d, 27° C.): δ=8.55 (d, J=5.6 Hz, 1H), 7.94 (s, 1H), 7.86 (dd, J=8.0, 1.4 Hz, 1H), 7.81 (d, J=8.7 Hz, 1H), 7.76 (br s, 1H), 7.61 (d, J=2.3 Hz, 1H), 7.48 (d, J=1.8 Hz, 1H), 7.45 (d, J=7.9 Hz, 1H), 7.40 (br s, 1H), 6.96 (dd, J=8.6, 2.0 Hz, 1H), 6.86 (dd, J=5.6, 2.3 Hz, 1H), 3.93 (s, 3H), 2.48-2.50 (overlapped with solvent DMSO peak, 3H), 2.45 (br s, 3H). LCMS Method 3A: r.t. =1.01 min, m/z (M+H+)=373.2 observed mass, exact mass 372.16.

### Example 26

### 2-ethyl-4-(7-((1-methyl-1H-indazol-6-yl)oxy) imidazo[1,2-a]pyridin-5-yl)benzamide

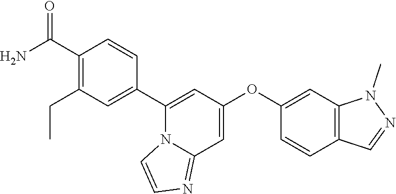 [SMILES: CCc1cc(-c2cc(Oc3ccc4cnn(C)c4c3)cc3nccn23)ccc1C(N)=O]

### Synthesis of 2-ethyl-4-(7-((1-methyl-1H-indazol-6-yl)oxy) imidazo[1,2-a]pyridin-5-yl)benzamide

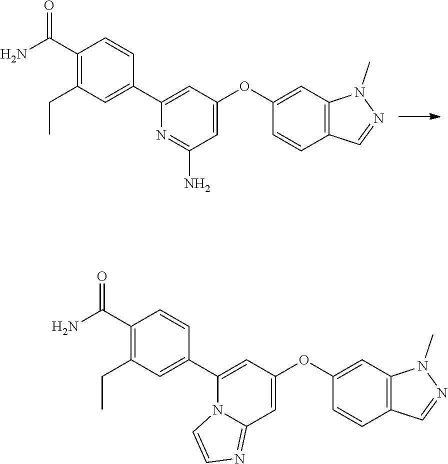 [SMILES: CCc1cc(-c2cc(Oc3ccc4cnn(C)c4c3)cc(N)n2)ccc1C(N)=O ; CCc1cc(-c2cc(Oc3ccc4cnn(C)c4c3)cc3nccn23)ccc1C(N)=O]

4-(6-amino-4-((1-methyl-1H-indazol-6-yl)oxy)pyridin-2-yl)-2-ethylbenzamide (50 mg) was dissolved in MeOH (0.5 mL) and chloroacetaldehyde (50 Wt % in Water, 200 μL) was added followed by 10 mg solid NaHCO₃ and the mixture was heated in a sealed vial for 4 h at 80° C. The reaction was concentrated, and the crude product purified by chromatography on silica gel eluting with DCM/Methanol. After drying, the product was crystallized from MeOH/EtOAc/Ether to give 2-ethyl-4-(7-((1-methyl-1H-indazol-6-yl)oxy) imidazo[1,2-a]pyridin-5-yl)benzamide as a white solid (25 mg). LCMS Method 2A: r.t. =0.8 min, m/z (M+H+)=412.2 observed mass, exact mass 411.17.

The following examples of table 12 were made according to the procedures in the Examples, above, using the appropriate starting materials

TABLE 12

| | |
|---|---|
| 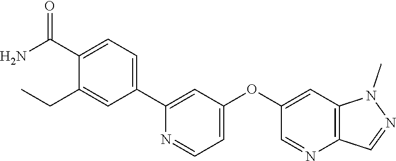 [SMILES: CCc1cc(-c2cc(Oc3cnc4cnn(C)c4c3)ccn2)ccc1C(N)=O] | 2-ethyl-4-(4-((1-methyl-1H- pyrazolo[4,3-b]pyridin-6- yl)oxy)pyridin-2- yl)benzamide LCMS Method 3A: r.t. = 0.84 min, m/z (M + H+) = 374.2 observed mass, exact mass 373.15. |
| Example 27 | |
| 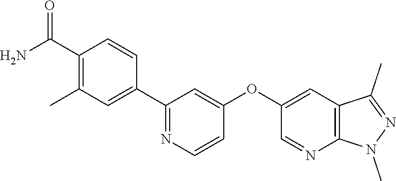 [SMILES: Cc1cc(-c2cc(Oc3cnc4c(c3)c(C)nn4C)ccn2)ccc1C(N)=O] | 4-(4-((1,3-dimethyl-1H- pyrazolo[4,3-b]pyridin-6- yl)oxy)pyridin-2-yl)-2- methylbenzamide 1H NMR (400 MHz, DMSO-d, 27° C.): δ = 8.59 (d, J = 5.6 Hz, 1H), 8.43 (d, J = 2.3 Hz, 1H), 8.05 (d, J = 2.3 Hz, 1H), 7.97 (s, 1H), 7.91 (d, J = 7.9 Hz, 1H) 7.76 (br s, 1H), 7.70 (d, J = |
| Example 28 | 2.2 Hz, 1H), 7.45 (d, J = 8.1 |
| | Hz, 1H), 7.40 (br s, 1H), |
| | 6.97 (dd, J = 5.6, 2.3 Hz, |
| | 1H), 3.97 (s, 3H), 2.55 (s, |
| | 3H), 2.45 ppm (s, 3H) |
| | LCMS Method 1A: r.t. = |
| | 1.25 min, m/z (M + H+) = |
| | 374.1 observed mass, exact |
| | mass 373.15. |
| 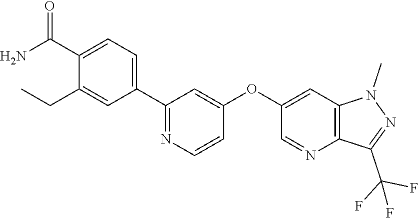 [SMILES: CCc1cc(-c2cc(Oc3cnc4c(C(F)(F)F)nn(C)c4c3)ccn2)ccc1C(N)=O] | 2-ethyl-4-(4-((1-methyl-3- (trifluoromethyl)-1H- pyrazolo[4,3-b]pyridin-6- yl)oxy)pyridin-2- yl)benzamide 1H NMR (400 MHz, DMSO-d, 27° C.): δ = 8.71 (d, J = 2.4 Hz, 1H), 8.63 (d, J = 5.6 Hz, 1H), 8.32 (d, J = 2.3 Hz, 1H), 8.02 (d, J = 1.6 Hz, 1H), 7.92 (dd, J = 7.9, 1.8 Hz, 1H), 7.79 (d, J = 2.2 Hz, 2H), 7.42 (d, J = 8.1 Hz, 2H), 7.07 (dd, J = 5.6, 2.3 |
| Example 29 | Hz, 1H), 4.16 (s, 3H), 2.83 |
| | (q, J = 7.6 Hz, 2H), 1.21 (t, J = |
| | 7.5 Hz, 3H) |
| | LCMS Method 3A: r.t. = |
| | 1.28 min, m/z (M + H+) = |
| | 442 observed mass, exact |
| | mass 441.14. |
| 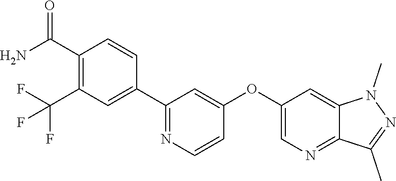 [SMILES: Cc1nn(C)c2cc(Oc3ccnc(-c4ccc(C(N)=O)c(C(F)(F)F)c4)c3)cnc12] | 4-(4-((1,3-dimethyl-1H- pyrazolo[4,3-b]pyridin-6- yl)oxy)pyridin-2-yl)-2- (trifluoromethyl)benzamide LCMS Method 3A: r.t. = 1.89 min, m/z (M + H+) = 428.2 observed mass, exact mass 427.13. |
| Example 30 | |
| 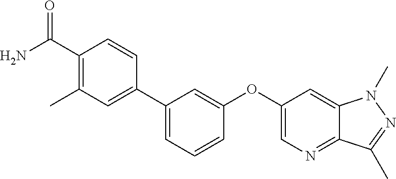 [SMILES: Cc1cc(-c2cccc(Oc3cnc4c(C)nn(C)c4c3)c2)ccc1C(N)=O] | 3′-((1,3-dimethyl-1H- pyrazolo[4,3-b]pyridin-6- yl)oxy)-3-methyl-[1,1′- biphenyl]-4-carboxamide LCMS Method 3A: r.t. = 2.16 min, m/z (M + H+) = 373.2 observed mass, exact mass 372.16. |
| Example 31 | |
| 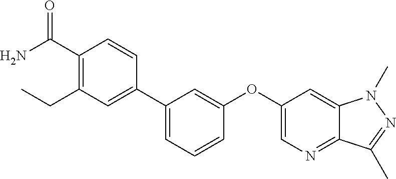 [SMILES: CCc1cc(-c2cccc(Oc3cnc4c(C)nn(C)c4c3)c2)ccc1C(N)=O] | 3′-((1,3-dimethyl-1H- pyrazolo[4,3-b]pyridin-6- yl)oxy)-3-ethyl-[1,1′- biphenyl]-4-carboxamide LCMS Method 3A: r.t. = 2.26 min, m/z (M + H+) = 387.2 observed mass, exact mass 386.17. |
| Example 32 | |
| 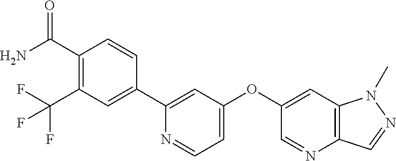 [SMILES: Cn1ncc2ncc(Oc3ccnc(-c4ccc(C(N)=O)c(C(F)(F)F)c4)c3)cc21] | 4-(4-((1-methyl-1H- pyrazolo[4,3-b]pyridin-6- yl)oxy)pyridin-2-yl)-2- (trifluoromethyl)benzamide LCMS Method 3A: r.t. = 1.03 min, m/z (M + H+) = 414.2 observed mass, exact mass 413.11. |
| Example 33 | |
| 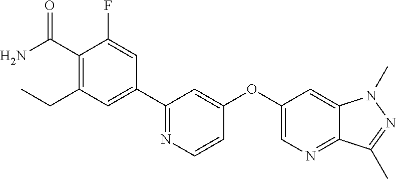 [SMILES: CCc1cc(-c2cc(Oc3cnc4c(C)nn(C)c4c3)ccn2)cc(F)c1C(N)=O] | 4-(4-((1,3-dimethyl-1H- pyrazolo[4,3-b]pyridin-6- yl)oxy)pyridin-2-yl)-2- ethyl-6-fluorobenzamide LCMS Method 3A: r.t. = 1.81 min, m/z (M + H+) = 406.2 observed mass, exact mass 405.16. |
| Example 34 | |
| 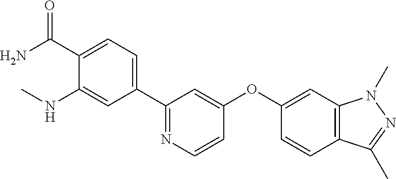 [SMILES: CNc1cc(-c2cc(Oc3ccc4c(C)nn(C)c4c3)ccn2)ccc1C(N)=O] | 4-(4-((1,3-dimethyl-1H- indazol-6-yl)oxy)pyridin-2- yl)-2- (methylamino)benzamide LCMS Method 3A: r.t. = 1.92 min, m/z (M + H+) = 388.2 observed mass, exact mass 387.17. |
| Example 35 | |
| 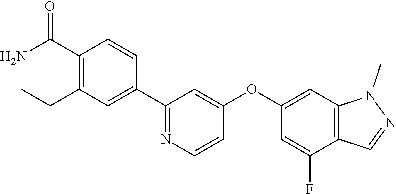 [SMILES: CCc1cc(-c2cc(Oc3cc(F)c4cnn(C)c4c3)ccn2)ccc1C(N)=O] | 2-ethyl-4-(4-((4-fluoro-1- methyl-1H-indazol-6- yl)oxy)pyridin-2- yl)benzamide LCMS Method 2A: r.t. = 0.9 min, m/z (M + H+) = 391.2 observed mass, exact mass 390.15. |
| Example 36 | |
| 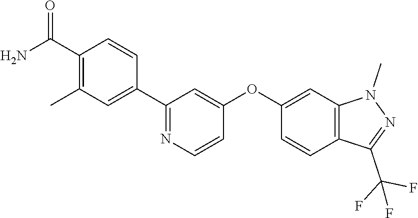 [SMILES: Cc1cc(-c2cc(Oc3ccc4c(C(F)(F)F)nn(C)c4c3)ccn2)ccc1C(N)=O] | 2-methyl-4-(4-((1-methyl-3- (trifluoromethyl)-1H- indazol-6-yl)oxy)pyridin-2- yl)benzamide 1H NMR (400 MHz, DMSO-d, 27° C.): δ = 8.59 (d, J = 5.6 Hz, 1H), 7.97 (s, 1H), 7.88-7.93 (m, 2H), 7.79 (d, J = 1.5 Hz, 1H), 7.76 (br s, 1H), 7.69 (d, J = 2.3 Hz, 1H), 7.46 (d, J = 7.9 Hz, 1H), 7.40 (br s, 1H), 7.25 (dd, J = 8.9, 2.0 Hz, 1H), 6.95 (dd, J = 5.6, 2.3 |
| Example 37 | Hz, 1H), 4.14 (s, 3H), 2.28- |
| | 2.39 (m, 3H). |
| | LCMS Method 3A: r.t. = |
| | 2.13 min, m/z (M + H+) = |
| | 427.2 observed mass, exact |
| | mass 426.13. |
| 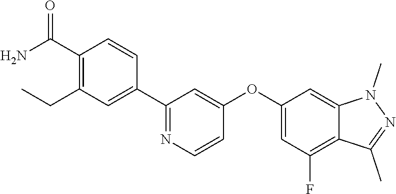 [SMILES: CCc1cc(-c2cc(Oc3cc(F)c4c(C)nn(C)c4c3)ccn2)ccc1C(N)=O] | 2-ethyl-4-(4-((4-fluoro-1,3- dimethyl-1H-indazol-6- yl)oxy)pyridin-2- yl)benzamide LCMS Method 1A: r.t. = 1.58 min, m/z (M + H+) = 405.2 observed mass, exact mass 404.16. |
| Example 38 | |
| 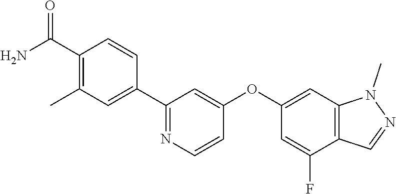 [SMILES: Cc1cc(-c2cc(Oc3cc(F)c4cnn(C)c4c3)ccn2)ccc1C(N)=O] | 4-(4-((4-fluoro-1-methyl- 1H-indazol-6- yl)oxy)pyridin-2-yl)-2- methylbenzamide LCMS Method 2A: r.t. = 0.85 min, m/z (M + H+) = 377.2 observed mass, exact mass 376.13. |
| Example 39 | |
| 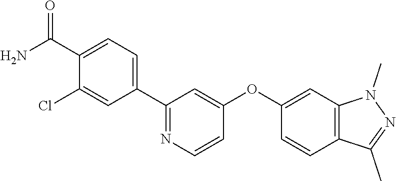 [SMILES: Cc1nn(C)c2cc(Oc3ccnc(-c4ccc(C(N)=O)c(Cl)c4)c3)ccc12] | 2-chloro-4-(4-((1,3- dimethyl-1H-indazol-6- yl)oxy)pyridin-2- yl)benzamide LCMS Method 3A: r.t. = 1.18 min, m/z (M + H+) = 393.2 observed mass, exact mass 392.1. |
| Example 40 | |
| 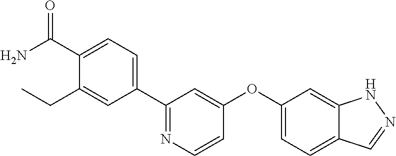 [SMILES: CCc1cc(-c2cc(Oc3ccc4cn[nH]c4c3)ccn2)ccc1C(N)=O] | 4-(4-((1H-indazol-6- yl)oxy)pyridin-2-yl)-2- ethylbenzamide LCMS Method 3A: r.t. = 0.19 min, m/z (M + H+) = 359.2 observed mass, exact mass 358.14. |
| Example 41 | |
| 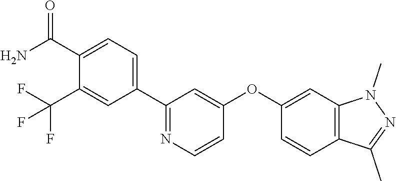 [SMILES: Cc1nn(C)c2cc(Oc3ccnc(-c4ccc(C(N)=O)c(C(F)(F)F)c4)c3)ccc12] | 4-(4-((1,3-dimethyl-1H- indazol-6-yl)oxy)pyridin-2- yl)-2- (trifluoromethyl)benzamide LCMS Method 3A: r.t. = 1.31 min, m/z (M + H+) = 427.2 observed mass, exact mass 426.13. |
| Example 42 | |
| 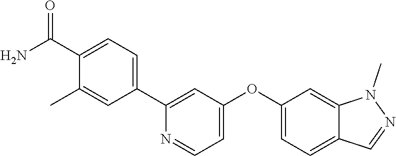 [SMILES: Cc1cc(-c2cc(Oc3ccc4cnn(C)c4c3)ccn2)ccc1C(N)=O] | 2-methyl-4-(4-((1-methyl- 1H-indazol-6- yl)oxy)pyridin-2- yl)benzamide 1H NMR (400 MHz, DMSO-d, 27° C.): δ = 8.69 (d, J = 6.6 Hz, 1H), 8.16 (d, J = 0.9 Hz, 1H), 8.01 (s, 1H), 7.91-7.96 (m, 3H), 7.89 (br s, 1H), 7.72 (s, 1H), |
| Example 43 | 7.55 (d, J = 8.1 Hz, 2H), |
| | 7.22 (dd, J = 6.5, 2.4 Hz, |
| | 1H), 7.09 (dd, J = 8.6, 2.0 |
| | Hz, 1H), 4.05 (s, 3H), 2.46 |
| | (s, 3H) |
| | LCMS Method 3A: r.t. = |
| | 1.87 min, m/z (M + H+) = |
| | 358.9 observed mass, exact |
| | mass 358.14. |
| 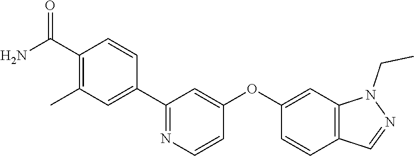 [SMILES: CCn1ncc2ccc(Oc3ccnc(-c4ccc(C(N)=O)c(C)c4)c3)cc21] | 4-(4-((1-ethyl-1H-indazol-6- yl)oxy)pyridin-2-yl)-2- methylbenzamide LCMS Method 3A: r.t. = 1.01 min, m/z (M + H+) = 373.2 observed mass, exact mass 372.16. |
| Example 44 | |
| 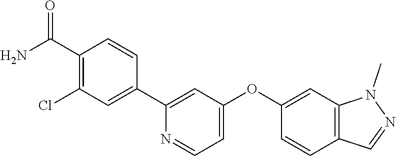 [SMILES: Cn1ncc2ccc(Oc3ccnc(-c4ccc(C(N)=O)c(Cl)c4)c3)cc21] | 2-chloro-4-(4-((1-methyl- 1H-indazol-6- yl)oxy)pyridin-2- yl)benzamide LCMS Method 3A: r.t. = 1.1 min, m/z (M + H+) = 379.1 observed mass, exact mass 378.09. |
| Example 45 | |
| 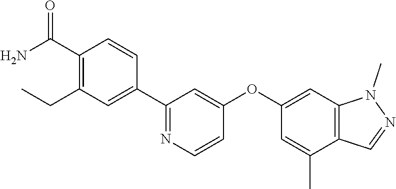 [SMILES: CCc1cc(-c2cc(Oc3cc(C)c4cnn(C)c4c3)ccn2)ccc1C(N)=O] | 4-(4-((1,4-dimethyl-1H- indazol-6-yl)oxy)pyridin-2- yl)-2-ethylbenzamide LCMS Method 3A: r.t. = 1.1 min, m/z (M + H+) = 387.2 observed mass, exact mass 386.17. |
| Example 46 | |
| 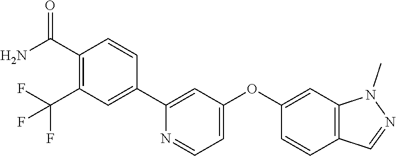 [SMILES: Cn1ncc2ccc(Oc3ccnc(-c4ccc(C(N)=O)c(C(F)(F)F)c4)c3)cc21] | 4-(4-((1-methyl-1H-indazol- 6-yl)oxy)pyridin-2-yl)-2- (trifluoromethyl)benzamide LCMS Method 3A: r.t. = 1.24 min, m/z (M + H+) = 413.1 observed mass, exact mass 412.11. |
| Example 47 | |
| 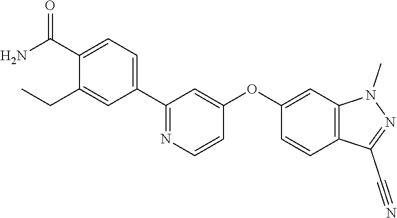 [SMILES: CCc1cc(-c2cc(Oc3ccc4c(C#N)nn(C)c4c3)ccn2)ccc1C(N)=O] | 4-(4-((3-cyano-1-methyl- 1H-indazol-6- yl)oxy)pyridin-2-yl)-2- ethylbenzamide LCMS Method 1A: r.t. = 1.81 min, m/z (M + H+) = 398 observed mass, exact mass 397.15. |
| Example 48 | |
| 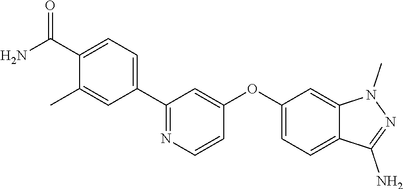 [SMILES: Cc1cc(-c2cc(Oc3ccc4c(N)nn(C)c4c3)ccn2)ccc1C(N)=O] | 4-(4-((3-amino-1-methyl- 1H-indazol-6- yl)oxy)pyridin-2-yl)-2- methylbenzamide LCMS Method 3A: r.t. = 0.72 min, m/z (M + H+) = 374.2 observed mass, exact mass 373.15. |
| Example 49 | |
| 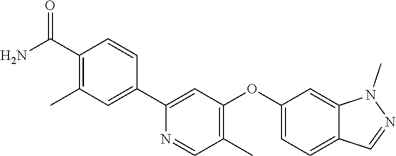 [SMILES: Cc1cnc(-c2ccc(C(N)=O)c(C)c2)cc1Oc1ccc2cnn(C)c2c1] | 2-methyl-4-(5-methyl-4-((1- methyl-1H-indazol-6- yl)oxy)pyridin-2- yl)benzamide LCMS Method 3A: r.t. = 0.99 min, m/z (M + H+) = 373.2 observed mass, exact mass 372.16. |
| Example 50 | |
| 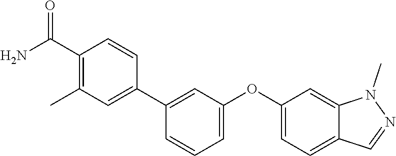 [SMILES: Cc1cc(-c2cccc(Oc3ccc4cnn(C)c4c3)c2)ccc1C(N)=O] | 3-methyl-3′-((1-methyl-1H- indazol-6-yl)oxy)-[1,1′- biphenyl]-4-carboxamide LCMS Method 3A: r.t. = 2.37 min, m/z (M + H+) = 358.2 observed mass, exact mass 357.15. |
| Example 51 | |
| 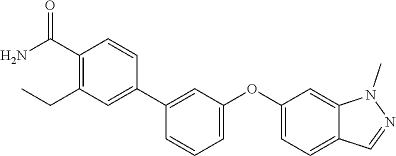 [SMILES: CCc1cc(-c2cccc(Oc3ccc4cnn(C)c4c3)c2)ccc1C(N)=O] | 3-ethyl-3′-((1-methyl-1H- indazol-6-yl)oxy)-[1,1′- biphenyl]-4-carboxamide LCMS Method 3A: r.t. = 2.46 min, m/z (M + H+) = 372.3 observed mass, exact mass 371.16. |
| Example 52 | |
| 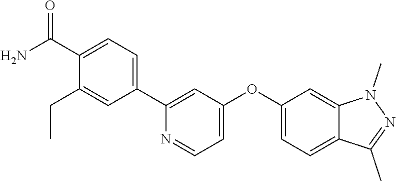 [SMILES: CCc1cc(-c2cc(Oc3ccc4c(C)nn(C)c4c3)ccn2)ccc1C(N)=O] | 4-(4-((1,3-dimethyl-1H- indazol-6-yl)oxy)pyridin-2- yl)-2-ethylbenzamide LCMS Method 3A: r.t. = 1.11 min, m/z (M + H+) = 387.2 observed mass, exact mass 386.17. |
| Example 53 | |
| 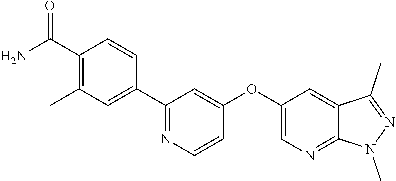 [SMILES: Cc1cc(-c2cc(Oc3cnc4c(c3)c(C)nn4C)ccn2)ccc1C(N)=O] | 4-(4-((1,3-dimethyl-1H- pyrazolo[3,4-b]pyridin-5- yl)oxy)pyridin-2-yl)-2- methylbenzamide LCMS Method 1A: r.t. = 1.3 min, m/z (M + H+) = 374.2 observed mass, exact mass 373.15. |
| Example 54 | |
| 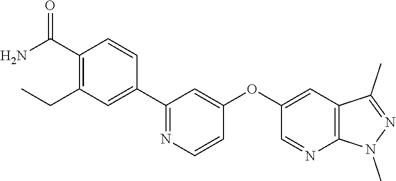 [SMILES: CCc1cc(-c2cc(Oc3cnc4c(c3)c(C)nn4C)ccn2)ccc1C(N)=O] | 4-(4-((1,3-dimethyl-1H- pyrazolo[3,4-b]pyridin-5- yl)oxy)pyridin-2-yl)-2- ethylbenzamide LCMS Method 3A: r.t. = 0.98 min, m/z (M + H+) = 388.3 observed mass, exact mass 387.17. |
| Example 55 | |
| 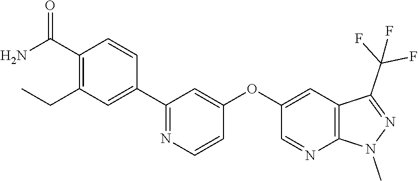 [SMILES: CCc1cc(-c2cc(Oc3cnc4c(c3)c(C(F)(F)F)nn4C)ccn2)ccc1C(N)=O] | 2-ethyl-4-(4-((1-methyl-3- (trifluoromethyl)-1H- pyrazolo[3,4-b]pyridin-5- yl)oxy)pyridin-2- yl)benzamide LCMS Method 3A: r.t. = 1.34 min, m/z (M + H+) = 442.2 observed mass, exact mass 441.14. |
| Example 56 | |
| 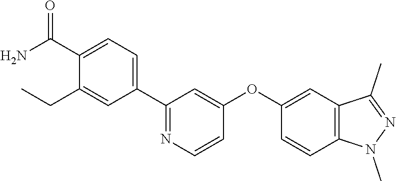 [SMILES: CCc1cc(-c2cc(Oc3ccc4c(c3)c(C)nn4C)ccn2)ccc1C(N)=O] | 4-(4-((1,3-dimethyl-1H- indazol-5-yl)oxy)pyridin-2- yl)-2-ethylbenzamide LCMS Method 3A: r.t. = 1.04 min, m/z (M + H+) = 387.2 observed mass, exact mass 386.17. |
| Example 57 | |
| 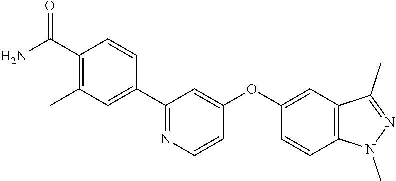 [SMILES: Cc1cc(-c2cc(Oc3ccc4c(c3)c(C)nn4C)ccn2)ccc1C(N)=O] | 4-(4-((1,3-dimethyl-1H- indazol-5-yl)oxy)pyridin-2- yl)-2-methylbenzamide LCMS Method 3A: r.t. = 0.94 min, m/z (M + H+) = 373.2 observed mass, exact mass 372.16. |
| Example 58 | |
| 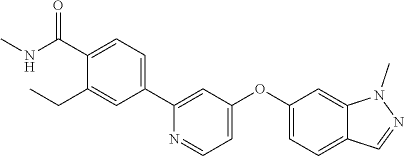 [SMILES: CCc1cc(-c2cc(Oc3ccc4cnn(C)c4c3)ccn2)ccc1C(=O)NC] | 2-ethyl-N-methyl-4-(4-((1- methyl-1H-indazol-6- yl)oxy)pyridin-2- yl)benzamide LCMS Method 3A: r.t. = 1.81 min, m/z (M + H+) = 387.3 observed mass, exact mass 386.17. |
| Example 59 | |
| 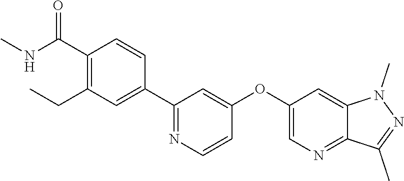 [SMILES: CCc1cc(-c2cc(Oc3cnc4c(C)nn(C)c4c3)ccn2)ccc1C(=O)NC] | 4-(4-((1,3-dimethyl-1H- pyrazolo[4,3-b]pyridin-6- yl)oxy)pyridin-2-yl)-2- ethyl-N-methylbenzamide LCMS Method 3A: r.t. = 1.73 min, m/z (M + H+) = 402.3 observed mass, exact mass 401.19. |
| Example 60 | |
| 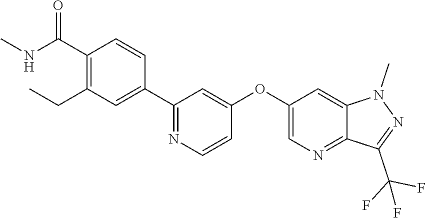 [SMILES: CCc1cc(-c2cc(Oc3cnc4c(C(F)(F)F)nn(C)c4c3)ccn2)ccc1C(=O)NC] | 2-ethyl-N-methyl-4-(4-((1- methyl-3-(trifluoromethyl)- 1H-pyrazolo[4,3-b]pyridin- 6-yl)oxy)pyridin-2- yl)benzamide LCMS Method 3A: r.t. = 2.06 min, m/z (M + H+) = 456.2 observed mass, exact mass 455.16. |
| Example 61 | |
| 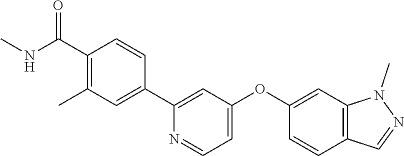 [SMILES: CNC(=O)c1ccc(-c2cc(Oc3ccc4cnn(C)c4c3)ccn2)cc1C] | N,2-dimethyl-4-(4-((1- methyl-1H-indazol-6- yl)oxy)pyridin-2- yl)benzamide LCMS Method 3A: r.t. = 0.99 min, m/z (M + H+) = 373.2 observed mass, exact mass 372.16. |
| Example 62 | |
| 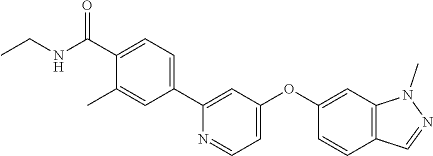 [SMILES: CCNC(=O)c1ccc(-c2cc(Oc3ccc4cnn(C)c4c3)ccn2)cc1C] | N-ethyl-2-methyl-4-(4-((1- methyl-1H-indazol-6- yl)oxy)pyridin-2- yl)benzamide LCMS Method 3A: r.t. = 1.06 min, m/z (M + H+) = 387.2 observed mass, exact mass 386.17. |
| Example 63 | |
| 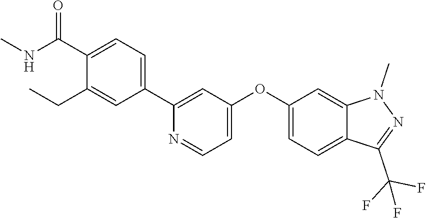 [SMILES: CCc1cc(-c2cc(Oc3ccc4c(C(F)(F)F)nn(C)c4c3)ccn2)ccc1C(=O)NC] | 2-ethyl-N-methyl-4-(4-((1- methyl-3-(trifluoromethyl)- 1H-indazol-6- yl)oxy)pyridin-2- yl)benzamide LCMS Method 3A: r.t. = 2.25 min, m/z (M + H+) = 455.2 observed mass, exact mass 454.16. |
| Example 64 | |
| 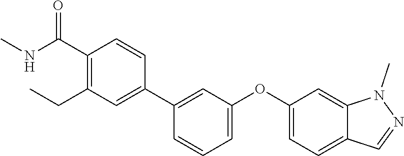 [SMILES: CCc1cc(-c2cccc(Oc3ccc4cnn(C)c4c3)c2)ccc1C(=O)NC] | 3-ethyl-N-methyl-3′-((1- methyl-1H-indazol-6- yl)oxy)-[1,1′-biphenyl]-4- carboxamide LCMS Method 3A: r.t. = 2.55 min, m/z (M + H+) = 386.3 observed mass, exact mass 385.18. |
| Example 65 | |
| 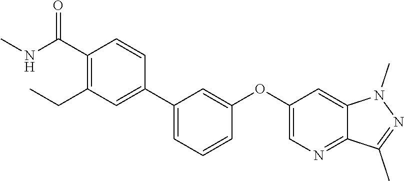 [SMILES: CCc1cc(-c2cccc(Oc3cnc4c(C)nn(C)c4c3)c2)ccc1C(=O)NC] | 3′-((1,3-dimethyl-1H- pyrazolo[4,3-b]pyridin-6- yl)oxy)-3-ethyl-N-methyl- [1,1′-biphenyl]-4- carboxamide LCMS Method 3A: r.t. = 2.33 min, m/z (M + H+) = 401.3 observed mass, exact mass 400.19. |
| Example 66 | |
| 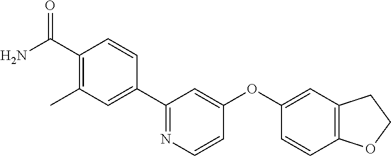 [SMILES: Cc1cc(-c2cc(Oc3ccc4c(c3)CCO4)ccn2)ccc1C(N)=O] | 4-(4-((2,3- dihydrobenzofuran-5- yl)oxy)pyridin-2-yl)-2- methylbenzamide LCMS Method 3A: r.t. = 1.73 min, m/z (M + H+) = 347.2 observed mass, exact mass 346.13. |
| Example 67 | |
| 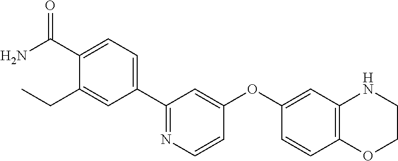 [SMILES: CCc1cc(-c2cc(Oc3ccc4c(c3)NCCO4)ccn2)ccc1C(N)=O] | 4-(4-((3,4-dihydro-2H- benzo[b][1,4]oxazin-6- yl)oxy)pyridin-2-yl)-2- ethylbenzamide LCMS Method 3A: r.t. = 1.05 min, m/z (M + H+) = 376.3 observed mass, exact mass 375.16. |
| Example 68 | |
| 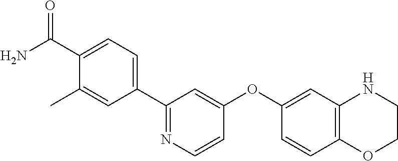 [SMILES: Cc1cc(-c2cc(Oc3ccc4c(c3)NCCO4)ccn2)ccc1C(N)=O] | 4-(4-((3,4-dihydro-2H- benzo[b][1,4]oxazin-6- yl)oxy)pyridin-2-yl)-2- methylbenzamide LCMS Method 3A: r.t. = 0.95 min, m/z (M + H+) = 362.3 observed mass, exact mass 361.14. |
| Example 69 | |
| 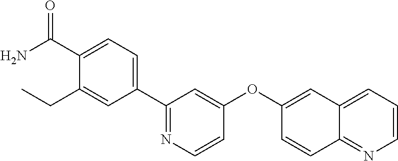 [SMILES: CCc1cc(-c2cc(Oc3ccc4ncccc4c3)ccn2)ccc1C(N)=O] | 2-ethyl-4-(4-(quinolin-6- yloxy)pyridin-2- yl)benzamide 1H NMR (400 MHz, DMSO-d, 27° C.): δ = 8.92 (dd, J = 4.2, 1.6 Hz, 1H), 8.61 (d, J = 5.6 Hz, 1H), 8.38 (d, J = 7.3 Hz, 1H), 8.15 (d, J = 9.0 Hz, 1H), 8.00 (s, 1H), 7.90 (dd, J = |
| Example 70 | 7.9, 1.7 Hz, 1H), 7.78-7.83 |
| | (m, 2H), 7.73 (d, J = 2.3 Hz, |
| | 1H), 7.68 (dd, J = 9.2, 2.7 |
| | Hz, 1H), 7.58 (dd, J = 8.3, |
| | 4.3 Hz, 1H), 7.37-7.47 (m, |
| | 2H), 6.98 (dd, J = 5.6, 2.3 |
| | Hz, 1H), 2.82 (q, J = 7.5 Hz, |
| | 2H), 1.20 (t, J = 7.5 Hz, 3H) |
| | LCMS Method 3A: r.t. = |
| | 0.81 min, m/z (M + H+) = |
| | 370.2 observed mass, exact |
| | mass 369.15. |
| 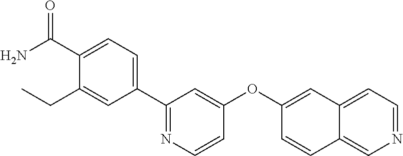 [SMILES: CCc1cc(-c2cc(Oc3ccc4cnccc4c3)ccn2)ccc1C(N)=O] | 2-ethyl-4-(4-(isoquinolin-6- yloxy)pyridin-2- yl)benzamide LCMS Method 3A: r.t. = 0.73 min, m/z (M + H+) = 370.2 observed mass, exact mass 369.15. |
| Example 71 | |
| 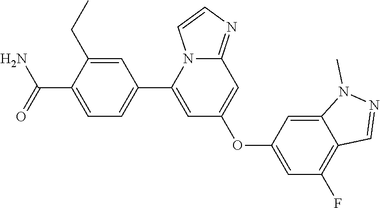 [SMILES: CCc1cc(-c2cc(Oc3cc(F)c4cnn(C)c4c3)cc3nccn23)ccc1C(N)=O] | 2-ethyl-4-(7-((4-fluoro-1- methyl-1H-indazol-6- yl)oxy)imidazo[1,2- a]pyridin-5-yl)benzamide LCMS Method 3A: r.t. = 1.07 min, m/z (M + H+) = 430.2 observed mass, exact mass 429.16. |
| Example 72 | |
| 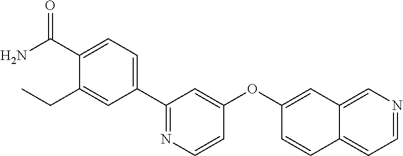 [SMILES: CCc1cc(-c2cc(Oc3ccc4ccncc4c3)ccn2)ccc1C(N)=O] | 2-ethyl-4-(4- (162yridine162ine-7- yloxy)162yridine-2- yl)benzamide LCMS Method 3A: r.t. = 0.71 min, m/z (M + H+) = 370.2 observed mass, exact mass 369.15. |
| Example 73 | |
| 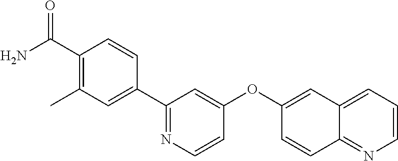 [SMILES: Cc1cc(-c2cc(Oc3ccc4ncccc4c3)ccn2)ccc1C(N)=O] | 2-methyl-4-(4-(quinolin-6- yloxy)pyridin-2- yl)benzamide 1H NMR (400 MHz, DMSO-d, 27° C.): δ = 8.92 (dd, J = 4.2, 1.7 Hz, 1H), 8.60 (d, J = 5.6 Hz, 1H), 8.39 (d, J = 7.5 Hz, 1H), 8.15 (d, J = 9.0 Hz, 1H), 7.97 (s, 1H), 7.90 (d, J = 7.7 |
| Example 74 | Hz, 1H), 7.81 (d, J = 2.7 Hz, |
| | 1H), 7.76 (br s, 1H), 7.72 (d, |
| | J = 2.2 Hz, 1H), 7.68 (dd, J = |
| | 9.1, 2.8 Hz, 1H), 7.58 (dd, |
| | J = 8.3, 4.3 Hz, 1H), 7.46 (d, |
| | J = 7.9 Hz, 1H), 7.40 (br s, |
| | 1H), 6.98 (dd, J = 5.6, 2.3 |
| | Hz, 1H), 2.44 (s, 3H) |
| | LCMS Method 3A: r.t. = 0.7 |
| | min, m/z (M + H+) = 356.2 |
| | observed mass, exact mass |
| | 355.13. |
| 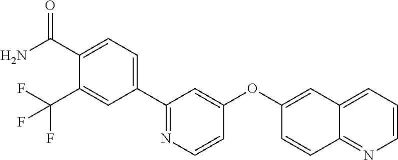 [SMILES: NC(=O)c1ccc(-c2cc(Oc3ccc4ncccc4c3)ccn2)cc1C(F)(F)F] | 4-(4-(quinolin-6- yloxy)pyridin-2-yl)-2- (trifluoromethyl)benzamide LCMS Method 3A: r.t. = 0.97 min, m/z (M + H+) = 410.1 observed mass, exact mass 409.1. |
| Example 75 | |
| 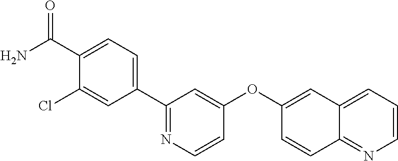 [SMILES: NC(=O)c1ccc(-c2cc(Oc3ccc4ncccc4c3)ccn2)cc1Cl] | 2-chloro-4-(4-(quinolin-6- yloxy)pyridin-2- yl)benzamide LCMS Method 3A: r.t. = 0.85 min, m/z (M + H+) = 376.1 observed mass, exact mass 375.08. |
| Example 76 | |
| 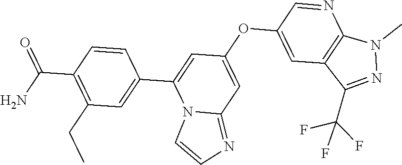 [SMILES: CCc1cc(-c2cc(Oc3cnc4c(c3)c(C(F)(F)F)nn4C)cc3nccn23)ccc1C(N)=O] | 2-ethyl-4-(7-((1-methyl-3- (trifluoromethyl)-1H- pyrazolo[3,4-b]pyridin-5- yl)oxy)imidazo[1,2- a]pyridin-5-yl)benzamide LCMS Method 3A: r.t. = 1.22 min, m/z (M + H+) = 481.2 observed mass, exact mass 480.15. |
| Example 77 | |
| 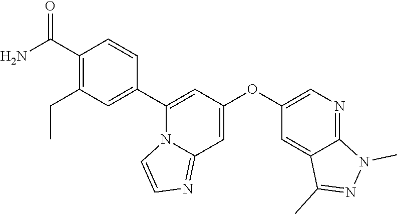 [SMILES: CCc1cc(-c2cc(Oc3cnc4c(c3)c(C)nn4C)cc3nccn23)ccc1C(N)=O] | 4-(7-((1,3-dimethyl-1H- pyrazolo[3,4-b]pyridin-5- yl)oxy)imidazo[1,2- a]pyridin-5-yl)-2- ethylbenzamide LCMS Method 3A: r.t. = 0.94 min, m/z (M + H+) = 427.2 observed mass, exact mass 426.18. |
| Example 78 | |
| 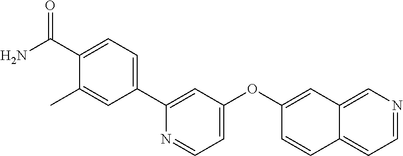 [SMILES: Cc1cc(-c2cc(Oc3ccc4ccncc4c3)ccn2)ccc1C(N)=O] | 4-(4-(isoquinolin-7- yloxy)pyridin-2-yl)-2- methylbenzamide LCMS Method 3A: r.t. = 0.58 min, m/z (M + H+) = 356.1 observed mass, exact mass 355.13. |
| Example 79 | |
| 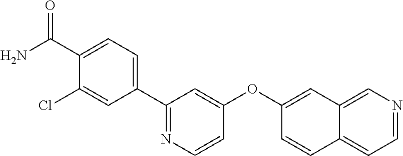 [SMILES: NC(=O)c1ccc(-c2cc(Oc3ccc4ccncc4c3)ccn2)cc1Cl] | 2-chloro-4-(4-(isoquinolin- 7-yloxy)pyridin-2- yl)benzamide LCMS Method 3A: r.t. = 0.75 min, m/z (M + H+) = 376.1 observed mass, exact mass 375.08. |
| Example 80 | |
| 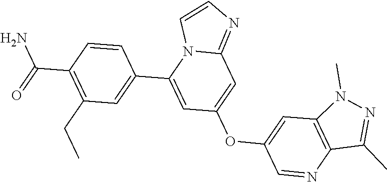 [SMILES: CCc1cc(-c2cc(Oc3cnc4c(C)nn(C)c4c3)cc3nccn23)ccc1C(N)=O] | 4-(7-((1,3-dimethyl-1H- pyrazolo[4,3-b]pyridin-6- yl)oxy)imidazo[1,2- a]pyridin-5-yl)-2- ethylbenzamide LCMS Method 2A: r.t. = 0.73 min, m/z (M + H+) = 427.2 observed mass, exact mass 426.18. |
| Example 81 | |
| 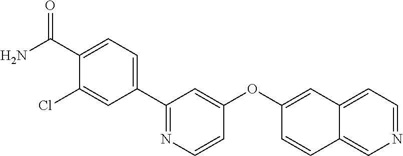 [SMILES: NC(=O)c1ccc(-c2cc(Oc3ccc4cnccc4c3)ccn2)cc1Cl] | 2-chloro-4-(4-(isoquinolin- 6-yloxy)pyridin-2- yl)benzamide LCMS Method 3A: r.t. = 0.78 min, m/z (M + H+) = 376.2 observed mass, exact mass 375.08. |
| Example 82 | |
| 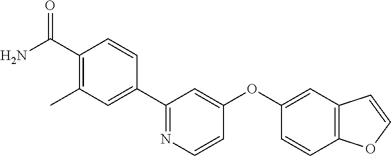 [SMILES: Cc1cc(-c2cc(Oc3ccc4occc4c3)ccn2)ccc1C(N)=O] | 4-(4-(benzofuran-5- yloxy)pyridin-2-yl)-2- methylbenzamide LCMS Method 3A: r.t. = 2.11 min, m/z (M + H+) = 344.9 observed mass, exact mass 344.12. |
| Example 83 | |
| 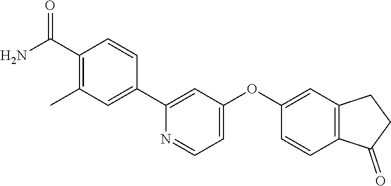 [SMILES: Cc1cc(-c2cc(Oc3ccc4c(c3)CCC4=O)ccn2)ccc1C(N)=O] | 2-methyl-4-(4-((1-oxo-2,3- dihydro-1H-inden-5- yl)oxy)pyridin-2- yl)benzamide LCMS Method 3A: r.t. = 1.85 min, m/z (M + H+) = 358.9 observed mass, exact mass 358.13. |
| Example 84 | |
| 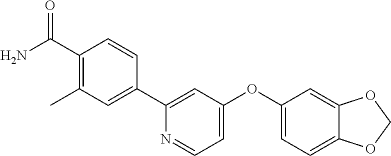 [SMILES: Cc1cc(-c2cc(Oc3ccc4c(c3)OCO4)ccn2)ccc1C(N)=O] | 4-(4-(benzo[d][1,3]dioxol-5- yloxy)pyridin-2-yl)-2- methylbenzamide LCMS Method 3A: r.t. = 1.93 min, m/z (M + H+) = 348.9 observed mass, exact mass 348.11. |
| Example 85 | |
| 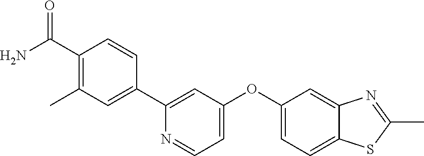 [SMILES: Cc1nc2cc(Oc3ccnc(-c4ccc(C(N)=O)c(C)c4)c3)ccc2s1] | 2-methyl-4-(4-((2- methylbenzo[d]thiazol-5- yl)oxy)pyridin-2- yl)benzamide LCMS Method 3A: r.t. = 1.99 min, m/z (M + H+) = 375.9 observed mass, exact mass 375.1. |
| Example 86 | |
| 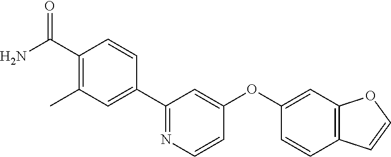 [SMILES: Cc1cc(-c2cc(Oc3ccc4ccoc4c3)ccn2)ccc1C(N)=O] | 4-(4-(benzofuran-6- yloxy)pyridin-2-yl)-2- methylbenzamide LCMS Method 3A: r.t. = 2.08 min, m/z (M + H+) = 344.9 observed mass, exact mass 344.12. |
| Example 87 | |
| 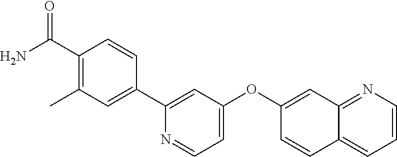 [SMILES: Cc1cc(-c2cc(Oc3ccc4cccnc4c3)ccn2)ccc1C(N)=O] | 2-methyl-4-(4-(quinolin-7- yloxy)pyridin-2- yl)benzamide LCMS Method 3A: r.t. = 1.64 min, m/z (M + H+) = 356 observed mass, exact mass 355.13. |
| Example 88 | |
| 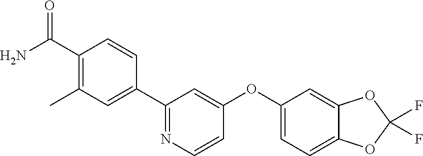 [SMILES: Cc1cc(-c2cc(Oc3ccc4c(c3)OC(F)(F)O4)ccn2)ccc1C(N)=O] | 4-(4-((2,2- difluorobenzo[d][1,3]dioxol- 5-yl)oxy)pyridin-2-yl)-2- methylbenzamide LCMS Method 3A: r.t. = 2.29 min, m/z (M + H+) = 384.9 observed mass, exact mass 384.09. |
| Example 89 | |
| 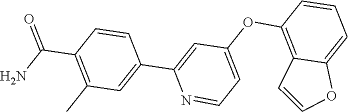 [SMILES: Cc1cc(-c2cc(Oc3cccc4occc34)ccn2)ccc1C(N)=O] | 4-(4-(benzofuran-4- yloxy)pyridin-2-yl)-2- methylbenzamide LCMS Method 3A: r.t. = 2.12 min, m/z (M + H+) = 344.9 observed mass, exact mass 344.12. |
| Example 90 | |
| 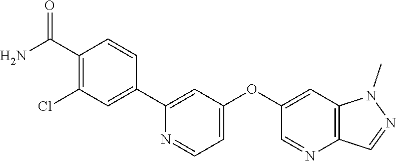 | 2-chloro-4-(4-((1-methyl- 1H-pyrazolo[4,3-b]pyridin- 6-yl)oxy)pyridin-2- yl)benzamide LCMS Method 3A: r.t. = 1.0 min, m/z (M + H+) = 373.2 observed mass, exact mass 372.16. |
| Example 91 | |
| 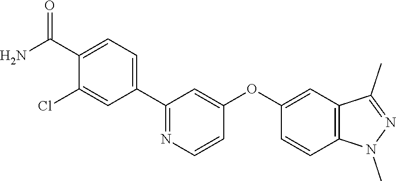 [SMILES: Cc1nn(C)c2ccc(Oc3ccnc(-c4ccc(C(N)=O)c(Cl)c4)c3)cc12] | 2-chloro-4-(4-((1,3- dimethyl-1H-indazol-5- yl)oxy)pyridin-2- yl)benzamide |
| Example 92 | |
| 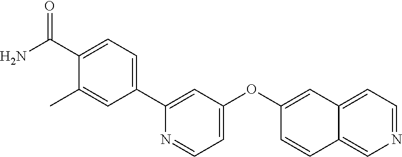 [SMILES: Cc1cc(-c2cc(Oc3ccc4cnccc4c3)ccn2)ccc1C(N)=O] | 4-(4-(isoquinolin-6- yloxy)pyridin-2-yl)-2- methylbenzamide LCMS Method 3A: r.t. = 0.62 min, m/z (M + H+) = 356.1 observed mass, exact mass 355.13. |
| Example 93 | |
| 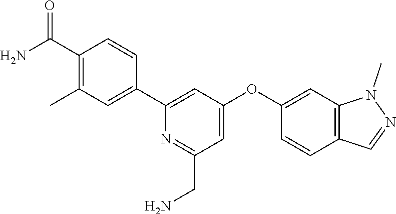 [SMILES: Cc1cc(-c2cc(Oc3ccc4cnn(C)c4c3)cc(CN)n2)ccc1C(N)=O] | 4-(6-(aminomethyl)-4-((1- methyl-1H-indazol-6- yl)oxy)pyridin-2-yl)-2- methylbenzamide LCMS Method 3A: r.t. = 0.93 min, m/z (M + H+) = 388.3 observed mass, exact mass 387.17. |
| Example 94 | |
| 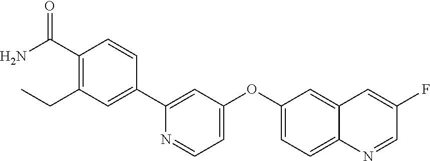 [SMILES: CCc1cc(-c2cc(Oc3ccc4ncc(F)cc4c3)ccn2)ccc1C(N)=O] | 2-ethyl-4-(4-((3- fluoroquinolin-6- yl)oxy)pyridin-2- yl)benzamide LCMS Method 3A: r.t. = 1.99 min, m/z (M + H+) = 388.30 observed mass, exact mass 387.14. |
| Example 95 | |
| 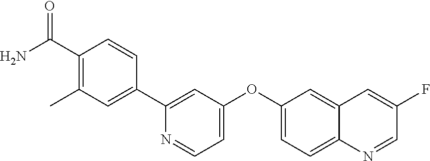 [SMILES: Cc1cc(-c2cc(Oc3ccc4ncc(F)cc4c3)ccn2)ccc1C(N)=O] | 4-(4-((3-fluoroquinolin-6- yl)oxy)pyridin-2-yl)-2- methylbenzamide LCMS Method 3A: r.t. = 1.94 min, m/z (M + H+) = 374.20 observed mass, exact mass 373.12. |
| Example 96 | |
| 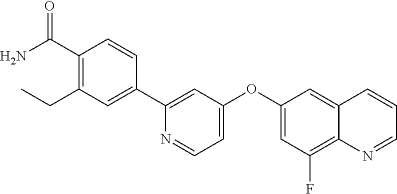 [SMILES: CCc1cc(-c2cc(Oc3cc(F)c4ncccc4c3)ccn2)ccc1C(N)=O] | 2-ethyl-4-(4-((8- fluoroquinolin-6- yl)oxy)pyridin-2- yl)benzamide LCMS Method 3A: r.t. = 1.88 min, m/z (M + H+) = 388.20 observed mass, exact mass 387.14. |
| Example 97 | |
| 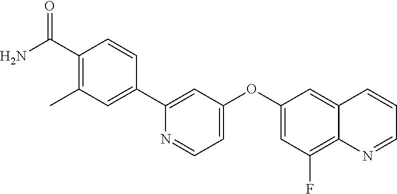 [SMILES: Cc1cc(-c2cc(Oc3cc(F)c4ncccc4c3)ccn2)ccc1C(N)=O] | 4-(4-((8-fluoroquinolin-6- yl)oxy)pyridin-2-yl)-2- methylbenzamide LCMS Method 3A: r.t. = 1.83 min, m/z (M + H+) = 374.20 observed mass, exact mass 373.12. |
| Example 98 | |
| 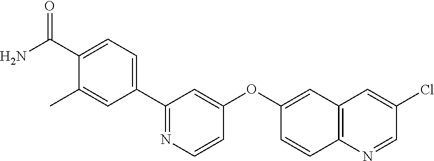 [SMILES: Cc1cc(-c2cc(Oc3ccc4ncc(Cl)cc4c3)ccn2)ccc1C(N)=O] | 4-(4-((3-chloroquinolin-6- yl)oxy)pyridin-2-yl)-2- methylbenzamide LCMS Method 3A: r.t. = 2.07 min, m/z (M + H+) = 390.20 observed mass, exact mass 389.09. |
| Example 99 | |
| 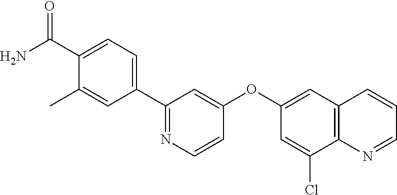 [SMILES: Cc1cc(-c2cc(Oc3cc(Cl)c4ncccc4c3)ccn2)ccc1C(N)=O] | 4-(4-((8-chloroquinolin-6- yl)oxy)pyridin-2-yl)-2- methylbenzamide LCMS Method 3A: r.t. = 1.93 min, m/z (M + H+) = 390.20 observed mass, exact mass 389.09. |
| Example 100 | |
| 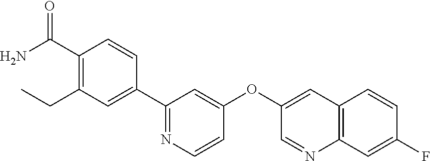 [SMILES: CCc1cc(-c2cc(Oc3cnc4cc(F)ccc4c3)ccn2)ccc1C(N)=O] | 2-ethyl-4-(4-((7- fluoroquinolin-3- yl)oxy)pyridin-2- yl)benzamide LCMS Method 3A: r.t. = 2.08 min, m/z (M + H+) = 388.10 observed mass, exact mass 387.14. |
| Example 101 | |
| 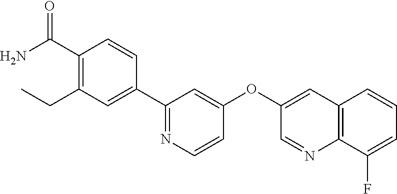 [SMILES: CCc1cc(-c2cc(Oc3cnc4c(F)cccc4c3)ccn2)ccc1C(N)=O] | 2-ethyl-4-(4-((8- fluoroquinolin-3- yl)oxy)pyridin-2- yl)benzamide LCMS Method 3A: r.t. = 2.05 min, m/z (M + H+) = 388.10 observed mass, exact mass 387.14. |
| Example 102 | |
| 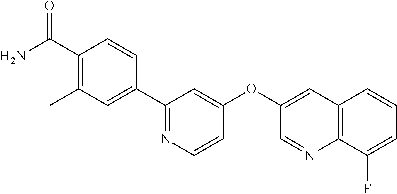 [SMILES: Cc1cc(-c2cc(Oc3cnc4c(F)cccc4c3)ccn2)ccc1C(N)=O] | 4-(4-((8-fluoroquinolin-3- yl)oxy)pyridin-2-yl)-2- methylbenzamide LCMS Method 3A: r.t. = 1.96 min, m/z (M + H+) = 374.20 observed mass, exact mass 373.12. |
| Example 103 | |
| 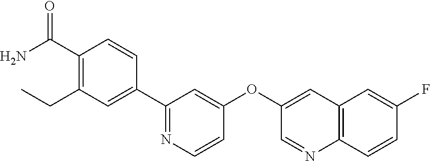 [SMILES: CCc1cc(-c2cc(Oc3cnc4ccc(F)cc4c3)ccn2)ccc1C(N)=O] | 2-ethyl-4-(4-((6- fluoroquinolin-3- yl)oxy)pyridin-2- yl)benzamide LCMS Method 3A: r.t. = 2.1 min, m/z (M + H+) = 388.10 observed mass, exact mass 387.14. |
| Example 104 | |
| 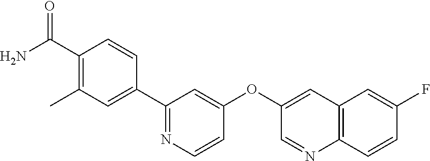 [SMILES: Cc1cc(-c2cc(Oc3cnc4ccc(F)cc4c3)ccn2)ccc1C(N)=O] | 4-(4-((6-fluoroquinolin-3- yl)oxy)pyridin-2-yl)-2- methylbenzamide LCMS Method 3A: r.t. = 1.98 min, m/z (M + H+) = 374.20 observed mass, exact mass 373.12. |
| Example 105 | |
| 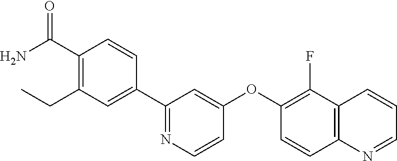 [SMILES: CCc1cc(-c2cc(Oc3ccc4ncccc4c3F)ccn2)ccc1C(N)=O] | 2-ethyl-4-(4-((5- fluoroquinolin-6- yl)oxy)pyridin-2- yl)benzamide LCMS Method 3A: r.t. = 1.98 min, m/z (M + H+) = 388.20 observed mass, exact mass 387.14. |
| Example 106 | |
| 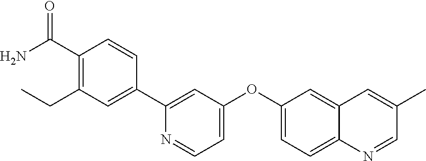 [SMILES: CCc1cc(-c2cc(Oc3ccc4ncc(C)cc4c3)ccn2)ccc1C(N)=O] | 2-ethyl-4-(4-((3- methylquinolin-6- yl)oxy)pyridin-2- yl)benzamide LCMS Method 3A: r.t. = 1.66 min, m/z (M + H+) = 384.20 observed mass, exact mass 383.16. |
| Example 107 | |
| 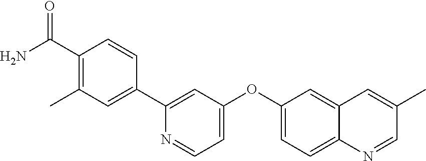 [SMILES: Cc1cnc2ccc(Oc3ccnc(-c4ccc(C(N)=O)c(C)c4)c3)cc2c1] | 2-methyl-4-(4-((3- methylquinolin-6- yl)oxy)pyridin-2- yl)benzamide LCMS Method 3A: r.t. = 1.57 min, m/z (M + H+) = 370.20 observed mass, exact mass 369.15. |
| Example 108 | |
| 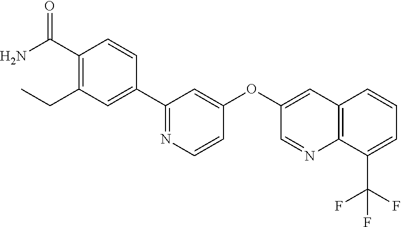 [SMILES: CCc1cc(-c2cc(Oc3cnc4c(C(F)(F)F)cccc4c3)ccn2)ccc1C(N)=O] | 2-ethyl-4-(4-((8- (trifluoromethyl)quinolin-3- yl)oxy)pyridin-2- yl)benzamide LCMS Method 3A: r.t. = 2.37 min, m/z (M + H+) = 438.10 observed mass, exact mass 437.13. |
| Example 109 | |
| 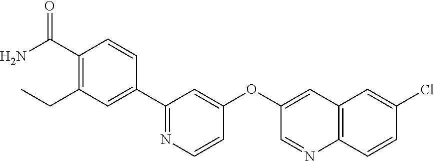 [SMILES: CCc1cc(-c2cc(Oc3cnc4ccc(Cl)cc4c3)ccn2)ccc1C(N)=O] | 4-(4-((6-chloroquinolin-3- yl)oxy)pyridin-2-yl)-2- ethylbenzamide LCMS Method 3A: r.t. = 2.26 min, m/z (M + H+) = 404.10 observed mass, exact mass 403.11. |
| Example 110 | |
| 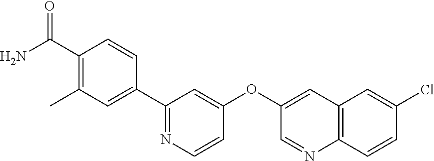 [SMILES: Cc1cc(-c2cc(Oc3cnc4ccc(Cl)cc4c3)ccn2)ccc1C(N)=O] | 4-(4-((6-chloroquinolin-3- yl)oxy)pyridin-2-yl)-2- methylbenzamide LCMS Method 3A: r.t. = 2.15 min, m/z (M + H+) = 390.20 observed mass, exact mass 389.09 |
| Example 111 | |
| 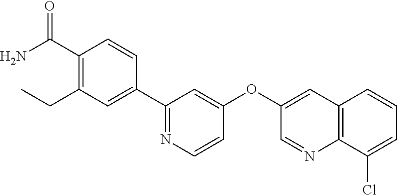 [SMILES: CCc1cc(-c2cc(Oc3cnc4c(Cl)cccc4c3)ccn2)ccc1C(N)=O] | 4-(4-((8-chloroquinolin-3- yl)oxy)pyridin-2-yl)-2- ethylbenzamide LCMS Method 3A: r.t. = 2.19 min, m/z (M + H+) = 404.00 observed mass, exact mass 403.11. |
| Example 112 | |
| 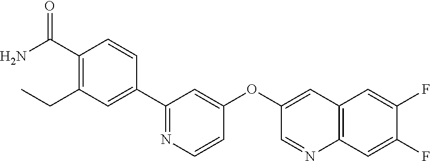 [SMILES: CCc1cc(-c2cc(Oc3cnc4cc(F)c(F)cc4c3)ccn2)ccc1C(N)=O] | 4-(4-((6,7-difluoroquinolin- 3-yl)oxy)pyridin-2-yl)-2- ethylbenzamide LCMS Method 3A: r.t. = 2.2 min, m/z (M + H+) = 406.00 observed mass, exact mass 405.13 |
| Example 113 | |
| 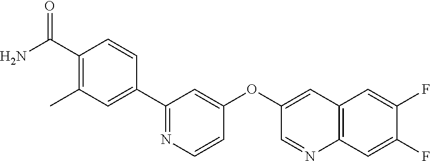 [SMILES: Cc1cc(-c2cc(Oc3cnc4cc(F)c(F)cc4c3)ccn2)ccc1C(N)=O] | 4-(4-((6,7-difluoroquinolin- 3-yl)oxy)pyridin-2-yl)-2- methylbenzamide LCMS Method 3A: r.t. = 2.1 min, m/z (M + H+) = 392.20 observed mass, exact mass 391.11 |
| Example 114 | |
| 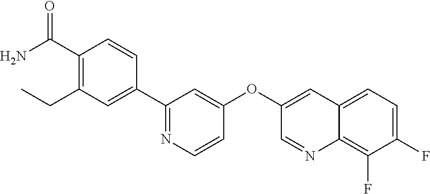 [SMILES: CCc1cc(-c2cc(Oc3cnc4c(F)c(F)ccc4c3)ccn2)ccc1C(N)=O] | 4-(4-((7,8-difluoroquinolin- 3-yl)oxy)pyridin-2-yl )-2- ethylbenzamide LCMS Method 3A: r.t. = 2.13 min, m/z (M + H+) = 406.20 observed mass, exact mass 405.13 |
| Example 115 | |
| 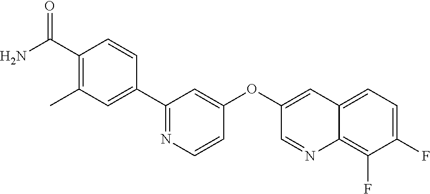 [SMILES: Cc1cc(-c2cc(Oc3cnc4c(F)c(F)ccc4c3)ccn2)ccc1C(N)=O] | 4-(4-((7,8-difluoroquinolin- 3-yl)oxy)pyridin-2-yl)-2- methylbenzamide LCMS Method 3A: r.t. = 2.02 min, m/z (M + H+) = 392.20 observed mass, exact mass 391.11 |
| Example 116 | |
| 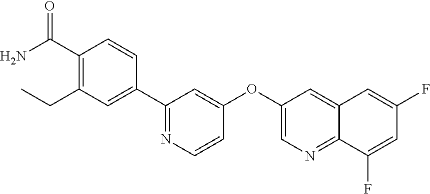 [SMILES: CCc1cc(-c2cc(Oc3cnc4c(F)cc(F)cc4c3)ccn2)ccc1C(N)=O] | 4-(4-((6,8-difluoroquinolin- 3-yl)oxy)pyridin-2-yl)-2- ethylbenzamide LCMS Method 1A: r.t. = 1.88 min, m/z (M + H+) = 406.20 observed mass, exact mass 405.13 |
| Example 117 | |
| 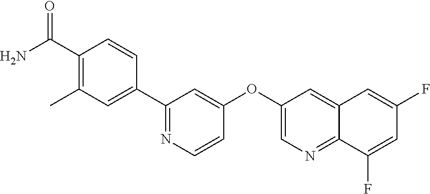 [SMILES: Cc1cc(-c2cc(Oc3cnc4c(F)cc(F)cc4c3)ccn2)ccc1C(N)=O] | 4-(4-((6,8-difluoroquinolin- 3-yl)oxy)pyridin-2-yl)-2- methylbenzamide LCMS Method 1A: r.t. = 1.71 min, m/z (M + H+) = 392.20 observed mass, exact mass 391.11 |
| Example 118 | |
| 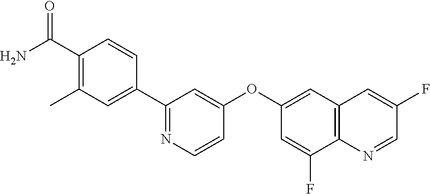 [SMILES: Cc1cc(-c2cc(Oc3cc(F)c4ncc(F)cc4c3)ccn2)ccc1C(N)=O] | 4-(4-((3,8-difluoroquinolin- 6-yl)oxy)pyridin-2-yl)-2- methylbenzamide LCMS Method 3A: r.t. = 1.98 min, m/z (M + H+) = 392.20 observed mass, exact mass 391.11 |
| Example 119 | |
| 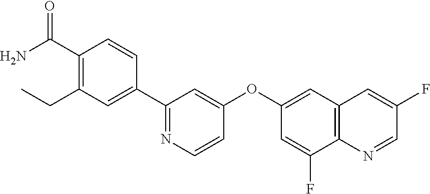 [SMILES: CCc1cc(-c2cc(Oc3cc(F)c4ncc(F)cc4c3)ccn2)ccc1C(N)=O] | 4-(4-((3,8-difluoroquinolin- 6-yl)oxy)pyridin-2-yl)-2- ethylbenzamide LCMS Method 3A: r.t. = 2.08 min, m/z (M + H+) = 406.20 observed mass, exact mass 405.13 |
| Example 120 | |
| 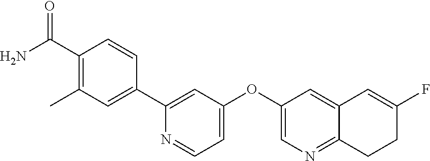 [SMILES: Cc1cc(-c2cc(Oc3cnc4c(c3)C=C(F)CC4)ccn2)ccc1C(N)=O] | 4-(4-((6-fluoro-7,8- dihydroquinolin-3- yl)oxy)pyridin-2-yl)-2- methylbenzamide LCMS Method 1A: r.t. = 1.57 min, m/z (M + H+) = 376.20 observed mass, exact mass 375.14 |
| Example 121 | |
| 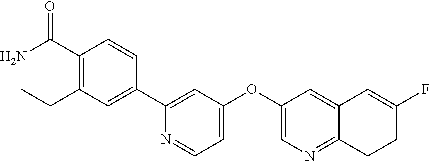 [SMILES: CCc1cc(-c2cc(Oc3cnc4c(c3)C=C(F)CC4)ccn2)ccc1C(N)=O] | 2-ethyl-4-(4-((6-fluoro-7,8- dihydroquinolin-3- yl)oxy)pyridin-2- yl)benzamide LCMS Method 1A: r.t. = 1.61 min, m/z (M + H+) = 390.20 observed mass, exact mass 389.15 |
| Example 122 | |
| 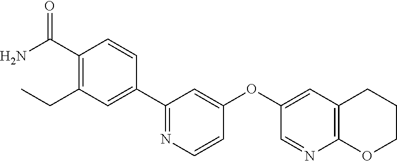 [SMILES: CCc1cc(-c2cc(Oc3cnc4c(c3)CCCO4)ccn2)ccc1C(N)=O] | 4-(4-((3,4-dihydro-2H- pyrano[2,3-b]pyridin-6- yl)oxy)pyridin-2-yl)-2- ethylbenzamide LCMS Method 3A: r.t. = 1.76 min, m/z (M + H+) = 376.10 observed mass, exact mass 375.16 |
| Example 123 | |
| 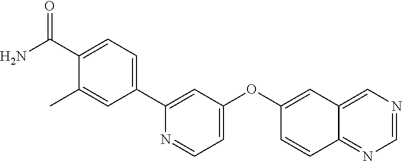 [SMILES: Cc1cc(-c2cc(Oc3ccc4ncncc4c3)ccn2)ccc1C(N)=O] | 2-methyl-4-(4-(quinazolin- 6-yloxy)pyridin-2- yl)benzamide LCMS Method 3A: r.t. = 0.61 min, m/z (M + H+) = 357.20 observed mass, exact mass 356.13 |
| Example 124 | |
| 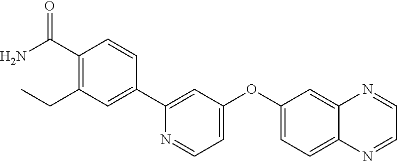 [SMILES: CCc1cc(-c2cc(Oc3ccc4nccnc4c3)ccn2)ccc1C(N)=O] | 2-ethyl-4-(4-(quinoxalin-6- yloxy)pyridin-2- yl)benzamide LCMS Method 3A: r.t. = 1.69 min, m/z (M + H+) = 371.20 observed mass, exact mass 370.14 |
| Example 125 | |
| 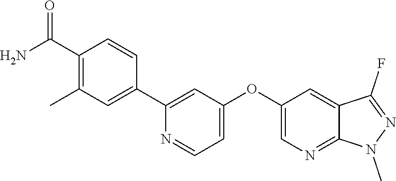 [SMILES: Cc1cc(-c2cc(Oc3cnc4c(c3)c(F)nn4C)ccn2)ccc1C(N)=O] | 4-(4-((3-fluoro-1-methyl- 1H-pyrazolo[3,4-b]pyridin- 5-yl)oxy)pyridin-2-yl)-2- methylbenzamide LCMS Method 3A: r.t. = 1.81 min, m/z (M + H+) = 378.0 observed mass, exact mass 377.13 |
| Example 126 | |
| 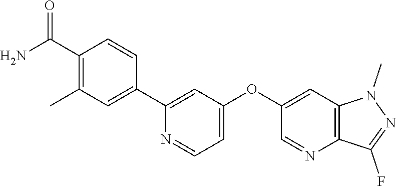 [SMILES: Cc1cc(-c2cc(Oc3cnc4c(F)nn(C)c4c3)ccn2)ccc1C(N)=O] | 4-(4-((3-fluoro-1-methyl- 1H-pyrazolo[4,3-b]pyridin- 6-yl)oxy)pyridin-2-yl)-2- methylbenzamide LCMS Method 3A: r.t. = 1.8 min, m/z (M + H+) = 378.3 observed mass, exact mass 377.13 |
| Example 127 | |
| 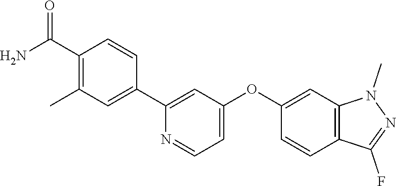 [SMILES: Cc1cc(-c2cc(Oc3ccc4c(F)nn(C)c4c3)ccn2)ccc1C(N)=O] | 4-(4-((3-fluoro-1-methyl- 1H-indazol-6- yl)oxy)pyridin-2-yl)-2- methylbenzamide LCMS Method 3A: r.t. = 1.95 min, m/z (M + H+) = 377.0 observed mass, exact mass 376.13 |
| Example 128 | |
| 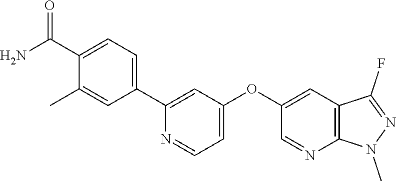 [SMILES: Cc1cc(-c2cc(Oc3cnc4c(c3)c(F)nn4C)ccn2)ccc1C(N)=O] | 4-(4-((3-fluoro-1-methyl- 1H-indazol-5- yl)oxy)pyridin-2-yl)-2- methylbenzamide LCMS Method 3A: r.t. = 1.94 min, m/z (M + H+) = 377.0 observed mass, exact mass 376.13 |
| Example 129 | |
| 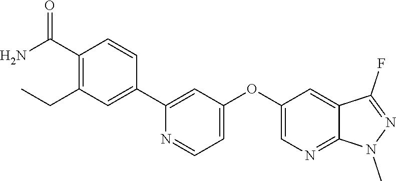 [SMILES: CCc1cc(-c2cc(Oc3cnc4c(c3)c(F)nn4C)ccn2)ccc1C(N)=O] | 2-ethyl-4-(4-((3-fluoro-1- methyl-1H-pyrazolo[3,4- b]pyridin-5-yl)oxy)pyridin- 2-yl)benzamide LCMS Method 3A: r.t. = 1.89 min, m/z (M + H+) = 392.0 observed mass, exact mass 391.14 |
| Example 130 | |
| 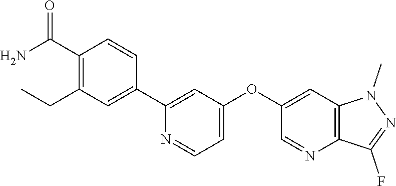 [SMILES: CCc1cc(-c2cc(Oc3cnc4c(F)nn(C)c4c3)ccn2)ccc1C(N)=O] | 2-ethyl-4-(4-((3-fluoro-1- methyl-1H-pyrazolo[4,3- b]pyridin-6-yl)oxy)pyridin- 2-yl)benzamide LCMS Method 3A: r.t. = 1.88 min, m/z (M + H+) = 392.3 observed mass, exact mass 391.14 |
| Example 131 | |
| 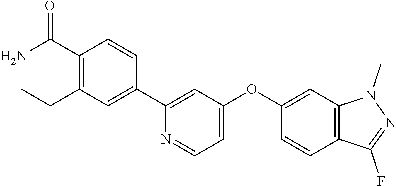 [SMILES: CCc1cc(-c2cc(Oc3ccc4c(F)nn(C)c4c3)ccn2)ccc1C(N)=O] | 2-ethyl-4-(4-((3-fluoro-1- methyl-1H-indazol-6- yl)oxy)pyridin-2- yl)benzamide LCMS Method 3A: r.t. = 2.08 min, m/z (M + H+) = 391.3 observed mass, exact mass 390.15 |
| Example 132 | |
| 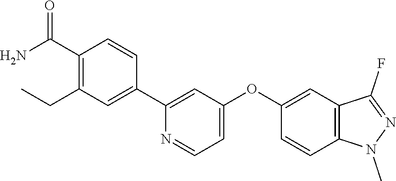 [SMILES: CCc1cc(-c2cc(Oc3ccc4c(c3)c(F)nn4C)ccn2)ccc1C(N)=O] | 2-ethyl-4-(4-((3-fluoro-1- methyl-1H-indazol-5- yl)oxy)pyridin-2- yl)benzamide LCMS Method 3A: r.t. = 2.03 min, m/z (M + H+) = 391.0 observed mass, exact mass 390.15 |
| Example 133 | |
| 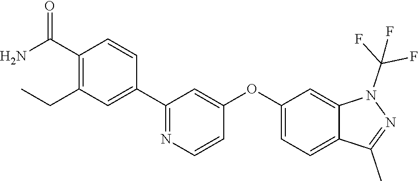 [SMILES: CCc1cc(-c2cc(Oc3ccc4c(C)nn(C(F)(F)F)c4c3)ccn2)ccc1C(N)=O] | 2-ethyl-4-(4-((3-methyl-1- (trifluoromethyl)-1H- indazol-6-yl)oxy)pyridin-2- yl)benzamide LCMS Method 3A: r.t. = 2.29 min, m/z (M + H+) = 441.0 observed mass, exact mass 440.15 |
| Example 134 | |
| 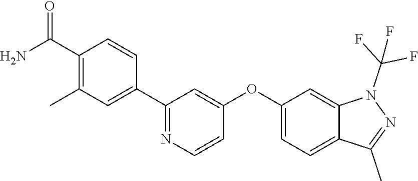 [SMILES: Cc1cc(-c2cc(Oc3ccc4c(C)nn(C(F)(F)F)c4c3)ccn2)ccc1C(N)=O] | 2-methyl-4-(4-((3-methyl-1- (trifluoromethyl)-1H- indazol-6-yl)oxy)pyridin-2- yl)benzamide LCMS Method 3A: r.t. = 2.21 min, m/z (M + H+) = 427.0 observed mass, exact mass 426.13 |
| Example 135 | |

### Example A

### GPR52 Activity

Compounds of Formula I were assessed for their ability to modulate GPR52 activity. HTRF CAMP assays were performed using a commercially available assay kit (CAMP Gs HiRange HTRF®, CisBio). Controls and compounds were solubilized in DMSO and 62.5 nanoliters of the diluted compounds were transferred into 384-well NBS assay plates via acoustic or precision low-volume dispensing. Compounds were further diluted to 1× with the addition of 20,000 cells per well. Flp-In™—CHO cells that stably express recombinant human GPR52 were used in the assay.

Cells were harvested with cell stripper and resuspended in stimulation buffer. After thirty minutes incubation at room temperature, detection reagents were added to each well. Plates were then incubated for an additional 30 minutes at room temperature. CAMP produced by the cells during the first incubation period competes with d2-labeled CAMP for binding to an anti-cAMP monoclonal antibody labeled with europium cryptate. The measured signal was inversely proportional to the concentration of cAMP produced by the cells and this signal was quantified using a PHERAstar® multi-mode plate reader.

Dose-response curves were generated from the HTRF counts that have been transformed based on a CAMP referenced curve, and then normalized to the positive control. EC₅₀ values were obtained using a nonlinear regression curve-fitting program. Table 13 provides average EC₅₀ values (n=1 to 15) where A is <25 nM; B is 25-100 nM; Cis 101-1000 nM; and D is >1000 nM and less than 2000 nM. The Emax (Table 13) is the maximum amount of cAMP (nM) produced by incubation of the cells with 10 μM or 31.6 μM of a test compound. The compound is defined by the top plateau of the sigmoid curve fit. The curve is then normalized to a control compound, in this case 4-(3-(3-fluoro-5-(trifluoromethyl)benzyl)-5-methyl-4,5-dihydro-1H-1,2,4-triazol-1-yl)-2-methylbenzamide (Tokumaru, K., et al., “Design, synthesis, and pharmacological evaluation of 4-azolyl-benzamide derivatives as novel GPR52 agonists”, Bioorganic & Medicinal Chemistry, Volume 25, Issue 12, June 2017, pages 3098-3115), and expressed as a percentage of the Emax of that control compound. For example, Example 1 has an average EC₅₀ of 270 nM (n=12) and an Emax of 106%, Example 4 has an average EC₅₀ of 83 nM (n=9) and an Emax of 106%, and Example 25 has an EC₅₀ of 83 nM (n=9) and an Emax of 103%.

TABLE 13

| Example | EC₅₀ (N=) | Eₘₐₓ (%) |
|---|---|---|
| 1 | C | 106 |
| 2 | C | 98 |
| 3 | C | 106 |
| 4 | B | 103 |
| 5 | C | 76 |
| 6 | C | 110 |
| 7 | D | 68 |
| 8 | D | 68 |
| 9 | D | 91 |
| 10 | C | 106 |
| 11 | C | 101 |
| 12 | C | 103 |
| 13 | C | 97 |
| 14 | C | 93 |
| 15 | D | 72 |
| 16 | C | 107 |
| 17 | C | 102 |
| 18 | C | 107 |
| 19 | C | 90 |
| 20 | C | 111 |
| 21 | C | 99 |
| 22 | C | 123 |
| 23 | C | 107 |
| 24 | C | 110 |
| 25 | B | 103 |
| 26 | B | 75 |
| 27 | C | 99 |
| 28 | C | 102 |
| 29 | C | 84 |
| 30 | D | 87 |
| 31 | C | 88 |
| 32 | C | 103 |
| 33 | C | 90 |
| 34 | D | 77 |
| 35 | C | 85 |
| 36 | A | 105 |
| 37 | C | 81 |
| 38 | B | 104 |
| 39 | B | 104 |
| 40 | B | 92 |
| 41 | C | 125 |
| 42 | C | 83 |
| 43 | C | 103 |
| 44 | C | 91 |
| 45 | C | 88 |
| 46 | C | 63 |
| 47 | C | 88 |
| 48 | C | 118 |
| 49 | C | 53 |
| 50 | C | 78 |
| 51 | D | 96 |
| 52 | D | 86 |
| 53 | B | 105 |
| 54 | C | 110 |
| 55 | B | 108 |
| 56 | B | 100 |
| 57 | B | 104 |
| 58 | B | 108 |
| 59 | C | 95 |
| 60 | C | 102 |
| 61 | C | 81 |
| 62 | C | 84 |
| 63 | C | 70 |
| 64 | C | 81 |
| 65 | D | 83 |
| 66 | C | 87 |
| 67 | C | 129 |
| 68 | A | 131 |
| 69 | A | 119 |
| 70 | C | 126 |
| 71 | B | 107 |
| 72 | B | 69 |
| 73 | C | 66 |
| 74 | C | 95 |
| 75 | C | 101 |
| 76 | C | 91 |
| 77 | C | 75 |
| 78 | C | 94 |
| 79 | C | 60 |
| 80 | C | 54 |
| 81 | C | 76 |
| 82 | C | 110 |
| 83 | C | 153 |
| 84 | C | 123 |
| 85 | C | 106 |
| 86 | C | 86 |
| 87 | C | 68 |
| 88 | C | 67 |
| 89 | C | 95 |
| 90 | D | 107 |
| 91 | | |
| 92 | | |
| 93 | C | 118 |
| 94 | C | 129 |
| 95 | C | 138 |
| 96 | C | 120 |
| 97 | C | 132 |
| 98 | C | 120 |
| 99 | C | 138 |
| 100 | C | 112 |
| 101 | C | 80 |
| 102 | C | 90 |
| 103 | C | 60 |
| 104 | C | 110 |
| 105 | C | 51 |
| 106 | C | 100 |
| 107 | B | 146 |
| 108 | C | 150 |
| 109 | C | 75 |
| 110 | C | 137 |
| 111 | C | 88 |
| 112 | C | 100 |
| 113 | C | 95 |
| 114 | B | 50 |
| 115 | C | 100 |
| 116 | B | 62 |
| 117 | C | 115 |
| 118 | C | 85 |
| 119 | C | 120 |
| 120 | C | 155 |
| 121 | D | 78 |
| 122 | C | 121 |
| 123 | C | 88 |
| 124 | D | 100 |
| 125 | C | 90 |
| 126 | C | 103 |
| 127 | C | 116 |
| 128 | B | 118 |
| 129 | C | 126 |
| 130 | C | 137 |
| 131 | C | 114 |
| 132 | B | 110 |
| 133 | C | 141 |
| 134 | C | 117 |
| 135 | C | 115 |

### Example B

### Selectivity Panel

The selectivity of compounds of Formula I were evaluated using the BioPrint® CEREP panel of 130+binding, enzyme, and uptake assays (Eurofins). Compounds of Formula I were tested at a single concentration (10 μM). Compound binding was calculated as a percentage inhibition of the binding of a radioactively labeled ligand specific for each target. Compound enzyme inhibition effect was calculated as a percentage inhibition of control enzyme activity.

Results showing an inhibition or stimulation higher than 50% were considered to represent significant effects of compounds of Formula I. Compounds of Formula I were highly selective demonstrating very clean profiles.

### Example C

### Induction of Compounds when Incubated with the PXR Nuclear Receptor

Induction of drug metabolizing enzymes or transporters via activation of the pregnane X receptor (PXR) was evaluated in vitro using a reporter gene assay. An expression vector harboring full-length human PXR plus the appropriate enhancers and promoters linked to the luciferase reporter gene were integrated into tumor cells. These transfected tumor cells were seeded onto a 96-well microtiter plate and placed in a tissue culture incubator. After 24 hours, cells were treated with either a single (10 μM) or 6 distinct concentrations of a compound of Formula I in duplicate wells, and returned to the incubator for an additional 24 hr. At the end of the incubation period, the number of viable cells/well are determined using Promega's Cell Titer Fluor cytotoxicity assay. Following the cytotoxicity assessment, Promega's ONE-Glo was added to the same wells and reporter gene activity assessed. Rifampicin was used as the positive control and was tested in the same manner as the test compounds; either a single concentration (10 mM) or six concentrations. Data reported were provided as the mean (n=2) of the fold receptor activation relative to vehicle-treated cells at 10 mM or each of the 6 doses. In addition, data was normalized to the number of viable cells/well and expressed as a percentage of the response given by Rifampicin at a 10 μM dose.

### Example D

### Stability of Compounds of Formula I in Mammalian Liver Microsomes

Compounds of Formula I were assessed for their stability in mammalian liver microsomes. A compound of Formula I (0.5 μM) was incubated with pooled mixed gender human liver microsomes (HLM) (0.5 mg/mL total protein) at 37° C. in the presence of an NADPH-generating system containing 50 mM, pH 7.4 potassium phosphate buffer, 3 mM magnesium chloride, 1 mM EDTA, 1 mM NADP, 5 mM glusose-6-phosphate, and 1 Unit/mL glucose-6-phosphate dehydrogenase. All concentrations were relative to the final incubation volume of 125 μL. Incubations were conducted at 37° C. for 0, 5, 10, 20, 40 and 60 minutes in a water bath and terminated by rapid mixing with 150 μL of ice-cold acetonitrile containing internal standard, and precipitated proteins were removed by centrifugation prior to LC-MS/MS analysis. Aliquots of the resulting supernatant fractions were analyzed by LC-MS/MS monitoring for depletion of parent compound. The resultant peak area ratio versus time data was fitted to a non-linear regression using XLfit Scientific Curve Fitting Software (IDBS Ltd., Surrey, UK) and half-life was calculated from the slope. Pharmacokinetic parameters were predicted using the method described by Obach et al (J. Pharmcol. Exp. Ther. 1997; 283:46.58). Briefly, values for intrinsic clearance were calculated from the in vitro half-life data and were then scaled to represent the clearance expected in the entire animal (human). Additional values calculated included predicted extraction ratio and predicted maximum bioavailability. For example, the O-linker has better metabolic stability relative to the methyl linker or the —CH₂O-linker, as shown by the following comparators

| Compound Structure | Microsomal Stability |
|---|---|
| 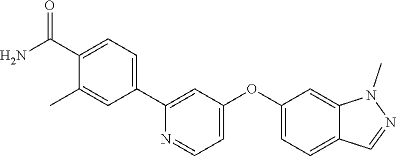 [SMILES: Cc1cc(-c2cc(Oc3ccc4cnn(C)c4c3)ccn2)ccc1C(N)=O] | Metabolic stability - predicted systemic clearance (example D, above) is 3.6 ml/min/kg |
| 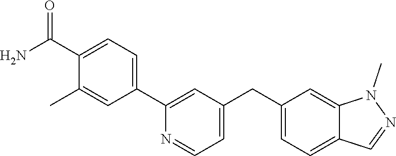 [SMILES: Cc1cc(-c2cc(Cc3ccc4cnn(C)c4c3)ccn2)ccc1C(N)=O] | Metabolic stability - predicted systemic clearance (example D, above) is 11.3 ml/min/kg |
| 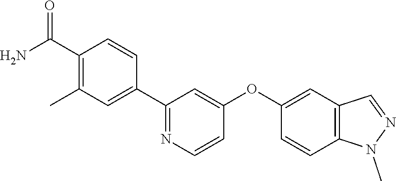 [SMILES: Cc1cc(-c2cc(Oc3ccc4c(cnn4C)c3)ccn2)ccc1C(N)=O] | Metabolic stability - predicted systemic clearance (example D, above) is 8.0 ml/min/kg |
| 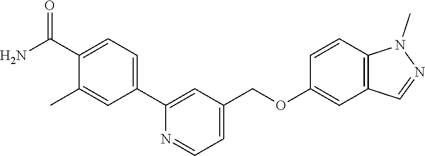 [SMILES: Cc1cc(-c2cc(COc3ccc4c(cnn4C)c3)ccn2)ccc1C(N)=O] | Metabolic stability - predicted systemic clearance (example D, above) is 12.3 ml/min/kg |

### Example E

### Permeability of Compounds of Formula I in MDR1-MDCK Cells

Compounds of Formula I were assessed to determine their permeability in MDR1-MDCK cells. Apparent permeability (Papp) and efflux ratios were generated using Madin-Darby canine kidney (MDCK) cells transfected with human multi-drug resistant protein 1 (MDR1). Cells were grown as monolayers on microporous membranes in 24-well assay plates. Each compound was evaluated at a single concentration equal to 5 μM. The assay buffer consisted of Hanks' balanced salt solution (Mediatech, Inc., Corning) pH 7.4 containing 10 mM HEPES and 15 mM glucose. Test articles were diluted in assay buffer then dosed to the apical chambers of cell monolayer plates to determine apical to basolateral (A to B) permeability. Basolateral to apical (B to A) permeability was determined by addition of dosing solution to the basolateral chambers. Cell monolayers dosed with test article were incubated for 1 hour at 37° C., 5% CO₂ in a humidified incubator. Samples were collected from both donor and receiver chambers at 1 hour then prepared for LC-MS/MS analysis using electrospray ionization. Triplicate measurements of A to B and B to A permeability were collected for each compound. Efflux ratio was calculated using the following formula: Pₐₚₚ B>A/Pₐₚₚ A>B. Controls were incorporated to ensure cell monolayer integrity (1 μM atenolol) and activity of MDR1 protein (digoxin).

### Example F

### Effects of Compounds of Formula I on the hERG Channel Current

Compounds of Formula I were assessed for their in vitro effects on the hERG channel current. The concentration-response relationship of all compounds on the hERG potassium channel current was evaluated at room temperature in stably transfected mammalian cells that express cloned hERG potassium channels, encoded by the KCNH2 gene. hERG potassium channels were expressed in Chinese Hamster's Ovary (CHO) cells that lack endogenous IKr.

Stock solutions of the positive control article were prepared in DMSO and stored at room temperature. Control 1, Dofetilide (Sigma; cat #PZ0016) has a molecular weight of 441.56. Control 2, Verapamil (Tocris; Cat #0654) has a molecular weight of 491.07. Both control 1 and 2 are stored at room temperature. CHO/hERG cell lines are from the Cricetulus griseus organism: tissue (ovary; transfected with ion channel cDNA); morphology (epithelial); age/stage (embryo); source strain (ATCC, Manassas, VA); and source substrain (Charles River Laboratories).

CHO cells were stably transfected with hERG cDNA. Stable transfectants were maintained in the culture medium with the appropriate selection pressure and antibiotics. All experiments were performed at room temperature. Each cell was treated as its own control. Full block was achieved with the addition of 20 μM Verapamil. Two groups were tested: test article treatment and positive control treatment.

Automated Patch Clamp Procedures. For all hERG testing, the 384-well based automated Patch Clamp System SyncroPatch 384PE (Nanion Technologies) with PatchControl software (data acquisition) and DataControl software (data analysis) was used. The recordings were performed at room temperature (22° C.) on planar NPC-384 multi-hole chips with 4 holes per well at a medium resistance. The recordings were executed in whole cell patch mode. The composition of the internal solution was: 10 mM EGTA, 10 mM HEPES, 10 mM KCl, 10 mM NaCl and 110 mM KF, pH 7.2, mOsm=285. The composition of the external solution was: 10 mM HEPES, 80 mM NaCl, 60 mm NMDG, 5 mM Glucose, 4 mM KCl, 5 mM CaCl₂ and 1 mM MgCl₂, pH 7.4, mOsm=298. Compounds of Formula I were dissolved in 100% DMSO. On the day of the experiment, a serial dilution in DMSO was prepared manually. The pre-diluted compounds of Formula I were further diluted into external solution with a dilution factor of 1:500 (0.2% DMSO by volume). Single application of compounds of Formula I was used with concentrations across the chip. Every well received once compound concentration followed by a full block of Verapamil to assess the leak current. Different concentrations of each compound to generate individual dose response relationships were spread across the chip.

Onset and block of hERG current was measured using a stimulus voltage pattern consisting of a 500 ms prepulse to −40 mV (leakage subtraction), a 2-second activating pulse to +40 mV followed by a 2-second test pulse to −40m V followed by a 2-second test pulse to −40 mV. The pulse pattern was repeated continuously at 6 s intervals from a holding potential of −80 mV. Peak tail current was calculated from the current amplitude evoked by the −40 mV prepulse and subtracted from the total membrane current record. A small hyperpolarizing voltage step from −80 to −90 mV was implemented during holding potential to calculate the resistance according to Ohm's law for quality control.

Data acquisition and analysis was performed using Nanion Data Control software. Steady state is defined by the limiting constant rate of change with time (linear time dependence). The steady state before and after test article application was used to calculate percentage of current inhibited at each concentration.

Compounds of Formula I are assessed for their ability to counteract the deficits in cognition and negative symptoms of schizophrenia. Animals exposed to repeated PCP dosing show deficits in cognition (measured in NOR) and sociability (measured with social interaction). These deficits are believed to map onto the cognition and negative symptoms of schizophrenia, respectively. For clarity, animals are dosed for 7 days, 2×/day with PCP and then after a washout period of at least 1 week are dosed once daily with a compound of Formula 1 for 6 days and once more before being tested in NOR (but do NOT have PCP onboard at this time), and then the following day are dosed with a compound of Formula I (but do NOT have PCP onboard at this time) and tested in Social Interaction. The experiments are set out in detail in the Examples, below.

### Example G

### Novel Object Recognition (NOR)

Compounds of Formula I were evaluated using the following sub-chronic phencyclidine (scPCP) protocol. The NOR test was performed as previously described in detail (Grayson, et al., “Atypical antipsychotics attenuate a sub-chronic PCP-induced cognitive deficit in the novel object recognition task in the rat”, Behavioral Brain Research, Vol.184, Issue 1, 2007; Snigdha et al., “Attenuation of Phencyclidine-Induced Object Recognition Deficits by the Combination of Atypical Antipsychotic Drugs and Pimavanserin (ACP 103), a 5-Hydroxytryptamineza Receptor Inverse Agonist”, Journal of Pharmacology and Experimental Therapeutics, February 2010, 332 (2)₆₂₂-631).

Prior to testing all animals were habituated to the empty test box. Habituation consists of placing all rats from one cage together in the empty test arena once for 20 min the day before the testing. Rats (scPCP and vehicle-treated) were given two 3-min trials separated by a 60-min interval in the home cage. In the first trial (acquisition), animals were placed in the test box and allowed to explore two identical objects (A1 and A2). In the second trial (retention), animals were placed in the test box with 1 duplicate familiar object from the acquisition phase (to avoid olfactory trails) and one novel object. A compound of Formula I or vehicle was administered once daily for six days prior to NOR and 120 min prior to acquisition. Behavior was filmed and scored by a trained experimenter who was blind to the treatment groups. Total object exploration time (defined as the duration of time animals spent licking, sniffing, or touching the object but not including time spent standing or sitting on or leaning against the object) was recorded for each of the familiar and novel objects in the acquisition and retention trials; locomotor activity (defined as movement, measured by the number of lines crossed in both trials) and discrimination index (defined as the difference in time spent exploring the novel and the familiar objects divided by total time spent exploring both objects) were also calculated.

All data were expressed as mean±s.e.m. (standard error of mean). Exploration times data from NOR in the acquisition and retention phases were analyzed separately via two-way analysis of variance (ANOVA) with factors of drug and exploration time of the 2 objects (2 identical objects in the acquisition phase and novel and familiar objects in the retention phase). Locomotor activity data (total number of line crossings) and the D1 were analyzed via one-way ANOVA. Time spent exploring the objects was analyzed by paired Student's t-test. Post-hoc analysis was conducted following a significant one-way ANOVA by Dunnett's t-test (for locomotor activity and DI).

### Example H

### Social Interaction

Compounds of Formula I are evaluated using the following social interaction test to assess an aspect of the avolition domain of negative symptoms in schizophrenia, social withdrawal using the sub-chronic administration of PCP.

One day after assessment in NOR, the rats were evaluated for social interaction using the same arena. Pairs of rats, weight matched (15-20 g) and unfamiliar to each other, receiving either no treatment (“conspecific” rats) or different treatments (PCP and Vehicle; “tested” rats or PCP+Compound of Formula I; “tested” rats) were placed in the test arena together for 10 min and behavior assessed as described below. A compound of Formula I or vehicle was administered for seven days prior to SI and 120 min prior to interaction evaluation.

An inanimate object such as an unopened drink can was also placed in the center of the arena to measure any differences in interaction of the test animal with an unfamiliar animal as opposed to an unfamiliar object. After each 10-minute trial, the object and arena were cleaned with 10% alcohol to remove traces of any olfactory cues. All testing was carried out under standard room illumination levels (70 cd/m²).

Immediately following the SI study, brains, and blood (n=12 per treatment group) was collected. Trunk blood was collected in Li-heparin coated tubes on ice prior to centrifugation. Blood was centrifuged for 10 min at 7,000 RPM at a temperature of 4° C. Plasma was then transferred to vials (˜400 μl) and immediately stored at −80° C. Whole brains were removed and immediately stored at −80° C. Frontal cortex was dissected and collected in vials and stored at −80° C.

Behavior was recorded on video for subsequent blind scoring. A behavioral scoring software program (Hindsight, Scientific programming services) was used to score the following (a) to (e) parameters:
(a) Investigative sniffing behavior: sniffing the conspecific's snout or parts of the body including the anogenital region;
(b) Following-rat moves after the conspecific, that is a vehicle treated rat of the same species, around the arena;
(c) Avoidances-actively turning away when approached by the conspecific animal;
(d) Investigation of object-exploration of object placed in center of the arena;
(e) Locomotor activity was recorded by counting the total number of sectors (that is, lines) crossed by the test rat.

All data are expressed as mean #s.e.m. Data were analyzed by an ANOVA followed by Dunnett's post-hoc test when appropriate. Statistical significance was assumed when P<0.05. All analysis was carried out in the SPSS statistical package (IBM).

The specification, including the examples, is intended to be exemplary only, and it will be apparent to those skilled in the art that various modifications and variations can be made in the present application without departing from the scope or spirit of the disclosure as defined by the appended claims. Each reference, including all patent, patent applications, and publications, cited in the present application is incorporated herein by reference in its entirety.

What is claimed is:

1. A compound of Formula I 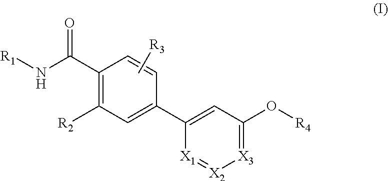
in which:
R₁ is selected from hydrogen and C₁₋₂alkyl;
R₂ is selected from C₁₋₂alkyl, halo, methyl-amino and halo-substituted-C₁₋₂alkyl;
R₃ is selected from hydrogen and halo;
R₄ is selected from 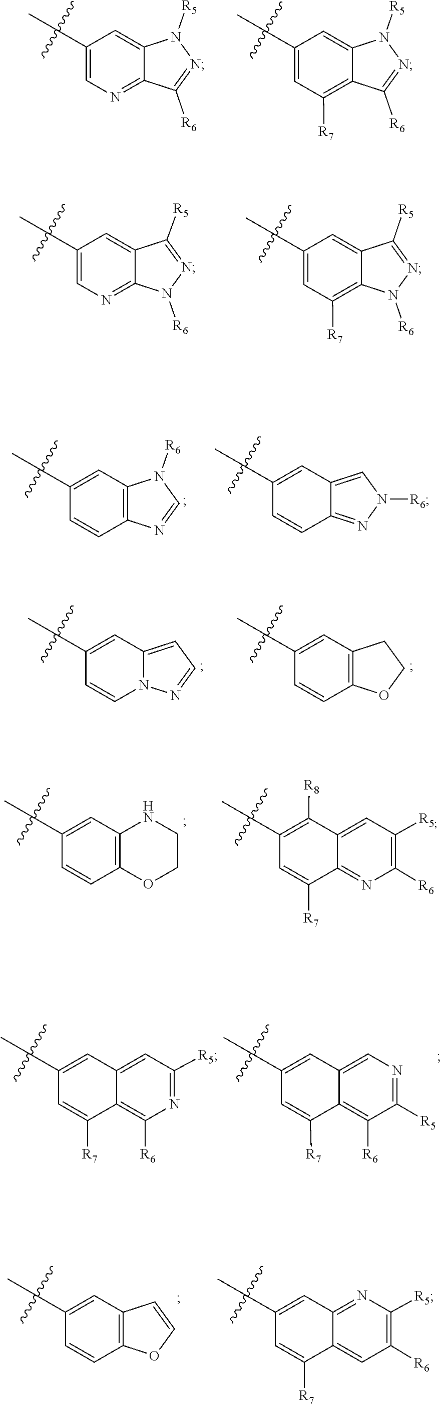 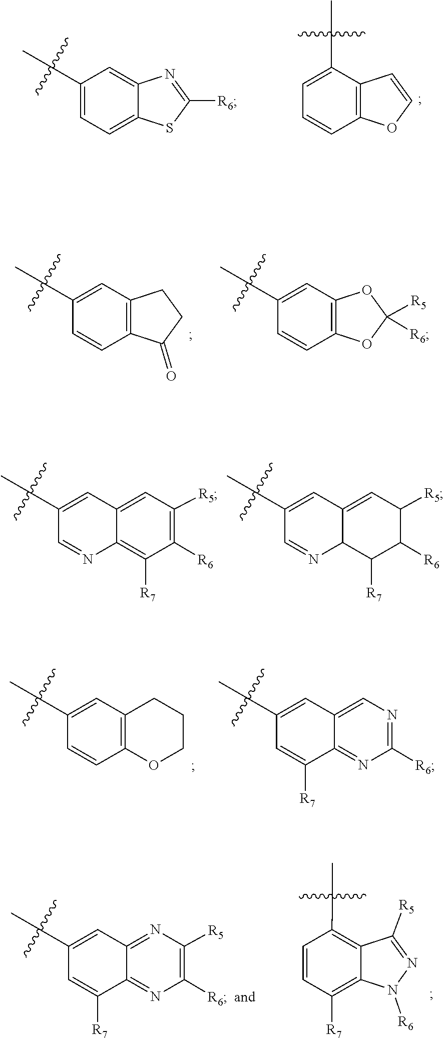
wherein:
R₅ when attached to a carbon atom is selected from hydrogen, C₁₋₂alkyl, halo and halo-substituted-C₁₋₂alkyl; and R₅ when attached to a nitrogen atom is selected from hydrogen, C₁₋₂alkyl, and halo-substituted-C₁₋₂alkyl;
R₆ when attached to a carbon atom is selected from hydrogen, amino, cyano, C₁₋₂alkyl, halo and halo-substituted-C₁₋₂alkyl; and R₆ when attached to a nitrogen atom is selected from hydrogen, C₁₋₂alkyl, and halo-substituted-C₁₋₂alkyl;
R₇ is selected from hydrogen, C₁₋₂alkyl, halo and halo-substituted-C₁₋₂alkyl;
R₈ is selected from hydrogen and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; or R₉ and the nitrogen of X₁ form a 5-member unsaturated ring containing up to two nitrogen atoms;
X₃ is selected from CR₉a, wherein R₉ₐ is selected from hydrogen and methyl; and
the pharmaceutically acceptable salts thereof.

2. The compound of claim 1 of Formula Ia 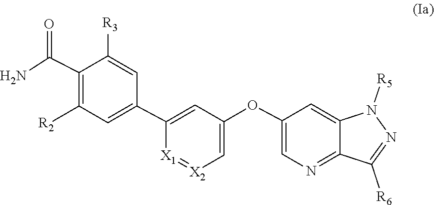
in which:
R₂ is selected from C₁₋₂alkyl, halo, methyl-amino and halo-substituted-C₁₋₂alkyl;
R₃ is selected from hydrogen and halo;
R₅ is selected from hydrogen and C₁₋₂alkyl;
R₆ is selected from hydrogen, amino, cyano, C₁₋₂alkyl, halo and halo-substituted-C₁₋₂alkyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

3. The compound of claim 2 wherein:
R₂ is selected from methyl, ethyl, methyl-amino, chloro and trifluoro-methyl;
R₃ is selected from hydrogen and halo;
R₅ is selected from hydrogen, methyl and ethyl;
R₆ is selected from hydrogen, methyl, fluoro, amino, cyano, and trifluoromethyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

4. The compound of claim 3 wherein:
R₂ is selected from methyl, ethyl and trifluoromethyl;
R₃ is selected from hydrogen and fluoro;
X₁ is selected from N and CH;
X₂ is CR₉; wherein R₉ is selected from hydrogen and amino; and the pharmaceutically acceptable salts thereof.

5. The compound of claim 4, or a pharmaceutically acceptable salt thereof, selected from

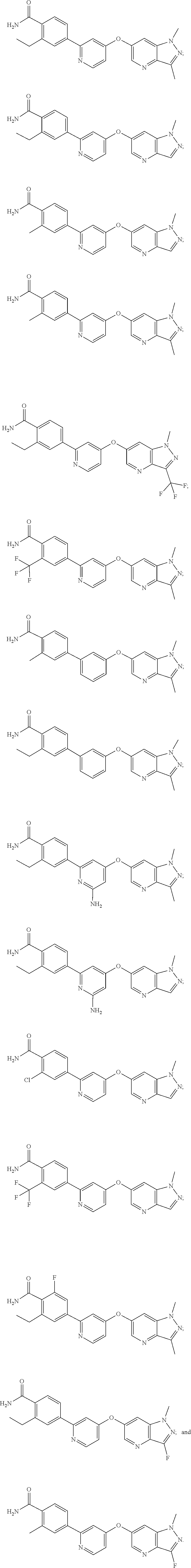

6. The compound of claim 1 of Formula Ib 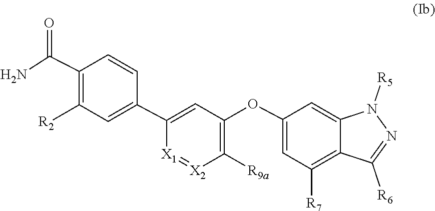
in which:
R₂ is selected from C₁₋₂alkyl, halo, methyl-amino and halo-substituted-C₁₋₂alkyl;
R₅ is selected from hydrogen, C₁₋₂alkyl and halo-substituted-C₁₋₂alkyl;
R₆ is selected from hydrogen, amino, cyano, halo, C₁-2alkyl and halo-substituted-C₁₋₂alkyl;
R₇ is selected from hydrogen, C₁₋₂alkyl and halo;
R₉ₐ is selected from hydrogen and methyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

7. The compound of claim 6 wherein:
R₂ is selected from methyl, ethyl, methyl-amino, chloro and trifluoro-methyl;
R₅ is selected from hydrogen, methyl, ethyl and trifluoromethyl;
R₆ is selected from hydrogen, methyl, amino, cyano, fluoro and trifluoromethyl;
R₇ is selected from hydrogen, methyl and fluoro;
R₉ₐ is selected from hydrogen and methyl;
X₁ is selected from N and CH;
X₂ is selected from N and CR₈; wherein R₅ is selected from hydrogen, amino, amino-methyl, methyl-amino-methyl, azetidin-2-yl and azetidin-3-yl; and the pharmaceutically acceptable salts thereof.

8. The compound of claim 7 wherein X₂ is CR₈; wherein R₈ is selected from hydrogen, amino, amino-methyl, methyl-amino-methyl, azetidin-2-yl and azetidin-3-yl; and the pharmaceutically acceptable salts thereof.

9. The compound of claim 8, or a pharmaceutically acceptable salt thereof, selected from

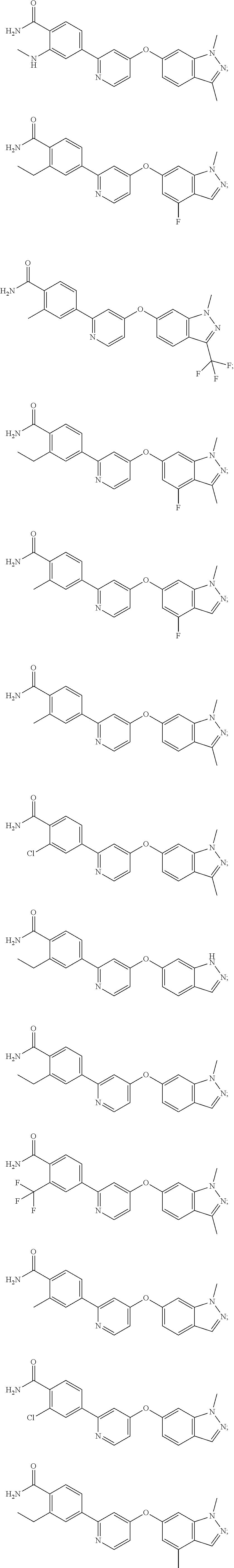

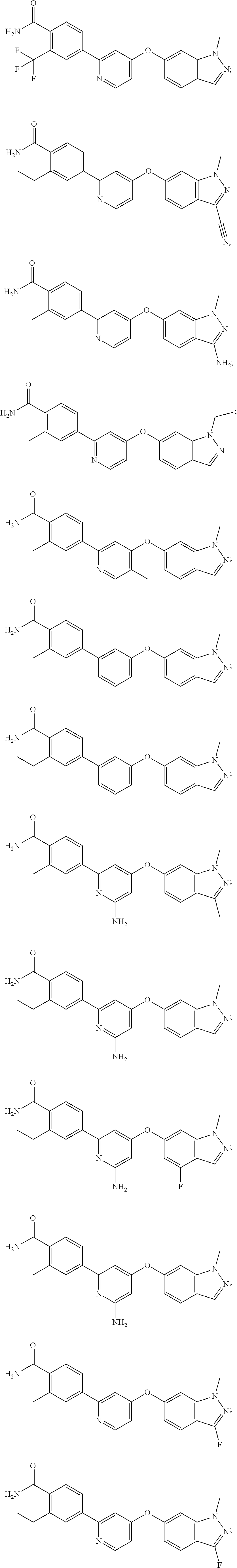

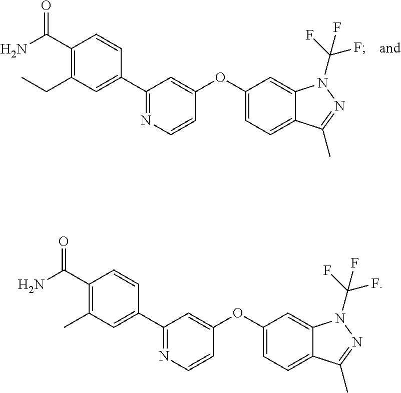 [SMILES: CCc1cc(-c2cc(Oc3ccc4c(C)nn(C(F)(F)F)c4c3)ccn2)ccc1C(N)=O ; Cc1cc(-c2cc(Oc3ccc4c(C)nn(C(F)(F)F)c4c3)ccn2)ccc1C(N)=O]

10. The compound of claim 1 of Formula Ic: 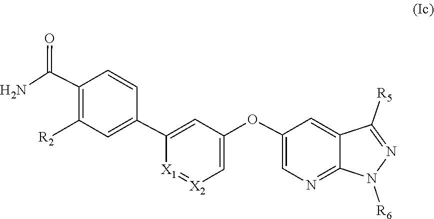
in which:
R₂ is selected from C₁₋₂alkyl, halo, methyl-amino and halo-substituted-C₁₋₂alkyl;
R₅ is selected from hydrogen, C₁₋₂alkyl and halo-substituted-C₁₋₂alkyl;
R₆ is selected from hydrogen and C₁₋₂alkyl;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

11. The compound of claim 10 wherein:
R₂ is selected from methyl, ethyl, methyl-amino, chloro and trifluoro-methyl;
R₅ is selected from hydrogen, fluoro, methyl, ethyl and trifluoromethyl;
R₆ is selected from hydrogen, methyl, amino, cyano, and trifluoromethyl;
X₁ is selected from N and CH;
X₂ is selected from N and CR₈; wherein R₅ is selected from hydrogen, amino, amino-methyl, methyl-amino-methyl, azetidin-2-yl and azetidin-3-yl; and the pharmaceutically acceptable salts thereof.

12. The compound of claim 11 wherein:
R₂ is selected from methyl and ethyl;
X₁ is N;
X₂ is CR₉; wherein R₉ is selected from hydrogen and amino; and the pharmaceutically acceptable salts thereof.

13. The compound of claim 12, or a pharmaceutically acceptable salt thereof, selected from

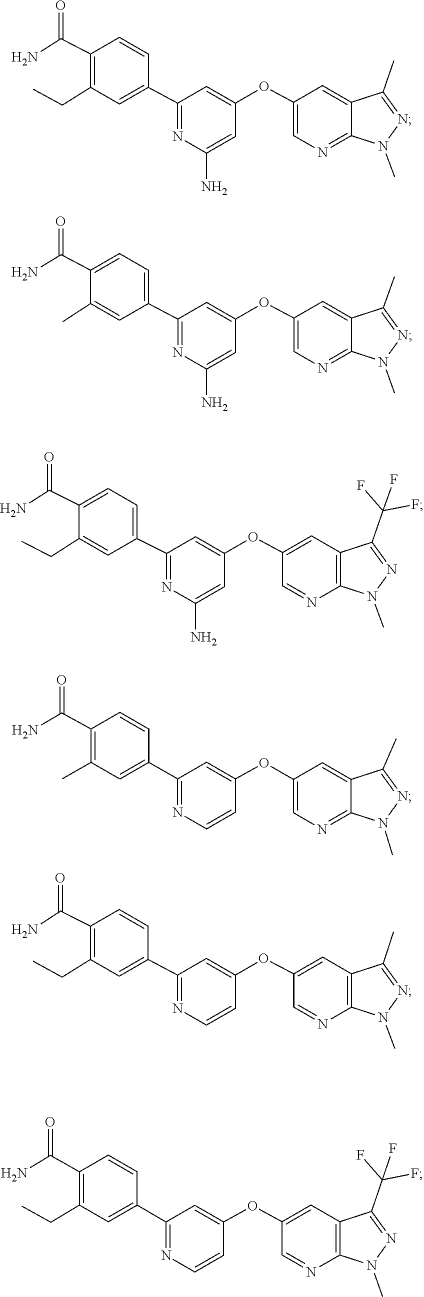 [SMILES: CCc1cc(-c2cc(Oc3cnc4c(c3)c(C)nn4C)cc(N)n2)ccc1C(N)=O ; Cc1cc(-c2cc(Oc3cnc4c(c3)c(C)nn4C)cc(N)n2)ccc1C(N)=O ; CCc1cc(-c2cc(Oc3cnc4c(c3)c(C(F)(F)F)nn4C)cc(N)n2)ccc1C(N)=O ; Cc1cc(-c2cc(Oc3cnc4c(c3)c(C)nn4C)ccn2)ccc1C(N)=O ; CCc1cc(-c2cc(Oc3cnc4c(c3)c(C)nn4C)ccn2)ccc1C(N)=O ; CCc1cc(-c2cc(Oc3cnc4c(c3)c(C(F)(F)F)nn4C)ccn2)ccc1C(N)=O]

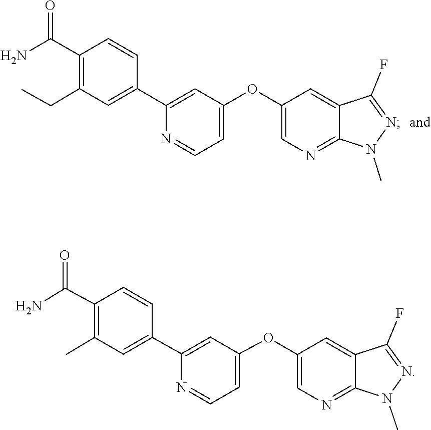 [SMILES: CCc1cc(-c2cc(Oc3cnc4c(c3)c(F)nn4C)ccn2)ccc1C(N)=O ; Cc1cc(-c2cc(Oc3cnc4c(c3)c(F)nn4C)ccn2)ccc1C(N)=O]

14. The compound of claim 1 of Formula Id 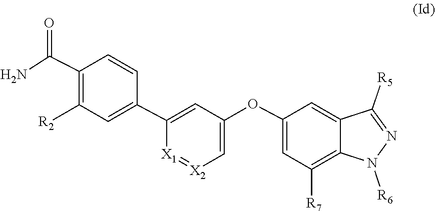
in which:
R₂ is selected from C₁₋₂alkyl, halo, methyl-amino and halo-substituted-C₁₋₂alkyl;
R₅ is selected from hydrogen, C₁₋₂alkyl, halo and halo-substituted-C₁₋₂alkyl;
R₆ is selected from hydrogen and C₁₋₂alkyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl;
and the pharmaceutically acceptable salts thereof.

15. The compound of claim 14 wherein:
R₂ is selected from methyl, ethyl, methyl-amino, chloro and trifluoro-methyl;
R₅ is selected from hydrogen, fluoro, methyl and ethyl;
R₆ is selected from hydrogen, methyl, amino, cyano, and trifluoromethyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; and the pharmaceutically acceptable salts thereof.

16. The compound of claim 15 wherein: R₂ is selected from methyl, ethyl and chloro; X₁ is N; X₂ is CH; and the pharmaceutically acceptable salts thereof.

17. The compound of claim 16, or a pharmaceutically acceptable salt thereof, selected from

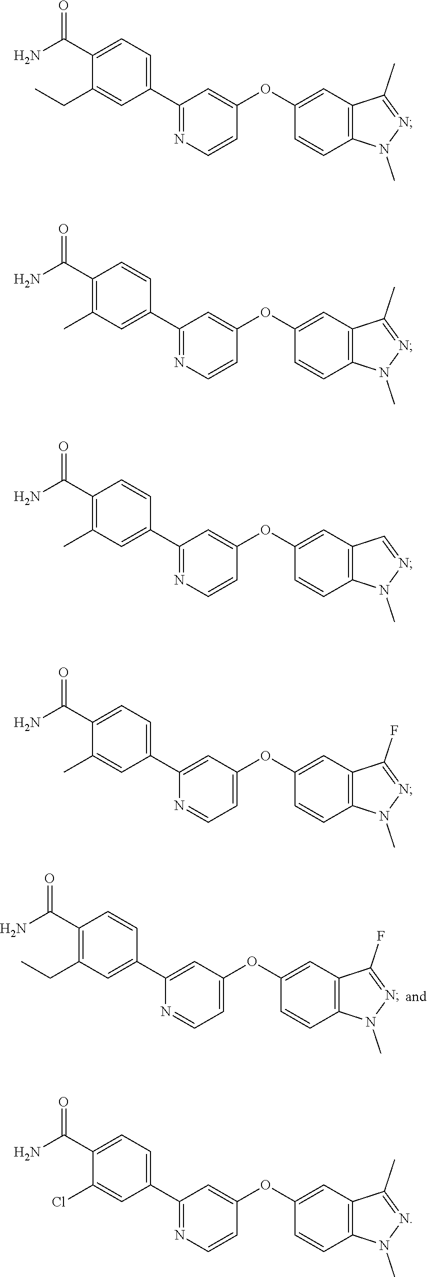 [SMILES: CCc1cc(-c2cc(Oc3ccc4c(c3)c(C)nn4C)ccn2)ccc1C(N)=O ; Cc1cc(-c2cc(Oc3ccc4c(c3)c(C)nn4C)ccn2)ccc1C(N)=O ; Cc1cc(-c2cc(Oc3ccc4c(cnn4C)c3)ccn2)ccc1C(N)=O ; Cc1cc(-c2cc(Oc3ccc4c(c3)c(F)nn4C)ccn2)ccc1C(N)=O ; CCc1cc(-c2cc(Oc3ccc4c(c3)c(F)nn4C)ccn2)ccc1C(N)=O ; Cc1nn(C)c2ccc(Oc3ccnc(-c4ccc(C(N)=O)c(Cl)c4)c3)cc12]

18. The compound of claim 1 of Formula Ie 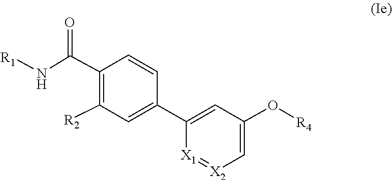
in which:
R₁ is selected from hydrogen and C₁₋₂alkyl;
R₂ is selected from C₁₋₂alkyl, halo, methyl-amino and halo-substituted-C₁₋₂alkyl;
R₄ is selected from 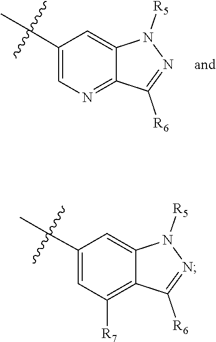
R₅ is selected from hydrogen and C₁₋₂alkyl;
R₆ is selected from hydrogen, amino, cyano, C₁₋₂alkyl and halo-substituted-C₁₋₂alkyl;
R₇ is selected from hydrogen, C₁₋₂alkyl and halo;
X₁ is selected from N and CH;
X₂ is selected from N and CR₉; wherein R₉ is selected from hydrogen, amino, amino-methyl, methyl-amino-methyl, azetidin-2-yl and azetidin-3-yl; or R₉ and the nitrogen of X₁ form a 5-member unsaturated ring containing up to two nitrogen atoms; and the pharmaceutically acceptable salts thereof.

19. The compound of claim 18 wherein:
R₁ is selected from hydrogen, methyl and ethyl;
R₂ is selected from methyl and ethyl;
R₄ is selected from 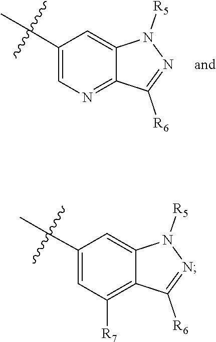
R₅ is methyl;
R₆ when attached to a carbon atom is selected from hydrogen, methyl and trifluoromethyl;
R₇ is hydrogen;
X₁ is selected from N and CH;
X₂ is CR₉; wherein R₉ is hydrogen; and the pharmaceutically acceptable salts thereof.

20. The compound of claim 19, or a pharmaceutically acceptable salt thereof, selected from

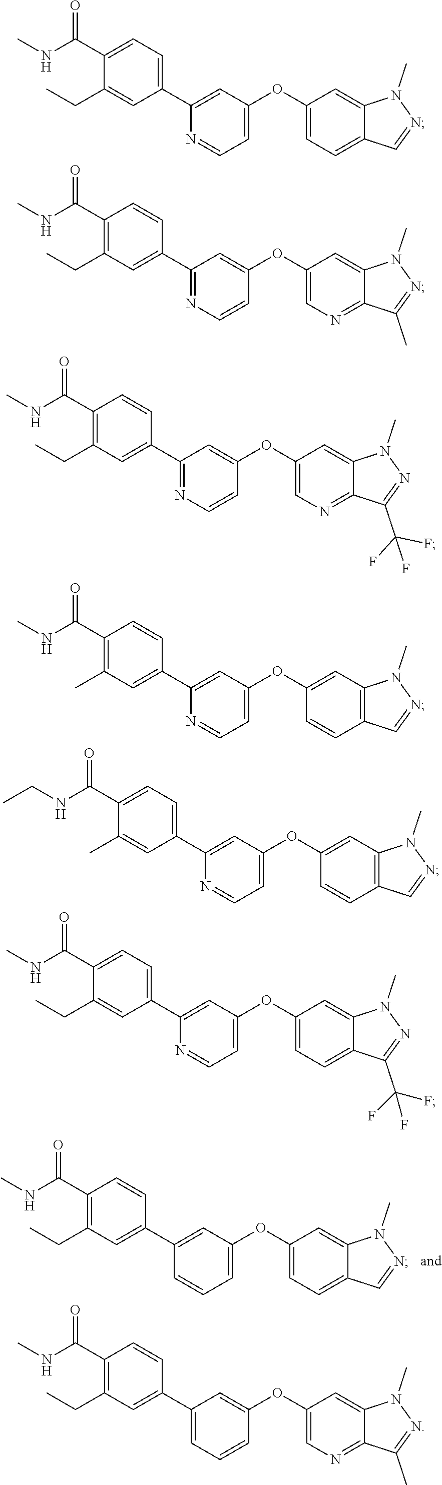

21. The compound of claim 1 of Formula If 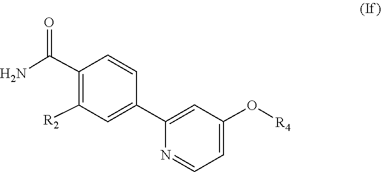
in which:
R₂ is selected from C₁₋₂alkyl, halo, methyl-amino and halo-substituted-C₁₋₂alkyl;
R₄ is selected from: 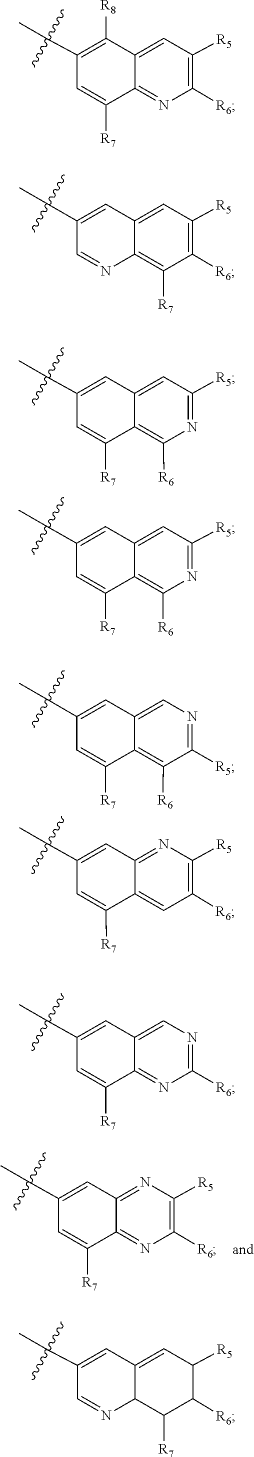
R₅ is selected from hydrogen, C₁₋₂alkyl, halo and halo-substituted-C₁₋₂alkyl;
R₆ is selected from hydrogen, amino, cyano, C₁₋₂alkyl, halo and halo-substituted-C₁₋₂alkyl;
R₇ is selected from hydrogen, C₁₋₂alkyl, halo and halo-substituted-C₁₋₂alkyl;
R₈ is selected from hydrogen and halo;
and the pharmaceutically acceptable salts thereof.

22. The compound of claim 21 wherein: R₂ is selected from methyl, ethyl, chloro, fluoro and trifluoromethyl; R₄ is selected from: 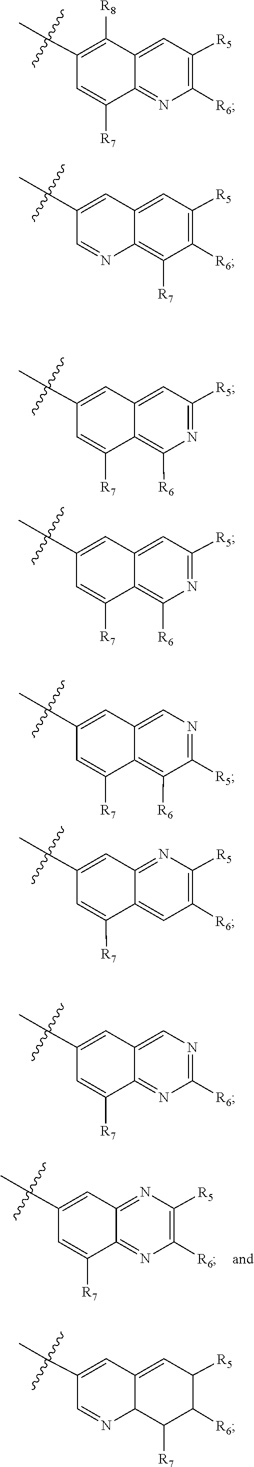
R₅ when attached to a carbon atom is selected from hydrogen, fluoro, chloro, and methyl;
R₆ is selected from hydrogen and fluoro;
R₇ is selected from hydrogen, fluoro, chloro and trifluoromethyl;
R₈ is selected from hydrogen and fluoro;
and the pharmaceutically acceptable salts thereof.

23. The compound of claim 22, or a pharmaceutically acceptable salt thereof, selected from

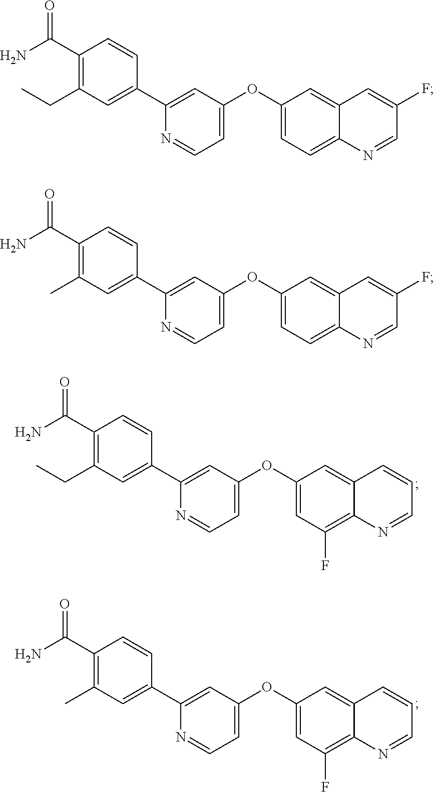 [SMILES: CCc1cc(-c2cc(Oc3ccc4ncc(F)cc4c3)ccn2)ccc1C(N)=O ; Cc1cc(-c2cc(Oc3ccc4ncc(F)cc4c3)ccn2)ccc1C(N)=O ; CCc1cc(-c2cc(Oc3cc(F)c4ncccc4c3)ccn2)ccc1C(N)=O ; Cc1cc(-c2cc(Oc3cc(F)c4ncccc4c3)ccn2)ccc1C(N)=O]

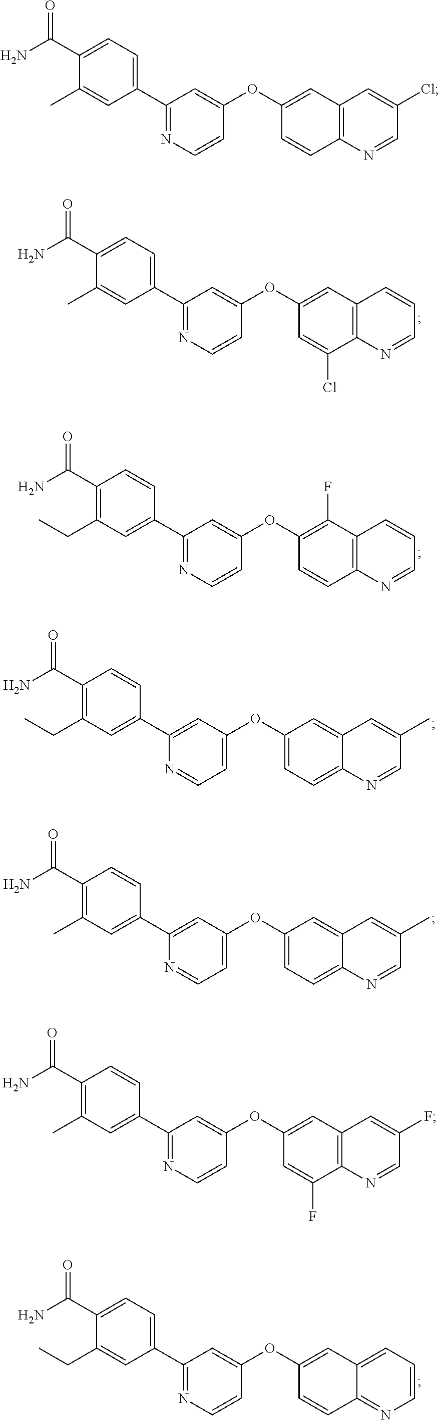

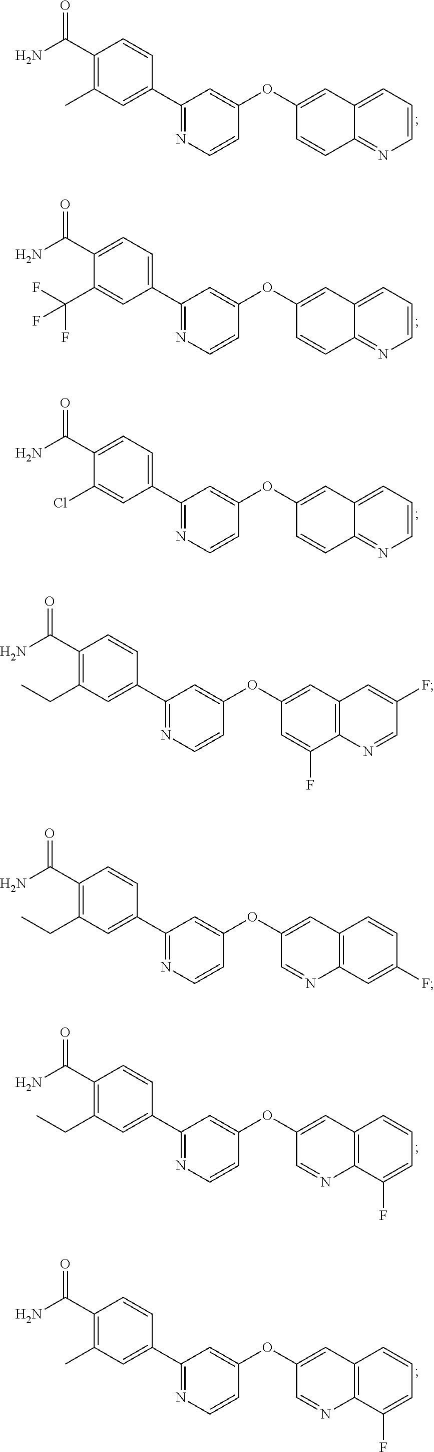

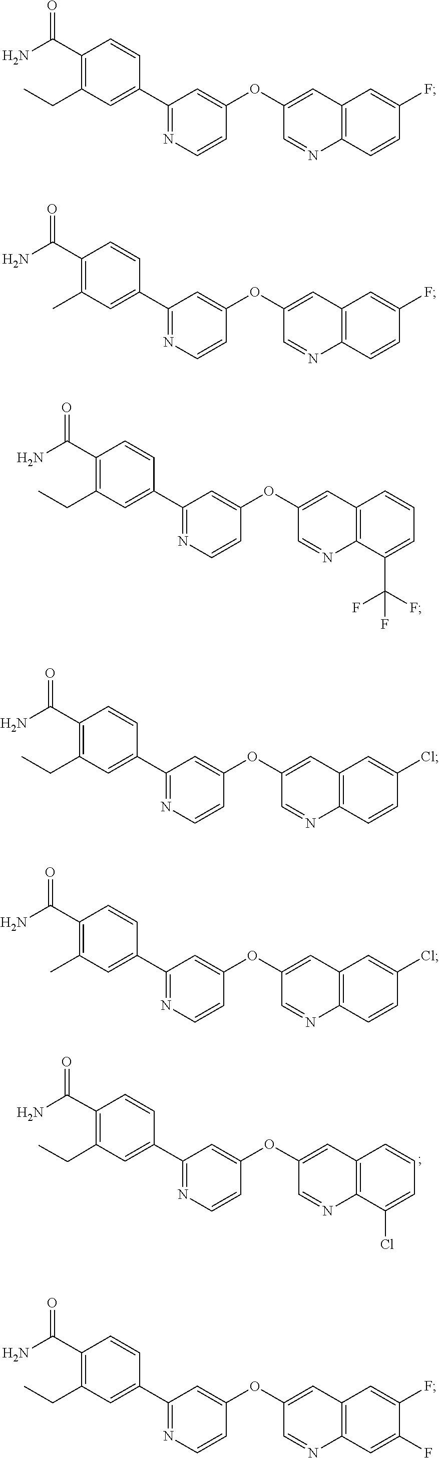

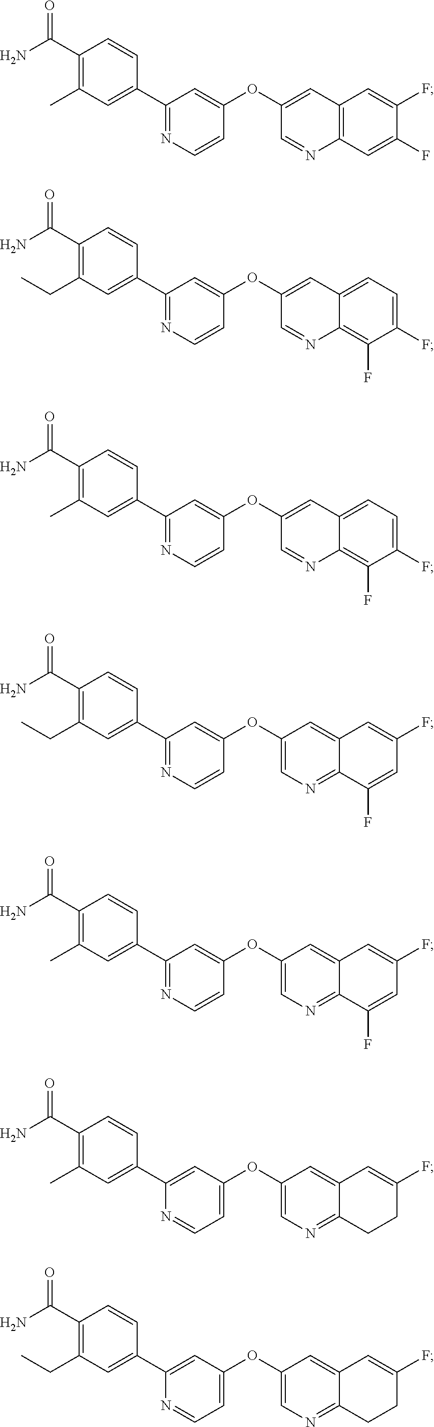

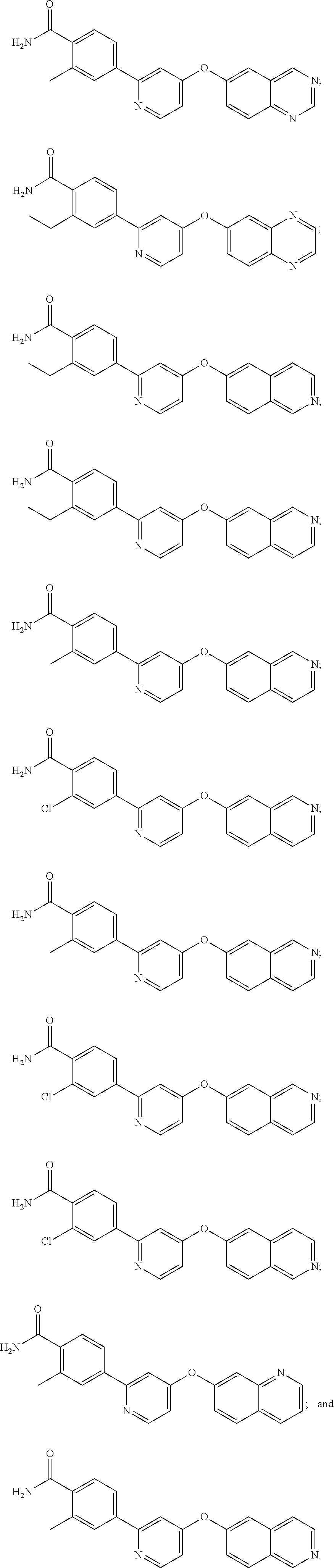

24. The compound of claim 1, or a pharmaceutically acceptable salt thereof, selected from

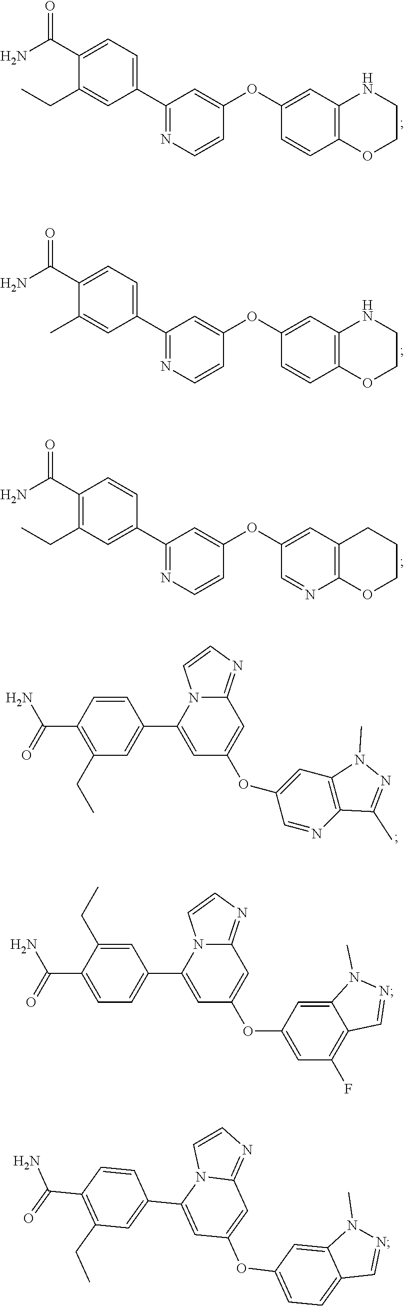 [SMILES: CCc1cc(-c2cc(Oc3ccc4c(c3)NCCO4)ccn2)ccc1C(N)=O ; Cc1cc(-c2cc(Oc3ccc4c(c3)NCCO4)ccn2)ccc1C(N)=O ; CCc1cc(-c2cc(Oc3cnc4c(c3)CCCO4)ccn2)ccc1C(N)=O ; CCc1cc(-c2cc(Oc3cnc4c(C)nn(C)c4c3)cc3nccn23)ccc1C(N)=O ; CCc1cc(-c2cc(Oc3cc(F)c4cnn(C)c4c3)cc3nccn23)ccc1C(N)=O ; CCc1cc(-c2cc(Oc3ccc4cnn(C)c4c3)cc3nccn23)ccc1C(N)=O]

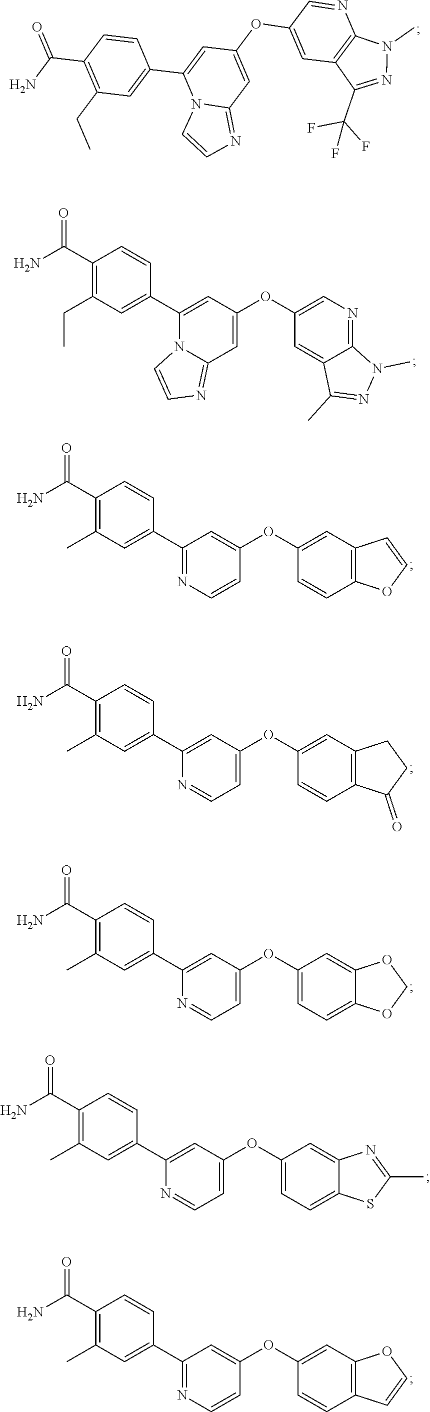

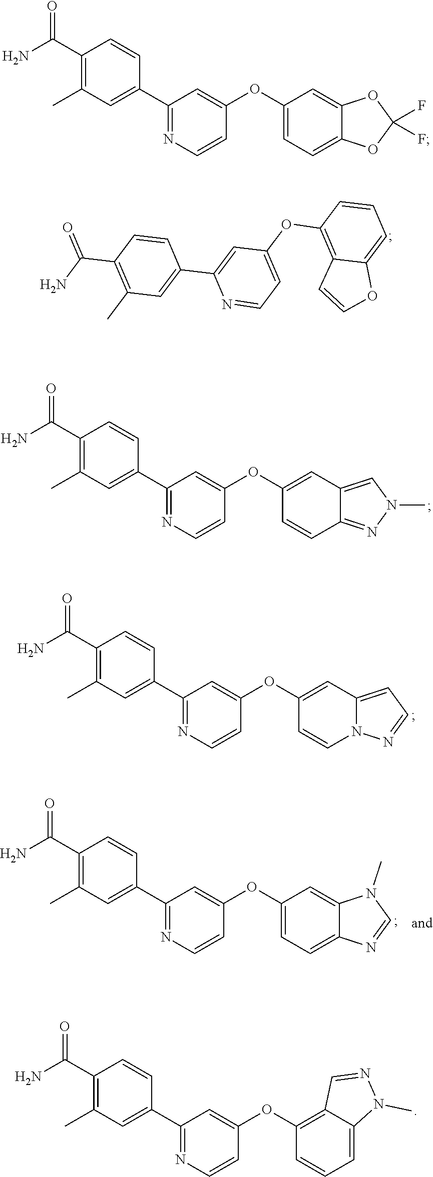 [SMILES: Cc1cc(-c2cc(Oc3ccc4c(c3)OC(F)(F)O4)ccn2)ccc1C(N)=O ; Cc1cc(-c2cc(Oc3cccc4occc34)ccn2)ccc1C(N)=O ; Cc1cc(-c2cc(Oc3ccc4nn(C)cc4c3)ccn2)ccc1C(N)=O ; Cc1cc(-c2cc(Oc3ccn4nccc4c3)ccn2)ccc1C(N)=O ; Cc1cc(-c2cc(Oc3ccc4ncn(C)c4c3)ccn2)ccc1C(N)=O ; Cc1cc(-c2cc(Oc3cccc4c3cnn4C)ccn2)ccc1C(N)=O]

25. A pharmaceutical composition comprising a compound of any one of claims 1 to 24, or a pharmaceutically acceptable salt thereof, and one or more excipients.

26. A method of treating a neurological disorder, comprising administering to a subject in need thereof an effective amount of at least one compound of any one of claims 1 to 24, or a pharmaceutically acceptable salt thereof, or the pharmaceutical composition of claim 25; wherein the neurological disorder is selected from the group consisting of schizophrenia, negative symptoms associated with schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder, an autism spectrum disorder, a prolactin-related disorder, hyperprolactinemia, a neurocognitive disorder, a trauma- or stressor-related disorder, post-traumatic stress disorder, a disruptive impulse-control, a disruptive conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia, Parkinson's Disease, drug induced Parkinsonism, dyskinesias, dystonia, chorea, levodopa induced dyskinesia, cerebral palsy and progressive supranuclear palsy, Huntington's disease and chorea associated with Huntington's disease.

27. The method of claim 26 wherein the neurological disorder is selected from schizophrenia, cognitive impairment associated with schizophrenia (CIAS), and vascular cognitive impairment.

28. The method of claim 27 wherein schizophrenia is selected from the negative symptoms associated with schizophrenia, the psychotic symptoms of schizophrenia, schizoaffective disorder, schizotypal disorder, schizophreniform disorder, treatment resistant schizophrenia, and attenuated psychosis syndrome.

29. A method of ameliorating one or more symptoms of a neurological disorder, comprising administering to a subject in need thereof an effective amount of at least one compound of any one of claims 1 to 24, or a pharmaceutically acceptable salt thereof, or the pharmaceutical composition of claim 25; wherein the neurological disorder is selected from the group consisting of schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder, an autism spectrum disorder, a prolactin-related disorder, hyperprolactinemia, a neurocognitive disorder, a trauma- or stressor-related disorder, post-traumatic stress disorder, a disruptive impulse-control, a disruptive conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia; Parkinson's Disease, drug induced Parkinsonism, dyskinesias, dystonia, chorea, levodopa induced dyskinesia, cerebral palsy and progressive supranuclear palsy, and Huntington's disease, and chorea associated with Huntington's disease.

30. A compound of any one of claims 1 to 24, or a pharmaceutically acceptable salt thereof, or the pharmaceutical product of claim 25 for use in treating a neurological disorder, wherein the neurological disorder is selected from the group consisting of schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder, an autism spectrum disorder, a prolactin-related disorder, hyperprolactinemia, a neurocognitive disorder, a trauma- or stressor-related disorder, post-traumatic stress disorder, a disruptive impulse-control, a disruptive conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia, Parkinson's Disease, drug induced Parkinsonism, dyskinesias, dystonia, chorea, levodopa induced dyskinesia, cerebral palsy and progressive supranuclear palsy, and Huntington's disease, and chorea associated with Huntington's disease.

31. A method of manufacturing a medicament of at least one compound of any one of claims 1 to 24, or a pharmaceutically acceptable salt thereof, or the pharmaceutical product of claim 25 for treating a neurological disorder, wherein the neurological disorder is selected from the group consisting of schizophrenia, cognitive impairment, a panic disorder, a phobic disorder, drug-induced psychotic disorder, delusional psychosis, neuroleptic-induced dyskinesia, Parkinson's disease, drug-induced Parkinson's syndrome, extrapyramidal syndrome, Alzheimer's Disease, Lewy Body Dementia, bipolar disorder, ADHD, Tourette's syndrome, an extrapyramidal or movement disorder, a motor disorder, a hyperkinetic movement disorder, a psychotic disorder, catatonia, a mood disorder, a depressive disorder, an anxiety disorder, obsessive-compulsive disorder, an autism spectrum disorder, a prolactin-related disorder, hyperprolactinemia, a neurocognitive disorder, a trauma- or stressor-related disorder, post-traumatic stress disorder, a disruptive impulse-control, a disruptive conduct disorder, a sleep-wake disorder, a substance-related disorder, an addictive disorder, a behavioral disorder, hypofrontality, an abnormality in the tuberoinfundibular, mesolimbic, mesocortical, or nigrostriatal pathway, decreased activity in the striatum, cortical dysfunction, neurocognitive dysfunction and the cognitive deficits associated with schizophrenia, Parkinson's Disease, drug induced Parkinsonism, dyskinesias, dystonia, chorea, levodopa induced dyskinesia, cerebral palsy and progressive supranuclear palsy, and Huntington's disease, and chorea associated with Huntington's disease.

32. A compound of any one of claims 1 to 24, or a pharmaceutically acceptable salt thereof, or the pharmaceutical product of claim 25, for use as a medicament.
